(19) United States
(12) Patent Application Publication
(10) Pub. No.: US 2021/0060185 A1
(43) Pub. Date: Mar. 4, 2021

(54) PROTEASE SENSITIVE GVPC AND RELATED GAS VESICLE GENE CLUSTERS, EXPRESSION SYSTEMS, CONSTRUCTS, VECTORS, GENETIC CIRCUITS, CELLS, COMPOSITIONS, METHODS AND SYSTEMS FOR CONTRAST-ENHANCED IMAGING

(71) Applicant: California Institute of Technology, Pasadena, CA (US)

(72) Inventors: Anupama LAKSHMANAN, Pasadena, CA (US); Mikhail SHAPIRO, Pasadena, CA (US); Suchita P. NETY, Cambridge, MA (US); Zhiyang JIN, Pasadena, CA (US)

(21) Appl. No.: 17/006,591

(22) Filed: Aug. 28, 2020

Related U.S. Application Data
(60) Provisional application No. 62/892,672, filed on Aug. 28, 2019.

(51) Int. Cl. A61K 49/22; C07K 14/195; C12Q 1/37

(52) U.S. Cl. CPC A61K 49/223; C07K 2319/50; C12Q 1/37; C07K 14/195

(57) ABSTRACT

Provided herein are engineered protease sensitive gas vesicles and related engineered protease genetically GvpC constructs, vectors, gas vesicles gene clusters, genetic circuits, cells, compositions, methods and systems, which in several embodiments can be used together with contrast-enhanced imaging technique, to detect and report protease activity and related biological events in an imaging target site.

### CROSS REFERENCE TO RELATED APPLICATIONS

The present application claims priority to U.S. Provisional Application No. 62/892,672, entitled “Acoustic Biosensors” filed on Aug. 28, 2019, with docket number CIT 8336, which is incorporated herein by reference in its entirety.

### STATEMENT OF GOVERNMENT GRANT

This invention was made with government support under Grant No. EB018975 awarded by the National Institutes of Health and under Grant No. W911NF-14-1-0111 awarded by the Army. The government has certain rights in the invention.

### FIELD

The present disclosure relates to engineered proteins of gas-filled structures, and in particular to a gas vesicle protein GvpC genetically engineered to be protease sensitive. More particularly the present disclosure relates to a protease sensitive GvpC and related gas vesicles, expression systems, constructs, vectors, gene clusters, genetic circuits, cells, compositions, methods and systems to produce gas filled structures and/or to image protease associated biological events in a target site.

### BACKGROUND

Proteases are enzymes involved in a several biological events in various organisms from blood-clotting to apoptosis pathways.

Reporting of protease associated events, and biological events in general, is however currently primarily based on fluorescent reporter genes.

Accordingly, reporting protease associated events in deep tissues, at nanomolar concentrations and/or producing dynamic contrast in response to local molecular signals remains challenging.

### SUMMARY

Provided herein is a protease sensitive gas vesicle protein GVPC genetically engineered to be protease sensitive and related protease sensitive gas vesicles (GVs), constructs, vectors, gas vesicle gene clusters, genetic circuits, cells, compositions, methods and systems, which in several embodiments can be used together with contrast-enhanced imaging technique, to detect and report protease activity and related biological events in an imaging target site.

According to a first aspect, a method to provide a protease sensitive Gas Vesicle is described, as well as a protease sensitive Gas Vesicles obtained thereby and related protease sensitive GvpC protein. The method comprises providing one or more engineered Gas Vesicles each comprising a gas enclosed by a protein shell comprising a Gas vesicle GvpA/B protein and an engineered GvpC protein.

In the method, the engineered GvpC is a gas vesicle protein comprising multiple repeat regions within a central portion of the GvpC flanked by an N-terminal region having an N-terminus and a C-terminal region having a C-terminus. The engineered gas vesicle protein GvpC, further comprises at least one protease recognition site inserted within the central portion and/or attached to at least one of the N-terminus and the C-terminus of the GvpC.

In the method each Gas Vesicle having, prior to exposure to a protease, an initial GV collapse pressure and an initial ultrasound response up to collapse, the initial ultrasound response having a baseline non linearity.

The method further comprises contacting the one or more engineered Gas Vesicles with a protease to allow cleavage of the protease recognition site of the engineered GvpC and detecting a protease induced GV collapse pressure and/or a protease induced GV ultrasound response of the one or more engineered Gas Vesicles following the contacting. The method also comprises selecting following the contacting the one or more engineered Gas Vesicles having a detected protease induced GV collapse pressure lower than the initial Gv collapse pressure and/or a protease induced ultrasound response having a non linearity increased with respect the baseline non-linearity, the selecting performed to provide a protease sensitive Gas Vesicle.

According to a second aspect, an engineered protease sensitive gas vesicle is described. The engineered protease sensitive gas vesicle comprises a gas enclosed by a protein shell in which a Gas vesicle GvpA/B protein and an engineered protease sensitive GvpC protein of the instant disclosure are arranged in a configuration in which the engineered protease sensitive GvpC protein binds the Gas vesicle GvpA/B protein to form the protein shell, the at least one protease recognition site is presented on the protein shell of the engineered protease sensitive gas vesicle.

In the engineered protease sensitive gas vesicle, the Gas Vesicle has an initial GV collapse pressure, an initial ultrasound response with a baseline non-linearity, a protease induced GV collapse pressure lower than the initial GV collapse pressure and a protease induced ultrasound response having an enhanced nonlinearity with respect to the initial ultrasound response. In the engineered protease sensitive gas vesicle, the protease induced GV collapse pressure and the protease induced ultrasound contrast signal are detected following processing of the at least one protease recognition site by a corresponding protease.

According to a third aspect, an engineered protease sensitive gas vesicle protein GvpC is described and a polynucleotide encoding therefor. The engineered protease sensitive GvpC is a gas vesicle protein comprising multiple repeat regions within a central portion of the GvpC flanked by an N-terminal region having an N-terminus and a C-terminal region having a C-terminus. The engineered gas vesicle protein GvpC, further comprises at least one protease recognition site inserted within the central portion and/or attached to at least one of the N-terminus and the C-terminus of the GvpC. In the engineered protease sensitive gas vesicle protein GvpC, the central portion, the N-terminal region and the C-terminal region are configured to bind Gas vesicle GvpA/B protein of a Gas Vesicle to form a Gas Vesicle protein shell and to present the at least one protease recognition site on the Gas Vesicle protein shell upon assembly.

In the engineered protease sensitive GvpC, the multiple repeat region, N-terminal region, C-terminal region and protease recognition site are in a configuration associated upon assembly of the engineered protease sensitive GvpC in a GV having an initial GV collapse pressure of the GV, an initial ultrasound response having a baseline non-linearity, a protease induced GV collapse pressure lower than the initial GV collapse pressure and a protease induced ultrasound response having an increased nonlinearity with respect the baseline non-linearity. In the engineered protease sensitive gas vesicle, the protease induced GV collapse pressure and the protease induced ultrasound response are detected following processing of the at least one protease recognition site by a corresponding protease.

According to a fourth aspect, protease sensitive Gas Vesicle Gene Cluster (GVGC) encoding for a protease sensitive gas vesicle of the present disclosure is described. The protease sensitive Gas Vesicle Gene Cluster (GVGC) comprises gas vesicle assembly (GVA) genes and gas vesicle structural (GVS) genes configured to form a GV type in a host cell, the GVS genes of the protease sensitive GVGC comprising Gas vesicle GvpA/B protein a genetically engineered protease sensitive gvpC gene encoding for a protease sensitive GvpC protein of the instant disclosure configured to bind the Gas vesicle GvpA/B protein and to present the at least one protease recognition site on the Gas Vesicle type upon assembly.

According to a fifth aspect, a method and a system are described to detect a protease and/or image a protease associated biochemical event in a host cell comprised in an imaging target site, the method comprising:
expressing a protease sensitive Gas Vesicle in the host cell; and
imaging the target site comprising the host cell by applying an ultrasound to obtain a nonlinear ultrasound image of the target site to image the protease associated chemical event.

The system to detect a protease and/or image a protease associated biochemical event in a host cell comprises a protease sensitive gvpC gene expression cassette herein described, a genetically engineered protease sensitive Gas Vesicle expression system (GVES), and/or a host cell in a combination with a device configured to apply ultrasound for simultaneous combined or sequential use in the imaging method to detect a protease and/or image a protease associated biochemical event in a host cell herein described.

According to a sixth aspect, a method and system are described to detect a protease and/or image a protease associated event in a target site, the method comprising:
introducing into the target site, a protease sensitive Gas Vesicle herein described and/or an engineered protease sensitive host cell configured for expression of a protease sensitive Gas Vesicle herein described, the introducing performed under conditions resulting in presence of protease sensitive gas vesicles herein described in a target site of the host organism; and
imaging the target site comprising the protease sensitive Gas Vesicle herein described and/or an engineered protease sensitive host cell by applying ultrasound to obtain a nonlinear ultrasound image of the target site. In preferred embodiments the target site is a tissue or an organ within a host organism.

The system to detect a protease and/or image a protease associated event in a target site, comprises the engineered protease sensitive Gas Vesicle herein described, and/or an engineered protease sensitive cell in a combination with a device configured to apply ultrasound for simultaneous combined or sequential use in the methods to image a protease an/or a protease associated event in a target site herein described.

Additional aspects comprise methods and systems to provide a protease sensitive GvpC, methods and systems to provide a protease sensitive Gas Vesicles and related expression cassettes, expression systems, vectors, genetically engineered protease sensitive GV host cells, compositions, methods and systems as will be understood by a skilled person upon reading of the present disclosure.

The protease sensitive gas vesicle protein GvpC and related Gas Vesicle, Gas Vesicle Gene Cluster (GVGC), gene cassettes, expression systems, vectors, cells, compositions, methods and systems herein described, can be used in several embodiments for reporting protease dependent biochemical events in a prokaryotic or eukaryotic cell in vitro, or in vivo, in particular using ultrasound imaging techniques a widely available techniques with high resolution and deep tissue penetration.

In several embodiments described herein, The protease sensitive gas vesicle protein GvpC and related Gas Vesicle, Gas Vesicle Gene Cluster (GVGC), gene cassettes, expression systems, vectors, cells, compositions, methods and systems herein described, can be used to report the location of a protease associated biological event within an imaging target site, and/or sense and report one or more biochemical events in prokaryotic or eukaryotic cells configured to express one or more protease sensitive GV types within an imaging target site.

The protease sensitive gas vesicle protein GvpC and related Gas Vesicle, Gas Vesicle Gene Cluster (GVGC), gene cassettes, expression systems, vectors, cells, compositions, methods and systems herein described, can be used in several embodiments to produce dynamic contrast in response to local protease associated molecular signals.

The protease sensitive gas vesicle protein GvpC and related Gas Vesicle, Gas Vesicle Gene Cluster (GVGC), gene cassettes expression systems, vectors, cells, compositions, methods and systems herein described, can be used in several embodiments to track over time and/or space protease associated biological events in target sites such as prokaryotic and/or eukaryotic cells, as well as tissues and organs within the body of an individual or other environments.

The protease sensitive gas vesicle protein GvpC and related Gas Vesicle, Gas Vesicle Gene Cluster (GVGC), gene cassettes expression systems, vectors, cells, compositions, methods and systems herein described, can be used in connection with various applications wherein reporting of protease activity and/or a protease associated biological events in a target site is desired. For example, the protease sensitive gas vesicle protein GvpC and related Gas Vesicle, Gas Vesicle Gene Cluster (GVGC), gene cassettes expression systems, vectors, cells, compositions, methods and systems herein described, can be used for visualization of biological events, such as cellular signaling, homeostasis, cellular migration, responses to external stimuli such as temperature and pH, onset of disease pathologies, and responses to drug treatments and therapy facilitating development of diagnostic and therapeutic cellular agents, among other advantages identifiable by a skilled person, in medical applications, as well diagnostics applications.

Additional exemplary applications include uses of the protease sensitive gas vesicle protein GvpC and related Gas Vesicle, Gas Vesicle Gene Cluster (GVGC), gene cassettes expression systems, vectors, cells, compositions, methods and systems herein described, in several fields including basic biology research, applied biology, bio-engineering, bio-energy, medical research, medical diagnostics, therapeutics, and in additional fields identifiable by a skilled person upon reading of the present disclosure.

The details of one or more embodiments of the disclosure are set forth in the accompanying drawings and the description below. Other features, objects, and advantages will be apparent from the description and drawings, and from the claims.

### BRIEF DESCRIPTION OF THE DRAWINGS

The accompanying drawings, which are incorporated into and constitute a part of this specification, illustrate one or more embodiments of the present disclosure and, together with the detailed description and example sections, serve to explain the principles and implementations of the disclosure. Exemplary embodiments of the present disclosure will become more fully understood from the detailed description and the accompanying drawings, wherein:

FIG. 1 shows an exemplary schematic representation of a Gas vesicles herein described. In particular FIG. 1 shows a rendition of a GVs showing GVS proteins forming the primary GV shell and in particular GvpA ribs (1) (gray) the outer scaffold protein GvpC (2) (black dark rectangles).

FIG. 2 shows amino acid GvpC sequences from 5 different organisms with the tandem repeat regions (Rep) within each gvpC protein aligned, preceded by the N-terminal region (N-term) and followed by the C-terminal region (C-term). Sequences and tandem repeats were obtained from Uniprot. In particular, FIG. 2 Panel a shows repeat sequence of GvpC from Halobacterium salinarum, FIG. 2 Panel b shows repeat sequence of GvpC from Anabaena flos-aquae, FIG. 2 Panel a shows repeat sequence of GvpC from Halobacterium mediterranei, FIG. 2 Panel d shows repeat sequence of GvpC from Microchaete diplosiphon, and FIG. 2 Panel e shows repeat sequence of GvpC from Nostoc sp.

FIG. 3 shows exemplary sequence alignment of GvpC repeat regions from different organisms. In particular, FIG. 3 Panel a shows repeat sequences of GvpC from Halobacterium salinarum, FIG. 3 Panel b shows repeat sequences of GvpC from Haloferax mediterranei, FIG. 3 Panel c shows repeat sequences of GvpC from Anabaena flos-aquae, FIG. 3 Panel d shows repeat sequences of GvpC from Microchaete diplosiphon, FIG. 3 Panel e shows repeat sequences of GvpC from shows Nostoc sp. FIG. 3 Panel f shows repeat sequences of GvpC from shows Microcystic aeruginosa, FIG. 3 Panel g shows repeat sequences of GvpC from shows a sequence alignment of the consensus sequences from Halobacterium salinarum and Haloferax mediterranei, FIG. 3 Panel h shows a sequence alignment of the consensus sequences from Anabaena flos-aquae, Microchaete diplosiphon and Nostoc sp.

FIG. 4 shows an exemplary schematic representation of a, GvpC protein and of protease sensitive GvpC herein described. In particular the schematic illustration of FIG. 4 panel A, shows a schematic representation of the structure of a GvpC with an N-terminal region (vertical stripes), the C-terminal region (horizontal stripes) flanking a Repeats region comprising repeat sequences (gray boxes) as indicated. In the illustration of FIG. 4 Panel B the exemplary structural schematic of FIG. 4 Panel A is shown further comprising an endoprotease cleavage site (white box) within a repeat sequence (gray boxes) and an exoprotease specific degradation tag (black box) at the C-terminus of the GvpC thus providing an exemplary protease sensitive GvpC according to the instant disclosure. FIG. 4 Panel C shows a schematic representation of the exemplary structural schematic of FIG. 4 Panel A wherein the insertion points of the protease recognition sites are indicated with arrows.

FIG. 5 shows a cartoon showing an exemplary embodiment of a method to provide a protease sensitive GV herein described.

FIG. 6 shows a cartoon showing an exemplary embodiment of a method to provide a protease sensitive GvpC herein described.

FIGS. 7A-7G illustrate an exemplary acoustic biosensor of TEV endopeptidase activity. FIG. 7A shows a Top: schematic of a gas vesicle (GV), including the primary shell protein GvpA (gray) and the reinforcing protein GvpC (blue). Bottom: schematic of GvpC structure, comprising five 33-amino acid repeats flanked by N- and C-terminal regions. FIG. 7B shows a schematic of GVS_{TEV}. FIG. 7C shows normalized OD₅₀₀ₙₘ of GVS_{TEV} as a function of hydrostatic pressure, after incubation with active TEV or heat-inactivated TEV (dTEV). The legend lists the midpoint collapse pressure for each condition (±95% confidence interval) determined from fitting a Boltzmann sigmoid function (N≥3 biological replicates). FIG. 7D shows Coomassie-stained SDS-PAGE gel of OD₅₀₀ₙₘ-matched samples of GVS_{TEV} incubated with dTEV or active TEV protease, before and after buoyancy purification (labeled pre b.p. and post b.p., respectively) FIG. 7E shows representative TEM images of GVS_{TEV} after incubation with dTEV or active TEV protease. FIG. 7F shows DLS measurements of the average hydrodynamic diameter of GVS_{TEV} and GV_{WT} samples after protease incubation (N=3 biological replicates for GVS_{TEV} and 4 for GV_{WT}; individual dots represent each N, and thick horizontal line indicates the mean). FIG. 7G shows representative ultrasound images of agarose phantoms containing GVS_{TEV} incubated with TEV or dTEV protease at OD₅₀₀ₙₘ. 2.2. The linear (B-mode) image was acquired at 132 kPa and the nonlinear (x-AM) image was acquired at 438 kPa. FIG. 7H shows Average ratio of x-AM to B-mode ultrasound signal as a function of applied acoustic pressure for GVS_{TEV}, after incubation with TEV or dTEV protease (N=3 biological replicates, with each N consisting of 2-3 technical replicates). FIG. 7I shows hydrostatic collapse pressure measurements for engineered Ana GVs with WT-GvpC (GV_{WT}) after protease incubation (N≥3 biological replicates). FIG. 7J shows representative ultrasound images of agarose phantoms containing GV_{WT} incubated with TEV or dTEV protease at OD₅₀₀ₙₘ. 2.2. The B-mode image was acquired at 132 kPa and the x-AM image at 569 kPa. FIG. 7K shows an average ratio of x-AM to B-mode ultrasound signal as a function of applied acoustic pressure for GV_{WT}, after incubation with TEV or dTEV protease (N=3 biological replicates, with each N consisting of 3 technical replicates). For ultrasound images in FIG. 7G and FIG. 7J, CNR stands for contrast-to-noise-ratio, and color bars represent relative ultrasound signal intensity on the dB scale. Error bars indicate SEM. Scale bars in e represent 100 nm. Scale bars in FIG. 7G and FIG. 7J represent 1 mm.

FIG. 8A-8K illustrates an exemplary acoustic biosensor of calcium-activated calpain protease. FIG. 8A shows a schematic illustration GVS_{calp}. FIG. 8B shows hydrostatic collapse curves of GVS_{calp} after incubations in the presence or absence of calpain and calcium. The legend lists the midpoint collapse pressure for each condition (±95% confidence interval) determined from fitting a Boltzmann sigmoid function (N≥5 biological replicates). FIG. 8C shows a Coomassie-stained SDS-PAGE gel of OD₅₀₀ₙₘ-matched samples of GVS_{calp} incubated in the presence (+) or absence (−) of calpain (first +/−) and calcium (second +/−), before and after buoyancy purification (labeled pre b.p. and post b.p. respectively). FIG. 8D shows representative TEM images of GVS_{calp} after incubations in the presence or absence of calpain and/or calcium. Scale bars represent 100 nm. FIGS. 8E, 8G, 8I show representative ultrasound images of agarose phantoms containing GVS_{calp} incubated with and without calpain and/or calcium at OD₅₀₀ₙₘ. 2.2. The B-mode images were taken at 132 kPa for FIGS. 8E, 8G and 8I, and the x-AM images were taken at 438 kPa for FIGS. 8E and 8G and at 425 kPa for FIG. 8I. CNR stands for contrast-to-noise-ratio, and color bars represent relative ultrasound signal intensity on the dB scale. Scale bars represent 1 mm FIGS. 8F, 8H, 8J show an average ratio of x-AM to B-mode ultrasound signal as a function of applied acoustic pressure for GVS_{calp} after incubation in the presence or absence of calpain and/or calcium (N=3 biological replicates, with each N consisting of 2 technical replicates). FIG. 8K shows a calcium-response curve for GVS_{calp} in the presence of μ-calpain, showing the ratio of x-AM to B-mode ultrasound signal at 425 kPa as a function of calcium concentration. The mean values are fitted to a Hill equation with a coefficient of 1, giving a half maximum response concentration (EC₅₀) of 140 μm (N=3 biological replicates, individual dots represent the mean values with the solid blue line showing the fitted curve). Error bars indicate SEM.

FIGS. 9A-9H illustrate an exemplary acoustic biosensor of ClpXP protease. FIG. 9A shows a schematic of GVS_{ClpXP}. FIG. 9B shows a Coomassie-stained SDS-PAGE gel of OD₅₀₀ₙₘ-matched GVS_{ClpXP} samples, incubated in a reconstituted cell-free transcription-translation (TX-TL) system containing a protease inhibitor cocktail or ClpXP. FIG. 9C shows representative TEM images of GVS_{ClpXP} after incubations in the presence of a protease inhibitor or ClpXP. FIG. 9D shows normalized optical density (OD₅₀₀ₙₘ) measurements of GVS_{ClpXP} as a function of hydrostatic pressure after protease incubation. The legend lists the midpoint collapse pressure for each condition (±95% confidence interval) determined from fitting a Boltzmann sigmoid function (N=5 biological replicates). FIG. 9E shows representative ultrasound images of agarose phantoms containing GVS_{ClpXP} incubated with the inhibitor cocktail or active ClpXP at OD₅₀₀ₙₘ 2.2. FIG. 9F shows a diagram showing average x-AM/B-mode ratio as a function of applied acoustic pressure for GVS_{ClpXP}, after incubation with the protease inhibitor or active ClpXP. FIG. 9G shows hydrostatic collapse pressure measurements for engineered Ana GVs with WT-GvpC (GV_{WT}) after protease incubation (N=3 biological replicates) FIG. 9H shows Representative ultrasound images of agarose phantoms containing GV_{WT} incubated with the inhibitor cocktail or active ClpXP at OD₅₀₀ₙₘ 2.2. FIG. 9I shows a diagram plotting average ratio of x-AM (nonlinear) to B-mode (linear) acoustic signal as a function of applied acoustic pressure for GV_{WT} after incubation with the inhibitor cocktail or ClpXP protease. For ultrasound images in FIG. 9E and FIG. 9H, CNR stands for contrast-to-noise-ratio, and color bars represent relative ultrasound signal intensity on the dB scale. The B-mode images were acquired at 132 kPa and the x-AM images were acquired at 477 kPa. For FIG. 9F and FIG. 9I, N=3 biological replicates, with each N having 3 technical replicates. Error bars indicate SEM. Scale bars in c represent 100 nm. Scale bars in For FIG. 9E and FIG. 9H, represent 1 mm.

FIGS. 10A-10J illustrate intracellular protease activity and circuit-driven gene expression in engineered cells. FIG. 10A shows a schematic of E. coli Nissle cells expressing the acoustic sensor gene construct for ClpXP. In some cases, the Nissle cells are genomically modified to lack the ClpX and ClpP⁻ genes (ΔClpXP), and co-transformed with a plasmid encoding L-arabinose (L-ara) driven ClpXP. FIG. 10B shows a normalized pressure-sensitive optical density at 600 nm of WT Nissle cells expressing either ARG_{WT} or ASG_{ClpXP}. The legend lists the midpoint collapse pressure for each cell type (±95% confidence interval) determined from fitting a Boltzmann sigmoid function (N≥3 biological replicates). FIG. 10C shows representative ultrasound images of WT Nissle cells expressing either ARG_{WT} or ASG_{ClpXP} at OD₆₀₀ₙₘ 1.5 (N=3 biological replicates, with each N having 3 technical replicates). FIG. 10D shows average x-AM/B-mode ratio as a function of applied acoustic pressure for FIG. 10E WT Nissle cells expressing either ARG_{WT} or ASG_{ClpXP} at OD₆₀₀ₙₘ 1.5 (N=3 biological replicates, with each N having 3 technical replicates). Normalized pressure-sensitive optical density at 600 nm of ΔClpXP Nissle cells expressing ASG_{ClpXP} with or without L-ara induction of ClpXP expression. The legend lists the midpoint collapse pressure for each cell type (±95% confidence interval) determined from fitting a Boltzmann sigmoid function (N≥3 biological replicates). FIG. 10F shows representative ultrasound images of ΔClpXP Nissle cells expressing ASG_{ClpXP} with or without L-ara induction of ClpXP expression at OD₆₀₀ₙₘ 1.5 (N=3 biological replicates, with each N having 3 technical replicates). FIG. 10G shows a diagram plotting average x-AM/B-mode ratio as a function of applied acoustic pressure for ΔClpXP Nissle cells expressing ASG_{ClpXP} with or without L-ara induction of ClpXP expression at OD₆₀₀ₙₘ 1.5 (N=3 biological replicates, with each N having 3 technical replicates). FIG. 10H shows a schematic of pT5-LacO driven ASG_{ClpXP} and pTet-TetO driven WT GvpC gene circuits co-transformed into Nissle cells for dynamic switching of nonlinear acoustic signals from the intracellular GV sensors in response to circuit-driven gene expression. FIG. 10I shows representative ultrasound images of Nissle cells (OD₆₀₀ₙₘ nm 1) expressing ASG_{ClpXP}, with or without aTc induction to drive expression of WT GvpC. FIG. 10J shows average x-AM/B-mode ratio as a function of applied acoustic pressure for Nissle cells expressing ASG_{ClpXP}, with or without aTc induction (N=5 biological replicates). For ultrasound images in FIGS. 10C, 10F, and 10I, CNR stands for contrast-to-noise-ratio. and color bars represent relative ultrasound signal intensity in the dB scale. The B-mode images were acquired at 132 kPa for FIGS. 10C and 10I and 309 kPa for FIG. 10F. The x-AM images were acquired at 1.11 MPa for FIG. 10C, 1.61 MPa for FIG. 10F, and 1.34 MPa for FIG. 10I. Error bars indicate SEM. Scale bars in FIGS. 10C, 10F, and 101 represent 1 mm.

FIG. 11 illustrates ultrasound imaging of bacteria expressing acoustic sensor genes in the gastrointestinal tract of mice. (Panel a) Schematic illustrating the in vivo ultrasound imaging experiment. (Panel b) Transverse ultrasound image of a mouse whose colon contains WT Nissle cells expressing ARG_{WT} at the center of the lumen and the same strain expressing ASG_{ClpXP} at the periphery of the lumen. (Panel c) B-mode and xAM contrast-to-noise ratio (CNR) in vivo, for WT Nissle cells expressing ARG_{WT} or ASG_{ClpXP}. N=9 mice. P<0.0001 for x-AM signal from cells expressing ASG_{ClpXP} versus the ARG_{WT} control. (Panel d) Transverse ultrasound image of a mouse whose colon contains ΔClpXP Nissle cells expressing ASG_{ClpXP} with L-ara induction of ClpXP expression at the center and without L-ara induction at the periphery of the lumen. Cells are injected in agarose gel at a final concentration of 1.5E9 cells ml⁻¹ for (Panel b) and (Panel d). Nonlinear (x-AM) images of the colon, acquired at 1.27 MPa for (Panel b) and 1.56 MPa for (Panel d) before and after acoustic collapse (hot color map), are superimposed on linear (B-mode) anatomical images (bone colormap). Color bars represent relative ultrasound signal intensity on the dB scale. Scale bars represent 2 mm. (Panel e) B-mode and xAM CNR in vivo, for ΔClpXP Nissle cells expressing ASG_{ClpXP} with or without L-ara induction of ClpXP expression. N=7 mice. P<0.0001 for x-AM signal from cells expressing ASG_{ClpXP} with ClpXP expression induced versus non-induced. Individual dots represent each N, and the thick horizontal line indicates the mean. Error bars indicate SEM.

FIG. 12A shows Coomassie-stained SDS-PAGE gel of OD₅₀₀ₙₘ-matched samples of GV_{WT} incubated with dTEV and TEV protease, before and after buoyancy purification (labeled pre b.p. and post b.p., respectively).

FIGS. 12B-12C show scatter plots representing the ratio of nonlinear (x-AM) to linear (B-mode) ultrasound signal as a function of applied acoustic pressure for all the replicate samples used in the x-AM voltage ramp imaging experiments for GVS_{TEV} (FIG. 12B) and for GV_{WT} (FIG. 12C). Total number of replicates is 8 for GV_{TEV} and 9 for GV_{WT}. Solid line represents the mean of all the replicates.

FIGS. 13A-13C shows scatter plots each representing the ratio of nonlinear (x-AM) to linear (B-mode) ultrasound signal as a function of applied acoustic pressure for all the GVS_{calp} replicate samples after incubation in the presence or absence of calpain and/or calcium. Total number of replicates is 6 for GVS_{calp}.

FIG. 13D represents the DLS measurements showing the average hydrodynamic diameter of GVS_{calp} and GV_{WT} samples after calpain/calcium incubations (N≥2 biological replicates, individual dots represent each N and horizontal line indicates the mean). Error bars indicate SEM.

FIGS. 14A-14C shows representative ultrasound images of agarose phantoms containing GV_{WT} incubated in the presence (+) or absence (−) of calpain (first +/−) and calcium (second +/−), at OD₅₀₀ₙₘ 2.2. The B-mode images were taken at 132 kPa for FIGS. 14A-14C and the x-AM images corresponding to the maximum difference in nonlinear contrast between the +calpain/+ calcium sample and the negative controls were taken at 438 kPa for FIGS. 14A-14B and at 425 kPa for FIG. 14C. CNR stands for contrast-to-noise-ratio and color bars represent ultrasound signal intensity in the dB scale. Scale bars represent 1 mm.

FIGS. 14D-14F represent scatter plots showing the ratio of x-AM to B-mode ultrasound signal as a function of increasing acoustic pressure for GV_{WT} after incubation in the presence or absence of calpain and/or calcium (N=2).

FIG. 14G plots hydrostatic collapse curves of GV_{WT} after incubations in the presence (+) or absence (−) of calpain and/or calcium. The legend lists the midpoint collapse pressure for each condition (±95% confidence interval) determined from fitting a Boltzmann sigmoid function (N≥5 biological replicates).

FIG. 14H shows the results of Coomassie-stained SDS-PAGE gel of OD₅₀₀ₙₘ matched samples of GV_{WT} incubated in the presence (+) or absence (−) of calpain/calcium, before and after buoyancy purification (labeled pre b.p. and post b.p., respectively).

FIG. 15A shows the results of Coomassie-stained SDS-PAGE gel of OD₅₀₀ₙₘ matched GV_{WT} samples incubated in a reconstituted cell-free transcription-translation (TX-TL) system containing a protease inhibitor cocktail or ClpXP.

FIG. 15B shows the results Coomassie-stained SDS-PAGE gel of 30× diluted content of TX-TL system containing ClpXP.

FIG. 15C represents DLS measurements showing the average hydrodynamic diameter of GVS_{ClpXP} and GV_{WT} samples, after incubations with protease inhibitor or ClpXP (N=2 biological replicates, individual dots represent each N and horizontal line indicates the mean, error bars indicate SEM).

FIGS. 15D-15E represent scatter plots showing the ratio of x-AM to B-mode acoustic signal as a function of applied acoustic pressure for all the replicate samples used in the x-AM voltage ramp experiments for GVS_{ClpXP} (FIG. 15D) and GV_{WT} (FIG. 15E). Total number of replicates is 9 for GVS_{ClpXP} and GV_{WT}.

FIG. 16 represents scatter plots showing the ratio of x-AM to B-mode acoustic signal as a function of acoustic pressure for all the replicate samples used in the x-AM voltage ramp experiments for WT Nissle cells expressing either ARG_{WT} or ASG_{ClpXP} (Panel a), ΔClpXP Nissle cells expressing ASG_{ClpXP} and araBAD driven ClpXP, with or without L-arabinose induction (Panel b) and WT Nissle cells expressing ASG_{ClpXP} and pTet-TetO driven WT GvpC, with or without aTc induction (Panel c). Total number of replicates is 9 for Panel a and 5 for Panel b.

FIG. 17 shows the ultrasound imaging of bacteria expressing acoustic sensor genes in the gastrointestinal tract of mice. (Panel a) Schematic illustrating two orientations of the wild type (WT) E. coli Nissle cells expressing ARG_{WT} or ASG_{ClpXP} introduced into the mouse colon as a hydrogel. (Panels b, c) Representative transverse ultrasound images of the colon for two mice used in the in vivo imaging experiments, with orientation #1 (Panel b) and with orientation #2. (Panel c). Cells are injected at a final concentration of 1.5E9 cells ml⁻¹. B-mode signal is displayed using the bone colormap and x-AM signal is shown using the hot colormap. Color bars represent B-mode and x-AM ultrasound signal intensity in the dB scale. Scale bars represent 2 mm. (Panels d, e) B-mode and xAM contrast-to-noise ratio (CNR) in vivo, for WT Nissle cells expressing ARG_{WT} or ASG_{ClpXP} in orientation #1 (Panel d) and orientation #2. (Panel e). N=5 mice for orientation #1 and N=4 mice for orientation #2. P=0.0014 for x-AM signal from cells expressing ASG_{ClpXP} versus the ARG_{WT} control in orientation #1, and P=0.0016 for that in orientation #2. (Panel f) B-mode and xAM contrast-to-tis sue ratio (CTR) in vivo, for WT Nissle cells expressing ARG_{WT} or ASG_{ClpXP} in both orientations. P<0.0001 for the CTR from xAM imaging of cells expressing ASG_{ClpXP} versus CTR from xAM imaging of cells expressing ARG_{WT}, B-mode imaging of cells expressing ASG_{ClpXP} and ARG_{WT}. Individual dots represent each N, and the thick horizontal line indicates the mean. Error bars indicate SEM.

FIG. 18 shows an absence of memory effect from imaging at sequentially increasing acoustic pressure. Ratio of sensor-specific signal (xAM/B-mode) acquired at the indicated acoustic pressures in the process of voltage ramping (comprising 36 points from 458 kPa to 1.6 MPa) or stepping the transducer output directly to corresponding pressure in a single step, for WT Nissle cells expressing either ARG_{WT} or ASG_{ClpXP}. N=3 biological replicates, with each N having 3 technical replicates. Individual dots represent each replicate, and the thick horizontal line indicates the mean. Error bars indicate SEM.

FIG. 19 shows an exemplary Clustal omega alignment of amino acid sequences of selected exemplary gvpA and gvpB proteins (SEQ ID NOs: 7-10 and 529-544).

FIG. 20 shows exemplary phylogenetic relationships of the gvpA protein sequences from the indicated prokaryotic species. [1]

FIG. 21 shows exemplary phylogenetic relationships of the gvpF and gvpL protein sequences from the indicated prokaryotic species. [1]

FIG. 22 shows exemplary phylogenetic relationships of the gvpN protein sequences from the indicated prokaryotic species. [1]

FIG. 23 shows diagrams illustrating the organization of exemplary gas vesicle gene clusters. Gas vesicle gene clusters from the indicated organisms are shown, with genes shown as block-shaped arrows, and genes of predicted similar function indicated in the same shade of grey. The direction of the transcription of genes within a gene cluster is indicated by the direction of the block-shaped arrows, and genes grouped together having block arrows pointed in the same direction are typically organized in the same operon. The scale bar indicates 1 kb. [1]

FIG. 24 shows diagrams illustrating organization of exemplary gyp gene clusters, wherein each letter indicates a gyp gene, and an arrow beneath a group of letters indicates an operon, with the direction of the arrow indicating the direction of transcription. [2]

FIG. 25 shows an example flowchart for a method to use differential imaging (nonlinear) of protease-sensitive GvpC GVs to determine the presence of a protease.

FIG. 26 shows an example flowchart for a method to test the buckling pressure of a GV type.

### DETAILED DESCRIPTION

Provided herein are protease sensitive genetically engineered gas vesicle gene clusters (GVGC), and related gas vesicles (GVs), genetic circuits, vectors, genetically engineered prokaryotic cells, compositions, methods and systems.

The wordings “gas vesicles”, GV″, “gas vesicles protein structure”, or “GVPS”, refer to a gas-filled protein structure natively intracellularly expressed by certain bacteria or archaea as a mechanism to regulate cellular buoyancy in aqueous environments [3]. In particular, gas vesicles are protein structures natively expressed almost exclusively in microorganisms from aquatic habitats, to provide buoyancy by lowering the density of the cells [3]. GVs have been found in over 150 species of prokaryotes, comprising cyanobacteria and bacteria other than cyanobacteria [4, 5], from at least 5 of the 11 phyla of bacteria and 2 of the phyla of archaea described by Woese (1987) [6]. Exemplary microorganisms expressing or carrying gas vesicle protein structures and/or related genes include cyanobacteria such as Microcystis aeruginosa, Aphanizomenon flos aquae Oscillatoria agardhii, Anabaena, Microchaete diplosiphon and Nostoc; phototropic bacteria such as Amoebobacter, T hiodiclyon, Pelodiclyon, and Ancalochloris; non phototropic bacteria such as Microcyclus aquaticus; Gram-positive bacteria such as Bacillus megaterium Gram-negative bacteria such as Serratia; and archaea such as Haloferax mediterranei, Methanosarcina barkeri, and Halobacteria salinarium, as well as additional microorganisms identifiable by a skilled person.

In particular, a GV in the sense of the disclosure is an intracellularly expressed structure forming a hollow structure wherein a gas is enclosed by a protein shell, which is a shell substantially made of protein (at least 95% protein). In gas vesicles in the sense of the disclosure, the protein shell is formed by a plurality of proteins herein also indicated as GV proteins or gyps, which form in the cytoplasm a gas permeable and liquid impermeable protein shell configuration encircling gas. Accordingly, a protein shell of a GV is permeable to gas but not to surrounding liquid such as water. In particular, GV protein shells exclude water but permit gas to freely diffuse in and out from the surrounding media [7] making them physically stable despite their usual nanometer size, unlike microbubbles, which trap pre-loaded gas in an unstable configuration.

GV structures are typically nanostructures with widths and lengths of nanometer dimensions (in particular with widths of 45-250 nm and lengths of 100-800 nm) but can have lengths up to 2 μm in prokaryotes but can have larger dimensions such as up to 8-10 as will be understood by a skilled person upon reading of the present disclosure. In certain embodiments, the gas vesicles protein structure have average dimensions of 1000 nm or less, such as 900 nm or less, including 800 nm or less, or 700 nm or less, or 600 nm or less, or 500 nm or less, or 400 nm or less, or 300 nm or less, or 250 nm or less, or 200 nm or less, or 150 nm or less, or 100 nm or less, or 75 nm or less, or 50 nm or less, or 25 nm or less, or 10 nm or less. For example, the average diameter of the gas vesicles may range from 10 nm to 1000 nm, such as 25 nm to 500 nm, including 50 nm to 250 nm, or 100 nm to 250 nm. By “average” is meant the arithmetic mean.

GVs in the sense of the disclosure have different shapes depending on their genetic origins [7]. For example, GVs in the sense of the disclosure can be substantially spherical, ellipsoid, cylindrical, or have other shapes such as football shape or cylindrical with cone shaped end portions depending on the type of bacteria providing the gas vesicles.

Representative examples of endogenously expressed GVs native to bacterial or archaeal species are the gas vesicle protein structure produced by the Cyanobacterium Anabaena flos-aquae (Ana GVs) [3], and the Halobacterium Halobacterium salinarum (Halo GVs) [8]. In particular, Ana GVs are cone-tipped cylindrical structures with a diameter of approximately 140 nm and length of up to 2 μm and in particular 200-800 nm or longer. Halo GVs are typically spindle-like structures with a maximal diameter of approximately 250 nm and length of 250-600 nm.

In bacteria or archaea expressing GVs, the genes (herein also gyp genes) encoding for the proteins forming the GVs (herein also GV proteins), are organized in a gas vesicle gene cluster of 8 to 14 different genes depending on the host bacteria or archaea, as will be understood by a skilled person.

The term “Gas Vesicle Genes Cluster” or “GVGC” as described herein indicates a gene cluster encoding a set of GV proteins capable of providing a GV upon expression within a bacterial or archaeal cell Since the ability of expressed GV proteins to assemble in a GV depends on the cell environment where GV proteins are expressed and a same group of gyp genes may or may not form a GV upon expression in a cell, gyp genes provide GVGCs in a cell dependent manner as will be understood by a skilled person (see on point U.S. application Ser. No. 15/663,635 published as US 2018/0030501 incorporated herein by reference).

The term “gene cluster” as used herein means a group of two or more genes found within an organism's DNA that encode two or more polypeptides or proteins, which collectively share a generalized function or are genetically regulated together to produce a cellular structure and are often located within a few thousand base pairs of each other. The size of gene clusters can vary significantly, from a few genes to several hundred genes [9]. Portions of the DNA sequence of each gene within a gene cluster are sometimes found to be similar or identical; however, the resulting protein of each gene is distinctive from the resulting protein of another gene within the cluster. Genes found in a gene cluster can be observed near one another on the same chromosome or native plasmid DNA, or on different, but homologous chromosomes. An example of a gene cluster is the Hox gene, which is made up of eight genes and is part of the Homeobox gene family. In the sense of the disclosure, gene clusters as described herein also comprise gas vesicle gene clusters, wherein the expressed proteins thereof together are able to form gas vesicles.

The term “gene” as used herein indicates a polynucleotide encoding for a protein that in some instances can take the form of a unit of genomic DNA within a bacteria, plant, or other organism. The term gene as used herein incudes naturally occurring polynucleotide encoding for a protein as well as engineered polynucleotide whose sequences have been modified from the original sequence for example to optimize expression, e.g. through codon changes (see Examples section) and/or through introduction of modified N- and/or C-terminal modifications, while still maintaining the ability to encode for the protein encoded by the naturally occurring polynucleotide or a or a functional variant thereof.

The term “polynucleotide” as used herein indicates an organic polymer composed of two or more monomers including nucleotides, nucleosides or analogs thereof. The term “nucleotide” refers to any of several compounds that consist of a ribose or deoxyribose sugar joined to a purine or pyrimidine base and to a phosphate group and that are the basic structural units of nucleic acids. The term “nucleoside” refers to a compound (as guanosine or adenosine) that consists of a purine or pyrimidine base combined with deoxyribose or ribose and is found especially in nucleic acids. The term “nucleotide analog” or “nucleoside analog” refers respectively to a nucleotide or nucleoside in which one or more individual atoms have been replaced with a different atom or a with a different functional group. Accordingly, the term polynucleotide includes nucleic acids of any length, and in particular DNA RNA analogs and fragments thereof.

The term “protein” as used herein indicates a polypeptide with a particular secondary and tertiary structure that can interact with another molecule and in particular, with other biomolecules including other proteins, DNA, RNA, lipids, metabolites, hormones, chemokines, and/or small molecules. The term “polypeptide” as used herein indicates an organic linear, circular, or branched polymer composed of two or more amino acid monomers and/or analogs thereof. The term “polypeptide” includes amino acid polymers of any length including full-length proteins and peptides, as well as analogs and fragments thereof. A polypeptide of three or more amino acids is also called a protein oligomer, peptide, or oligopeptide. In particular, the terms “peptide” and “oligopeptide” usually indicate a polypeptide with less than 100 amino acid monomers. In particular, in a protein, the polypeptide provides the primary structure of the protein, wherein the term “primary structure” of a protein refers to the sequence of amino acids in the polypeptide chain covalently linked to form the polypeptide polymer. A protein “sequence” indicates the order of the amino acids that form the primary structure. Covalent bonds between amino acids within the primary structure can include peptide bonds or disulfide bonds, and additional bonds identifiable by a skilled person. Polypeptides in the sense of the present disclosure are usually composed of a linear chain of alpha-amino acid residues covalently linked by peptide bond or a synthetic covalent linkage. The two ends of the linear polypeptide chain encompassing the terminal residues and the adjacent segment are referred to as the carboxyl terminus (C-terminus) and the amino terminus (N-terminus) based on the nature of the free group on each extremity. Unless otherwise indicated, counting of residues in a polypeptide is performed from the N-terminal end (NH₂-group), which is the end where the amino group is not involved in a peptide bond to the C-terminal end (—COOH group) which is the end where a COOH group is not involved in a peptide bond. Proteins and polypeptides can be identified by x-ray crystallography, direct sequencing, immunoprecipitation, and a variety of other methods as understood by a person skilled in the art. Proteins can be provided in vitro or in vivo by several methods identifiable by a skilled person. In some instances where the proteins are synthetic proteins in at least a portion of the polymer two or more amino acid monomers and/or analogs thereof are joined through chemically-mediated condensation of an organic acid (—COOH) and an amine (—NH₂) to form an amide bond or a “peptide” bond.

As used herein the term “amino acid”, “amino acid monomer”, or “amino acid residue” refers to organic compounds composed of amine and carboxylic acid functional groups, along with a side-chain specific to each amino acid. In particular, alpha- or α-amino acid refers to organic compounds composed of amine (—NH₂) and carboxylic acid (—COOH), and a side-chain specific to each amino acid connected to an alpha carbon. Different amino acids have different side chains and have distinctive characteristics, such as charge, polarity, aromaticity, reduction potential, hydrophobicity, and pKa. Amino acids can be covalently linked to form a polymer through peptide bonds by reactions between the amine group of a first amino acid and the carboxylic acid group of a second amino acid. Amino acid in the sense of the disclosure refers to any of the twenty naturally occurring amino acids, non-natural amino acids, and includes both D an L optical isomers.

In embodiments herein described identification of a gene cluster encoding GV proteins naturally expressed in bacteria or archaea as described herein can be performed for example by isolating the GVs from the bacteria or archaea, isolating the protein for the protein shell of the GV and deriving the related amino acidic sequence with methods and techniques identifiable by a skilled person (see e.g. procedures described in [10] [11]). The sequence of the genes encoding for the GV proteins can then be identified by methods and techniques identifiable by a skilled person. For example, gas vesicle gene clusters can also be identified by persons skilled in the art by performing gene sequencing or partial- or whole-genome sequencing of organisms using wet lab and in silico molecular biology techniques known to those skilled in the art. As understood by those skilled in the art, gas vesicle gene clusters can be located on the chromosomal DNA or native plasmid DNA of microorganisms. After performing DNA or cDNA isolation from a microorganism, the polynucleotide sequences or fragments thereof or PCR-amplified fragments thereof can be sequenced using DNA sequencing methods such as Sanger sequencing, DNASeq, RNASeq, whole genome sequencing, and other methods known in the art using commercially available DNA sequencing reagents and equipment, and then the DNA sequences analyzed using computer programs for DNA sequence analysis known to skilled persons.

In embodiments herein described, identification of a gene cluster encoding for GV proteins [8, 12, 13] can also be performed by screening DNA sequence databases such as GenBank, EMBL, DNA Data Bank of Japan, and others. Gas vesicle gene cluster gene sequences in databases such as those above can be searched using tools such as NCBI Nucleotide BLAST and the like, for gas vesicle gene sequences and homologs thereof, using gene sequence query methods known to those skilled in the art. For example, genes of the gene cluster for the exemplary haloarchael GVs (which have the largest number of different gyp genes) and their predicted function and features are illustrated in Example 26 of related U.S. application Ser. No. 15/613,104, filed on Jun. 2, 2017 which is incorporated herein by reference in its entirety. GV gene clusters can also be identified using a combination of genomic vicinity (e.g. antiSMASH), protein homology and prior GV gene annotation as will be understood by a skilled person.

In embodiments herein described, identification of a GVGC configured to express a gas vesicle in a target cell and of the ability of the corresponding combination gyp genes combination to result in production of functional GV proteins capable of assembling in a GV thus providing a corresponding detectable GV type can be performed through a testing method also directed to verify detectability of the GV by a detection method of choice. The testing method can be performed in the target cell where detection of the GV type is desired or in testing cells having a cell environment equivalent to the cell environment of the target cell in terms of expression of GV genes and GV formation and thus provide a model to verify ability of the gyp genes to provide a GVGC for the target cells. In the method to identify a desired GVGC the introducing can be performed using engineered polynucleotide constructs contacted with the target cell or testing cell for a time and under conditions to allow expression of the GVGC and formation of the GV type (e.g. using the methods described in U.S. application Ser. No. 15/663,635 published as US 2018/0030501 or the method described in U.S. application Ser. No. 16/736,683 filed on Jan. 7, 2020 and in PCT/US2020/012572 filed on Jan. 7, 2020 published as WO/2020/146379 each incorporated herein by reference in its entirety). The method further comprises detecting formation of a gas vesicle in the target cell or testing cell following the introducing with a pre-set method of detection. Preset methods of detection can be directed to detect acoustic and/or magnetic properties that are of interest in desired applications of the corresponding GV type. Preferably the testing can be performed in a target cell or testing cell, that have been modified, either chemically or genetically, to have the same cellular turgor pressure as the target cells according to methods identifiable by a skilled person.

Exemplary methods of detecting functional GVGC such as Transmission Electron Microscopy (TEM) and optical scattering, optical phase detection, xenon hyperCEST MRI can be used as will be understood by a skilled person.

A GV gene cluster encoding for GV proteins typically comprises Gas Vesicle Assembly (GVA) genes and Gas Vesicle Structural (GVS) genes.

The term Gas Vesicle Structural (GVS) proteins as used herein indicates proteins forming part of a gas-filled protein structure intracellularly expressed by certain bacteria or archaea and can be used as a mechanism to regulate cellular buoyancy in aqueous environments [7]. In particular, in naturally occurring GVs, a GVS shell comprises a GVS identified as gvpA or gvpB (herein also referred to as gvpA/B) and optionally also a GVS identified as gvpC.

In Gas Vesicle of the instant disclosure, Gas Vesicle Structural (GVS) proteins comprise GVS identified as gvpA/B and A protease sensitive gvpC which form the shell of a GV.

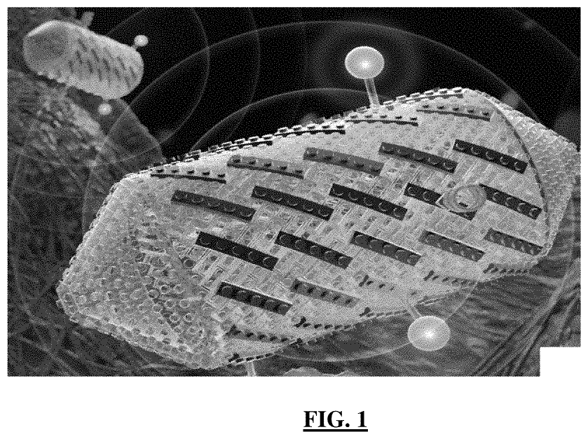

Reference is made to the illustration of FIG. 1 showing a schematic representation of the structure of an exemplary GV. In the illustration of FIG. 1 GvpA/B and GvpC are indicated as the two major structural constituents of the GV shell, with GvpA/B ribs (1) (gray) forming the primary GV shell and the outer scaffold protein GvpC (2) (black) conferring structural integrity. In particular, in the illustration of FIG. 1, the light gray elements represent the proteinaceous gas vesicle shell, comprising multiple copies of GvpA and other minor structural constituents. In the illustration of FIG. 1, the dark rectangles (2) bound to the surface of the gas vesicle shell represent GvpC, a protein that affects mechanical and acoustic properties of the gas vesicle.

In particular, gvpB gene is a gene encoding for gas vesicle structural protein B. gvpB genes is highly homologous to gvpA gene encoding for gas vesicle structural protein A. A gyp A/B is a protein of the GV shell that has a higher than 60% and possibly higher than 70% identity to the following consensus sequence: SSSLAEVLDRILDKGXVIDAWARVSLVGIEILTIEARVVIASVDTYLR (SEQ ID NO: 1) wherein X can be any amino acid. In particular in a gvpA/B of prokaryotes, the consensus sequence of SEQ ID NO: 1 typically forms a conserved secondary structure having an alpha-beta-beta-alpha structural motif formed by portions of the consensus sequence comprising the amino acids LDRILD (SEQ ID NO: 3) having an alpha helical structure, RILDKGXVIDAWARVS (SEQ ID NO: 4) wherein X can be any amino acid, having a beta strand, beta strand structure, and DTYLR (SEQ ID NO: 5) having an alpha helical structure, as will be understood by a skilled person.

As used herein, “homology”, “sequence identity” or “identity” in the context of two nucleic acid or polypeptide sequences makes reference to the nucleotide bases or residues in the two sequences that are the same when aligned for maximum correspondence over a specified comparison window. When percentage of sequence identity or similarity is used in reference to proteins, it is recognized that residue positions which are not identical often differ by conservative amino acid substitutions, where amino acid residues are substituted with a functionally equivalent residue of the amino acid residues with similar physiochemical properties and therefore do not change the functional properties of the molecule.

A functionally equivalent residue of an amino acid used herein typically refers to other amino acid residues having physiochemical and stereochemical characteristics substantially similar to the original amino acid. The physiochemical properties include water solubility (hydrophobicity or hydrophilicity), dielectric and electrochemical properties, physiological pH, partial charge of side chains (positive, negative or neutral) and other properties identifiable to a person skilled in the art. The stereochemical characteristics include spatial and conformational arrangement of the amino acids and their chirality. For example, glutamic acid is considered to be a functionally equivalent residue to aspartic acid in the sense of the current disclosure. Tyrosine and tryptophan are considered as functionally equivalent residues to phenylalanine. Arginine and lysine are considered as functionally equivalent residues to histidine.

A person skilled in the art would understand that similarity between sequences is typically measured by a process that comprises the steps of aligning the two polypeptide or polynucleotide sequences to form aligned sequences, then detecting the number of matched characters, i.e. characters similar or identical between the two aligned sequences, and calculating the total number of matched characters divided by the total number of aligned characters in each polypeptide or polynucleotide sequence, including gaps. The similarity result is expressed as a percentage of identity.

As used herein, “percentage of sequence identity” means the value determined by comparing two optimally aligned sequences over a comparison window, wherein the portion of the polynucleotide sequence in the comparison window may comprise additions or deletions (gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

As used herein, “reference sequence” is a defined sequence used as a basis for sequence comparison. A reference sequence may be a subset or the entirety of a specified sequence; for example, as a segment of a full-length protein or protein fragment. A reference sequence can comprise, for example, a sequence identifiable a database such as GenBank and UniProt and others identifiable to those skilled in the art.

As understood by those skilled in the art, determination of percent identity between any two sequences can be accomplished using a mathematical algorithm. Non-limiting examples of such mathematical algorithms are the algorithm of Myers and Miller [14], the local homology algorithm of Smith et al. [15]; the homology alignment algorithm of Needleman and Wunsch [16]; the search-for-similarity-method of Pearson and Lipman [17]; the algorithm of Karlin and Altschul [18], modified as in Karlin and Altschul [19]. Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA [17], and TFASTA in the Wisconsin Genetics Software Package, Version 8 (available from Genetics Computer Group (GCG), 575 Science Drive, Madison, Wis., USA). Alignments using these programs can be performed using the default parameters.

Thus, a gvpA/B protein can be identified for example by isolating GVs from a prokaryote, isolating the protein from the protein shell of the GV and obtaining the amino acid sequence of the isolated protein. In addition or in the alternative to the isolating the GVs and isolating the protein, the method can include obtaining amino acidic sequences of the shell proteins of the GV of the prokaryote of interest from available database. The method further comprises performing a sequence alignment of the obtained amino acidic sequences against the gvpA/B protein consensus sequence of SEQ ID NO: 1.

In particular the isolating GVs from the prokaryote can be performed following methods to isolate gas vesicles as described in U.S. application Ser. No. 15/613,104, filed on Jun. 2, 2017. The isolating the protein for the protein shell of the GV and obtaining the related amino acidic sequence can be performed with tandem liquid chromatography mass-spectrometry alone or in combination with obtaining amino acid sequences of the isolated protein with wet lab techniques or from available databases comprising the sequences of the prokaryote of interest as well as additional techniques and approaches identifiable by a skilled person. Obtaining amino acid sequences of GV shell proteins of the prokaryote of interest can be performed by screening available databases of gene and protein sequences identifiable by a skilled person. Performing a sequence alignment of the sequences of the isolated GV proteins or proteins encoded in the genome of a prokaryote of interest can be performed (using Protein BLAST or other alignment algorithms known in the art) against the gvpA/B protein consensus sequence of SEQ ID NO: 1. In particular, a sequence alignment can be performed using gvpA/B protein sequences from the closest phylogenetic relative to the prokaryote of interest. Reference is made to Example 7 showing exemplary phylogenetic relationships between gvpA/B proteins of exemplary prokaryotic species.

In GVs of the disclosure, a gvpC gene encodes for a gvpC protein which is a hydrophilic protein of a GV shell, including multiple repeats regions within a central portions flanked by an N-terminal region and a C terminal region.

The term “repeat region” or “repeat” as used herein with reference to a protein refers to the minimum sequence (herein also repeat sequence) that is present within the protein in multiple repetitions along the protein sequence without any gaps. Accordingly, in a gvpC multiple repetitions of a same repeat form a central portion flanked by an N-terminal region and a C-terminal region. In a same gvpC, repetitions of a same repeat in the gvpC protein can have different lengths and different sequence identity one with respect to another.

Repeat regions within any given gvpC sequence ‘X’ from organism ‘Y’ can be identified by comparing the related sequence with the sequence of a known gvpC (herein e.g. reference gvpC sequence “Z”). In particular, the comparing can be performed by aligning sequence ‘X’ to the reference gvpC sequence ‘Z’ using a sequence alignment tools such as BLASTP or other sequence alignment tools identifiable by a skilled person at the date of filing of the application upon reading of the present disclosure. In particular, a reference sequence ‘Z’ is chosen from a host that is the closest phylogenetic relative of ‘Y’, from a list of Anabaena flos-aquae, Halobacterium salinarum, Haloferax mediditerranei, Microchaetae diplosiphon and Nostoc sp. The sequence alignment of ‘X’ and ‘Z’ (e.g. a BLASTP) is performed by performing a first alignment of sequence X and sequence Z to identify a beginning and an end of a repeat in ‘X as well as a number of repetitions of the identified repeat, in accordance with the known repeats in 7’. The first alignment results in at least one first aligned portion of X with respect to reference sequence Z. The aligning can also comprises performing a second alignment between the at least one first aligned portion of X identified following the first alignment and additional portions of X to identify at least one repeat ‘R1’ in X. Other repeats in ‘X’ (i.e. R2, R3, R4 . . . ) can subsequently be identified with respect to R1. In performing alignment steps sequence are identified as repeat when the sequence shows at least 3 or more of the characteristics described in U.S. application Ser. No. 15/663,635 published as US 2018/0030501 (incorporated herein by reference in its entirety) which also include additional features of gvpC proteins and the related identification.

In performing alignment steps, sequences are identified as repeat when the sequence shows at least 3 or more of the following characteristics:

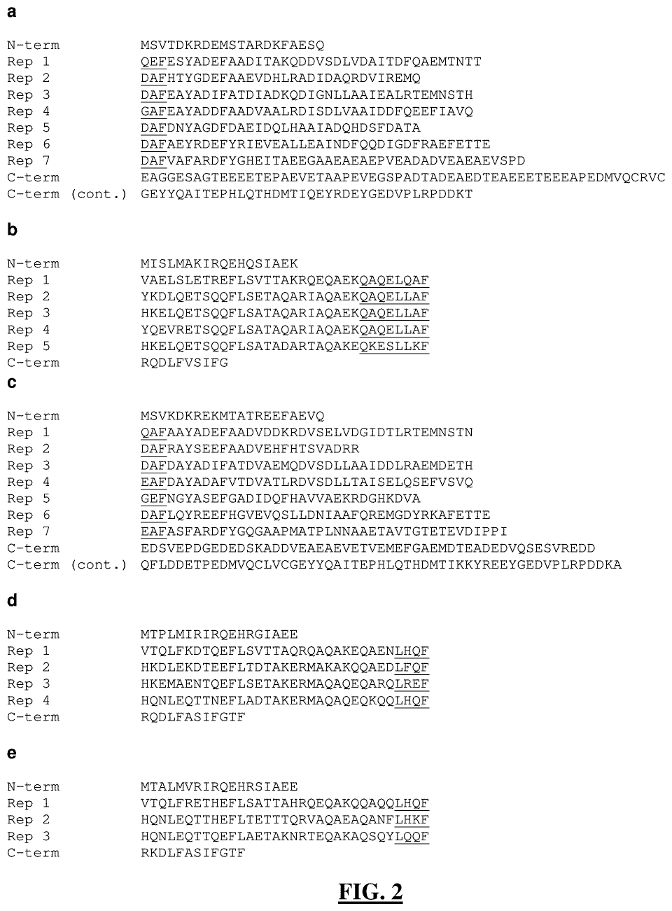

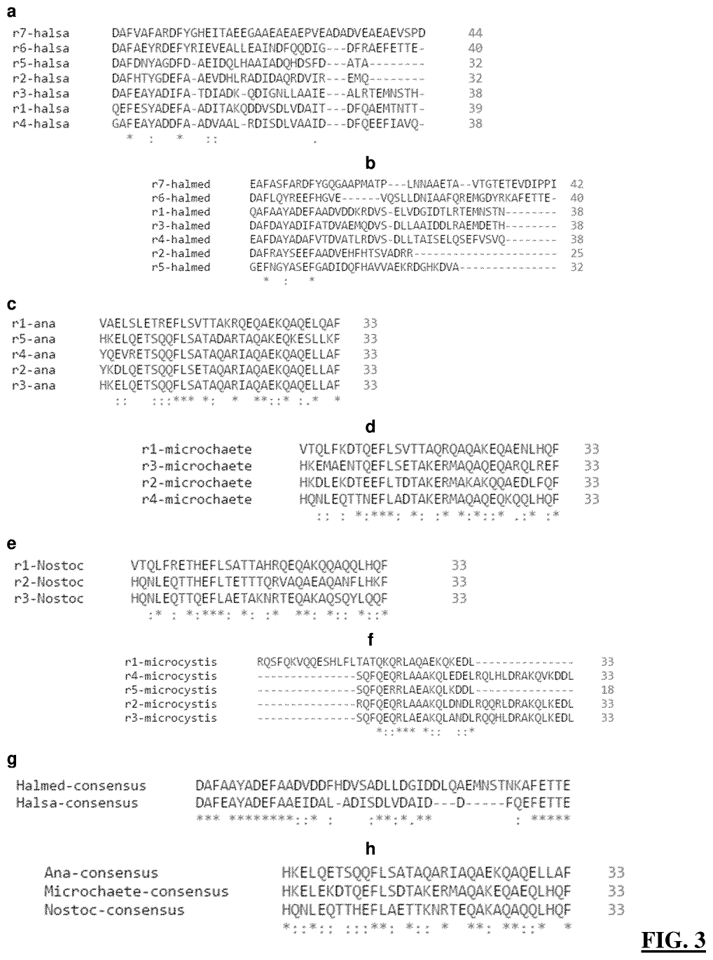

1) There are no gaps or spacer amino acids between any two adjacent repetition of a repeat (see e.g. the repeat sequences of exemplary GvpC of FIG. 2 and FIG. 3).
2) Each repetition of a repeat has a sequence length between 18-45 amino acids, e.g. 33 amino acids seen for 100% of the repeats in Anabaena flos-aquae, Microchaetae diplosiphon and Nostoc sp. (see e.g. the repeat sequences of exemplary GvpC of FIG. 3).
3) Upon alignment of all the repeats within a given GvpC sequence, there exists for every position in more than 50% of the total number of repeats, greater than 50% sequence similarity of the amino acid residues in each repeat (see e.g. the repeat sequences of exemplary GvpC of FIG. 3).
4) Sub-sequences of at least 3 or more amino acids at the beginning or end of the repeat that are conserved across 50% or more of the repeats in a given GvpC sequence, also referred to as “consensus sequences”. Exemplary embodiments of such consensus sequences are QAQELLAF (SEQ ID NO: 6) at the end of repeats in Anabaena flos-aquae, LHQF (SEQ ID NO: 11) at the end of repeats in Microchaete diplosiphon, LSQF (SEQ ID NO: 12) at the end of repeats in Microcystis aeruginosa and DAF (SEQ ID NO: 13) at the beginning of repeats in Halobacterium salinarum. (e.g. see e.g. the repeat sequences of exemplary GvpC of FIGS. 2 and 3).
5) The consensus sequence of all the repeats within a given GvpC sequence show greater than 60% identity to the consensus sequence of all the repeats within another GvpC from a different microbial host of the same phylogenetic order (see e.g. the repeat sequences of exemplary GvpC of FIG. 3, panels g-h).

Accordingly, in a GvpC herein described the central portion comprises repeats characterized by comprising at least three of the above characteristics as will be understood by a skilled person.

In some exemplary embodiments, the repeat has at least 90% sequence identity with another repeat within the same GvpC sequence.

In GvpC of GV herein described, the central portion comprising the multiple repeats is flanked by an N-terminal region and a C-terminal region. In a GvpC the N-terminal region comprises the amino acid residues upstream (towards the N-terminus) of the first repeated sequence of the GvpC's repeat and including the N-terminus, while the C-terminal region comprises the amino acid residues downstream (towards the C-terminus) of the last repeated sequence of the GvpC's repeat and comprises the C-terminus. Therefore, the N-terminal region is the region adjacent to the N-terminal portion of the first repeat of the GvpC and the C-terminal region is adjacent to the C-terminal portion of the last repeat and comprises the C-terminus of the protein)

The term the “amino terminus” or “N-terminus” indicate the amino acid residue of a linear polypeptide chain at one of the extremities of the linear polypeptide chain which, when not involved in a peptide bond, presents an amino group. The term the “carboxyl terminus” “C-terminus” indicate the amino acid residue of a linear polypeptide chain at one of the extremities of the linear polypeptide chain which, when not involved in a peptide bond, presents a carboxyl group. An N terminus or a C-terminus of a polypeptide is typically comprised within a “tail” of the protein which indicates a segment or fragment at the related end of the protein. In a GvpC “tail” of the N-terminus is provided by the N-terminal region and the “tail” of the C-terminus is provided by the C-terminal region of the GvpC>

In some embodiments, the central portion of the GvpC comprises repeat regions adjacent one with another and/or with the N-terminal region and C-terminal region. In some embodiments, the central portion of the GvpC comprises repeat regions separated by gaps. The region between repeats are herein also indicated as “junction region” or “junction” and can be formed by a bond (in case of repeat regions adjacent one with another) or a by a series of residues e interspersed between repeats as will be understood by a skilled person, depending on the specific type and configuration of the GvpC.

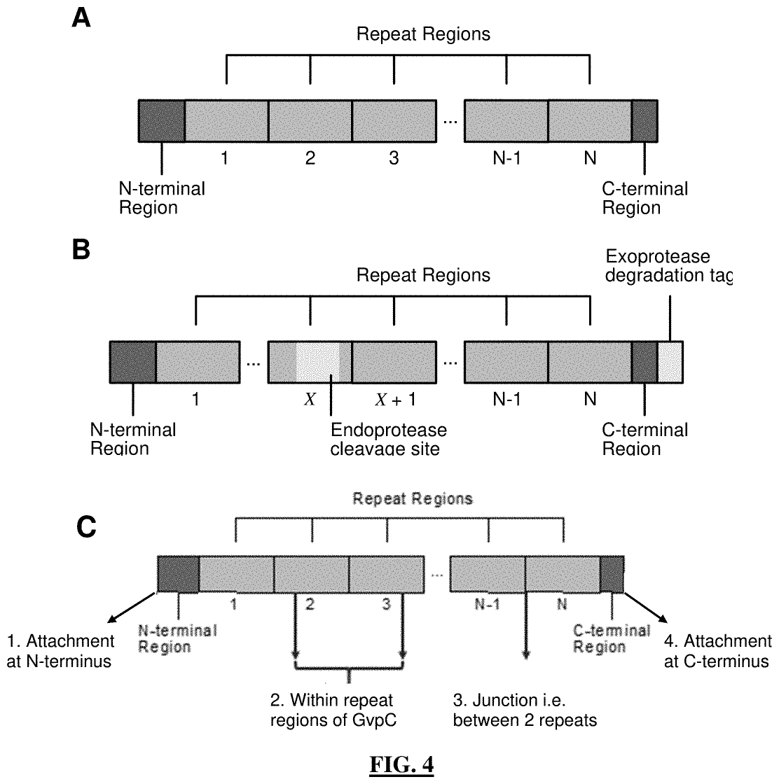

Reference is made in this connection to the exemplary schematic illustration of FIG. 4 panel A wherein a schematic illustration of an exemplary configuration of a gvpC is show in which repeat regions of different lengths indicated as (1) (2) (3) (N) and (N−1) form a central portions flanked by the N terminal and C terminal sequence. In the exemplary schematic illustration of FIG. 4 panel A, an N number of repeat regions schematically illustrated by labeling repeat regions (1) (2) (3) and (N−1) and are shown. In GvpC according to the present disclosures N can typically range from 1 to 7 as will be understood by a skilled person. In the exemplary schematic illustration of FIG. 4 panel A repeat regions are adjacent one with the other with no gaps between the repeats, while repeat regions are separated by junctions of different length interspersed between repeats as will be understood by a skilled person.

Exemplary GvpC sequences and related N-terminal region, C-terminal region and repeats are reported in Tables 1 to 5 below

TABLE 1
GvpC in Anabaena flos-aquae

| Position | Sequence | SEQ ID NO |
|---|---|---|
| N-terminus | MISLMAKIRQEHQSIAEK | 14 |
| Repeat 1 | VAELSLETREFLSVTTAKRQEQAEKQAQELQAF | 15 |
| Repeat 2 | YKDLQETSQQFLSETAQARIAQAEKQAQELLAF | 16 |
| Repeat 3 | HKELQETSQQFLSATAQARIAQAEKQAQELLAF | 17 |
| Repeat 4 | YQEVRETSQQFLSATAQARIAQAEKQAQELLAF | 18 |
| Repeat 5 | HKELQETSQQFLSATADARTAQAKEQKESLLKF | 19 |
| C-terminus | RQDLFVSIFG | 20 |

TABLE 2
GvpC in Halobacterium Salinarum

| Position | Sequence | SEQ ID NO |
|---|---|---|
| N-terminus | MSVTDKRDEMSTARDKFAESQ | 21 |
| Repeat 1 | QEFESYADEFAADITAKQDDVSDLVDAITDFQAEMTNTT | 22 |
| Repeat 2 | DAFHTYGDEFAAEVDHLRADIDAQRDVIREMQ | 23 |
| Repeat 3 | DAFEAYADIFATDIADKQDIGNLLAAIEALRTEMNSTH | 24 |
| Repeat 4 | GAFEAYADDFAADVAALRDISDLVAAIDDFQEEFIAVQ | 25 |
| Repeat 5 | DAFDNYAGDFDAEIDQLHAAIADQHDSFDATA | 26 |
| Repeat 6 | DAFAEYRDEFYRIEVEALLEAINDFQQDIGDFRAEFETTE | 27 |
| Repeat 7 | DAFVAFARDFYGHEITAEEGAAEAEAEPVEADADVEAEAEVSPD | 28 |
| C-terminus | EAGGESAGTEEEETEPAEVETAAPEVEGSPADTADEAEDTEAEEE | 29 |
| | TEEAPEDMVQCRVCGEYYQAITEPHLQTHDMTIQEYRDEYGEDV | |
| | PLRPDDKT | |

TABLE 3
GvpC in Haloferax Mediterranei

| Position | Sequence | SEQ ID NO |
|---|---|---|
| N-terminus | MSVKDKREKMTATREEFAEVQ | 30 |
| Repeat 1 | QAFAAYADEFAADVDDKRDVSELVDGIDTLRTEMNSTN | 31 |
| Repeat 2 | DAFRAYSEEFAADVEHFHTSVADRR | 32 |
| Repeat 3 | DAFDAYADIFATDVAEMQDVSDLLAAIDDLRAEMDETH | 33 |
| Repeat 4 | EAFDAYADAFVTDVATLRDVSDLLTAISELQSEFVSVQ | 34 |
| Repeat 5 | GEFNGYASEFGADIDQFHAVVAEKRDGHKDVA | 35 |
| Repeat 6 | DAFLQYREEFHGVEVQSLLDNIAAFQREMGDYRKAFETTE | 36 |
| Repeat 7 | EAFASFARDFYGQGAAPMATPLNNAAETAVTGTETEVDIPPI | 37 |
| C-terminus | EDSVEPDGEDEDSKADDVEAEAEVETVEMEFGAEMDTEADED | 38 |
| | VQSESVREDDQFLDDETPEDMVQCLVCGEYYQAITEPHLQTHD | |
| | MTIKKYREEYGEDVPLRPDDKA | |

TABLE 4
GvpC in Microchaete diplosiphon

| Position | Sequence | SEQ ID NO |
|---|---|---|
| N-terminus | MTPLMIRIRQEHRGIAEE | 39 |
| Repeat 1 | VTQLFKDTQEFLSVTTAQRQAQAKEQAENLHQF | 40 |
| Repeat 2 | HKDLEKDTEEFLTDTAKERMAKAKQQAEDLFQF | 41 |
| Repeat 3 | HKEMAENTQEFLSETAKERMAQAQEQARQLREF | 42 |
| Repeat 4 | HQNLEQTTNEFLADTAKERMAQAQEQKQQLHQF | 43 |
| C-terminus | RQDLFASIFGTF | 44 |

TABLE 5
GvpC in Nostoc sp.

| Position | Sequence | SEQ ID NO |
|---|---|---|
| N-terminus | MTALMVRIRQEHRSIAEE | 45 |
| Repeat 1 | VTQLFRETHEFLSATTAHRQEQAKQQAQQLHQF | 46 |
| Repeat 2 | HQNLEQTTHEFLTETTTQRVAQAEAQANFLHKF | 47 |
| Repeat 3 | HQNLEQTTQEFLAETAKNRTEQAKAQSQYLQQF | 48 |
| C-terminus | RKDLFASIFGTF | 49 |

A GvpC protein in the sense of the disclosure is typically rich in glutamine, alanine and glutamic acid residues, which account for >40% of the residues. In the exemplary Anabaena flos-aqaue, GvpC comprises five highly conserved 33-amino acid repeats with predicted alpha-helical structure, and is believed to bind across GvpA ribs to provide structural reinforcement [3], which aligns with experimental data. In biochemical studies, removal of GvpC and truncations to its sequence were shown to result in a reduced threshold for Ana GV collapse under hydrostatic pressure. In addition, previous studies in other species have demonstrated that GvpC can tolerate fusions of bacterial and viral polypeptides.

GvpC sequences in different bacteria or archaea producing GVs typically have a greater than 15% sequence identity and are produced by genes found in the gas vesicle gene cluster.

In a GVGC, the GVS genes are comprised with Gas Vesicle Assembly genes. The Gas Vesicle Assembly genes are genes encoding for GVA proteins. GVA proteins comprise proteins with various putative functions such as nucleators and/or chaperons as well as proteins with an unknown specific function related to the assembly of the GV.

In a prokaryotic cell GVA genes are all the genes within one or more operons comprising at least one of a gvpN and a gvpF excluding any gvpA/B and gvpC gene possibly present within said one or more operons. Therefore GVA genes can be identified by identifying an operon in a prokaryote including at least one of a gvpN and a gvpF excluding any gvpA/B and gvpC gene.

Preferably the one or more operons comprising all the GVA genes of a prokaryote can be identified and detected by detecting a gvpN gene encoding for a GV protein consensus sequence RALXYLQAGYXVHXRGPAGTGKTTLAMHLAXXLXRPVMLIXGDDEFXTSDLIGSE SGYXXKKVVDNYIHSVVKVEDELRQNWVDNRLTXACREGFTLVYDEFNRSRPEXN NVLLSVLEEKILXLP (SEQ ID NO: 50) wherein X indicates any amino acid or a sequence of any length having at least 50%, and more preferably 60% or higher, most preferably from 50% to 83% identity.

gvpN genes of various microorganisms have a sequence encoding for a gvpN protein within the consensus SEQ ID NO: 50. In particular, gvpN gene in the sense of the disclosure can be a gene encoding for sequence MTVLTDKRKKGSGAFIQDDETKEVLSRALSYLKSGYSIHFTGPAGGGKTSLARALA KKRKRPVMLMHGNHELNNKDLIGDFTGYTSKKVIDQYVRSVYKKDEQVSENWQD GRLLEAVKNGYTLIYDEFTRSKPATNNIFLSILEEGVLPLYGVKMTDPFVRVHPDFR VIFTSNPAEYAGVYDTQDALLDRLITMFIDYKDIDRETAILTEKTDVEEDEARTIVTL VANVRNRSGDENSSGLSLRASLMIATLATQQDIPIDGSDEDFQTLCIDILHHPLTKCL DEENAKSKAEKIILEECKNIDTEEK (SEQ ID NO: 51) or a sequence of any length having at least 30% sequence identity with respect to SEQ ID NO: 51, preferably at least 50%, and more preferably 60% or higher.

gvpF gene in the sense of the disclosure can be a gene encoding for sequence MSETNETGIYIFSAIQTDKDEEFGAVEVEGTKAETFLIRYKDAAMVAAEVPMKIYHP NRQNLLMHQNAVAAIMDKNDTVIPISFGNVFKSKEDVKVLLENLYPQFEKLFPAIK GKIEVGLKVIGKKEWLEKKVNENPELEKVSASVKGKSEAAGYYERIQLGGMAQKM FTSLQKEVKTDVFSPLEEAAEAAKANEPTGETMLLNASFLINREDEAKFDEKVNEA HENWKDKADFHYSGPWPAYNFVNIRLKVEEK (SEQ ID NO: 52) or a sequence of any length having at least 20% sequence identity with respect to SEQ ID NO: 52, preferably at least 50%, more preferably 60%, and at least 70% or higher.

The term “operon” as described herein indicates a group of genes arranged in tandem in a prokaryotic genome as will be understood by a skilled person. Operons typically encode proteins participating in a common pathway are organized together as understood by those skilled in the art. Typically, genes of an operon are transcribed together into a single mRNA molecule referred to as polycistronic mRNA. Polycistronic mRNA comprises several open reading frames (ORFs), each of which is translated into a polypeptide. These polypeptides usually have a related function and their coding sequence is grouped and regulated together in a regulatory region, containing a promoter and an operator. Typically, repressor proteins bound to the operator sequence can physically obstruct the RNA polymerase enzyme from binding the promoter, preventing transcription. An example of a prokaryotic operon is the lac operon, which natively regulates transport and metabolism of lactose in E. coli and many other enteric bacteria.

In an operon, each ORF typically has its own ribosome binding site (RBS) so that ribosomes simultaneously translate ORFs on the same mRNA. Some operons also exhibit translational coupling, where the translation rates of multiple ORFs within an operon are linked. This can occur when the ribosome remains attached at the end of an ORF and translocates along to the next ORF without the need for a new RBS. Translational coupling is also observed when translation of an ORF affects the accessibility of the next RBS through changes in RNA secondary structure.

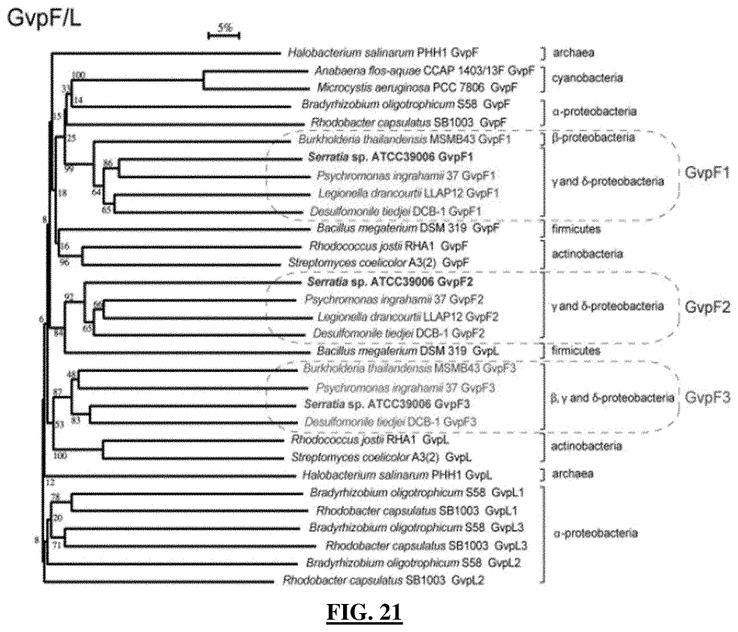

In some embodiments, a GV cluster comprises one of gvpN or gvpF. In several embodiments GV clusters include both gvpN and gvpF as will be understood by a skilled person. In this connection, reference is made to Example 12 and FIGS. 20 and 21 of related application U.S. application Ser. No. 15/663,635 published as US 2018/0030501 incorporated herein by reference in its entirety, showing exemplary gas vesicle gene clusters operons [1, 2] comprising GVS and GVA genes and related exemplary configuration. In particular, as shown in Example 12 of related application U.S. Application Ser. No. 15/663,635 published as US 2018/0030501, typically a native GV gene cluster has GVA genes comprising both gvpN and gvpF genes, even if native GV gene clusters are known having a gvpN gene or a gvpF gene, as understood by skilled persons.

Accordingly, for a certain prokaryote, GVA genes in the sense of the disclosure indicate all the genes that are comprised in the one or more operons having at least one of a gvpN and/or a gvpF herein described and excluding any Gas Vesicle Structural (GVS) genes of the prokaryotes possibly comprised within the one or more operons.

Thus, GVA genes comprised in a gas vesicle gene cluster in a prokaryote can be identified for example by obtaining genome sequence of the prokaryote of interest and performing a sequence alignment of the protein sequences encoded in the genome of the prokaryote of interest against a gvpN protein sequence and/or a gvpF protein sequence.

In particular, obtaining the genome sequence of the prokaryote of interest, can be performed either using wet lab techniques identifiable by a skilled person upon reading of the present disclosure, or obtained from databases of gene and protein sequences also identifiable by a skilled person upon reading of the present disclosure. Performing a sequence alignment of the protein sequences encoded in the genome of the prokaryote of interest can per performed using Protein BLAST or other alignment algorithms identifiable by a skilled person. Exemplary gvpN protein sequence and/or a gvpF protein sequence that can be used in performing the alignment are sequences SEQ ID NO: 51 and/or SEQ ID NO: 52. In particular, a sequence alignment can be performed using gvpN and/or gvpF protein sequences from the closest phylogenetic relative to the prokaryote of interest. Reference is made to Example 7 showing exemplary phylogenetic relationships between gvpF and gvpN proteins of exemplary prokaryotic species. Accordingly, one or more operons that comprise the gvpN and/or gvpF genes can be identified, and any other gyps within the one or more operons can also be identified, wherein the other gyps are comprised in ORFs within the one or more operons, excluding any ORFs encoding gvpA/B or gvpC genes comprised in the one or more operons of the GV gene cluster.

Accordingly, GVA genes can also be identified based on the configuration of operon and Gene Clusters identified through homology (see e.g. Example 6), phylogenesis (see e.g. Example 7) also using the gvpA/B, gvpN and/or gvpF consensus of SEQ ID Nos: 1, 50-52 herein provided, preferably gvpA/B consensus of SEQ ID NO: 1 and gvpN consensus of SEQ ID NOs: 50-51. Reference is also made in this connection to the indication of Example 8 reporting exemplary GVGC configurations of naturally occurring Gas Vesicle gene clusters identified with method herein described and additional methods identifiable by a skilled person.

GVS genes of a GVGC of the disclosure, identified with methods herein indicated, typically comprise gvpA or gvpB which have similar sequences and are equivalent in their purpose and optionally gvpC. Exemplary sequences for gvpA and gvpB genes of GV gene clusters in the sense of the disclosure, which can also be used to identify additional GVS and GVGC through homology and alignment in addition to the use of the consensus sequence SEQ ID NO: 1, are reported in Example 9.

GVGC of the disclosure can comprise hybrid Gas Vesicles Gene Clusters. The term “hybrid gene cluster” or “hybrid cluster” as used herein indicates a cluster comprising at least two genes native to different species and resulting in a cluster not natively in any organisms. Typically, a hybrid gene cluster comprises a subset of gas vesicle genes native to a first bacterial species and another subsets of gas vesicle genes native to one or more bacterial species, with at least one of the one or more bacterial species different from the first bacterial specie Accordingly, a hybrid GV gene clusters includes a combination of GV genes which is not native in any naturally occurring prokaryotes.

GVA genes of a GVGC of the disclosure, identified with methods herein indicated, typically comprise proteins identified as gvpF, gvpG, gvpL, gvpS, gvpK, gvpJ, and gvpU. GVA genes and proteins can also comprise gvpR and gvpT (see e.g. B. megateriurn GVA) gvpV, gvpW (se Anaboena flos ague and Serratia GVA) and/or gyp X, gyp Y and gyp Z (see e.g. Serratiai GVA. Preferably GVGC of the disclosure further comprise gvpN which result in a more robust detection with many detection methods herein described. Exemplary sequences for GVA genes of GV gene clusters in the sense of the disclosure which can also be used to identify additional GVAs and GVGC through homology and alignment are reported in Example 9.

In GVGC herein described co-expression of the GVS genes including the protease sensitive GVPC and the GVA genes in connection with regulatory sequence capable of operating in a host cell are configured to provide a GV type, with a different GVGC typically resulting in a different GV type.

The wording “GV type” in the sense of the disclosure indicates a gas vesicle having dimensions and shape resulting in distinctive mechanical, acoustic, surface and/or magnetic properties as will be understood by a skilled person upon reading of the present disclosure. In particular, a skilled person will understand that different shapes and dimensions will result in different properties in view of the indications in provided in U.S. Ser. No. 15/613,104 published as US2018/0028693 and U.S. Ser. No. 15/663,600 published as US2018/0038922 and additional indications identifiable by a skilled person Typically, larger volume results in stronger per-particle scattering, smaller diameter generally results in higher collapse pressure after removal of gvpC, and different dimensions result in different ratios of T2/T2* relaxivity per volume-averaged magnetic susceptibility ([20]).

In particular, each GV type typically has mechanical and acoustic properties which result in an associated collapse pressure profile and ultrasound response as will be understood by a skilled person.

The wording “collapse pressure profile” or “pressure profile” in connection with GV of the disclosure indicate a curve of pressure vs % of GV collapse detectable by applying a ramping value of pressures to a GV type and measuring the % collapse at each pressure. The point (pressure) of the curve (profile) where 50% or more collapsed GV are detected provides the “collapse pressure” or “collapse threshold” of the GV type. An imaging pressure amplitude can be selected for each GV type based on the collapse pressure as will be understood by a skilled person. Accordingly, for a GV type, a collapsing ultrasound is typically provided at a high ultrasound pressure amplitude in order to collapse the GV, while the imaging ultrasound is typically provided at a low ultrasound pressure amplitude to avoid collapsing of the GVs. The imaging ultrasound is typically a low-pressure ultrasound, applied at an imaging ultrasound pressure lower than a selectable acoustic collapse pressure value as will be understood by a skilled person.

The term “ultrasound response” as used herein indicates ultrasound waves provided as an ultrasound reflection (echo) of ultrasound-range acoustic energy applied to a reference area. Accordingly, the ultrasound response of the area is based on the mechanical and acoustic properties of items in the referenced area, which herein include GVs (e.g. acting as a contrast agent). An ultrasound response can take the form of a linear or non linear. The term “nonlinear signal” refers to a signal that does not obey superposition and scaling properties, with regards to the input. The term “linear signal” refers to a signal that does obey those properties. One example of nonlinearity is the production of 2^{nd}+ order harmonic signals in response to ultrasound excitation at a certain fundamental frequency. Another example is a nonlinear response to acoustic pressure. An example of a nonlinear signal is the increase in both fundamental and harmonic signals with increasing pressure of the transmitted imaging pulse, wherein certain GVs exhibit a highly nonlinear relationship between these signals and the pulse pressure. [21]

In particular, as part of the ultrasound response, some GV types are configured to buckle at an ultrasound pressure indicated as buckling pressure. The buckling pressure is an ultrasound pressure above which the GVs would provide a distinctively increased nonlinear ultrasound signal due to certain morphology and shell mechanical properties, in this case for GVs with shortened or removed GvpCs due to being cleaved/degraded by protease. Those GVs buckle more easily under acoustic pressure. As already observed in Maresca et al 2017 [22] and in Maresca et al 2018 [23]. (see also US Pat. Pub. 2019/0314000], incorporated herein by reference in their entirety, presence of buckling produces a significant nonlinear contrast in response to applied ultrasound pressure which can be distinguished from nearly linear or less nonlinear signals produced by non-buckling GVs and background tissue in response to applied ultrasound pressure. Given that such nonlinear ultrasound contrast senses buckling and buckling becomes activated in the GVs engineered to have protease-cleavable GvpCs, according to the present disclosure, by protease activity, the present disclosure allows to sense presence of such activity.

Accordingly, in embodiments herein described, GVGC can be selected based on desired properties of the corresponding GV type and in particular based on the collapse pressure and pressure profile. In particular, to this extent, a skilled person can use naturally occurring GVGC, can provide engineered GVGC wherein some of the naturally occurring gyp genes are omitted, and/or can provide hybrid GVGC in which GVAs and GVS genes of naturally occurring GVGCs are combined to provide GV types having the shape and dimensions resulting in the desired properties.

Gas vesicles of the present disclosure and related GvpC, GVGC as well as polynucleotides, gene cassettes, vectors, compositions methods and systems, herein described are provided based on the surprising finding that a gas vesicle can be engineered to modify the related collapse pressure, pressure profile, and nonlinear ultrasound contrast signal in response to a protease.

The term “protease”, also called a peptidase or proteinase or proteolytic enzyme, indicates any enzyme capable of performing proteolysis by hydrolysis of the peptide bonds that link amino acids together in a polypeptide chain. A protease catalyzes breaking of peptide bonds linking amino acid residues of a polypeptide chain thus converting it into shorter fragments. In proteases in the sense of the disclosure comprise proteases capable of detaching the terminal amino acids from the protein chain (exopeptidases, such as aminopeptidases, carboxypeptidase A); and proteases that capable of breaking internal peptide bonds of a protein (endopeptidases, such as trypsin, chymotrypsin, pepsin, papain, and elastase).

Proteases in the sense of the disclosure, specifically proteolyze protein substrates at corresponding sequences of amino acid residues, herein also identified as “protease recognition site”.

The wording “specific” “specifically” or “specificity” as used herein with reference to the binding of a first molecule to second molecule refers to the recognition, contact and formation of a stable complex between the first molecule and the second molecule, together with substantially less to no recognition, contact and formation of a stable complex between each of the first molecule and the second molecule with other molecules that may be present. Exemplary specific bindings are antibody-antigen interaction, cellular receptor-ligand interactions, polynucleotide hybridization, enzyme substrate interactions and additional interaction identifiable by a skilled person. In some instances specific binding can be performed by a molecule following activation (e.g. through binding to ions or other cofactors) as will be understood by a skilled person.

In particular, the term “specific” as used herein with reference to binding between protease and a corresponding recognition sites refers to the stable binding of the protease with the region forming one or more recognition site with substantially less to no recognition, contact and formation of a stable complex with other regions of the protein comprising the one or more recognition sites. The term “specific” as used herein with reference to a molecular component of a complex, refers to the unique association of that component to the specific complex which the component is part of. The term “specific” as used herein with reference to a sequence of a polynucleotide refers to the unique association of the sequence with a single polynucleotide which is the complementary sequence. By “stable complex” is meant a complex that is detectable and does not require any arbitrary level of stability, although greater stability is generally preferred.

Typically, proteases in the sense of the disclosure are capable to specifically cleave corresponding recognition sites within a protein. However proteases can also specifically recognize more than one corresponding recognition site which can be presented in in more than one protein substrate. in some cases, wherein the more than one recognition site comprises a plurality of recognition sites, the related sequences have a consensus recognition sequence. Accordingly, proteases in the sense of the disclosure comprise promiscuous proteases capable of reacting with wide range of protein substrates including one or more recognition sequences. This is the case for example of digestive enzymes such as trypsin which have to be able to cleave the array of proteins ingested into smaller peptide fragments. Promiscuous proteases typically bind to a single amino acid on the substrate and so only have specificity for that residue. For example, trypsin is specific for the sequences . . . K\ . . . or R\ . . . (‘\’=cleavage site). Proteases in the sense of the disclosure comprise specific proteases capable to only cleave substrates with a certain sequence or amino acid structure. Proteases, being themselves proteins, can be cleaved by other protease molecules, sometimes of the same variety. This acts as a method of regulation of protease activity. Some proteases are less active after autolysis (e.g. TEV protease) whilst others are more active (e.g. trypsinogen).

Proteases in the sense of the disclosure can also be categorized based on the location of the corresponding recognition sites, as endoprotease, exoproteases and processive proteases.

The term “endoproteases” as used herein indicates a type of proteases capable of breaking internal peptide bonds in a polypeptide portion remote from the C- or N-terminus. Endoproteases can be classified into seven broad groups based on the amino acid at the (protease's) active site used to perform a nucleophilic attack on the substrate: Serine proteases —using a serine alcohol; Cysteine proteases—using a cysteine thiol; Threonine proteases—using a threonine secondary alcohol; Aspartic proteases—using an aspartate carboxylic acid; Glutamic proteases—using a glutamate carboxylic acid; Metalloproteases—using a metal, usually zinc; Asparagine peptide lyases—using an asparagine to perform an elimination reaction (not requiring water), as would be understood by a skilled person. In particular, Aspartic, glutamic and metallo-proteases activate a water molecule which performs a nucleophilic attack on the peptide bond to hydrolyze it. Serine, threonine and cysteine proteases use a nucleophilic residue in attack (usually in a catalytic triad). That residue performs a nucleophilic attack to covalently link the protease to the substrate protein, releasing the first half of the product. This covalent acyl-enzyme intermediate is then hydrolyzed by activated water to complete catalysis by releasing the second half of the product and regenerating the free enzyme.

An endoprotease “cleavage site” as used herein indicates an amino acid sequence configured to be cleaved by the endoprotease/endopeptidase in the sense of the disclosure. Cleavage sites are specific peptide sequences, or more often, peptide motifs at which site-specific proteases will cleave or cut the protein.

An endoproteinase in the sense of the disclosure is typically a specific endoprotease that only cleave substrates with a certain sequence or amino acid structure. For example, Trypsin cleave peptide bonds after Arg or Lys, unless followed by Pro; Chymotrypsin cleave peptide bonds after Phe, Trp, or Tyr, unless followed by Pro; Elastase cleave peptide bonds after Ala, Gly, Ser, or Val, unless followed by Pro; Thermolysin cleave peptide bonds before Ile, Met, Phe, Trp, Tyr, or Val, unless preceded by Pro; Pepsin cleave peptide bonds before Leu, Phe, Trp or Tyr, unless preceded by Pro; Glutamyl endopeptidase cleave peptide bonds after Glu.

The term “exopeptidases” as used herein act at or near the ends of the peptide chains, delineated as aminopeptidases and carboxypeptidases to indicate their action is at the N- or C-terminals of the peptide substrates. These enzymes can be further differentiated depending on the size of the moiety that is cleaved off, such as an amino acid, a dipeptide, or a tripeptide.

Microorganisms known to produce aminopeptidases include Aspergillus oryzae, Bacillus licheniformis, B. otulinum stearothermophilus, and Escherichia coli.

Microorganisms known to produce carboxypeptidases include Aspergillus, Penicillium, and Saccharomyces species. Carboxypeptidases can be differentiated further into three groups based on the presence of certain amino acid substituents at their active sites, namely the serine carboxyproteases, the metallocarboxyproteases, and the cysteine carboxyproteases.

Exoproteases recognizes protein substrates containing specific terminal peptide sequences called degrons. Degrons can be N-degrons or C-degrons which are degradation tags at the N-terminal and C-terminal residues of target protein substrates, respectively. For example, exoproteases of the carboxypeptidase G class are defined by their specificity of release of C-terminal glutamate residues from a wide range of N-acylating moieties, including peptidyl, aminoacyl, benzoyl, benzyloxycarbonyl, folyl and pteroyl groups. Carboxypeptidase is an exopeptidase that cleaves C-terminal aromatic and aliphatic amino acid residues from proteins/peptides.

Some exoproteases target unacetylated N-terminal Arg, -Lys, -His, -Leu, -Phe, -Tyr, -Trp, -Ile, and -Met. These exoproteases include the Saccharomyces cerevisiae Ubr1 E3; the mammalian Ubr1, Ubr2, Ubr4, and Ubr5 E3s; the Prt1 and Prt6 E3s of plants; and the mammalian non-E3 autophagy regulator p62/Sqstm1.

Exemplary exoproteases and their targeted C-degrons further include, ubiquitin ligases which recognize -GG, -RG, -PG, -XR, -RXXG, -EE, -RXX, -VX, -AX and -Z, Lon protease from the Gram-positive M. forum (rnf-Lon) which recognize the M. forum ssrA tag (mf-ssrA) and synthetic degrons [24].

Exemplary exoproteases and their targeted N-degrons include mammalian Ub-proteasome system that recognize certain N-terminal amino acid such as Arg, Trp, His, and a series of synthetic degrons [25].

The term “processive protease” refers to a protease that catalyze multiple rounds of proteolysis consecutively by hydrolysis of the peptide bonds that link amino acids together in a polypeptide chain while the polypeptide chain stays bound with the protease. The processive protease can be endoprotease and/or exoproteases. Exemplary processive proteases include bacterial Clp proteases and the mammalian proteasome.

Exemplary processive proteases and their recognition sites include proteasome-like ClpAP protease which recognizes bulky hydrophobic amino acids, such Phe, Leu, Trp and Tyr [26, 27], and proteasome-like bacterial protease ClpXP which recognizes -ANDENYALAA ttps://pubmed.ncbi.nlm.nih.gov/1962196/], on terminal as well as internal peptide bonds as will be understood by a skilled person.

Gas vesicles of the present disclosure and related GvpC, GVGC as well as polynucleotides, gene cassettes, vectors, compositions methods and systems, herein described are provided based on the surprising finding that GvpC engineered to include a protease recognition site can be configured to bind a GvpA/B of a GV type thus forming the protein shell of the GV type and at the same time present the protease recognition site for binding with a protease following assembly with the GvpA/B in a protease sensitive configuration. Such protease sensitive configuration of the engineered GvpC and GV results in a decrease of the collapse pressure, a shift of the corresponding pressure profile to lower pressure values and/or a change in the ultrasound contrast signal of the GV from a low or baseline nonlinear contrast signal to a higher/enhanced nonlinear contrast signal following cleavage by the protease of the protease recognition site presented on the protease sensitive GV type.

In protease sensitive GvpC herein described the GvpC is engineered to comprise at least one endoprotease cleavage site, an exoprotease specific degradation tag and/or processive protease recognition site.

In protease sensitive GvpC the protein recognition site can be attached to the GvpC sequence in various attachment sites within the GvpC.

The term “attach” or “attached” as used herein, refers to connecting or uniting by a bond, link, force, or tie in order to keep two or more components together, which encompasses either direct or indirect attachment. For example, “direct attachment” refers to a first molecule directly bound to a second molecule or material, while “indirect attachment” in refers to one or more intermediate molecules being disposed between the first molecule and the second molecule or material. Attachment between two referenced molecules therefore comprises connecting or uniting the two referenced molecules by covalent bonds, or non-covalent bonds between the two molecules introduced and by chemical modification of the molecules (such as with a maleimide-cysteine conjugation) or by creation of a precursor of the two molecules which will provide the two molecules attached one to the other (e.g. by creating a fusion gene comprising polynucleotides encoding for two polypeptide to be attached).

Attachments of polypeptides can be performed at their corresponding N-terminus and/or C-terminus or by insertion of a polypeptide within a reference polypeptide.

As used herein, in relation to proteins, the term “insertion” of a first protein (e.g. a peptide) in a second protein refers to the introduction of the first protein in between two adjacent amino acids of the second protein. As a result, an inserted first protein is located in between a first segment of the second protein having one of the adjacent amino acids attached to its C-terminus and a second segment of the second protein having the other one of the adjacent amino acids attached to its N-terminus.

In particular, an insertion of a first protein in a second protein is performed by forming a first covalent bond between the N-terminal amino acid of the first protein (which is typically a peptide) with a first amino acid of the two adjacent amino acids the second protein, and a second covalent bond between the C-terminal amino acid of the first protein with a second amino acid of the two adjacent amino acids of the second protein. As would be understood by a skilled person, a covalent bond between two amino acids in a protein is typically a peptide bond, which is a covalent bond between a carboxyl group and an amino group of two molecules or portions thereof, which results in releasing a molecule of water.

Accordingly, an insertion of a second protein in a first protein when performed at a protein level typically results in breaking the peptide bond between the two adjacent amino acids of the first protein and forming two new peptide bonds: one between one of the two adjacent amino acids of the first protein and the N-terminal amino acid of the second protein and the other peptide bond formed between the other one of the two adjacent amino acid of the first protein and the C-terminal amino acid of the second protein.

In engineered GvpC herein described, one or more protease recognition sites can be attached in the GvpC in a configuration which maintains the ability of the GvpC to assemble with other GV proteins of a GVGC to form a gas vesicle as described herein and allow presentation of the protease recognition site for binding on the shell of the correspondent GV type.

A method is herein described which allows a skilled person to identify gas vesicles and related GvpC proteins (herein also protease sensitive GV and GvpC) which comprise one or more protease recognition site in a protease sensitive configuration allowing formation of GV and cleavage of the GvpC protein by protease able to cleave the one or more recognition site, the cleavage detectable by ultrasound imaging.

The method to provide a protease sensitive GV and related GvpC comprises providing one or more a engineered gas vesicles each having an initial GV collapse pressure and an initial low or baseline nonlinear ultrasound contrast signal, each gas vesicle comprising a gas enclosed by a protein shell comprising a Gas vesicle GvpA/B protein and an engineered GvpC protein.

In particular in the method to provide a protease sensitive GV and GvpC, the engineered GvpC is a gas vesicle protein comprising multiple repeat regions within a central portion of the GvpC flanked by an N-terminal region having an N-terminus and a C-terminal region having a C-terminus. The engineered gas vesicle protein GvpC, further comprises at least one protease recognition site inserted within the central portion and/or attached to at least one of the N-terminus and the C-terminus of the GvpC.

In particular, in embodiments herein described a GvpC herein described is engineered to comprise at least one recognition site within at least one repeat region, a junction and/or at at least one of the N-terminus and the C-terminus.

Reference is made in this connection to the exemplary illustration of FIG. 4 panel B showing a schematic of an exemplary configuration of a GvpC and related repeat regions within a central portion flanked by the N-terminal region and C-terminal regions (shown FIG. 4 Panel a) and further including protease recognition sites. In particular, in the exemplary illustration of FIG. 4 panel C the insertion of one endoprotease cleavage site within repeat regions and a C-terminus degradation tag are schematically shown.

Additional attachment points for protease recognition sites are schematically shown, in the illustration of FIG. 4 panel C wherein exemplary attachment sites at the N-terminus, C-terminus within a repast region and/or junction are shown. In particular, it has been surprisingly found that attachment of a protease recognition site within a repeat region and/or at least one of the N-terminus and C-terminus of the GvpC can result in a protease sensitive GvpC and GV (see Examples 1 and Example 2 as well as Example 3 providing proof of principle). It is also expected in view of the results provided in Examples 1 to 3, that insertion of at least a protease recognition site within a junction of the central portion of the GvpC in view of the GvpC configurations as will be understood by a skilled person upon review of the present disclosure.

In order to that the multiple regions, N-terminal region, C-terminal region and protease recognition sites are in a protease sensitive the method further comprises contacting the one or more engineered gas vesicles with a protease to allow cleavage of the protease recognition site of the the engineered GvpC, and detecting the GV collapse pressure and/or the ultrasound contrast signal of the one or more engineered gas vesicles following the contacting.

In embodiments of the disclosure, contacting a GV with a protease can be performed either in vitro or in vivo and preferably is performed on a same environment where the protease sensitive GV is used according to the experimental design.

In some embodiments, contacting the proteas with the protease-sensitive GV can be performed by incubating the protease-sensitive GV with the protease in vitro under certain condition for a certain period of time (Examples 1-2). In some embodiments, the contacting in vitro occurs in a cell transcription-translation system comprising cell extract and plasmids encoding the protease and the contacting can be performed by incubating the cell-free extract with the protease-sensitive GV (Example 3). Additional methods and techniques to contact a GV with a protease in a suitable environment are identifiable by a skilled person upon reading of the present disclosure and/or the disclosures incorporated herein by reference.

The terms “detect” or “detection” as used herein indicates the determination of the existence, presence or fact of a target in a limited portion of space, including but not limited to a sample, a reaction mixture, a molecular complex and a substrate. The “detect” or “detection” as used herein can comprise determination of chemical and/or biological properties of the target, comprising ability to interact, and in particular bind other compounds, ability to activate another compound and additional properties identifiable by a skilled person upon reading of the present disclosure. The detection can be quantitative or qualitative. A detection is “quantitative” when it refers, relates to, or involves the measurement of quantity or amount of the target or signal (also referred as quantitation), which includes but is not limited to any analysis designed to determine the amounts or proportions of the target or signal. A detection is “qualitative” when it refers, relates to, or involves identification of a quality or kind of the target or signal in terms of relative abundance to another target or signal, which is not quantified. In particular, in embodiments herein described detection of the reportable molecular component comprising a GV type is performed through contrast enhanced imaging techniques and in particular through ultrasound imaging as will be understood by a skilled person.

In embodiments of the method to provide protease sensitive GV and related GvpC detecting the GV collapse pressure and/or the ultrasound contrast signal of the one or more engineered gas vesicles following the contacting. is performed by applying ultrasound to perform ultrasound imaging of the GV as will be understood by a skilled person upon reading of the instant disclosure.

The term “ultrasound imaging” or “ultrasound scanning” or “sonography” as used herein indicate imaging performed with techniques based on the application of ultrasound. Ultrasound refers to sound with frequencies higher than the audible limits of human beings, typically over 20 kHz. Ultrasound devices typically can range up to the gigahertz range of frequencies, with most medical ultrasound devices operating in the 1 to 18 MHz range. The amplitude of the waves relates to the intensity of the ultrasound, which in turn relates to the pressure created by the ultrasound waves. Applying ultrasound can be accomplished, for example, by sending strong, short electrical pulses to a piezoelectric transducer directed at the target. Ultrasound can be applied as a continuous wave, or as wave pulses as will be understood by a skilled person.

Accordingly, the wording “ultrasound imaging” as used herein refers in particular to the use of high frequency sound waves, typically broadband waves in the megahertz range, to image structures in the body. The image can be up to 3D with ultrasound. In particular, ultrasound imaging typically involves the use of a small transducer (probe) transmitting high-frequency sound waves to a target site and collecting the sounds that bounce back from the target site to provide the collected sound to a computer using sound waves to create an image of the target site. Ultrasound imaging allows detection of the function of moving structures in real-time. Ultrasound imaging works on the principle that different structures/fluids in the target site will attenuate and return sound differently depending on their composition. A contrast agent sometimes used with ultrasound imaging are microbubbles created by an agitated saline solution, which works due to the drop in density at the interface between the gas in the bubbles and the surrounding fluid, which creates a strong ultrasound reflection. Ultrasound imaging can be performed with conventional ultrasound techniques and devices displaying 2D images as well as three-dimensional (3-D) ultrasound that formats the sound wave data into 3-D images. In addition to 3D ultrasound imaging, ultrasound imaging also encompasses Doppler ultrasound imaging, which uses the Doppler Effect to measure and visualize movement, such as blood flow rates. Types of Doppler imaging includes continuous wave Doppler, where a continuous sinusoidal wave is used; pulsed wave Doppler, which uses pulsed waves transmitted at a constant repetition frequency, and color flow imaging, which uses the phase shift between pulses to determine velocity information which is given a false color (such as red=flow towards viewer and blue=flow away from viewer) superimposed on a grey-scale anatomical image. Ultrasound imaging can use linear or nonlinear propagation depending on the signal level. Harmonic and harmonic transient ultrasound response imaging can be used for increased axial resolution, as harmonic waves are generated from nonlinear distortions of the acoustic signal as the ultrasound waves insonate tissues in the body. Other ultrasound techniques and devices suitable to image a target site using ultrasound would be understood by a skilled person.

Applying ultrasound refers to sending ultrasound-range acoustic energy to a target. The sound energy produced by the piezoelectric transducer can be focused by beamforming, through transducer shape, lensing, or use of control pulses. The soundwave formed is transmitted to the body, then partially reflected or scattered by structures within a body; larger structures typically reflecting, and smaller structures typically scattering. The return sound energy reflected/scattered to the transducer vibrates the transducer and turns the return sound energy into electrical signals to be analyzed for imaging. The frequency and pressure of the input sound energy can be controlled and are selected based on the needs of the particular imaging task and, in some methods described herein, collapsing GVs. To create images, particularly 2D and 3D imaging, scanning techniques can be used where the ultrasound energy is applied in lines or slices which are composited into an image.

In particular, in method to provide a protease sensitive GV and GvpC ultrasound imaging is applied to detect the collapse pressure of the GV and/or the ultrasound contrast signal following contacting of the GV with the protease. Protease sensitive GV and related GvpC are then selected when the collapse pressure of the GV type is decreased and/or a protease induced ultrasound response ultrasound response having a higher non linearity than the initial ultrasound response, are detected following the contacting of the GvpC with the protease.

In some embodiments, the nonlinear ultrasound image can be an image produced by cross-modulation ultrasound imaging. In some embodiments, the method and system includes determining a buckling pressure of the protease sensitive gas vesicle.

Exemplary detection of decreased collapse pressure and/or ultrasound response having an higher nonlinearity in protease sensitive GV following contacting with protease are described in the Examples section wherein exemplary methodology and configurations are provided as proof of principle as would be understood by a skilled person.

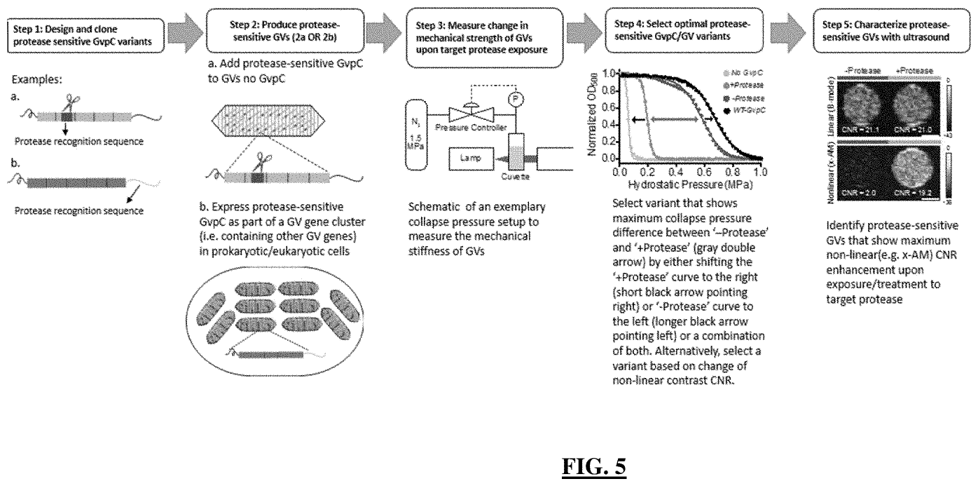

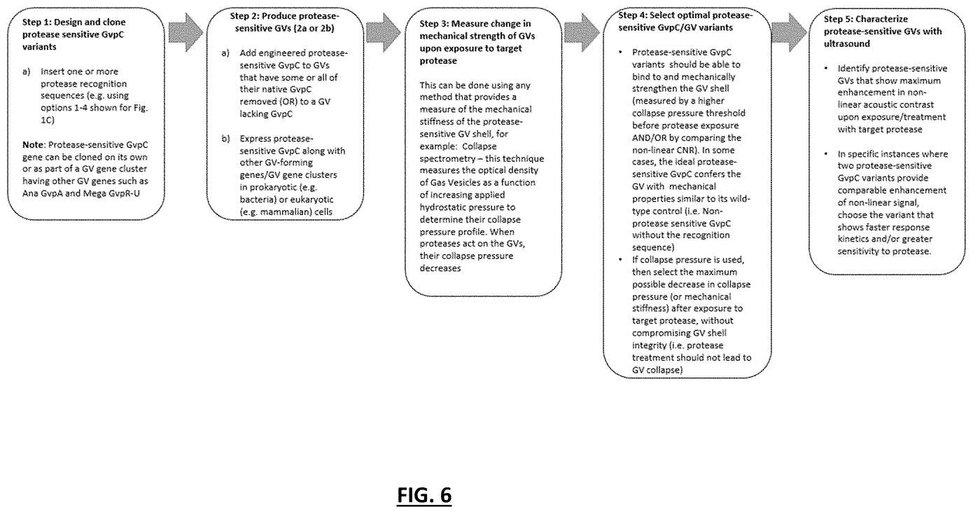

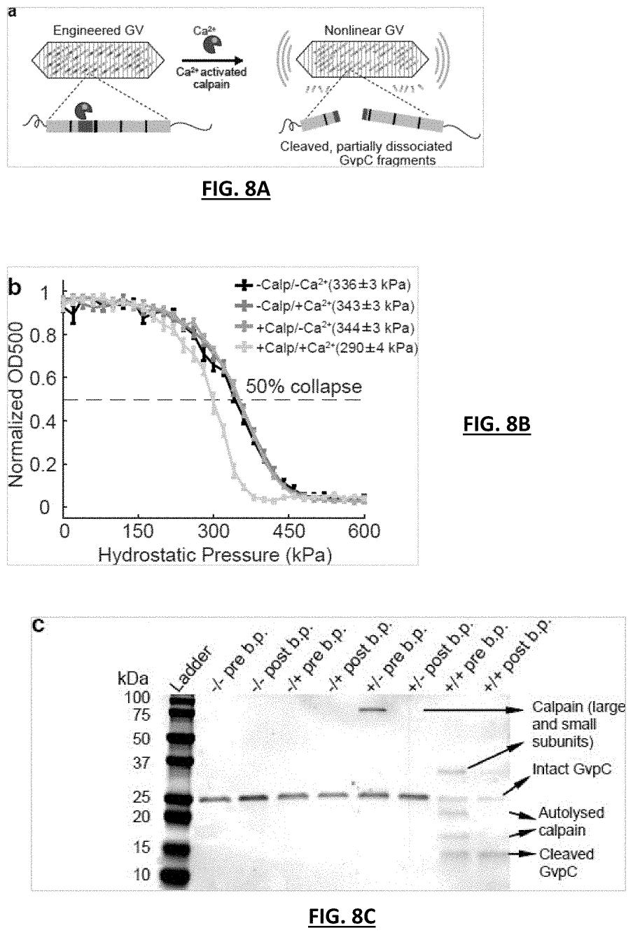

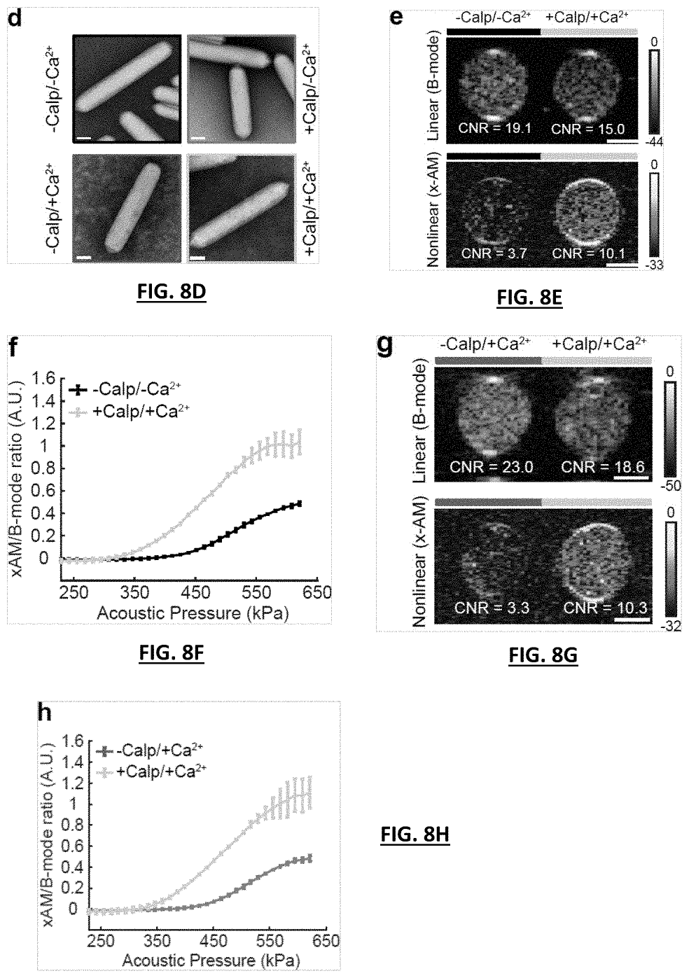

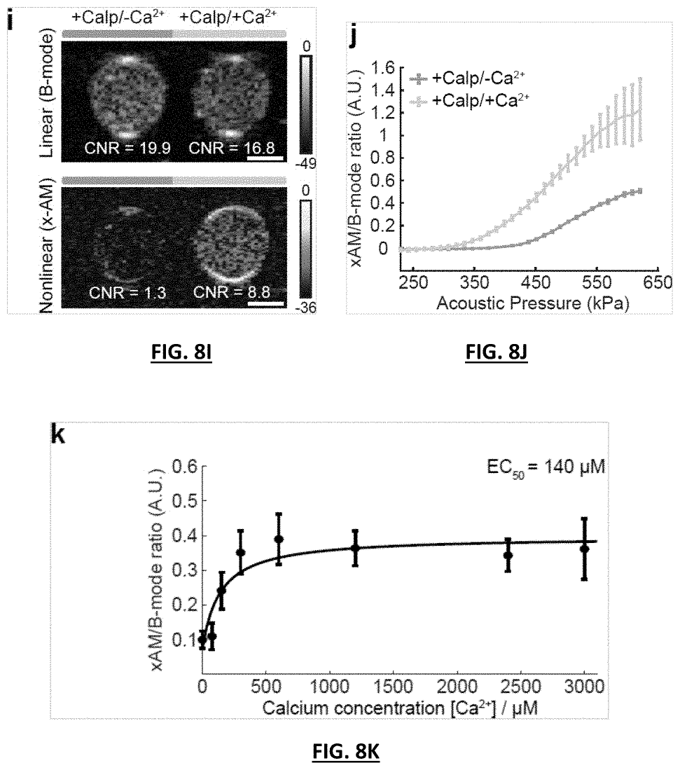

In particular, in some embodiments, the protease sensitive GVs can be screened by identifying a decrease in collapse pressure threshold or profile (see e.g. step 4 of FIGS. 5 and 6). As a cleaving or degradation of the GvpC on a GV causes a marked decrease in the collapse profile/threshold of the GV, comparing the GV prior to protease exposure to the GV after protease exposure provides proof that the GvpC was cleaved (that is, that the GV is a protease sensitive GV). See, for example, FIG. 8B. In that example, the non-sensitive GV (−Calp) shows no significant difference in pressure profile between the non-protease exposure (—Ca²⁺) and the protease exposure (+Ca²⁺) situations. Likewise, the GvpC altered (protease sensitive) GV (+Calp) shows no distinct shift in pressure profile (e.g. similar 50% collapse pressure) from the unaltered GVs. However, the altered GV being exposed to the protease (−Calp/+Ca²⁺) shows a shifted collapse profile where the collapse pressures at each % collapse is reduced (˜290 kPa compared to 336-344 kPa). This shows that the protease exposure significantly weakened the GV shell (which is due to the cleavage/degradation of the altered GvpC by the protease because the −Calp/+Ca²⁺ (non-sensitive GV exposed to the protease) profile did not show a similar reduction in collapse pressure profile).

In some embodiments, the multiple GVs can be tested and the GV with the maximum decrease in pressure profile, while still preventing GV collapse by mere exposure to the protease, can be selected as the protease sensitive GV for imaging contrast.

In some embodiments, the GVs can be further screened by testing the nonlinear effects of the cleaved GVs by nonlinear ultrasound imaging (see, e.g. FIGS. 8E and 8G) and seeing which protease sensitive GV among a group of different protease sensitive GVs shows the strongest nonlinear contrast (highest contrast to noise ratio, or “CNR”) (see e.g. step 5 of FIGS. 5 and 6).

In some embodiments, the protease sensitive GVs can be screened by identifying an increase in nonlinear contrast in the GVs after exposure to the protease. See, for example, FIG. 8G. The non-sensitive GV exposed to the protease (−Calp/+Ca²⁺) shows a strong linear contrast to noise ratio (CNR=23.0) but a weak nonlinear ratio (CNR=3.3). The sensitive GV exposed to protease (+Calp/+Ca²⁺) shows a stronger nonlinear ratio (CNR=10.3). Since the only difference between the low contrast and high contrast situations is the alteration of the GvpC of the GV, then this shows that the GV has been made protease sensitive. In some embodiments, the GVs can be further screened by comparing the CNRs of various protease sensitive GVs and seeing which is the highest. In the case of a tie (or close candidates) other factors can be used for selection, such as faster response kinetics and/or greater sensitivity to protease.

Typically a protease sensitive GV following protease contacting provide an increased nonlinear ultrasound response and are capable of buckling. In particular, protease sensitive GVs when modified by protease present in a host cell and/or target site, buckle and scatter higher harmonics at acoustic pressures above a certain applied ultrasound pressure threshold (buckling threshold). Detection and sensing of such nonlinear behavior occurs, for example, by amplitude modulation (AM) ultrasound pulse sequences in order to image and differentiate the less nonlinear behavior of non-buckling GVs from the enhanced nonlinear behavior of buckling GVs.

In some embodiments, detection of a protease induced ultrasound response can be performed to select a protease sensitive GV having a desired decrease in collapse pressure and/or a desired increase in nonlinear response (e.g. at/above a desired buckling pressure) to identify protease sensitive GV configured to be used as an acoustic biosensor of protease and/or protease associated event.

Reference is made to FIG. 5 which shows an exemplary method of functional screening/characterization to identify a protease-sensitive GV configured to act as an acoustic biosensor of enzyme activity. In Step 1, the protease sensitive GvpC variants are designed and cloned as will be understood by a skilled person upon reading of the present disclosure. Once a viable GvpC is identified, Step 2 is the production of protease sensitive GV. This can be done either by replacing or adding protease-sensitive GvpCs to GVs directly (e.g. by stripping the GvpC of a formed GV with urea and then replacing it with the engineered protease-sensitive GvpC), or by expressing protease-sensitive GvpCs as part of a GV gene cluster in a host cell as will be understood by a skilled person upon reading of the present disclosure. Then in Step 3 a change in the mechanical strength (hydrostatic collapse pressure) of the GVs is measured before and after protease exposure. This can be done by preparing a cuvette with the GVs and applying an increasing pressure (in this example, through a nitrogen gas canister controlled by a pressure valve) and observing the OD500 at each pressure increment. Then, in Step 4, the pressure profiles of pressure vs. OD500 (or normalized OD500) can be compared for different variants. The profiles can also be compared to controls such as GVs without GvpC (“no GvpC”) and wild type GvpC GVs (“WT-GvpC”). An GvpC variant can be selected which provides a maximum collapse pressure (e.g. 0.5 normalized OD500) difference between the protease sensitive GV being exposed to protease (+Protease on graph) vs. the protease sensitive GV prior to exposure (−Protease on graph). Alternatively, a minimal difference between no GvpC and +Protease can be selected. Alternatively, a greatest difference between −Protease and wild type can be selected. Alternatively, the minimal difference between the no GvpC and +Protease distance and/or the wild type and -Protease distance can be selected. Alternatively to Steps 3 and 4, one could just test the nonlinear characteristics (e.g. with xAM as described herein) and determine which variant has a nonlinear response above some pressure value (buckling threshold) A refinement of the selection can be made as shown in Step 5, where the GVs are characterized with nonlinear ultrasound imaging. This can identify the protease-sensitive GVs that show a maximum nonlinear CNR enhancement upon exposure to protease.

Reference is also made to the exemplary illustration of FIG. 6 shows an example refinement of the example given in FIG. 5 of functional screening/characterization to identify an optimal protease-sensitive GV to act as an acoustic biosensor of enzyme activity. For Step 1, one can insert one or more protease recognition sequences (e.g. using options 1-4 shown for FIG. 4C). In some embodiments, protease-sensitive GvpC gene can be cloned on its own or as part of a GV gene cluster having other GV genes such as Ana GvpA and Mega GvpR-U. For Step 2, engineered protease-sensitive GvpC can be added to GVs that have some or all of their native GvpC removed or to a GV lacking a GvpC. Also, protease-sensitive GvpCs can be expressed along with other GV-forming genes/GV gene clusters in prokaryotic (e.g. bacteria) or eukaryotic (e.g. mammalian) cells. Step 3 can be performed using any method that provides a measure of the mechanical stiffness of the protease-sensitive GV shell, for example: Collapse spectrometry—this technique measures the optical density of Gas Vesicles as a function of increasing applied hydrostatic pressure to determine their collapse pressure profile. When proteases act on the GVs, their collapse pressure decreases. For Step 4, the protease-sensitive GvpC variants should be able to bind to and mechanically strengthen the GV shell (measured by a higher collapse pressure threshold before protease exposure AND/OR by comparing the nonlinear CNR). In some cases, the ideal protease-sensitive GvpC confers the GV with mechanical properties similar to its wild-type control (Non-protease sensitive GvpC without the recognition sequence). If collapse pressure is used, then select the maximum possible decrease in collapse pressure (or mechanical stiffness) after exposure to target protease, without compromising GV shell integrity (protease treatment should not lead to GV collapse. For Step 5, identify protease-sensitive GVs that show maximum enhancement in nonlinear acoustic contrast upon exposure/treatment with target protease. In specific instances where two protease-sensitive GvpC variants provide comparable enhancement of nonlinear signal, choose the variant that shows faster response kinetics and/or greater sensitivity to protease. Accordingly, in some embodiment the method of providing protease sensitive Gas Vesicles can by producing and screening protease sensitive gas vesicles (GVs), In those embodiments the method comprises:
designing a plurality of protease sensitive GvpC;
cloning the plurality of protease sensitive GvpC;
producing GVs with the plurality of protease sensitive GvpC, creating GV and GvpC combinations;
measuring the mechanical stiffness of the GVs over a range of pressures for each of the plurality of GvpCs;
and determining which GV and GvpC combination provides a largest shift in collapse pressure based on the measuring with techniques and methodologies identifiable by a skilled person upon reading of the present disclosure. In some of those embodiments, the method can further comprise identifying which GV and GvpC combination has a maximum nonlinear contrast to noise ratio under nonlinear ultrasound imaging.

In some embodiment the producing and screening protease sensitive gas vesicles (GVs), can comprise:
designing a plurality of protease sensitive GvpC;
cloning the plurality of protease sensitive GvpC;
producing GVs with the plurality of protease sensitive GvpC, creating GV and GvpC combinations;
measuring the nonlinear ultrasound response over a range of pressures for each of the plurality of GvpCs;
and determining which GV and GvpC combination provides the maximum nonlinear ultrasound imaging contrast to noise ratio before and after exposure to the protease.
   with techniques and methodologies identifiable by a skilled person upon reading of the present disclosure. In some of those embodiments, the method can further comprise identifying which GV and GvpC combination has a maximum nonlinear contrast to noise ratio under nonlinear ultrasound imaging.

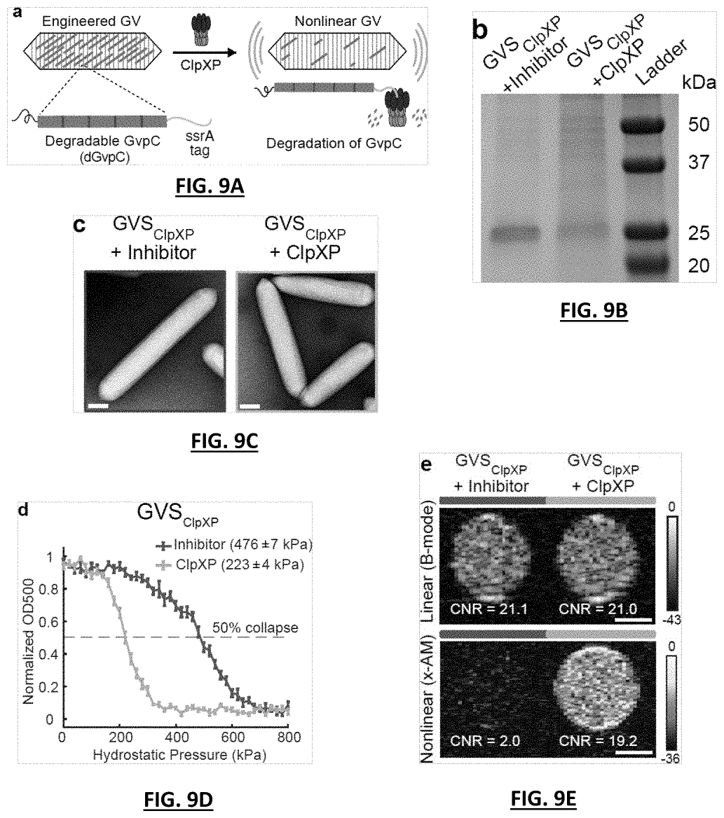

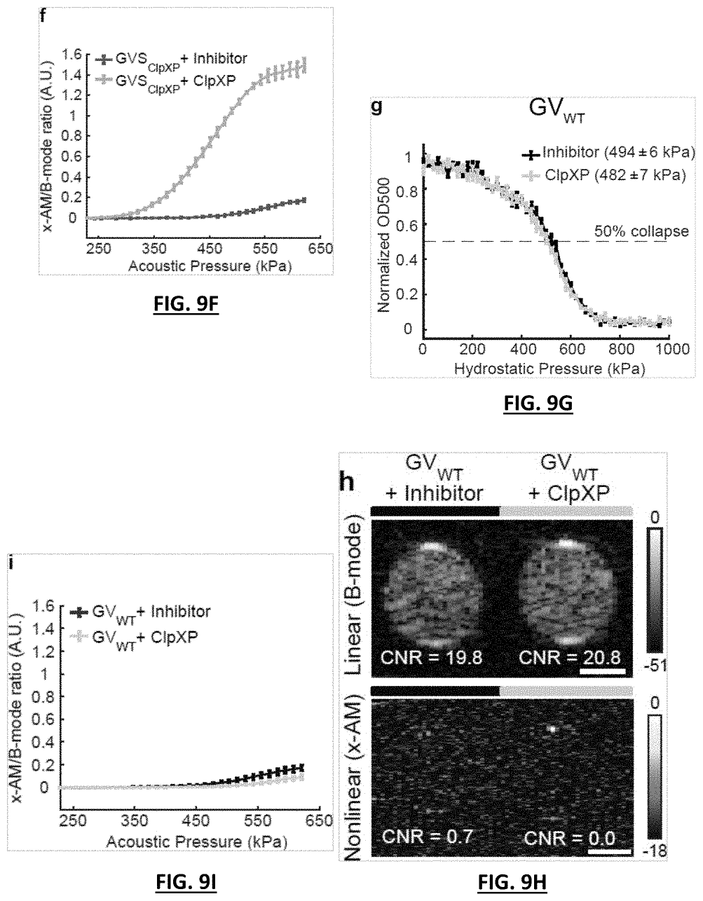

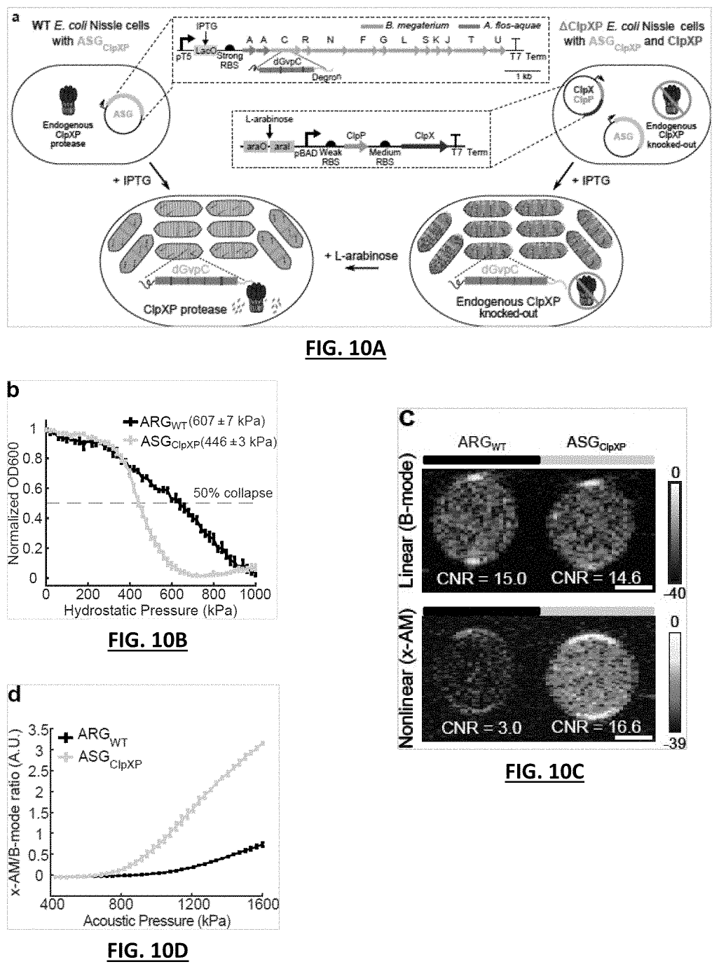

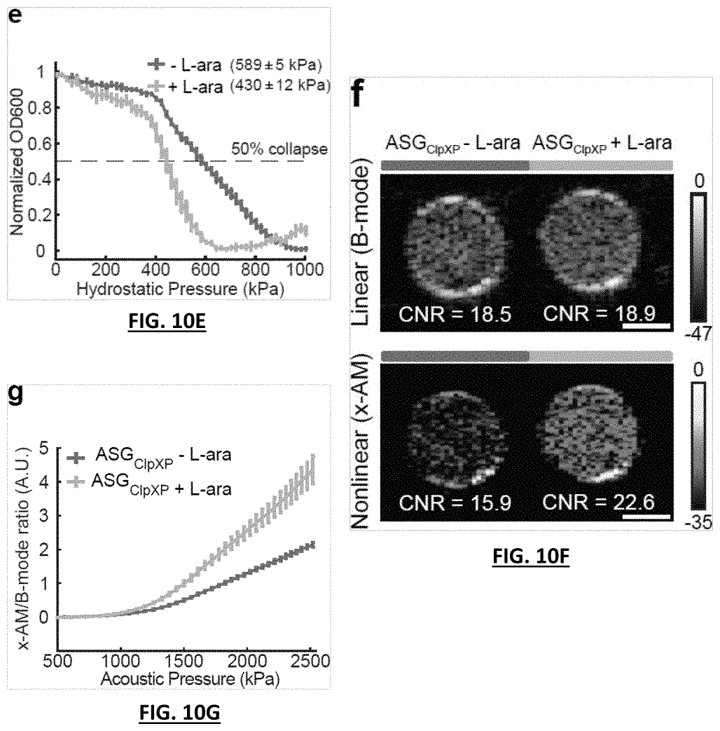

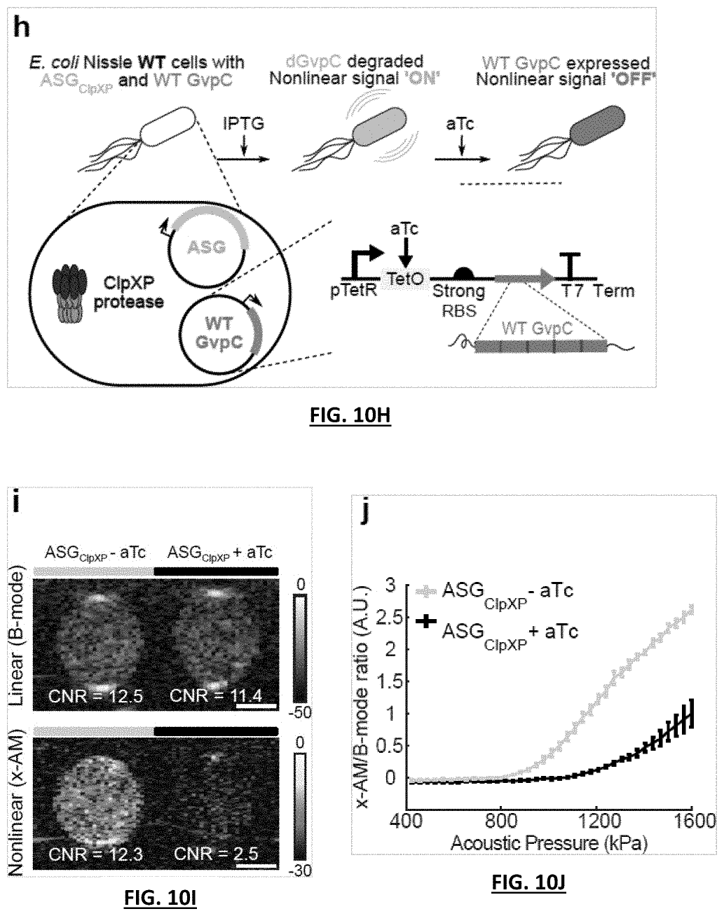

In some embodiments GVs engineered for GvpC cleaving by protease can be selected to presents optimal nonlinear acoustic response imaging or optimal change in collapse pressure. For example, for nonlinear acoustic response FIG. 10F shows a nonlinear CNR increase from 15.9 to 22.6, or over a 40% increase in CNR. Other examples show even higher CNR increase—FIG. 9E shows an 860% increase in CNR. For the collapse pressure example, FIG. 9D shows a reduction of “50% collapse” pressure from about 476 kPa to about 223 kPa or over a 50% decrease in collapse pressure. These strong imaging differences between pre- and post-protease exposure allow these particular type of GVs to be used as ultrasound contrast agents for detection of protease activity.

Additional screening approaches that can be used to perform the detecting the GV collapse pressure and/or the ultrasound contrast signal of the one or more engineered Gas Vesicles following the contacting and the selecting can be identified by a skilled person.

Exemplary recognition sequences and cleavage sites known or expected to be included in protease sensitive GV and GvpC are shown in Table 6. / forward slash (/) indicates where protease cleaves the protein sequence.

TABLE 6
Recognition sequences and cleavage sites of exemplary proteases

| Enzyme Name | Sequence and Cleavage | SEQ ID NO |
|---|---|---|
| Human Rhinovirus (HRV) 3C Protease | LEVLFQ/GP | 53 |
| Enterokinase | DDDDK/ | 54 |
| Factor Xa | IEGR/ | 55 |
| Tobacco etch virus protease | ENLYFQ/G | 56 |
| (TEV protease) | | |
| Thrombin | LVPR/GS | 57 |
| Calpain | QQEVY/GMMPRD | 58 |
| MMP2/9 | PLG/LAG or PQG/IAAQ, GPLGVRGY | 59-61 |
| | [28, 29] | |
| Urokinase | SGR/SAG or LGGSGR/SANAILEGSG | 62-63 |

Additional exemplary protease cleavage sites include caspases 1-10 and matrix metalloproteinases (MMPs), neprilysin, cathepsins, tissue plasminogen activator, plasmin, prostate specific antigen (//web.expasy.org/peptide_cutter/peptidecutter_enzymes.html) and additional proteases having one or more correspondence recognition sites identifiable by a skilled person upon reading of the present disclosure.

In particular, protease recognition sites can be identified in view of known cleavage specificities of the related protease that can be found in public databases such as Expasy at web.expasy.org/peptide_cutter/peptidecutter_enzymes.html. Table 7 provides an exemplary list of the cleavage specificities of exemplary proteases in the sense of the disclosure as will be understood by a skilled person.

TABLE 7
Exemplary list of the cleavage specificities of selected enzymes

| Enzyme Name | P4 | P3 | P2 | P1 | P1′ | P2′ |
|---|---|---|---|---|---|---|
| Arg-C proteinase | — | — | — | R | — | — |
| Asp-N endopeptidase | — | — | — | — | D | — |
| BNPS-Skatole | — | — | — | W | — | — |
| Caspase 1 | F, W, Y, or L | — | H, A or T | D | not P, E, D, | — |
| | | | | | Q, K or R | |
| Caspase 2 | D | V | A | D | not P, E, D, | — |
| | | | | | Q, K or R | |
| Caspase 3 | D | M | Q | D | not P, E, D, | — |
| | | | | | Q, K or R | |
| Caspase 4 | L | E | V | D | not P, E, D, | — |
| | | | | | Q, K or R | |
| Caspase 5 | L or W | E | H | D | — | — |
| Caspase 6 | V | E | H or I | D | not P, E, D, | — |
| | | | | | Q, K or R | |
| Caspase 7 | D | E | V | D | not P, E, D, | — |
| | | | | | Q, K or R | |
| Caspase 8 | I or L | E | T | D | not P, E, D, | — |
| | | | | | Q, K or R | |
| Caspase 9 | L | E | H | D | — | — |
| Caspase 10 | I | E | A | D | — | — |
| Chymotrypsin-high | — | — | — | F or Y | Not P | — |
| specificity (C-term to | — | — | — | W | Not M or P | — |
| [FYW], not before P) | — | — | — | F, L or Y | Not P | — |
| Chymotrypsin-low | — | — | — | W | not M or P | — |
| specificity (C-term to | — | — | — | M | not P or Y | — |
| [FYWML], not | — | — | — | H | not D, M, | — |
| before P) | | | | | P or W | |
| Clostripain | — | — | — | R | — | — |
| (Clostridiopeptidase B) | | | | | | |
| CNBr | — | — | — | M | — | — |
| Enterokinase | D or E | D or E | D or E | K | — | — |
| Factor Xa | A, F, G, I, L, | D or E | G | R | — | — |
| | T, V or M | | | | | |
| Glutamyl | — | — | — | E | — | — |
| endopeptidase | | | | | | |
| GranzymeB | I | E | P | D | — | — |
| LysC | — | — | — | K | — | — |
| Neutrophil elastase | — | — | — | A or V | — | — |
| Pepsin (pH 1.3) | — | not H, K, or R | NOT P | NOT R | F or L | Not P |
| | — | Not H, K, or R | Not P | F or L | — | Not P |
| Pepsin (pH >2) | — | Not H, K or R | Not P | Not R | F, L, W or Y | Not P |
| | | Not H, K or R | Not P | F, L, W or Y | | Not P |
| Proline- | — | — | H, K or R | P | Not P | — |
| endopeptidase | | | | | | |
| Proteinase K | — | — | — | A, E, F, I, L, | — | — |
| | | | | T, V, W or Y | | |
| Staphylococcal | — | — | NOT E | E | — | — |
| peptidase I | | | | | | |
| Thermolysin | — | — | — | Not D or E | A, F, I, L, | — |
| | | | | | M or V | |
| | — | — | G | R | G | — |
| Thrombin | A, F, G, I, L, | A, F, G, I, L, | P | R | not D or E | Not DE |
| | T, V or M | T, V, W or A | | | | |
| Trypsin | — | — | W | K | P | — |
| | — | — | M | R | P | — |

Note:
Amino acid residues in a substrate undergoing cleavage are designated P1, P2, P3, P4 etc. in the N-terminal direction from the cleaved bond. Likewise, the residues in C-terminal direction are designated P1′, P2′, P3′, P4′. etc.

In preferred embodiments, the proteases and corresponding recognition site comprise the MMP family of proteases, the Caspase family of proteases, mflon, ubiquitin, TEV, Calpain and ClpXP.

In embodiments, herein described the point of insertion and modality of insertion of the cleavage site with or without linkers is chosen such that it meets the following criteria: 1) it does not destroy the ability of GvpC to bind to the GV 2) It does not destroy the ability of GvpC to strengthen the GV shell 3) It provides the maximum difference (decrease) in GV collapse pressure before and after exposure to the protease—which can cut the inserted cleavage site/recognition motif.

Accordingly, in some embodiments, the one or more protease recognition site are preferably inserted within one or more repeats regions of the GvpC protein. More preferably the protease recognition site can be an endoprotease and/or a processive protease recognition site inserted within the second repeat in an N-terminus to C-terminus direction (see the schematic of FIG. 4C), in between repeat regions and/or after the last repeat before the C-terminal region.

In preferred embodiments, the protease recognition site (e.g. cleavage sites and/or degradation tag) are attached to the GvpC at at least one of the related N-terminus or C-terminus through a linker polypeptide.

The term “linker polypeptide” or “linker” as used herein indicates a short peptide sequences that occur between protein domains. Linkers are often composed of flexible residues such as glycine and serine so that the adjacent protein domains are free to move relative to one another.

A linker polypeptide in accordance with the disclosure can have a length that can be selected in view of the target environment and the construct where the gas vesicles of the instant disclosure are to be included and the experimental design. In particular, in the engineered gas vesicles herein described linkers are typically peptide of 2-20 residues as will be understood by a skilled person.

Exemplary linkers include GSGSGSG(SEQ ID NO: 64), GGGGS (SEQ ID NO: 65), GSGSG (SEQ ID NO: 66), GGGG (SEQ ID NO: 67), GGG(SEQ ID NO: 68), GG(SEQ ID NO 69), GS (SEQ ID NO: 70), GSGS(SEQ ID NO: 71), GGGS(SEQ ID NO: 72), GGS(SEQ ID NO: 73), GTS (SEQ ID NO: 74) GGSGGS (SEQ ID NO: 75), GGG (SEQ ID NO: 76), GGGGGG (SEQ ID NO: 77), GGGGGGGGG (SEQ ID NO: 78), GGGGGGGGGGGG (SEQ ID NO: 79), GGGGGGGGGGGGGGG (SEQ ID NO: 80), GGS(SEQ ID NO: 81), GGSGGS(SEQ ID NO: 82), GGSGGSGGS (SEQ ID NO: 83), GGSGGSGGSGGS (SEQ ID NO: 84), GGSGGSGGSGGSGGS (SEQ ID NO: 85), GSG (SEQ ID NO: 86), GSGGSG (SEQ ID NO: 87), GSGGSGGSG(SEQ ID NO: 88), GSGGSGGSGGSG (SEQ ID NO: 89), GSGGSGGSGGSGGSG (SEQ ID NO: 90), GGGGS(SEQ ID NO: 91), GGGGSGGGGS (SEQ ID NO: 92), GGGGSGGGGSGGGGS (SEQ ID NO: 93), GGGGSGGGGSGGGGSGGGGS (SEQ ID NO: 94), GGGGSGGGGSGGGGSGGGGSGGGGS (SEQ ID NO: 95) and additional polypeptide linkers identifiable by a skilled person.

In protease sensitive GvpC, at least one linker polypeptides can be used to perform an indirect attachment of the target site and/or degradation tag to the GvpC. In particular, a cleavage site can be inserted in a repeat region, by attaching two adjacent amino acids in the repeat region directly or indirectly through at least one linker attaching at the N-terminal region of the cleavage site to a first of the two adjacent amino acid and the C-terminal region of the cleavage site to the second of the two adjacent amino acid.

In some embodiments, the protease recognitions site can be engineered in the GvpC in addition or in place of sequences of the repeat regions depending on the GvpC and the configuration required to allow formation of a GV.

In some embodiments, in particular, when the protease recognition site and optionally the linker are not greater than 20 residues, the one or more protein recognition site can be inserted in the GvpC without removal of existing residues. In some examples, the one or more protein recognition site with or without linker is inserted while removing part of the internal repeat sequence such that the overall length of the modified repeat remains unchanged. In other examples, the one or more protein recognition site with or without linker is inserted while removing part of the internal repeat sequence such that the overall length of the modified repeat is changed.

In some embodiments, where the protein recognition site are attached to the N-terminus and/or the C-terminus, degrons of various kinds can be attached to either terminus of the GvpC directly or through linkers. Preferably, degrons tethered to GvpC are not larger than the GvpC itself. Preferably linkers of different length and amino acid composition are recommended to be screened according to the aforementioned method for optimal tethering of larger recognition sequences. Commonly used degrons can be tethered to GvpC with any mutations which maintain the degrons ability to be cleaved by the corresponding protease.

Exemplary GvpC that can be engineered to provide protease sensitive GvpC herein described are reported in Table 8 which shown amino acid sequences of exemplary GvpC proteins from several exemplary prokaryotic species. In particular, these exemplary amino acid sequences can be used as reference amino acid sequences in some embodiments for homology-based searches for related GvpC proteins.

TABLE 8
Protein sequences of gvpC from exemplary species:

| | UniProt | | SEQ |
|---|---|---|---|
| Species | ID No. | Amino acid Sequence | ID NO: |
| Anabaena flos- | P09413 | MISLMAKIRQEHQSIAEKVAELSLETREFLSVTTAKRQEQAEKQAQEL | 96 |
| aquae | | QAFYKDLQETSQQFLSETAQARIAQAEKQAQELLAFHKELQETSQQF | |
| | | LSATAQARIAQAEKQAQELLAFYQEVRETSQQFLSATAQARIAQAEK | |
| | | QAQELLAFHKELQETSQQFLSATADARTAQAKEQKESLLKFRQDLFV | |
| | | SIFG | |
| Halobacterium | P24574 | MSVTDKRDEMSTARDKFAESQQEFESYADEFAADI | 97 |
| salinarurn | | TAKQDDVSDLVDAITDFQAEMTNTTDAFHTYGDE | |
| | | FAAEVDHLRADIDAQRDVIREMQDAFEAYADIFAT | |
| | | DIADKQDIGNLLAAIEALRTEMNSTHGAFEAYADD | |
| | | FAADVAALRDISDLVAAIDDFQEEFIAVQDAFDNY | |
| | | AGDFDAEIDQLHAAIADQHDSFDATADAFAEYRD | |
| | | EFYRIEVEALLEAINDFQQDIGDFRAEFETTEDAFV | |
| | | AFARDFYGHEITAEEGAAEAEAEPVEADADVEAE | |
| | | AEVSPDEAGGESAGTEEEETEPAEVETAAPEVEGSP | |
| | | ADTADEAEDTEAEEETEEEAPEDMVQCRVCGEYY | |
| | | QAITEPHLQTHDMTIQEYRDEYGEDVPLRPDDKT | |
| | | MSVKDKREKMTATREEFAEVQQAFAAYADEFAA | |
| Halobacterium | Q02228 | DVDDKRDVSELVDGIDTLRTEMNSTNDAFRAYSE | 98 |
| mediterranei | | EFAADVEHFHTSVADRRDAFDAYADIFATDVAEM | |
| | | QDVSDLLAAIDDLRAEMDETHEAFDAYADAFVTD | |
| | | VATLRDVSDLLTAISELQSEFVSVQGEFNGYASEFG | |
| | | ADIDQFHAVVAEKRDGHKDVADAFLQYREEFHGV | |
| | | EVQSLLDNIAAFQREMGDYRKAFETTEEAFASFAR | |
| | | DFYGQGAAPMATPLNNAAETAVTGTETEVDIPPIE | |
| | | DSVEPDGEDEDSKADDVEAEAEVETVEMEFGAEM | |
| | | DTEADEDVQSESVREDDQFLDDETPEDMVQCLVC | |
| | | GEYYQAITEPHLQTHDMTIKKYREEYGEDVPLRPD | |
| | | DKA | |
| Microchaete | P08041 | MTPLMIRIRQEHRGIAEEVTQLFKDTQEFLSVTTAQ | 99 |
| diplosiphon | | RQAQAKEQAENLHQFHKDLEKDTEEFLTDTAKER | |
| | | MAKAKQQAEDLFQFHKEMAENTQEFLSETAKER | |
| | | MAQAQEQARQLREFHQNLEQTTNEFLADTAKERM | |
| | | AQAQEQKQQLHQFRQDLFASIFGTF | |
| Nostoc sp. | Q8YUS9 | MTALMVRIRQEHRSIAEEVTQLFRETHEFLSATTA | 100 |
| | | HRQEQAKQQAQQLHQFHQNLEQTTHEFLTETTTQ | |
| | | RVAQAEAQANFLHKFHQNLEQTTQEFLAETAKNR | |
| | | TEQAKAQSQYLQQFRKDLFASIFGTF | |

Exemplary engineering of the exemplary GvpC of Anaboena flos aquae are reported in Table 9 below.

TABLE 9
Protein sequences of gvpC from exemplary species:

| | UniProt | | SEQ |
|---|---|---|---|
| Species | ID No. | Amino acid Sequence | ID NO: |
| Anabaena flos- | | MISLMAKIRQEHQSIAEKVAELSLETREFLSVTTAK | 101 |
| aquae (TEV | | RQEQAEKQAQELQAFYKDLQETSQQFLSETAGSGS | |
| sensitive) | | GSGENLYFQGSGSGSGFHKELQETSQQFLSATAQA | |
| | | RIAQAEKQAQELLAFYQEVRETSQQFLSATAQARI | |
| | | AQAEKQAQELLAFHKELQETSQQFLSATADARTA | |
| | | QAKEQKESLLKFRQDLFVSIFG | |
| Anabaena flos- | | MGISLMAKIRQEHQSIAEKVAELSLETREFLSVTTA | 102 |
| aquae (calpain | | KRQEQAEKQAQELQAFYKDLQETSQGSGSGQQEV | |
| sensitive) | | YGMMPRDGSGSGQAQELLAFHKELQETSQQFLSA | |
| | | TAQARIAQAEKQAQELLAFYQEVRETSQQFLSATA | |
| | | QARIAQAEKQAQELLAFHKELQETSQQFLSATADA | |
| | | RTAQAKEQKESLLKFRQDLFVSIFG | |
| Anabaena flos- | | MG [symbol] SGISLMAKIRQEHQSIAEKVAELSLET | 2 |
| aquae (ClpXP | | REFLSVTTAKRQEQAEKQAQELQAFYKDLQETSQ | |
| sensitive) | | QFLSETAQARIAQAEKQAQELLAFHKELQETSQQF | |
| | | LSATAQARIAQAEKQAQELLAFYQEVRETSQQFLS | |
| | | ATAQARIAQAEKQAQELLAFHKELQETSQQFLSAT | |
| | | ADARTAQAKEQKESLLKFRQDLFVSIFGSGAANDE | |
| | | NYALAA | |

Underlined fonts: inserted sequence; bold fonts: protease recognition sequences; italic fonts: linker; bold italic fonts: tag sequence

A representation of the exemplary GvpC of Table 9 also showing the N-terminal region, C-terminal region and repeats is provided in Tables 10-11 below.

TABLE 10
GvpC in engineered Anabaena flos-aquae GvpC (TEV-sensitive)

| Position | Sequence | SEQ ID NO |
|---|---|---|
| N-terminus | MISLMAKIRQEHQSIAEK | 103 |
| Repeat 1 | VAELSLETREFLSVTTAKRQEQAEKQAQELQAF | 104 |
| Repeat 2 | YKDLQETSQQFLSETAGSGSGSGENLYFQGSGSGSGF | 105 |
| Repeat 3 | HKELQETSQQFLSATAQARIAQAEKQAQELLAF | 106 |
| Repeat 4 | YQEVRETSQQFLSATAQARIAQAEKQAQELLAF | 107 |
| Repeat 5 | HKELQETSQQFLSATADARTAQAKEQKESLLKF | 108 |
| C-terminus | RQDLFVSIFG | 109 |

ENLYFQ/G-TEV recognition sequence; GSGSGSG-linker

TABLE 11
Sequence alignment of the repeat sequences of GvpC in engineered
Anabaena flos-aquae GvpC (calpain-sensitive)

| Position | Sequence | SEQ ID NO |
|---|---|---|
| N-terminus | MISLMAKIRQEHQSIAEK | 110 |
| Repeat 1 | VAELSLETREFLSVTTAKRQEQAEKQAQELQAF | 111 |
| Repeat 2 | YKDLQETSQGSGSGQQEVYGMMPRDGSGSGQAQELLAF | 112 |
| Repeat 3 | HKELQETSQQFLSATAQARIAQAEKQAQELLAF | 113 |
| Repeat 4 | YQEVRETSQQFLSATAQARIAQAEKQAQELLAF | 114 |
| Repeat 5 | HKELQETSQQFLSATADARTAQAKEQKESLLKF | 115 |
| C-terminus | RQDLFVSIFG | 116 |

QQEVY/GMMPRD-Calpain recognition sequence; GSGSG-linker

In some embodiments, engineered protease sensitive GvpC can be further engineered to include additional tags or labels (e.g. reporter protein GFP) to confirm aspects such as protein translocation or other functions with techniques such as the ones described in U.S. application Ser. No. 15/663,635 published as US 2018/0030501 incorporated herein by reference in its entirety.

An exemplary engineered protease sensitive GvpC further comprising a HIS tag is reported in Table 12 below.

TABLE 12
GvpC in engineered Anabaena flos-aquae GvpC (ClpXP-sensitive)

| Position | Sequence | SEQ ID NO |
|---|---|---|
| N-terminus | MGHHHHHHSGISLMAKIRQEHQSIAEK | 117 |
| Repeat 1 | VAELSLETREFLSVTTAKRQEQAEKQAQELQAF | 118 |
| Repeat 2 | YKDLQETSQQFLSETAQARIAQAEKQAQELLAF | 119 |
| Repeat 3 | HKELQETSQQFLSATAQARIAQAEKQAQELLAF | 120 |
| Repeat 4 | YQEVRETSQQFLSATAQARIAQAEKQAQELLAF | 121 |
| Repeat 5 | HKELQETSQQFLSATADARTAQAKEQKESLLKF | 122 |
| C-terminus | RQDLFVSIFGSGAANDENYALAA | 123 |

AANDENYALAA-ClpXP degron/ClpXP recognition sequence; SG-linker; HHHHHH-Histidine tag for purification

In general, protease sensitive GvpC can be provided with methods and techniques such as the ones indicated in U.S. application Ser. No. 15/663,635 published as US 2018/0030501 incorporated herein by reference in its entirety in in U.S. application Ser. No. 16/736,683 filed on Jan. 7, 2020 and in PCT/US2020/012572 filed on Jan. 7, 2020 published as WO/2020/146379 each incorporated herein by reference in its entirety as well as additional techniques known and identifiable by a skilled person.

In some embodiments, the engineered GvpC variants are obtained by further linking the native GvpC protein to one or more proteases and/or liner polypeptides herein described to form a recombinant fusion protein.

Recombinant fusion proteins can be created artificially using recombinant DNA technology identifiable by a person skilled in the art of molecular biology. In general, the methods for producing recombinant fusion proteins comprise removing the stop codon from a cDNA or genomic sequence, such as a polynucleotide coding for a GvpC protein or a derivative thereof, then appending the cDNA or genomic sequence of the second protein in frame through ligation or overlap extension PCR. Optionally, PCR primers can further encode a linker of one or more amino acids residues and/or a PCR primer-encoded protease cleavage site placed between two proteins, polypeptides, or domains or parts thereof. The resulting DNA sequence will then be expressed by a prokaryotic cell as a single protein. A fusion protein can also comprise a linker of one or more amino acids residues, which can enable the proteins to fold independently and retain functions of the original separate proteins or polypeptides or domains or parts thereof. Linkers in protein or peptide fusions can be engineered with protease cleavage sites that can enable the separation of one or more proteins, polypeptides, domains or parts thereof from the rest of the fusion protein. Other methods for genetically engineering these recombinant fusion proteins include Site Directed Mutagenesis (e.g. using Q5 Site-Directed Mutagenesis Kit from NEB or the QuickChange Lightning Kit from Agilent), Gibson Assembly (e.g. using the NEB Hi-Fi DNA Assembly Kit), Error-prone PCR (e.g. Mutazyme from Agilent) and Golden-Gate assembly (e.g. using the NEB Golden Gate Assembly Mix).

Accordingly, an engineered gene encoding for a GvpC herein described can be used to provide the protease sensitive GvpC e.g. through inclusion in a suitable gene expression cassette and its use for expression in a host cell, e.g. a prokaryotic cell.

A “gene expression cassette” is a gene cassette comprising regulatory sequence to be expressed by a transfected cell. Following transformation, the expression cassette directs the cell's machinery to make RNA and proteins. Some expression cassettes are designed for modular cloning of protein-encoding sequences so that the same cassette can easily be altered to make different proteins. An expression cassette is composed of one or more genes and the sequences controlling their expression. An expression cassette typically comprises at least three components: a promoter sequence, an open reading frame, and a 3′ untranslated region that, in eukaryotes, usually contains a polyadenylation site. An expression cassette can be formed by manipulable fragment of DNA carrying, and capable of expressing, one or more genes of interest optionally located between one or more sets of restriction sites Gene expression cassettes as used herein typically comprise further regulatory sequences additional to the prompter to regulated the expression of the gene or genes within the open reading frame herein also indicated as coding region of the cassette.

In some embodiments, a protease sensitive GvpC can be produced by engineering a gvpC protein from any species that encodes a gvpC protein in its genome, or a synthetically designed gvpC protein. In some embodiments, the gvpC protein is a gvpC protein from Anabaena flos-aquae, Halobacterium salinarum, Halobacterium mediterranei, Microchaete diplosiphon or Nostoc sp., or homologs thereof, and others identifiable by a skilled person.

In some embodiments, addition of a degradation tag can be performed an in-frame insertion or C- or N-terminal fusion of the degradation tag to a gvpC. An in-frame insertion can be performed in several steps, by first providing the gvpC-coding and the degradation tag-coding polynucleotides and performing the insertion by breaking a bond (typically a phosphodiester bond) between two adjacent nucleotide bases of the first polynucleotide and then forming new bonds between the gvpC-coding polynucleotide and the degradation tag-coding polynucleotide. For example, the gvpC coding polynucleotide can be digested with one or more restriction endonucleases and then the degradation tag-coding polynucleotide inserted by ligation (e.g., using T7 DNA ligase) into compatible site(s) allowing formation of phosphodiester bonds between the first and second polynucleotide bases. Compatible DNA ligation sites can be “sticky” ends, digested with restriction endonuclease producing an overhang (e.g. EcoRI), or can be “blunt ends” with no overhang, as would be understood by those skilled in the art. A fusion of a polynucleotide encoding a tag can also be ligated to an N- or C-terminus of a gvpC or a variant gvpC polynucleotide by ligation (e.g., using T7 DNA ligase) into compatible site(s).

In some embodiments, the gvpC- and the degradation tag-coding polynucleotides can be provided within a single polynucleotide by design. For example, a tag can be added by inserting the polynucleotide encoding a protein of interest in a plasmid or vector that has the tag ready to fuse at the N-terminus or C-terminus. The tag can be added using PCR primers encoding the degradation tag; using PCR the tag can be fused to the N-terminus or C-terminus of the protein-coding polynucleotide, or can be inserted at an internal location, using internal epitope tagging (see e.g. ref [30]), among other methods known to those skilled in the art. Other methods such as overlap extension PCR and infusion HD cloning can be used to insert a tag at a site between the N-terminus and C-terminus of a protein-coding polynucleotide (see Examples of U.S. application Ser. No. 15/613,104). Optionally, a polynucleotide encoding a ‘linker’ (such as a sequence encoding a short polypeptide or protein sequence, e.g., gly-gly-gly or gly-ser-gly can be placed between the protein of interest and the tag; this can be useful to prevent the tag from affecting the activity of the protein being tagged.

In embodiments herein described, the Gas Vesicle that is engineered can be a naturally occurring gas vesicle formed by naturally occurring Gv proteins or can be a hybrid gas vesicle formed by Gv proteins from different naturally occurring GVs. In embodiments herein described, the GV can be engineered so that a protease sensitive GvpC can be included in a GV in place of an existing GvpC of the cluster or in addition to GVA and GVS of clusters which do not include GvpCs. The location and configuration of the cleavage site and/or degradation tag in engineered protease sensitive GvpC of the present disclosure results in inclusion of the GvpC in the formed GV in a configuration in which the at least one endoprotease cleavage site and/or the exoprotease specific protease recognition sites are presented on the protein shell of the engineered protease sensitive gas vesicle as will be understood by a skilled person.

The term “present” as used herein with reference to a compound or moiety indicates attachment performed to maintain the chemical reactivity of the compound or moiety as attached. Accordingly, a cleavage site or degradation tag presented on an GV shell, is able to perform under the appropriate conditions the one or more chemical reactions that chemically characterize the cleavage site or degradation which here comprise at least ability to be cleaved by a protease in the sense of the disclosure.

In some embodiments, addition of the GvpC can be performed at a protein level by isolating a GV, removing a GvpC (e.g. through urea stripping) and performing GvpC re-addition as described in [31, 32] (see also Examples section). In some other embodiments, GvpC can be added to a formed GV that does not have a GvpC (e.g. Mega GV) through similar process without the step of removing a GvpC.

In some embodiments, a protease sensitive GV can be provided by providing an engineered protease sensitive GVGC, in which the GVS genes of the protease sensitive GVGC comprise a genetically engineered protease sensitive gvpC gene encoding for a protease sensitive GvpC protein of the instant disclosure.

Accordingly, in embodiments herein described, GVGC can be selected based on desired properties of the corresponding GV type. In particular, to this extent, a skilled person can use naturally occurring GVGC, can provide engineered GVGC wherein some of the naturally occurring gyp genes are omitted, and/or can provide hybrid GVGC in which GVAs and GVS genes of naturally occurring GVGCs are combined to provide GV types having the shape and dimensions resulting in the desired properties in particular acoustic properties as will be understood by a skilled person upon reading of the present disclosure.

Several detectable GVGC with one or more detection method of interests have been identified and can be used for production of GV types in various cells through various genetically engineered constructs as will be understood by a skilled person upon reading of the present disclosure and U.S. application Ser. No. 15/663,635 published as US 2018/0030501 herein incorporated by reference in its entirety.

In some embodiments described herein GVGC of the instant disclosure can be naturally occurring combination of gyp genes which can have a naturally occurring sequence or a sequence modified to optimize the expression in the cell where detection is to be performed. For example GVGC clusters of the instant disclosure comprise a GVGC of B. megaterium formed by the gvpA or gvpB genes, gvpR, gvpN gvpF, gvpG, gvpL gvpS, gvpK, gvpJ, gvpT, gvpU of B. megaterium, or the GVGC of Anaboena Flos Aquae formed by the gvpA or gvpB genes of Anaboena Flos Aquae (see e.g. the sequences in Table 14 of Example 9) and the GVA gvpC, gvpN, gvpJ, gvpK, gvpF, gvpG, gvpV, gvpW of Anaboena Flos Aquae (see e.g. sequences in Table 18 of Example 9).

The gyp genes in one or more genes of the GVGC cluster of the present disclosure can have a naturally occurring sequence or a sequence modified to optimize the expression in the cell where detection is to be performed. For example a B. megaterium GVGC can have a gvpA or gvpB genes having the sequences in Table 14 of Example 9, and/or any one of the gvpR, gvpN gvpF, gvpG, gvpL gvpS, gvpK, gvpJ, gvpT, gvpU genes having the sequences in Table 16 of Example 9. Similarly, an Anaboena Flos Aquae GVGC can have the gvpA or gvpB genes having the sequences reported in Table 14 of Example 9 and/or any one of the gvpC, gvpN, gvpJ, gvpK, gvpF, gvpG, gvpV, gvpW having the. sequences reported in Table 18 of Example 9.

In some embodiments, described herein, GVGC of the instant disclosure can be engineered protease sensitive version of naturally occurring GV gene clusters. An example is provided by the GVGC of B. megaterium comprising gvpB, gvpR, gvpN gvpF, gvpG, gvpL gvpS, gvpK, gvpJ, gvpT, gvpU wherein the gvpR and gvpT genes of the naturally occurring GVGC from B. megaterium have been omitted (see e.g. the sequences reported in Example 6 of the co-pending U.S. application Ser. No. 16/736,683 and Example 3 of the instant disclosure). Another example is provided by GV gene clusters comprising gvpA, Ana-gvpC gvpN, gpvJ, gvpK, gvpF, gvpG, gvpW, and gvpV from Anabaena flos-aquae or GV gene clusters comprising gvpA+gvpN, gpvJ, gvpK, gvpF, gvpG, gvpW, gvpV from Anabaena flos-aquae (see Anabaena flos-aquae genes in Table 18 of Example 9 of the present disclosure).

In other embodiments described herein, GVGC of the instant disclosure can be modified protease sensitive variant of a hybrid GV gene cluster in a Gas Vesicle expression system of the disclosure, can comprise a combination of genes from A. flos-aquae (herein also Ana-gyp) and genes from B. megaterium (herein also Mega-gyp). In particular, in exemplary embodiments, the hybrid GV gene cluster can comprise B. megaterium GVA assembly genes gvpR, gvpN, gvpF, gvpG, gvpL, gvpS, gvpK, gvpJ, gvpT and gvpU and further comprise structural gvpA gene from Anabaena flos-aquae. In some of those embodiments, the hybrid GV gene cluster can comprise gvpA, gvpC from Anabaena flos-aquae and GVA genes from B. megaterium possibly excluding gvpR and/or gvpT. In some of those embodiments, the hybrid GV gene cluster can comprise Ana-gvpA and mega GVA genes possibly excluding gvpR and/or gvpT. In some embodiments GVGC of the instant disclosure can include gvpA, gvpC, gvpN from Anabaena flos-aquae and GVA genes from B. megaterium, as well as other combinations identifiable by a skilled person upon reading of the present disclosure.

In some embodiments herein described, a GVGC comprising gyp genes A/B, C and N (gvpA/B, gvpC, gvpN genes) from a same or different prokaryote. Preferably the GVGC comprises a gvpN gene as presence of gvpN protein results in an increased detectability of the related GV type.

For example, in one exemplary embodiment, all the gyp genes B, N, F, G, L, S, K, J and U are from B. megaterium. GVs from B. megaterium are typically cone-tipped cylindrical structures with a diameter of approximately 73 nm and length of 100-600 nm, encoded by a cluster of eleven or fourteen different genes, including the primary structural protein, gvpB, and several putative minor components and putative chaperones [33, 34] as would be understood by a person skilled in the art.

In some embodiments, some of the set of nine gyp genes can be from Bacillus megaterium and the rest genes are from Anabaena flos-aquae such as the GVGC comprising Ana-A, Ana-C, Ana-N, mega: gvpF, gvpG, gvpL, gvpS, gvpK, gvpJ, gvpT and gvpU with/without gvpR and gvpT, and additional examples identifiable by a skilled person upon reading of the present disclosure (see Example 9 of the present disclosure and Example 5 of the co-pending U.S. application Ser. No. 16/736,683).

In some embodiments, the protease sensitive GVGC can comprise Serratia gyp genes as Serratia GVs can express functional GV proteins in E. coli, as would be understood by a skilled person (see [35] [36]).

In some embodiments, the protease sensitive GVGC is gvpA, gvpC, gvpN, gvpJ, gvpK, gvpF, gvpG, gvpV, gvpW from Anabaena flos-aquae, in which the gvpC has been engineered to be protease sensitive.

In some embodiments, the protease sensitive GVGC is a hybrid gas vesicle gene cluster comprising gvpR, gvpN, gvpF, gvpG, gvpL, gvpS, gvpK, gvpJ, gvpT and gvpU from B. megaterium and gvpA and gvpC gene from Anabaena flos-aquae in which the gvpC has been engineered to be protease sensitive.

In some embodiments, the protease sensitive GVGC is a hybrid gas vesicle gene cluster comprising -gvpA, and gvpC from Anabaena flos-aquae, and gvpN, gvpF, gvpG, gvpL, gvpS, gvpK, gvpJ, and gvpU from B. megaterium in which the gvpC has been engineered to be protease sensitive.

In some embodiments, the protease sensitive GVGC is a hybrid gas vesicle gene cluster comprising—gvpA, gvpC and gvpN from Anabaena flos-aquae, gvpF, gvpG, gvpL, gvpS, gvpK, gvpJ, and gvpU from B. megaterium in which the gvpC has been engineered to be protease sensitive.

In embodiments herein described protease sensitive GvpC and/or GVs and related GVGC can be expressed in a host cell as will be understood by a skilled person.

The term “cell” as described herein indicates basic structural, functional, and biological unit of all known organisms. A cell consists of cytoplasm enclosed within a membrane, which contains biomolecules such as proteins and nucleic acids. Cells are two types: prokaryotic and eukaryotic.

The term “prokaryotic cell” used herein refers to a microbial species which contains no nucleus or other organelles in the cell, which includes but is not limited to Bacteria and Archaea.

The term “bacteria” as used herein refers to several prokaryotic microbial species which include but are not limited to Gram-positive bacteria, Proteobacteria, Cyanobacteria, Spirochetes and related species, Planctomyces, Bacteroides, Flavobacteria, Chlamydia, Green sulfur bacteria, Green non-sulfur bacteria including anaerobic phototrophs, Radioresistant micrococci and related species, Thermotoga and Thermosipho thermophiles. More specifically, the wording “Gram positive bacteria” refers to cocci, nonsporulating rods and sporulating rods, such as, for example, Actinomyces, Bacillus, Clostridium, Corynebacterium, Erysipelothrix, Lactobacillus, Listeria, Mycobacterium, Myxococcus, Nocardia, Staphylococcus, Streptococcus and Streptomyces. The term “Proteobacteria” refers to purple photosynthetic and non-photosynthetic gram-negative bacteria, including cocci, nonenteric rods and enteric rods, such as, for example, Neisseria, Spirillum, Pasteurella, Brucella, Yersinia, Francisella, Haemophilus, Bordetella, Escherichia, Salmonella, Shigella, Klebsiella, Proteus, Pseudomonas, Bacteroides, Acetobacter, Aerobacter, Agrobacterium, Azotobacter, Spirilla, Serratia, Vibrio, Rhizobium, Chlamydia, Rickettsia, Treponema and Fusobacterium. Cyanobacteria, e.g., oxygenic phototrophs.

The term “Archaea” as used herein refers to prokaryotic microbial species of the division Mendosicutes, such as Crenarchaeota and Euryarchaeota, and include but is not limited to methanogens (prokaryotes that produce methane); extreme halophiles (prokaryotes that live at very high concentrations of salt (NaCl); and extreme (hyper) thermophiles (prokaryotes that live at very high temperatures).

In some embodiments the prokaryotic host is a bacteria and in particular a Gram Negative Bacteria. As understood by those skilled in the art, Gram-negative bacteria are a group of bacteria that do not retain the crystal violet stain used in the Gram staining method of bacterial differentiation. They are characterized by their cell envelopes, which are composed of a thin peptidoglycan cell wall sandwiched between an inner cytoplasmic cell membrane and a bacterial outer membrane.

Exemplary Gram-negative bacteria that can be genetically engineered with GVGC genetic circuits described herein configured to allow heterologous expression of GVs comprise E. coli, Nissle 1997, Salmonella, and others identifiable by those skilled in the art.

The term “eukaryotic cell” refers to cells that contain a nucleus and organelles and are enclosed by a plasma membrane as will be understood by a person skilled in the art. Organisms that have eukaryotic cells include protozoa, fungi, plants, and animals.

The term “mammalian cell” are a type of eukaryotic cells that refer to cells from a mammal tissue comprising cell within a mammal host and cell isolated from and expanded in culture for use as therapeutic and research tools. Exemplary mammalian cells that can express GVES of the disclosure are primary cells (cells that are directly harvested from an animal and genetically engineered with GVs. Exemplary mammalian cell culture that can be genetically engineered with GV constructs described herein configured to allow expression of GVs comprise HEK 293T, CHO-K1 cells, HEK293, CHO-K1, N2A cells, HeLa, Jurkat, NIH3T3, and other identifiable by those skilled in the art.

In particular, in embodiments herein described, a protease sensitive GvpC and/or a GVGC herein described can be expressed in a host cell through gene expression casssettes of a protease sensitive Gas Vesicle Expression System (GVES) wherein in the gene expressions cassettes the GvpC protein are comprised under control of a promoter and additional regulatory regions in a configuration allowing expression in the host cell.

The term “regulatory sequence” or “regulatory regions” as described herein indicate a segment of a nucleic acid molecule which is capable of increasing or decreasing transcription or translation of a gene within an organism either in vitro or in vivo. In particular, coding regions of the GV genes herein described comprise one or more protein coding regions which when transcribed and translated produce a polypeptide. Regulatory regions of a gene herein described comprise promoters, transcription factor binding sites, operators, activator binding sites, repressor binding sites, enhancers, protein-protein binding domains, RNA binding domains, DNA binding domains, silencers, insulators and additional regulatory regions that can alter gene expression in response to developmental and/or external stimuli as will be recognized by a person skilled in the art.

The term “operative connection” as used herein indicate an arrangement of elements in a combination enabling production of an appropriate effect. With respect to genes and regulatory sequences an operative connection indicates a configuration of the genes with respect to the regulatory sequence allowing the regulatory sequences to directly or indirectly increase or decrease transcription or translation of the genes.

Regulatory sequences used in gene expression cassettes herein described identified herein also as mammalian regulatory regions are configured to operate in a mammalian cell.

Exemplary regulatory regions capable of operating in mammalian cells comprise promoters, enhancers, silencers, terminators, regulators, operators, ribosome binding/entry sites, and riboswitches, among others known in the art. Regulatory regions capable of operating in a mammalian host can be selected by a skilled person following selection of the mammalian host of interest. Exemplary constitutive and inducible mammalian promoters and operators suitable for regulating expression of GVs in a mammalian host comprise and others identifiable by those skilled in the art and described herein.

Mammalian regulatory regions comprised in a gene expression cassette herein described, typically comprise a mammalian promoter, 5′UTR regions, 3′UTR regions, and a terminator as will be understood by a skilled person.

A “mammalian promoter” in the sense of the disclosure suitable for gene expression in a mammalian cell is a region of DNA that leads to initiation of transcription of a particular gene. Exemplary are typically located on a same strand and upstream on a DNA sequence (towards the 5′ region of the sense strand), adjacent to the transcription start site of the genes whose transcription they initiate. In mammalian cells organisms, promoters typically comprise the eukaryotic TATA (SEQ ID NO: 124) box. Promoters are located near the transcription start sties of genes, upstream on the DNA. Promoters can typically be about 100-1000 base pairs long. In particular promoters that can be used in gene expression cassette herein described can be a constitutive promoter or a conditional promoter.

The term “conditional promoter” refers to a promoter with activity regulatable or controlled by endogenous transcription factors or exogenous inputs such as chemical, or thermal inducers or optical induction. Examples of mammalian constitutive promoters include inducible promoters based on exogenous agents such as TET (tetracycline-response elements, TET-ON/TET-OFF), Lac, dCas-transactivator, Zinc-finger-TF, TALENs-ZF Gal4-uas, synNotch and inducible promoters based on endogenous signals TNF-alpha, cFOS and others identifiable to a skilled person.

The term “constitutive promoter” refers to an unregulated promoter that allows for continual transcription of its associated genes. Exemplary mammalian constitutive promoters that can be used for expression in mammalian cell include CMV from human cytomegalovirus, EF1a from human elongation factor 1 alpha, SV40 from the simian vacuolating virus 40, PGK1 from phosphoglycerate kinase gene, Ubc from human ubiquitin C gene, human beta actin, CAAG, Syn1 and others identifiable to those skilled in the art.

The wording “5′UTR region” refers to the region upstream from the initiation codon as will be understood by a person of ordinary skill in the art and is therefore outside the coding region of the cassette. The 5′UTR region can contain a Kozak sequence. The Kozak sequence used herein refers to a nucleic acid motif that functions as the protein translation initiation site in most eukaryotic mRNA transcripts as will be understood by a person skilled in the art. The Kozak sequence locates approximately 6 nucleotide sequence upstream of the ATG start codon. Exemplary Kozak sequence include GCCACCATG (SEQ ID NO: 125), TTCACCATG (SEQ ID NO: 126), (CCC)TTCACCATG (SEQ ID NO: 127) consensus sequence XXX[A/G]XXATG (SEQ ID NO: 128) wherein X indicates any nucleotide, and additional sequences identifiable by a skilled person.

The “3′ UTR region” refers to an untranslated region that immediately follows the translation termination codon and is therefore outside the coding region of the cassette. 3′UTR region often contains regulatory regions that post-transcriptionally influence gene expression. Regulatory regions within the 3′UTR can influence polyadenylation, translation efficiency, localization, and stability of the mRNA as will be understood by a person skilled in the art. In some embodiments, the 3′UTR contains silencer regions which are configured to bind to repressor proteins and inhibit the expression of the mRNA.

A “terminator” as used herein indicates a sequence-based element that defines the end of a transcriptional unit and initiates the process of releasing the synthesized mRNA. Exemplary mammalian terminators include polyadenylation sites. A “polyadenylation site” indicates an element target by the polyadenylation enzymes such as CPSF and typically comprises the sequence AAUAAA (SEQ ID NO: 129) on the RNA. Polyadenylation sites will result in cleavage of the construct 10-30 nucleotides downstream the site, and addition of a poly(A) tail located at the end of 3′UTR as will be understood by a person skilled in the art. In gene expression cassette the poly(A) site can include SV40 polyadenylation element, hGH poly(A) signal, and other poly(A) signal that have the canonical AAUAAA (SEQ ID NO: 129) region as will be understood by a skilled person.

In some embodiments, a gene expression cassette can include additional mammalian regulatory regions configured to increase or decrease the expression of the GV coding regions of the cassette, as will also be understood by a skilled person.

Exemplary mammalian regulatory sequences increasing transcription of the operatively linked gene comprise enhancers that can be located more distally from the transcription start site compared to promoters, and either upstream or downstream from the regulated genes, as understood by those skilled in the art. Enhancers are typically short (50-1500 bp) regions of DNA that can be bound by transcriptional activators to increase transcription of a particular gene. Typically, enhancers can be located up to 1 Mbp away from the gene, upstream or downstream from the start site. An exemplary additional mammalian regulatory regions directed to enhance the expression levels of the GV genes, include Woodchuck Hepatitis Virus Posttranscriptional Regulatory Element (WPRE) placed downstream of the genes between GV gene and the poly(A) tail. The WPRE and WPRE-like (e.g. RE of Hepatitis B virus (HPRE)) element is known to increase transgene expression from a variety of viral vectors.

Exemplary mammalian regulatory sequences decreasing transcription of the operatively linked gene comprise RNAi/miRNA/shRNA sites that can be located upstream or downstream of the GV genes to control mRNA translation or degradation. For example, by binding to specific sites within the 3′UTR, miRNAs can decrease gene expression of various mRNAs by either inhibiting translation or directly causing degradation of the transcript.

Additional mammalian regulatory sequences that can be included in a gene expression cassette include post transcriptional regulatory sequences such as riboswitches typically present in eukaryotic untranslated regions (UTRs) of encoded RNAs. These sequences are configured to switch between alternative secondary structures in the RNA depending on the concentration of key metabolites. The secondary structures then either block or reveal other regulatory sequence regions such as RNA binding proteins. A further examples of additional post transcriptional regulatory sequences regulatory sequences comprise aptazymes fusions composed of an aptamer domain and a self-cleaving ribozyme which can be used for conditional gene expression to control mRNA levels with small molecules (e.g. tetracycline).

In general, selection of promoter and other regulatory sequences to be included in expression polynucleotidic constructs comprised in GVES of the present disclosure can be performed by one or more of the following: detecting functionality of a promoter and/or additional regulatory sequence in the host cells, selecting promoters and/or additional regulatory sequences known to be functional in the host cells; detecting the strength of the promoters and/or additional regulatory sequences in connection with protein production and/or selecting promoter and/or additional regulatory sequences of known strength; and selecting inducible promoters and/or additional regulatory sequence to control GV expression.

Mammalian regulatory sequences can be provided in any configuration which is directed to provide a desired expression of the GV protein in the coding regions. For example, a gene expression cassette can an end of UTR with polyA site only, or can be with WPRE and polyA site, or it can be with WPRE only. A combination of WPRE and polyA tail is expected to result in highest expression (highest copy of translated protein). Additional configuration can be identified by a skilled person.

In some embodiments herein described, the sequences of at least one gyp gene can be modified with respect to the natural occurring sequence to improve the related expression (e.g. to be codon optimized) and/or the inclusion in the GVES of the disclosure (e.g. by modification of the N- and/or C-terminal portions to allow the use of linker or other elements to be included in a cassette or construct of the disclosure).

Exemplary GVES are described in U.S. application Ser. No. 16/736,683 filed on Jan. 7, 2020 and in PCT/US2020/012572 filed on Jan. 7, 2020 published as WO/2020/146379 each incorporated herein by reference in its entirety.

Protease sensitive GvpC gene expression cassettes alone or in combination with other protease sensitive GVES component can be used to express a protease sensitive Gas Vesicles in a host cell herein described. The related method comprises introducing into the cell the protease sensitive gvpC gene expression cassette herein described configured for expression in the host cell, the introducing performed to allow expression of the protease sensitive GvpC herein described in the host cell in combination with other genes of a protease sensitive Gas Vesicle expression system (GVES) of the present disclosure under control of a promoter and additional regulatory regions allowing expression of the protease sensitive GVGC in the host cell and production of a protease sensitive gas vesicle type in the host cell.

The introducing can be performed with a vector is described comprising at least one of the protease sensitive gvpC gene expression cassette and protease sensitive Gas Vesicle expression system herein described.

Accordingly, in some embodiments, a vector comprising one or more GV genes encoding for GV proteins possibly with gene expression cassettes, wherein the vector is configured to introduce the one or more GV genes into a cell or eukaryotic cell such as a mammalian cell.

The term “vector” indicates a molecule configured to be used as a vehicle to artificially carry foreign genetic material into a cell, where it can be replicated and/or expressed. An expression vector is configured to carry and express the material in a cell under appropriate conditions. In some embodiments, a suitable vector can comprise a recombinant plasmid, a recombinant non-viral vector, or a recombinant viral vector. Vectors described herein can comprise suitable promoters, enhancers, post-transcriptional and post-translational elements for expression in mammalian that are identifiable by those skilled in the art.

Vectors suitable for transduction of prokaryotic cells, and in particular various Gram-negative bacterial cell types are known to those skilled in the art. In exemplary embodiments herein described, bacterial expression plasmids contain all the necessary components to allow cloning methods using E. coli, and comprise elements such as a bacterial origin of replication (ORI) and elements for plasmid maintenance such as antibiotic selection markers and toxin-antitoxin systems, and also optionally to allow incorporating the genes into the bacterial genome using recombinases such as Lambda Red, and others identifiable by those skilled in the art.

Exemplary vectors for bacterial transformation of E. coli and S. typhimurium with genetic molecular components comprising GV gene clusters are described herein in the Examples of U.S. application Ser. No. 15/663,635.

Vectors suitable for transduction of mammalian cells, are known to those skilled in the art. Exemplary vectors for transformation of a mammalian cell with genetic molecular components comprising GV gene clusters are described herein in the Examples of U.S. application Ser. No. 16/736,683.

Accordingly, a system to express a protease sensitive Gas Vesicles in a host cell with methods herein described can therefore comprises a protease sensitive gvpC gene expression cassette herein described, a e genetically engineered protease sensitive Gas Vesicle expression system (GVES), possibly within one or more vectors, and/or host cells for simultaneous combined or sequential use in the method to express a protease sensitive gas vesicle herein described.

In some embodiments at least one of the protease sensitive gas vesicle protein GvpC, the protease sensitive gas vesicle, the protease sensitive Gas Vesicle Gene Cluster (GVGC), the protease sensitive gvpC gene expression cassette, protease sensitive Gas Vesicle expression system, the vector and the cell described herein, can be comprised within a composition together with a suitable vehicle.

The term “vehicle” as used herein indicates any of various media acting usually as solvents, carriers, binders or diluents for the one or more genetic molecular components, vectors, or cells herein described that are comprised in the composition as an active ingredient. In particular, the composition including the one or more genetic molecular components, vectors, or cells herein described can be used in one of the methods or systems herein described.

In embodiments herein described, the protease sensitive Gas Vesicles herein described can be used together with contrast-enhanced imaging techniques to detect and report a protease sensitive biological event the location of and/or biochemical events in genetically engineered host cells in an imaging target site.

The term “contrast enhanced imaging” or “imaging”, as used herein indicates a visualization of a target site performed with the aid of a contrast agent present in the target site, wherein the contrast agent is configured to improve the visibility of structures or fluids by devices process and techniques suitable to provide a visual representation of a target site. Accordingly a contrast agent is a substance that enhances the contrast of structures or fluids within the target site, producing a higher contrast image for evaluation. In particular, as used herein, the term “contrast agent” refers to GVs expressed in prokaryotic cells comprised in the target site, the GVs comprised in GVGC genetic circuits in the prokaryotic cells when the GVGC genetic circuit operates according to a circuit design in response to a biochemical event, as described herein.

The term “target site” as used herein indicates an environment comprising one or more targets intended as a combination of structures and fluids to be contrasted, such as cells. In particular the term “target site” refers to biological environments such as cells, tissues, organs in vitro in vivo or ex vivo that contain at least one target. A target is a portion of the target site to be contrasted against the background (e.g. surrounding matter) of the target site. Accordingly, as used herein a target comprises one or more prokaryotic cells genetically engineered to comprise one or more GVGC genetic circuits as described herein within any suitable environment in vitro, in vivo or ex vivo as will be understood by a skilled person. Exemplary target sites include collections of microorganisms, including, bacteria or archaea in a solution or other medium in vitro, as well as cells grown in an in vitro culture, including, primary mammalian, cells, immortalized cell lines, tumor cells, stem cells, and the like. Additional exemplary target sites include tissues and organs in an ex vivo culture and tissue, organs, or organ systems in a subject, for example, lungs, brain, kidney, liver, heart, the central nervous system, the peripheral nervous system, the gastrointestinal system, the circulatory system, the immune system, the skeletal system, the sensory system, within a body of an individual and additional environments identifiable by a skilled person. The term “individual” or “subject” or “patient” as used herein in the context of imaging includes a single plant, fungus or animal and in particular higher plants or animals and in particular vertebrates such as mammals and more particularly human beings.

In embodiments herein described, imaging the target site and/or the host cell can be performed by applying ultrasound to obtain an ultrasound image of the target site.

The term “ultrasound imaging” or “ultrasound scanning” or “sonography” as used herein indicate imaging performed with techniques based on the application of ultrasound. Ultrasound refers to sound with frequencies higher than the audible limits of human beings, typically over 20 kHz. Ultrasound devices typically can range up to the gigahertz range of frequencies, with most medical ultrasound devices operating in the 1 to 18 MHz range. The amplitude of the waves relates to the intensity of the ultrasound, which in turn relates to the pressure created by the ultrasound waves. Applying ultrasound can be accomplished, for example, by sending strong, short electrical pulses to a piezoelectric transducer directed at the target. Ultrasound can be applied as a continuous wave, or as wave pulses as will be understood by a skilled person.

Accordingly, the wording “ultrasound imaging” as used herein refers in particular to the use of high frequency sound waves, typically broadband waves in the megahertz range, to image structures in the body. The image can be up to 3D with ultrasound. In particular, ultrasound imaging typically involves the use of a small transducer (probe) transmitting high-frequency sound waves to a target site and collecting the sounds that bounce back from the target site to provide the collected sound to a computer using sound waves to create an image of the target site. Ultrasound imaging allows detection of the function of moving structures in real-time. Ultrasound imaging works on the principle that different structures/fluids in the target site will attenuate and return sound differently depending on their composition. A contrast agent sometimes used with ultrasound imaging are microbubbles created by an agitated saline solution, which works due to the drop in density at the interface between the gas in the bubbles and the surrounding fluid, which creates a strong ultrasound reflection. Ultrasound imaging can be performed with conventional ultrasound techniques and devices displaying 2D images as well as three-dimensional (3-D) ultrasound that formats the sound wave data into 3-D images. In addition to 3D ultrasound imaging, ultrasound imaging also encompasses Doppler ultrasound imaging, which uses the Doppler Effect to measure and visualize movement, such as blood flow rates. Types of Doppler imaging includes continuous wave Doppler, where a continuous sinusoidal wave is used; pulsed wave Doppler, which uses pulsed waves transmitted at a constant repetition frequency, and color flow imaging, which uses the phase shift between pulses to determine velocity information which is given a false color (such as red=flow towards viewer and blue=flow away from viewer) superimposed on a grey-scale anatomical image. Ultrasound imaging can use linear or nonlinear propagation depending on the signal level. Harmonic and harmonic transient ultrasound response imaging can be used for increased axial resolution, as harmonic waves are generated from nonlinear distortions of the acoustic signal as the ultrasound waves insonate tissues in the body. Other ultrasound techniques and devices suitable to image a target site using ultrasound would be understood by a skilled person.

Types of ultrasound imaging of biological target sites include abdominal ultrasound, vascular ultrasound, obstetrical ultrasound, hysterosonography, pelvic ultrasound, renal ultrasound, thyroid ultrasound, testicular ultrasound, and pediatric ultrasound as well as additional ultrasound imaging as would be understood by a skilled person.

Applying ultrasound refers to sending ultrasound-range acoustic energy to a target. The sound energy produced by the piezoelectric transducer can be focused by beamforming, through transducer shape, lensing, or use of control pulses. The soundwave formed is transmitted to the body, then partially reflected or scattered by structures within a body; larger structures typically reflecting, and smaller structures typically scattering. The return sound energy reflected/scattered to the transducer vibrates the transducer and turns the return sound energy into electrical signals to be analyzed for imaging. The frequency and pressure of the input sound energy can be controlled and are selected based on the needs of the particular imaging task and, in some methods described herein, collapsing GVs. To create images, particularly 2D and 3D imaging, scanning techniques can be used where the ultrasound energy is applied in lines or slices which are composited into an image.

In some embodiments, imaging the target site can be performed by scanning an ultrasound image of the target site in a subject. In some cases, imaging the target site includes transmitting an imaging ultrasound signal from an ultrasound transmitter to the target site, and receiving a set of ultrasound data at a receiver. The visible image is formed by ultrasound signals backscattered from the target site. The ultrasound data can be analyzed using a processor, such as a processor configured to analyze the ultrasound data and produce an ultrasound image from the ultrasound data. In certain embodiments, the ultrasound data detected by the receiver includes an ultrasound signal, an ultrasound signal reflected by the target site of the subject.

In certain embodiments, the method includes applying a set of imaging pulses from an ultrasound transmitter to the target site, and receiving ultrasound signal at a receiver. In certain instances, the ultrasound signal detected by the receiver includes an ultrasound echo signal. Additional information of ultrasound systems and methods can be found in related publications as will be understood by a person skilled in the art.

Methods for performing ultrasound imaging are known in the art and can be employed in methods of the current disclosure. In certain aspects, an ultrasound transducer, which comprises piezoelectric elements, transmits an ultrasound imaging signal (or pulse) in the direction of the target site. Variations in the acoustic impedance (or echogenicity) along the path of the ultrasound imaging signal causes backscatter (or echo) of the imaging signal, which is received by the piezoelectric elements. The received echo signal is digitized into ultrasound data and displayed as an ultrasound image. Conventional ultrasound imaging systems comprise an array of ultrasonic transducer elements that are used to transmit an ultrasound beam, or a composite of ultrasonic imaging signals that form a scan line. The ultrasound beam is focused onto a target site by adjusting the relative phase and amplitudes of the imaging signals. The imaging signals are reflected back from the target site and received at the transducer elements. The voltages produced at the receiving transducer elements are summed so that the net signal is indicative of the ultrasound energy reflected from a single focal point in the subject. An ultrasound image is then composed of multiple image scan lines.

In embodiments herein described, imaging the target site is performed by applying or transmitting an imaging ultrasound signal from an ultrasound transmitter to the target site and receiving a set of ultrasound data at a receiver. The ultrasound data can be obtained using a standard ultrasound device, or can be obtained using an ultrasound device configured to specifically detect the contrast agent used. Obtaining the ultrasound data can include detecting the ultrasound signal with an ultrasound detector. In some embodiments, the imaging step further comprises analyzing the set of ultrasound data to produce an ultrasound image.

In certain embodiments, the ultrasound signal has a transmit frequency of at least 1 MHz, 5 MHz, 10 MHz, 20 MHz, 30 MHz, 40 MHz or 50 MHz. For example, an ultrasound data is obtained by applying to the target site an ultrasound signal at a transmit frequency from 4 to 11 MHz, or at a transmit frequency from 14 to 22 MHz.

In the embodiments herein described, the imaging ultrasound is selected to have a pressure on the protease sensitive GV based on the pressure profile of the protease sensitive GV type and the experimental design.

In particular, in embodiments, herein described, the imaging ultrasound is selected to provide the target site with a pressure below a buckling pressure of the protease sensitive GV type. In particular, ultrasound is applied to obtain detection and sensing of such nonlinear behavior, when it occurs (in presence of a protease), for example, by providing amplitude modulation (AM) ultrasound pulse sequences in order to image and differentiate the baseline nonlinear behavior of non-buckling GVs from the increased nonlinear behavior of buckling GVs.

In such amplitude modulation technique, as noted in Maresca et al 2017 [22] and in Maresca et al 2018 [23], backscattered echoes of two half-amplitude transmissions at applied pressures below the buckling threshold of the engineered GVs trigger basically linear scattering. Such echoes are digitally subtracted from echoes of a third, full-amplitude transmission at pressures above the buckling threshold of the engineered GVs and thus triggers harmonic, nonlinear scattering in case of engineered GVs. By way of example, the half-amplitude transmissions can occur with a B-mode pulse sequence, while the full-amplitude transmission can occur with a cross-amplitude modulation (x-AM) pulse sequence which uses pairs of cross-propagating plane waves to elicit highly specific nonlinear scattering from buckling GVs at the wave intersection, while subtracting the signal generated by transmitting each wave on its own (which have linear characteristics or noticeably lower nonlinear characteristics than the combined transmission of both plane waves produce at their intersection) and quantifying the resulting contrast. While it is advantageous to have the single plane wave pulses not produce any buckling, since the nonlinearity of the buckling increases with applied pressure up to an optimal point, all is required is that the harmonic responses from the individual plane waves (e.g. half pressure) be distinguishable from those of the combined intersection of the plane waves together (e.g. full pressure). Other nonlinear acoustical imaging techniques could also be used, such as cross-phase modulation imaging and harmonic imaging. Any imaging technique that allows imaging of the 2^{nd} (and/or higher) harmonics with the fundamental (first harmonic) signal subtracted out can be used to detect the buckling of the GvpC cleaved GVs (known as “differential nonlinear imaging” herein).

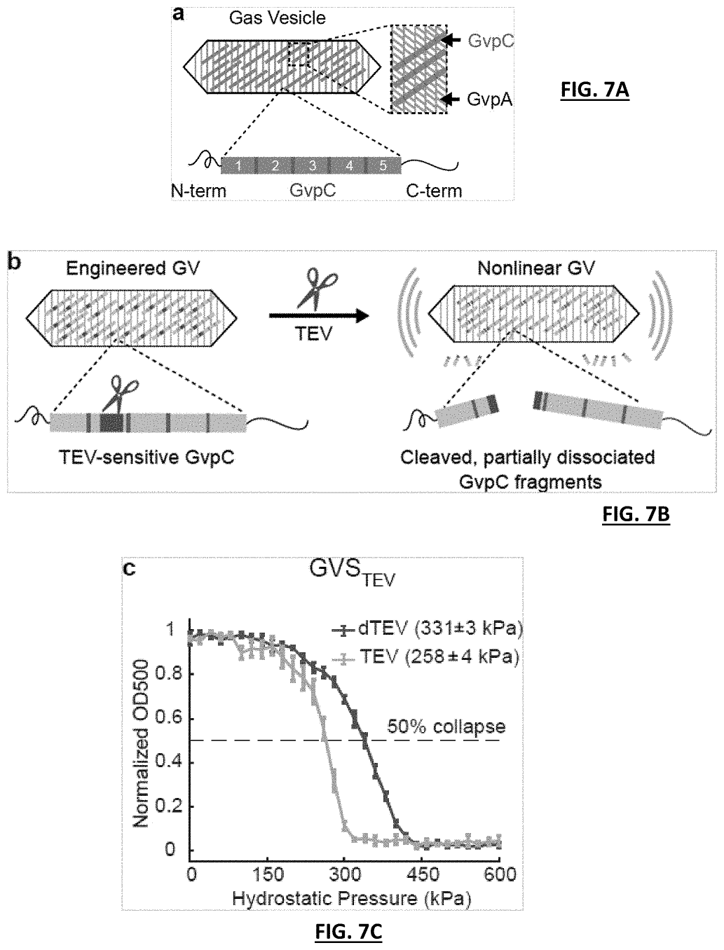

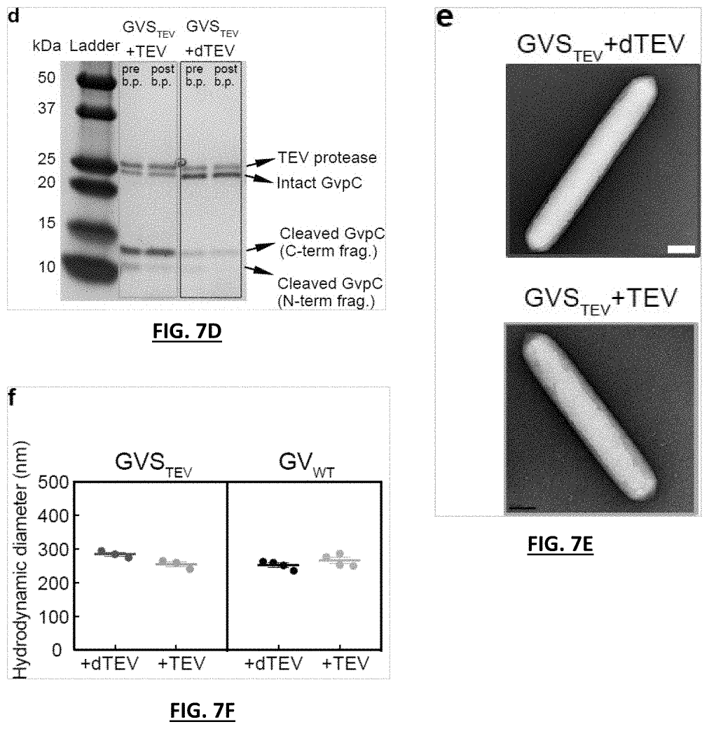

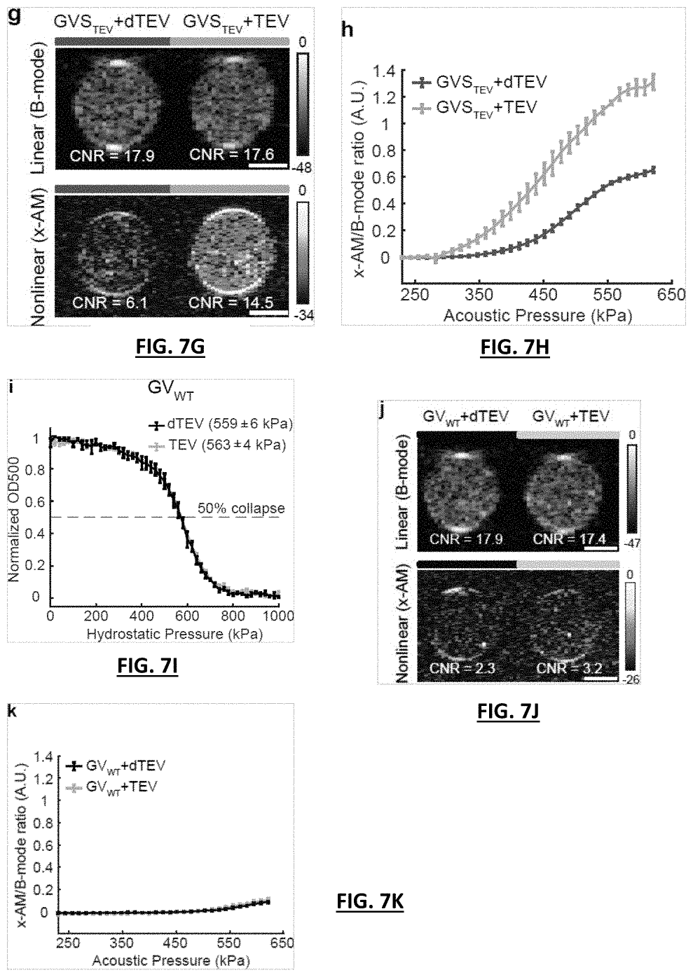

Reference is made in this connection to Example 1 and the related exemplary illustration of FIGS. 7H-7K. In particular, on one hand with reference to FIG. 7H, an example is shown where an engineered GVS_{TEV} sample is exposed to a TEV protease and produces a strong nonlinear acoustic response, with a maximal contrast-to-noise ratio (CNR) enhancement of about 7 dB (14. 5−6.1) at an applied acoustic pressure of about 438 kPa. On the other hand, with reference to FIG. 7K, substantially less nonlinear contrast is observed. As expected, both samples produce similar linear scattering. As also shown in FIG. 7H, consistent with the pressure-dependent mechanics of the GV shell, the differential nonlinear acoustic response of the engineered GVS_{TEV} samples becomes evident at pressures above about 295 kPa, and keeps increasing until about 556 kPa, at which point the GVs begin to collapse.

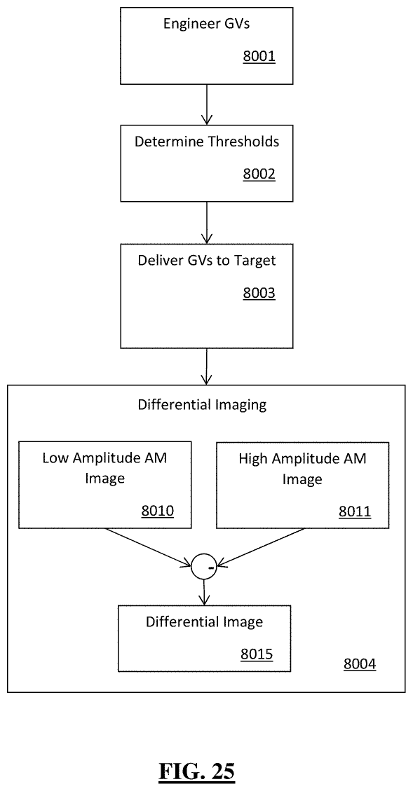

Therefore, a method to detect the presence of a protease is realized by the use of protease sensitive GVs as described herein and nonlinear acoustic imaging techniques. An example method is shown in FIG. 25.

For a given protease to be detected (either to determine its presence at a target area itself, or to detect if a certain biological event occurred that involves the action of protease) a GV is engineered (8001) to have a GvpC that would be compromised (e.g. cleaved) in the presence of that protease. In some embodiments, the engineered GvpC could also be degraded or chewed up by the protease (as for example ClpXP).

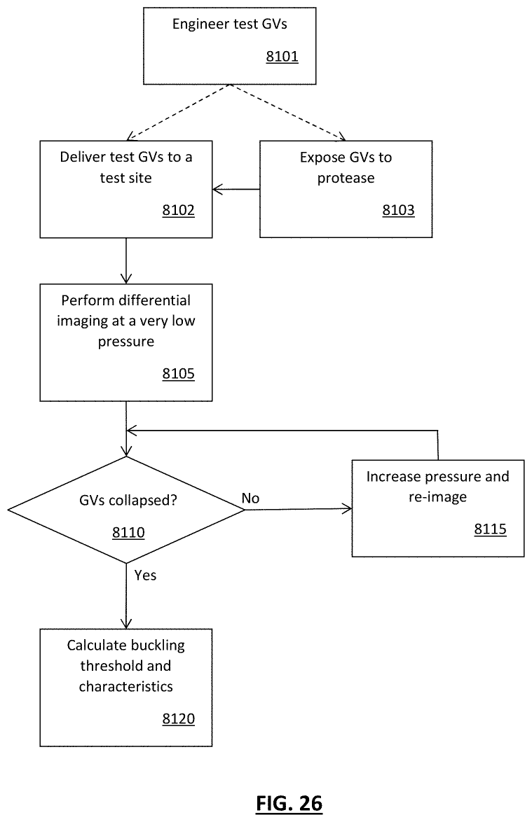

A buckling threshold and collapse threshold are determined (8002) for the GV with cleaved GvpC (exposed to protease). The collapse threshold is determined experimentally by known methods (see e.g. US 2018/0028693 “Gas-Filled Structures and Related Compositions, Methods and Systems to Image a Target Site”, incorporated by reference herein in its entirety). The buckling threshold is determined by the minimal acoustic pressure below the collapse threshold for the protease sensitive GVs to show detectable nonlinear signals, which is experimentally characterized by imaging the protease sensitive GVs under sequentially increasing acoustic pressure. See, for example, Figure. 26. The GVs are defined non-buckling if no nonlinear signals can be detected even when the collapse threshold is reached. By performing ultrasound imaging under sequentially increasing acoustic pressure, the optimum imaging pressure for the protease sensor is identified—at which there is maximum buckling of the protease sensitive GVs, giving rise to the highest nonlinear signal without collapsing the GVs. This is known herein as the “optimum buckling pressure”.

The GVs are delivered (8003) to the site, either by expressing the GVs outside the site and transporting the GVs to the site, or by having the GVs expressed at the site by a host cell.

The target site is then imaged (8004) using a differential nonlinear imaging technique, such as x-AM, such that the low amplitude (each plane wave transmitted individually) imaging (8010) is, for example, at a pressure below the buckling threshold (or, at least, at a pressure significantly below the high amplitude imaging, e.g. half) and the nonlinear high amplitude (both plane waves transmitted together) imaging (8011) is performed at a pressure above the buckling threshold. The differential image (8015) reveals the presence of the GVs that have had their GvpC weakened by the presence of the protease. Since the contrast-to-noise-ration (CNR) depend on many variables, e.g. transducers, surrounding media, electronic interference, the nonlinear signal is defined to be present when the x-AM CNR is higher than the x-AM CNR acquired with post-collapse GVs or GV-expressing cell samples, imaged under the same condition (e.g. GV/cell concentration, imaging acoustic pressure).

In some embodiments, the dynamic range of the nonlinear signal is further tuned with further manipulations of GvpC, e.g. linkers connecting the tag and GvpC, positioning of the cleavage sites, and the number of repeats in the repeating region.

The method of differential imaging based the nonlinear characteristics induced by buckling can be combined with imaging based on the GV bubble cavitation.

The buckling pressure of a given GvpC cleaved GV type can be determined by testing. FIG. 26 shows an example of how to test a GV for its buckling pressure. First, some test GVs are procured/engineered (8101) for the testing for a given GV type. These GVs are then administered (8102) to an in vitro or in vivo test site. The GvpCs of these GVs are cleaved/degraded (8103) by exposure to the correct protease, either before or after administration to the test site. The test site does not have to be the same as the target site the GVs will be eventually used for, but more accurate results may be produced if the background scattering of the test site is close to that of the target site (same nonlinear effects). Differential imaging (e.g. xAM) is performed (8105) at a low pressure. This could be the lowest pressure setting of the imaging device, or it can be an arbitrary low value well below the collapse threshold of the GVs. A determination of if the imaging steps should stop is made, for example by seeing if the GVs have collapsed from the pressure (8110). If testing should continue, the pressure (or voltage) is incrementally increased and the imaging is performed again (8115). The check (8110) and increased pressure imaging (8115) repeats until the imaging is determined to stop, then the data from all the imaging is used to get a profile (8120) of the differential imaging nonlinear response, such as shown in the examples herein. This profile shows when the nonlinear buckling effects begin to be noticeable compared the more linear low amplitude signal effects (“buckling starts”), when the effects are at a maximum (“optimal buckling pressure”), and when it ends (“collapse pressure”). A buckling pressure for the given GV type can be selected (8120) anywhere in the range from when buckling starts to the collapse pressure, including at the optimal buckling pressure. In some embodiments, information from a profile taken from the GV type without exposure to protease (uncleaved GvpC) is used to determine the characteristics of the buckling GVs by comparing at what pressure point (buckling starts value) the cleaved GvpC GVs show increased (nonlinear) echo compared to the noncleaved GvpC GVs.

Accordingly, protease sensitive GV herein described can be used to detect proteases and/or protease associated biological event in target sites and/or in a cell within a target site.

The term “biological event” or “biochemical event” as used herein refers to an activating, inhibiting, binding or converting reaction between two or more molecule in a biological environment inside or outside a cell. A “protease associated” biological event as used herein indicates a biological event involving a protease in the sense of the disclosure. For example, u-calpain activated by calcium influx cleaves the signaling proteins (e.g. RhoA) during the induction of long-term potentiation [//www.jneurosci.org/content/35/5/2269]. Another example is activated matrix metalloproteinases (MMPs) cleaving proteins in the extracellular microenvironment of cancerous cells, mediating tumor progression [//www.ncbi.nlm.nih.gov/pmc/articles/PMC2862057/].

Non-protease sensitive GvpC can be expressed under genetic circuits of interest, along with the protease sensitive GV inside the cells, where the non-protease sensitive GvpC would be expressed and replace the protease-sensitive GvpC upon induction of the genetic circuits that tune down the nonlinear signal and inactivate the protease-dependent response of the GV.

In some embodiments, the protease sensitive GV can be used as a conditional collapse protease detector. See e.g. FIG. 9D. The ClpXP exposed GV has a collapse threshold of around 223 kPa, whereas the inhibitor exposed GV (no ClpXP) has a collapse threshold of around 476 kPa. Knowing this, an ultrasound imaging at a point between the two thresholds (e.g. 300 kPa) contrasted by a following image above the 476 kPa would show if the GVs were exposed to the protease: if the first image shows collapse, then there was protease; if only the second image shows collapse, then there was no protease; and if neither image shows collapse, then the GV was not present.

In some embodiments, the protease sensitive GV can be used as a conditional cavitation nucleus. Only upon activity of specific protease and resulting decreased collapse threshold, the protease sensitive GV can be collapsed under a certain acoustic pressure and function as a cavitation nucleus, while with no such protease activity the collapse threshold would be higher than the applied acoustic pressure and the GV would not function as a cavitation nucleus efficiently. This is similar to the conditional collapse protease detector in that it depends on the GV showing different shell mechanics under cleaved vs. non-cleaved GvpC.

Accordingly, in methods herein described, administration of one or more engineered protease sensitive GV type and/or protease sensitive GV cell to a target site to be imaged, can be performed in any way suitable to deliver the one or more engineered protease sensitive GV type and/or protease sensitive GV cell to the target site to be imaged.

In some embodiments, in which the target site is the body of an individual or a part thereof, the one or more engineered protease sensitive GV type and/or protease sensitive GV cell can be administered to the target site locally or systemically.

The wording “local administration” or “topic administration” as used herein indicates any route of administration by which the one or more genetically engineered one or more engineered protease sensitive GV type and/or protease sensitive GV cell are brought in contact with the body of the individual, so that the resulting location of the one or more engineered protease sensitive GV type and/or protease sensitive GV cell in the body is topic (limited to a specific tissue, organ or other body part where the imaging is desired). Exemplary local administration routes include injection into a particular tissue by a needle, gavage into the gastrointestinal tract, and spreading a solution containing the one or more engineered protease sensitive GV type and/or protease sensitive GV cell on a skin surface.

The wording “systemic administration” as used herein indicates any route of administration by which the one or more engineered protease sensitive GV type and/or protease sensitive GV cell are brought in contact with the body of the individual, so that the resulting location of the one or more engineered protease sensitive GV type and/or protease sensitive GV cell in the body is systemic (not limited to a specific tissue, organ or other body part where the imaging is desired). Systemic administration includes enteral and parenteral administration. Enteral administration is a systemic route of administration where the substance is given via the digestive tract, and comprise oral administration, administration by gastric feeding tube, administration by duodenal feeding tube, gastrostomy, enteral nutrition, and rectal administration. Parenteral administration is a systemic route of administration where the substance is given by route other than the digestive tract and includes but is not limited to intravenous administration, intra-arterial administration, intramuscular administration, subcutaneous administration, intradermal, administration, intraperitoneal administration, and intravesical infusion.

Accordingly, in some embodiments of methods herein described, administering one or more engineered protease sensitive GV type and/or protease sensitive GV cell can be performed topically or systemically by intradermal, intramuscular, intraperitoneal, intravenous, subcutaneous, intranasal, intracerebroventricular, rectal, vaginal, and oral routes. In particular, the one or more genetically engineered bacterial cell types comprising a GVR genetic circuit can be administered by infusion or bolus injection, and can optionally be administered together with other biologically active agents. In some embodiments of methods herein described, administering one or more engineered protease sensitive GV type and/or protease sensitive GV cell can be performed by injecting one or more engineered protease sensitive GV type and/or protease sensitive GV cell such as in a body cavity or lumen.

As mentioned above, protease sensitive gas vesicle protein GvpC and related Gas Vesicle, Gas Vesicle Gene Cluster (GVGC), gene cassettes expression systems, vectors, cells, and compositions, herein described can be provided as a part of systems to perform any of the above mentioned methods. The systems can be provided in the form of kits of parts. In a kit of parts, one or more protease sensitive gas vesicle protein GvpC and related Gas Vesicle, Gas Vesicle Gene Cluster (GVGC), gene cassettes expression systems, vectors, cells, compositions, and other reagents to perform the methods herein described are comprised in the kit independently. The protease sensitive gas vesicle protein GvpC and related Gas Vesicle, Gas Vesicle Gene Cluster (GVGC), gene cassettes expression systems, vectors, and cells, can be included in one or more compositions together with a suitable vehicle.

In embodiments herein described, the components of the kit can be provided, with suitable instructions and other necessary reagents, in order to perform the methods here disclosed. The kit will normally contain the compositions in separate containers. Instructions, for example written or audio instructions, on paper or electronic support such as tapes, CD-ROMs, flash drives, or by indication of a Uniform Resource Locator (URL), which contains a pdf copy of the instructions for carrying out the assay, will usually be included in the kit. The kit can also contain, depending on the particular method used, other packaged reagents and materials (i.e. wash buffers and the like).

Further details concerning protease sensitive gas vesicle protein GvpC and related Gas Vesicle, Gas Vesicle Gene Cluster (GVGC), gene cassettes expression systems, vectors, cells, and compositions, herein described and related methods and systems of the present disclosure will become more apparent hereinafter from the following detailed disclosure of examples by way of illustration only with reference to an experimental section.

### Examples

The protease sensitive gas vesicle protein GvpC and related Gas Vesicle, Gas Vesicle Gene Cluster (GVGC), gene cassettes expression systems, vectors, cells of the disclosure and related methods, and systems herein described are further illustrated in the following examples, which are provided by way of illustration and are not intended to be limiting.

The following materials and methods were used.

### Design and Cloning of Genetic Constructs

All gene sequences were codon optimized for E. Coli expression and inserted into their plasmid backbones via Gibson Assembly or KLD Mutagenesis using enzymes from New England Biolabs and custom primers from Integrated DNA Technologies. The protease recognition sequences for TEV protease and μ-calpain, flanked by flexible linkers, were introduced by substitution-insertion into the second repeat of the wild-type Ana GvpC sequence in a pET28a expression vector (Novagen) driven by a T7 promoter and lac operator. The ssrA degradation tag for the ClpXP bacterial proteasome was appended to the C-terminus of Ana GvpC using a short flexible linker. The acoustic sensor gene for intracellular protease sensing of ClpXP was constructed by modifying of the acoustic reporter gene cluster ARG1[10], by addition of the ssrA degradation tag to the C-terminal of GvpC using a linker sequence. For expression in E. coli Nissle 1917 cells, the pET28a T7 promoter was replaced by the T5 promoter. For inducible expression of ClpX and ClpP, the genes encoding those two proteins were cloned from the E. coli Nissle 1917 genome into a modified pTARA backbone under a pBAD promoter and araBAD operon. For dynamic regulation of intracellular sensing, the wild-type GvpC sequence was cloned into a modified pTARA backbone under a pTet promoter and tetracycline operator. The complete list and features of plasmids used in this study is given in Table 13.

TABLE 13
List and features of genetic constructs used in this study

| Description of | Plasmid | Transcriptional | Output gene | Insertions/Tags | |
|---|---|---|---|---|---|
| genetic construct | Backbone | regulators | product(s) | (including linkers) | Resistance |
| WT Ana GvpC | pET28a | pT7, LacO | WT C-His | SLE-His6 at C-terminus | Kanamycin |
| used as control | | | Ana GvpC | | |
| for TEV/calpain | | | | | |
| sensor | | | | | |
| WT Ana GvpC | pET28a | pT7, LacO | WT N-His- | G-His6-SG at N-terminus | Kanamycin |
| used as control | | | Ana-GvpC | | |
| for ClpXP sensor | | | | | |
| Ana GvpC with | pET26b | pT7, LacO | C-His Ana | SLE-His6 at C-terminus, | Kanamycin |
| TEV cleavage | | | GvpC with | GSGSGSG-ENLYFQG- | |
| site | | | TEV | SGSGSG in GvpC repeat 2 | |
| | | | cleavage | | |
| | | | site | | |
| Ana GvpC with | pET28a | pT7, LacO | C-His Ana | SLE-His6 at C-terminus, | Kanamycin |
| calpain cleavage | | | GvpC with | GSGSG-QQEVYGMMPRD- | |
| site | | | calpain | GSGSG in GvpC repeat 2 | |
| | | | cleavage | | |
| | | | site | | |
| Ana GvpC with | pET28a | pT7, LacO | N-His Ana | G-His6-SG at N-terminus, | Kanamycin |
| ssrA | | | GvpC with | SG-AANDENYALAA at C- | |
| degradation tag | | | ssrA | terminus | |
| | | | degradation | | |
| | | | tag | | |
| ClpP plasmid for | pBEST | OR2-OR1-Pr | ClpP | | Ampicillin |
| use in the cell- | | | | | |
| free TX-TL | | | | | |
| system | | | | | |
| Original | pET28a | pT5, LacO | Ana GvpA, | | Kanamycin |
| acoustic | | | WT Ana | | |
| reporter gene | | | GvpC, Mega | | |
| construct | | | GvpR-U | | |
| (ARG_{WT}) | | | | | |
| Acoustic sensor | pET28a | pT5, LacO | Ana GvpA, | SG-AANDENYALAA at C- | Kanamycin |
| gene for ClpXP | | | dGvpC, | terminus | |
| (ASG_{ClpXP}) | | | Mega GvpR-U | | |
| Frt-flanked cat | pANTSγ | | CmR | | Ampicillin |
| cassette for | | | | | |
| recombineering | | | | | |
| (pKD3) | | | | | |
| Plasmid that | pBAD18 | pBAD, araBAD | Gam, Beta, | | Ampicillin |
| carries the | | operon | Exo | | |
| Lambda Red | | | | | |
| recombineering | | | | | |
| system | | | | | |
| (pKD46) | | | | | |
| Flippase to | pBAD18 | pE | FLP | | Ampicillin |
| remove the | | | | | |
| integration | | | | | |
| module in | | | | | |
| reombineering | | | | | |
| ClpX ClpP | pTARA | pBAD, araBAD | ClpX, ClpP | | Chloramphenicol |
| expression | (modified | operon | | | |
| under araBAD | by deletion | | | | |
| promoter | of pTet) | | | | |
| WT Ana GvpC | pTARA | pTet, TetO | WT Ana | | Chloramphenicol |
| under Tet | (modified | | GvpC | | |
| promoter | by deletion | | | | |
| | of pAra) | | | | |

The plasmids are available on Addgene. Plasmid constructs were cloned using NEB Turbo E. Coli (New England Biolabs) and sequence-validated.

Construction of clpX⁻ clpP⁻ Strain of E. coli Nissle 1917 (ΔClpXP)

The knockout of clpX and clpP in E. coli Nissle (ECN) was accomplished by Lamda Red recombineering using a published protocol[37]. A Frt-flanked CmR gene was recombined into ECN genome to replace the clpX and clpP genes, and the integrated CmR gene was then removed by the FLP flippase from pE-FLP⁵² to yield the ΔClpXP strain. Recombineering plasmids pKD3[37], pE-FLP[38] were obtained from Addgene, and pKD46[37] was obtained from the Coli Genetic Stock Center.

### GV Expression, Purification and Quantification

For in vitro assays, GVs were harvested and purified from confluent Ana cultures using previously published protocols[31, 32]. Briefly, Ana cells were grown in Gorham's media supplemented with BG-11 solution (Sigma) and 10 mM sodium bicarbonate at 25° C., 1% CO₂ and 100 rpm shaking, under a 14 h light and 10 h dark cycle. Confluent cultures were transferred to sterile separating funnels and left undisturbed for 2-3 days to allow buoyant Ana cells expressing GVs to float to the top and for their subnatant to be drained. Hypertonic lysis with 10% Solulyse (Genlantis) and 500 mM sorbitol was used to release and harvest the Ana GVs. Purified GVs were obtained through 3-4 rounds of centrifugally assisted floatation, with removal of the subnatant and resuspension in phosphate buffered saline (PBS, Corning) after each round.

For expression of acoustic reporter/sensor genes (ARG/ASG) in bacteria, wild-type E. Coli Nissle 1917 cells (Ardeypharm GmbH) were made electrocompetent and transformed with the genetic constructs. After electroporation, cells were rescued in SOC media supplemented with 2% glucose for 1 h at 37° C. Transformed cells were grown for 12-16 hours at 37° C. in 5 mL of LB medium supplemented with 50 μg/mL kanamycin and 2% glucose. Large-scale cultures for expression were prepared by a 1:100 dilution of the starter culture in LB medium containing 50 μg/mL kanamycin and 0.2% glucose. Cells were grown at 37° C. to an OD600 nm of 0.2-0.3, then induced with 3 μM Isopropyl β-D-1-thiogalactopyranoside (IPTG) and allowed to grow for 22 hrs at 30° C. Buoyant E. coli Nissle cells expressing GVs were isolated from the rest of the culture by centrifugally assisted floatation in 50 mL conical tubes at 300 g for 3-4 hrs, with a liquid column height less than 10 cm to prevent GV collapse by hydrostatic pressure.

The concentration of Ana GVs was determined by measurement of their optical density (OD) at 500 nm (OD₅₀₀) using a Nanodrop spectrophotometer (Thermo Fisher Scientific), using the resuspension buffer or collapsed GVs as the blank. As established in previous work[32], the concentration of GVs at OD₅₀₀=1 is approximately 114 pM and the gas fraction is 0.0417%. The OD of buoyant cells expressing GVs were quantified at 600 nm using the Nanodrop.

### Bacterial Expression and Purification of GvpC Variants

For expression of Ana GvpC variants, plasmids were transformed into chemically competent BL21(DE3) cells (Invitrogen) and grown overnight for 14-16 h at 37° C. in 5 mL starter cultures in LB medium with 50 μg/mL kanamycin. Starter cultures were diluted 1:250 in Terrific Broth (Sigma) and allowed to grow at 37° C. (250 rpm shaking) to reach an OD₆₀₀ₙₘ of 0.4-0.7. Protein expression was induced by addition of 1 mM IPTG, and the cultures were transferred to 30° C. Cells were harvested by centrifugation at 5500 g after 6-8 hours. For the GvpC-ssrA variant, expression was carried out at 25° C. for 8 hours to reduce the effect of protease degradation and obtain sufficient protein yield.

GvpC was purified from inclusion bodies by lysing the cells at room temperature using Solulyse (Genlantis), supplemented with lysozyme (400 μg/mL) and DNAseI (10 μg/mL). Inclusion body pellets were isolated by centrifugation at 27,000 g for 15 mins and then resuspended in a solubilization buffer comprising 20 mM Tris-HCl buffer with 500 mM NaCl and 6 M urea (pH: 8.0), before incubation with Ni-NTA resin (Qiagen) for 2 h at 4° C. The wash and elution buffers were of the same composition as the solubilization buffer, but with 20 mM and 250 mM imidazole respectively. The concentration of the purified protein was assayed using the Bradford Reagent (Sigma). Purified GvpC variants were verified to be >95% pure by SDS-PAGE analysis.

### Preparation of Gas Vesicles for In Vitro Protease Assays

Engineered GVs having protease-sensitive or wild-type GvpC were prepared using urea stripping and GvpC re-addition[31, 32]. Briefly, Ana GVs were stripped of their native outer layer of GvpC by treatment with 6M urea solution buffered with 100 mM Tris-HCl (pH:8-8.5). Two rounds of centrifugally assisted floatation with removal of the subnatant liquid after each round were performed to ensure complete removal of native GvpC. Recombinant Ana GvpC variants purified from inclusion bodies were then added to the stripped Ana GVs in 6 M urea a 2-3× molar excess concentration determined after accounting for 1:25 binding ratio of GvpC: GvpA. For a twofold stoichiometric excess of GvpC relative to binding sites on an average Ana GV, the quantity of recombinant GvpC (in nmol) to be added to stripped GVs was calculated according to the formula: 2*OD*198 nM*volume of GVs (in liters). The mixture of stripped GVs (OD₅₀₀ₙₘ=1-2) and recombinant GvpC in 6 M urea buffer was loaded into dialysis pouches made of regenerated cellulose membrane with a 6-8 kDa M.W. cutoff (Spectrum Labs). The GvpC was allowed to slowly refold onto the surface of the stripped GVs by dialysis in 4 L PBS for at least 12 h at 4° C. Dialyzed GV samples were subjected to two or more rounds of centrifugally assisted floatation at 300 g for 3-4 h to remove any excess unbound GvpC. Engineered GVs were resuspended in PBS after subnatant removal and quantified using pressure-sensitive OD measurements at 500 nm using a Nanodrop.

### Pressurized Absorbance Spectroscopy

Purified, engineered Ana GVs were diluted in experimental buffers to an OD₅₀₀ₙₘ ˜0.2-0.4, and 400 μL of the diluted sample was loaded into a flow-through quartz cuvette with a pathlength of 1 cm (Hellma Analytics). Buoyant E. coli Nissle cells expressing GVs were diluted to an OD₆₀₀ₙₘ of ˜1 in PBS for measurements. A 1.5 MPa nitrogen gas source was used to apply hydrostatic pressure in the cuvette through a single valve pressure controller (PC series, Alicat Scientific), while a microspectrometer (STS-VIS, Ocean Optics) measured the OD of the sample at 500 nm (for Ana GVs) or 600 nm (for Nissle cells). The hydrostatic pressure was increased from 0 to 1 MPa in 20 kPa increments with a 7 second equilibration period at each pressure before OD measurement. Each set of measurements was normalized by scaling to the Min-Max measurement value, and the data was fitted using the Boltzmann sigmoid function f(p)=(1+e^{(P-P}^{_{c}}^{)/ΔP})⁻¹, with the midpoint of normalized OD change (P_{cc}) and the 95% confidence intervals, rounded to the nearest integer, reported in the figures.

### TEM Sample Preparation and Imaging

Freshly diluted samples of engineered Ana GVs (OD₅₀₀ₙₘ ˜0.3) in 10 mM HEPES buffer containing 150 mM NaCl (pH 8) were used for TEM. 2 μL of the sample was added to Formvar/carbon 200 mesh grids (Ted Pella) that were rendered hydrophilic by glow discharging (Emitek K100X). 2% uranyl acetate was added for negative staining. Images were acquired using the FEI Tecnai T12 LaB6 120 kV TEM equipped with a Gatan Ultrascan 2 k×2 k CCD and ‘Leginon’ automated data collection software suite.

### Dynamic Light Scattering (DLS) Measurements

Engineered Ana GVs were diluted to an OD₅₀₀ₙₘ ˜0.2 in experimental buffers. 150-200 μL of the sample was loaded into a disposable cuvette (Eppendorf UVette®) and the particle size was measured using the ZetaPALS particle sizing software (Brookhaven instruments) with an angle of 90° and refractive index of 1.33.

### Denaturing Polyacrylamide Gel Electrophoresis (SDS-PAGE)

GV samples were OD₅₀₀ₙₘ matched and mixed 1:1 with 2× Laemmli buffer (Bio-Rad), containing SDS and 2-mercaptoethanol. The samples were then boiled at 95° C. for 5 minutes and loaded into a pre-made polyacrylamide gel (Bio-Rad) immersed in 1× Tris-Glycine-SDS Buffer. 10 uL of Precision Plus Protein™ Dual Color Standards (Bio-Rad) was loaded as the ladder. Electrophoresis was performed at 120V for 55 minutes, after which the gel was washed in DI water for 15 minutes to remove excess SDS and commassie-stained for 1 hour in a rocker-shaker using the SimplyBlue SafeStain (Invitrogen). The gel was allowed to de-stain overnight in DI water before imaging using a Bio-Rad ChemiDoc™ imaging system.

### In Vitro Protease Assays

For in vitro assays with the TEV endopeptidase, recombinant TEV protease (R&D Systems, Cat. No. 4469-TP-200) was incubated (25% v/v fraction) with engineered Ana GVs resuspended in PBS (final OD₅₀₀ₙₘ in reaction mixture=5-6) at 30° C. for 14-16 h. This corresponds to a TEV concentration of 0.1-0.125 mg/mL (depending on the lot), within the range used in previous studies with this enzyme[39, 40]. Engineered GVs with wild-type GvpC and TEV protease heat-inactivated at 80° C. for 20-30 mins were used as the controls.

For in vitro assays with calpain, calpain-1 from porcine erythrocytes (Millipore Sigma, Cat. No. 208712) was incubated in a 10% v/v fraction with engineered Ana GVs in a reaction mixture containing 50 mM Tris-HCl, 50 mM NaCl, 5 mM 2-mercaptoethanol, 1 mM EDTA and 1 mM EGTA and 5 mM Ca²⁺ (pH: 7.5) This corresponds to a caplain concentration of >0.168 units per μl, with 1 unit defined by the manufacturer as sufficient to cleave 1 pmol of a control fluorogenic substrate in 1 min at 25° C. The final concentration of engineered GVs in the reaction mixture was OD₅₀₀ₙₘ ˜6 and the protease assay was carried out at 25° C. for 14-16 h. Negative controls included the same reaction mixture without calpain, without calcium, or without calpain and calcium. Engineered GVs with WT-GvpC were used as additional negative controls.

For in vitro assays with ClpXP, a reconstituted cell-free transcription-translation (TX-TL) system adapted for ClpXP degradation assays (gift of Zachary Sun and Richard Murray) was used. Briefly, cell-free extract was prepared by lysis of ExpressIQ E. coli cells (New England Biolabs), and mixed in a 44% v/v ratio with an energy source buffer, resulting in a master mix of extract and buffer comprising: 9.9 mg/mL protein, 1.5 mM each amino acid except leucine, 1.25 mM leucine, 9.5 mM Mg-glutamate, 95 mM K-glutamate, 0.33 mM DTT, 50 mM HEPES, 1.5 mM ATP and GTP, 0.9 mM CTP and UTP, 0.2 mg/mL tRNA, 0.26 mM CoA, 0.33 mM NAD, 0.75 mM cAMP, 0.068 mM folinic acid, 1 mM spermidine, 30 mM 3-PGA and 2% PEG-8000. For purified ClpX protein, a monomeric N-terminal deletion variant Flag-ClpXdeltaNLinkedHexamer-His6 (Addgene ID: 22143) was used. Post Ni-NTA purification, active fractions of ClpX hexamers with sizes above 250 kDa were isolated using a Supradex 2010/300 column, flash frozen at a concentration of 1.95 μM and stored at −80° C. in a storage buffer consisting of: 50 mM Tris-Cl (pH 7.5), 100 mM NaCl, 1 mM DTT, 1 mM EDTA and 2% DMSO. The final reaction mixture was prepared as follows: 75% v/v fraction of the master mix, 10% v/v of purified ClpX, 1 nm of the purified pBEST-ClpP plasmid and engineered Ana GVs (concentration of OD₅₀₀ₙₘ=2.5-2.7 in the reaction mixture). The mixture was made up to the final volume using ultrapure H₂O. The reaction was allowed to proceed at 30° C. for 14-16 h. As a negative control, a protease inhibitor cocktail mixture (SIGMAFAST™, Millipore Sigma) was added to the reaction mixture at 1.65× the manufacturer-recommended concentration and pre-incubated at room temperature for 30 mins.

### Dynamic Sensing of ClpXP Activity in ΔClpXP E. Coli Nissle 1917 Cells

ClpXP E. Coli Nissle 1917 cells were made electrocompetent and co-transformed with the pET expression plasmid (Lac-driven) containing the ASG for ClpXP and a modified pTARA plasmid (pBAD-driven) containing the clpX and clpP genes. Electroporated cells were rescued in SOC media supplemented with 2% glucose for 2 h at 37° C. Transformed cells were grown overnight at 37° C. in 5 mL LB medium supplemented with 50 μg/mL kanamycin, 25 μg/mL chloramphenicol and 2% glucose. Starter cultures were diluted 1:100 in LB medium with 50 μg/mL kanamycin, 25 μg/mL chloramphenicol and 0.2% glucose and allowed to grow at 37° C. to reach an OD₆₀₀ₙₘ of 0.2-0.3. ASG expression was induced with 3 μM IPTG and the bacterial culture was transferred to the 30° C. incubator with 250 rpm shaking for 30 minutes. The culture was then split into two halves of equal volume, and one half was induced with 0.5% (weight fraction) L-arabinose for expression of ClpXP. Cultures with and without L-arabinose induction were allowed to grow for an additional 22 h at 30° C. Cultures were then spun down at 300 g in a refrigerated centrifuge at 4° C. for 3-4 h in 50 mL conical tubes to isolate buoyant cells expressing GVs from the rest of the culture. The liquid column height was maintained at less than 10 cm to prevent GV collapse by hydrostatic pressure.

Dynamic Sensing of Circuit-Driven Gene Expression in E. coli Nissle 1917 Cells

Electrocompetent E. coli Nissle cells were co-transformed with the pET expression plasmid (Lac-driven) containing the ASG for ClpXP and a modified pTARA plasmid (Tet-driven) containing the WT Ana GvpC gene. Electroporated cells were rescued in SOC media supplemented with 2% glucose for 2 h at 37° C. Transformed cells were grown overnight at 37° C. in 5 mL LB medium supplemented with 50 μg/mL kanamycin, 50 μg/mL chloramphenicol and 2% glucose. Starter cultures were diluted 1:100 in LB medium with 50 μg/mL kanamycin, 50 μg/mL chloramphenicol and 0.2% glucose and allowed to grow at 37° C. to reach an OD₆₀₀ₙₘ of 0.2-0.3. ASG expression was induced with 3 μM IPTG and the bacterial culture was transferred to 30° C. incubator with 250 rpm shaking for 1.5-2 h. The culture was then split into two halves of equal volume, and one half was induced with 50 ng/mL aTc for expression of WT GvpC. Cultures with and without aTc induction were allowed to grow for an additional 20 h at 30° C. Cultures were then spun down at 300 g in a refrigerated centrifuge at 4° C. for 3-4 h in 50 mL conical tubes to isolate buoyant cells expressing GVs from the rest of the culture. The liquid column height was maintained at less than 10 cm to prevent GV collapse by hydrostatic pressure.

### In Vitro Ultrasound Imaging

Imaging phantoms were prepared by melting 1% agarose (w/v) in PBS and casting wells using a custom 3D-printed template mold containing a 2-by-2 grid of cylindrical wells with 2 mm diameter and 1 mm spacing between the outer radii in the bulk material. Ana GV samples from in vitro assays or buoyant Nissle cells expressing GVs were mixed 1:1 with 1% molten agarose solution at 42° C. and quickly loaded before solidification into the phantom wells. All samples and their controls were OD-matched using the Nanodrop prior to phantom loading, with the final concentration being OD₅₀₀ₙₘ=2.2 for Ana GVs and OD_{600 nm}=1.0-1.5 for buoyant Nissle cells. Wells not containing sample were filled with plain 1% agarose. Hydrostatic collapse at 1.4 MPa was used to determine that the contribution to light scattering from GVs inside the cells was similar for those expressing the acoustic sensor gene and its wild-type ARG counterpart. The phantom was placed in a custom holder on top of an acoustic absorber material and immersed in PBS to acoustically couple the phantom to the ultrasound imaging transducer.

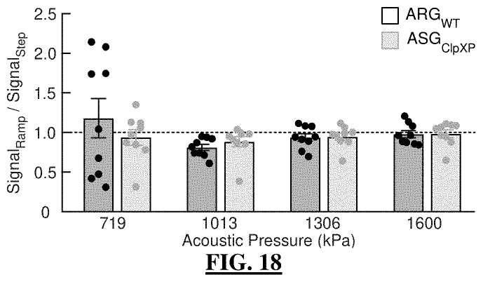

Imaging was performed using a Verasonics Vantage programmable ultrasound scanning system and a L22-14v 128-element linear array Verasonics transducer, with a specified pitch of 0.1 mm, an elevation focus of 8 mm, an elevation aperture of 1.5 mm and a center frequency of 18.5 MHz with 67%−6 dB bandwidth. Linear imaging was performed using a conventional B-mode sequence with a 128-ray-lines protocol. For each ray line, a single pulse was transmitted with an aperture of 40 elements. For nonlinear image acquisition, a custom cross-amplitude modulation (x-AM) sequence detailed in an earlier study[41], with an x-AM angle (0) of 19.5° and an aperture of 65 elements, was used. Both B-mode and x-AM sequences were programmed to operate at 15.625 MHz, corresponding to ¼ the sampling rate of the Vantage system. The center of the sample wells were aligned to the set transmit focus of 5 mm. Transmitted pressure at the focus was calibrated using a Precision Acoustics fiber-optic hydrophone system. Each image was a coherent average of 50 accumulations. B-mode images were acquired at a transmit voltage of 1.6V (132 kPa), and an automated voltage ramp imaging script (programmed in MATLAB) was used to sequentially toggle between B-mode and x-AM acquisitions. The script acquired x-AM signals at each specified voltage step, immediately followed by a B-mode acquisition at 1.6V (132 kPa), before another x-AM acquisition at the next voltage step. For engineered Ana GVs subjected to in vitro protease assays, an x-AM voltage ramp sequence from 4V (230 kPa) to 10V (621 kPa) in 0.2V increments was used. For wild-type Nissle cells expressing GVs, an x-AM voltage ramp sequence from 7.5V (458 kPa) to 25V (1.6 MPa) in 0.5V increments was used. Samples were subjected to complete collapse at 25V with the B-mode sequence for 10 seconds, and the subsequent B-mode image acquired at 1.6V and x-AM image acquired at the highest voltage of the voltage ramp sequence was used as the blank for data processing. There was no significant difference between the signals acquired at specific acoustic pressures during a voltage ramp or after directly stepping to the same pressure (FIG. 18).

Due to transducer failure, a replacement Verasonics transducer (L22-14vX) with similar specifications was used in experiments with ΔClpXP cells. The transmitted pressure at the focus was calibrated in the same way as the L22-14v. B-mode images were acquired at a transmit voltage of 1.6V (309 kPa), and an x-AM voltage ramp sequence from 6V (502 kPa) to 25V (2.52 MPa) was used. The imaging protocol was otherwise unchanged.

### In Vivo Ultrasound Imaging

All in vivo experiments were performed on C57BL/6J male mice, aged 14-34 weeks, under a protocol approved by the Institutional Animal Care and Use Committee of the California Institute of Technology. No randomization or blinding were necessary in this study. Mice were anesthetized with 1-2% isoflurane, maintained at 37° C. on a heating pad, depilated over the imaged region, and enema was performed by injecting PBS to expel gas and solid contents in mice colon. For imaging of E. coli in the gastrointestinal tract, mice were placed in a supine position, with the ultrasound transducer positioned on the lower abdomen, transverse to the colon such that the transmit focus of 5 mm was close to the center of the colon lumen. Prior to imaging, two variants of buoyancy-enriched E. coli Nissle 1917 were mixed in a 1:1 ratio with 4% agarose in PBS at 42° C., for a final bacterial concentration of 1.5E9 cells ml⁻¹. An 8-gauge gavage needle was filled with the mixture of agarose and bacteria of one cell population. Before it solidified, a 14-gauge needle was placed inside the 8-gauge needle to form a hollow lumen within the gel. After the agarose-bacteria mixture solidified at room temperature for 10 min, the 14-gauge needle was removed. The hollow lumen was then filled with the agarose-bacteria of the other cell population. After it solidified, the complete cylindrical agarose gel was injected into the colon of the mouse with a PBS back-filled syringe. For the colon imaging, imaging planes were selected to avoid gas bubbles in the field of view. In all in vivo experiments, three transducers were used, including two L22-14v and one L22-14vX, due to transducer failures unrelated to this study. B-mode images were acquired at 1.9V (corresponding to 162 kPa in water) for L22-14v, and 1.6V (309 kPa in water) for L22-14vX. x-AM images were acquired at 20V (1.27 MPa in water) for L22-14v and 15V (1.56 MPa in water) for L22-14vX, with other parameters being the same as those used for in vitro imaging. B-mode anatomical imaging was performed at 7.4V using the ‘L22-14v WideBeamSC’ script provided by Verasonics.

### Image Processing and Data Analysis

All in vitro and in vivo ultrasound images were processed using MATLAB. Regions of interest (ROIs) were manually defined so as to adequately capture the signals from each sample well or region of the colon. The sample ROI dimensions (1.2 mm×1.2 mm square) were the same for all in vitro phantom experiments. The noise ROI was manually selected from the background for each pair of sample wells. For the in vivo experiments, circular ROIs were manually defined to avoid edge effects from the skin or colon wall, and the tissue ROIs were defined as the rest of the region within the same depth range of the signal ROIs. For each ROI, the mean pixel intensity was calculated, and the pressure-sensitive ultrasound intensity (ΔI=I_{intact}−I_{collapsed}) was calculated by subtracting the mean pixel intensity of the collapsed image from the mean pixel intensity of the intact image. The contrast-to-noise ratio (CNR) was calculated for each sample well by taking the mean intensity of the sample ROI over the mean intensity of the noise ROI. The x-AM by B-mode ratio at a specific voltage (or applied acoustic pressure) was calculated with the following formula:

$$\frac{Δ {I}_{x - AM} ( V )}{Δ {I}_{B \text{-} mode} ( V )}$$

where ΔI_{x-AM} (V) is the pressure-sensitive nonlinear ultrasound intensity acquired by the x-AM sequence at a certain voltage V, and ΔI_{B-mode}(V) is the pressure-sensitive linear ultrasound intensity of the B-mode acquisitions at 1.6V (132 kPa) following the x-AM acquisitions at the voltage V. All images were pseudo-colored (bone colormap for B-mode images, hot colormap for x-AM images), with the maximum and minimum levels indicated in the accompanying color bars.

### Statistical Analysis

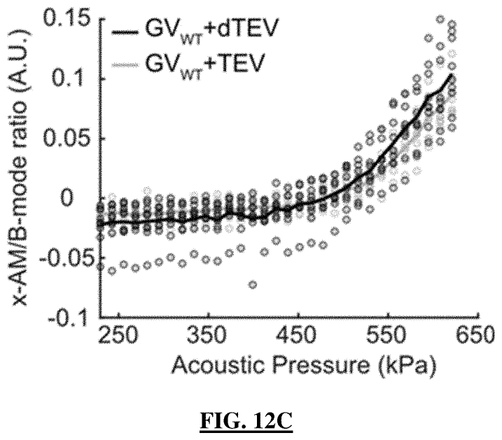

Data is plotted as the mean±standard error of the mean (SEM). Sample size is N=3 biological replicates in all in vitro experiments unless otherwise stated. For each biological replicate, there were technical replicates to accommodate for variability in experimental procedures such as sample loading and pipetting. SEM was calculated by taking the values for the biological replicates, each of which was the mean of its technical replicates. The numbers of biological and technical replicates were chosen based on preliminary experiments such that they would be sufficient to report significant differences in mean values. Individual data for each replicate is given in FIGS. 12A-16 in the form of scatter plots. P values, for determining the statistical significance for the in vivo data, were calculated using a two-tailed paired t-test.

### Example 1: Engineering an Acoustic Sensor of TEV Endopeptidase Activity

This example describes the process of engineering an acoustic sensor of TEV endopeptidase activity.

TEV endopeptidase was selected as the first sensing target because of its well-characterized recognition sequence and widespread use in biochemistry and synthetic biology[39, 40].

To sense TEV activity, a GvpC variant containing the TEV recognition motif ENLYFQ′G (FIG. 7B) was engineered based on the hypothesis that the cleavage of GvpC into two smaller segments would cause the GV shell to become less stiff, thereby allowing it to undergo buckling and produce enhanced nonlinear ultrasound contrast. This design was implemented in vitro using GVs from Anabaena flos-aque (Ana), whose native GvpC can be removed after GV isolation and replaced with new versions expressed heterologously in Escherichia coli[31, 32]. Ana GvpC comprises five repeats of a predicted alpha-helical polypeptide (FIG. 7A), and insertions of the TEV recognition sequence were tested, with and without flexible linkers of different lengths, at several locations within this protein.

After incubating the engineered GVs with active TEV protease or a heat-inactivated “dead” control (dTEV), their hydrostatic collapse was measured using pressurized absorbance spectroscopy. This technique measures the optical density of GVs (which scatter 500 nm light when intact) under increasing hydrostatic pressure, providing a quick assessment of GV shell mechanics: GVs that collapse at lower pressures also produce more nonlinear contrast[10, 31, 32, 42]. Using this approach, we identified an engineered GV variant that showed ˜70 kPa reduction in its collapse pressure midpoint upon incubation with the active TEV protease (FIG. 7C). This GV sensor for TEV, hereafter referred to as GVS_{TEV}, has the TEV cleavage site on the second repeat of GvpC, flanked by flexible GSGSGSG linkers on both sides.

TEV cleavage of the GvpC on GVS_{TEV} is expected to produce N- and C-terminal fragments with molecular weights of approximately 9 and 14 kDa, respectively. Indeed, gel electrophoresis of GVS_{TEV} after exposure to active TEV resulted in the appearance of the two cleaved GvpC fragments and a significant reduction in the intact GvpC band (FIG. 7D). In addition, removal from solution of unbound fragments via buoyancy purification of the GVs resulted in a reduced band intensity for the N-terminal cleavage fragment, indicating its partial dissociation after cleavage (FIG. 7D). No significant changes in the GvpC band intensity were observed after incubation with dTEV. Transmission electron microscopy (TEM) images showed intact GVs with similar appearance under both conditions, confirming that protease cleavage did not affect the structure of the underlying GV shell (FIG. 7E). Dynamic light scattering (DLS) showed no significant difference in the hydrodynamic diameter of the engineered GVs after incubation with dTEV and active TEV protease, confirming that the GVs remain dispersed in solution (FIG. 7F).

After the desired mechanical and biochemical properties of GVS_{TEV} were confirmed, the GVS_{TEV} was imaged with ultrasound. Nonlinear imaging was performed in hydrogel samples containing the biosensor, using a recently developed cross-amplitude modulation (x-AM) pulse sequence. x-AM uses pairs of cross-propagating plane waves to elicit highly specific nonlinear scattering from buckling GVs at the wave intersection, while subtracting the linear signal generated by transmitting each wave on its own[41]. Linear images were acquired using a conventional B-mode sequence. As hypothesized, exposing the GVS_{TEV} samples to TEV protease produced a strong nonlinear acoustic response, with a maximal contrast-to-noise ratio (CNR) enhancement of ˜7 dB at an applied acoustic pressure of 438 kPa (FIG. 7G). Substantially less nonlinear contrast was observed in controls exposed to dTEV, while, as expected, both samples produced similar linear scattering. Consistent with the pressure-dependent mechanics of the GV shell, the differential nonlinear acoustic response of GVS_{TEV} became evident at pressures above 295 kPa, and kept increasing until 556 kPa, at which point the GVs began to collapse (FIG. 7H). As an additional control, it was found that GVs with the wild-type GvpC sequence (GV_{WT}) showed no difference in their hydrostatic collapse pressure or nonlinear acoustic contrast in response to TEV protease (FIGS. 7I-7K), and no wild-type GvpC cleavage was seen upon gel electrophoresis (FIGS. 12A-12C).

These results established GVS_{TEV} as an acoustic biosensor of the TEV protease enzyme and provided a template for developing additional sensors.

### Example 2: Engineering an Acoustic Sensor of Mammalian Calpain

This example describes the process of engineering an acoustic sensor of mammalian calpain.

After validating the basic acoustic biosensor design using the model TEV protease in Example 1, Applicant then examined its generalizability to other endopeptidases, selecting as the second target the calcium-dependent cysteine protease calpain, a mammalian enzyme with critical roles in a wide range of cell types [43-45]. The two most abundant isoforms of this protease, known as μ-calpain and m-calpain, are expressed in many tissues and involved in processes ranging from neuronal synaptic plasticity to cellular senescence[43, 44].

An acoustic biosensor of μ-calpain was designed by inserting the α-spectrin-derived recognition sequence QQEVY′GMMPRD[46] into Ana GvpC (FIG. 8A). Several versions of GvpC incorporating this cleavage sequence were screened, flanked by GSG or GSGSG linkers, at different positions within the second helical repeat. Pressurized absorbance spectroscopy performed in buffers with and without calpain and Ca²⁺ allowed us to identify a GV sensor for calpain (GVS_{calp}), showing an approximately 50 kPa decrease in hydrostatic collapse pressure in the presence of the enzyme and its ionic activator (FIG. 8B). Electrophoretic analysis confirmed cleavage and partial dissociation of the cleaved fragments from the GV surface (FIG. 8C), while TEM showed no change in GV morphology (FIG. 8D).

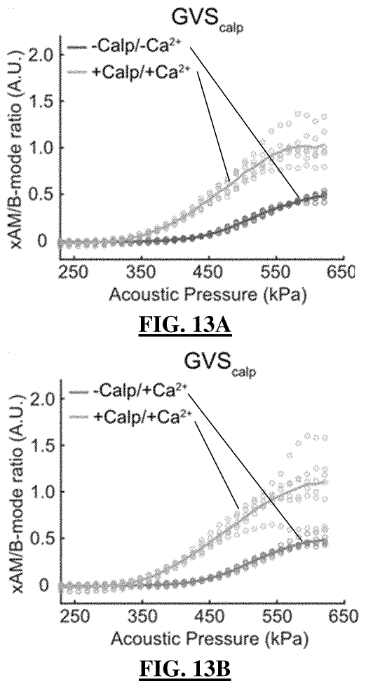

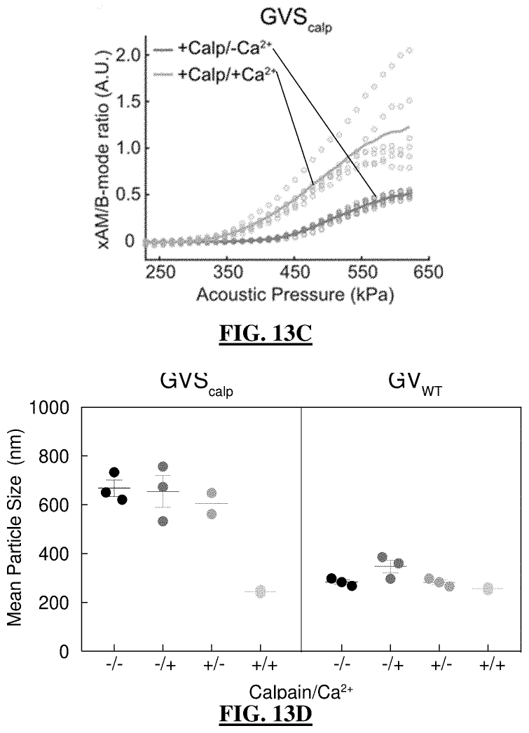

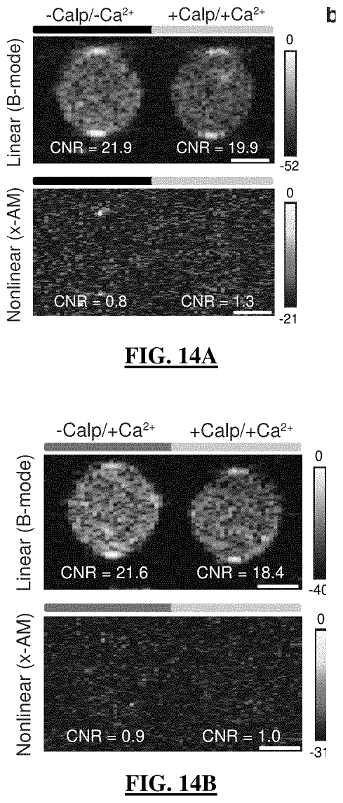

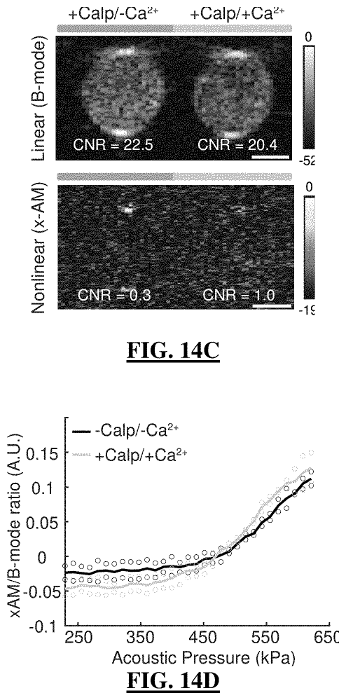

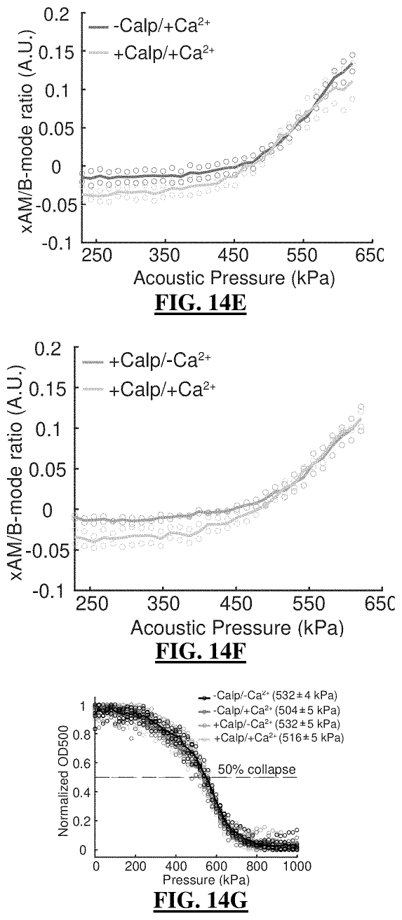

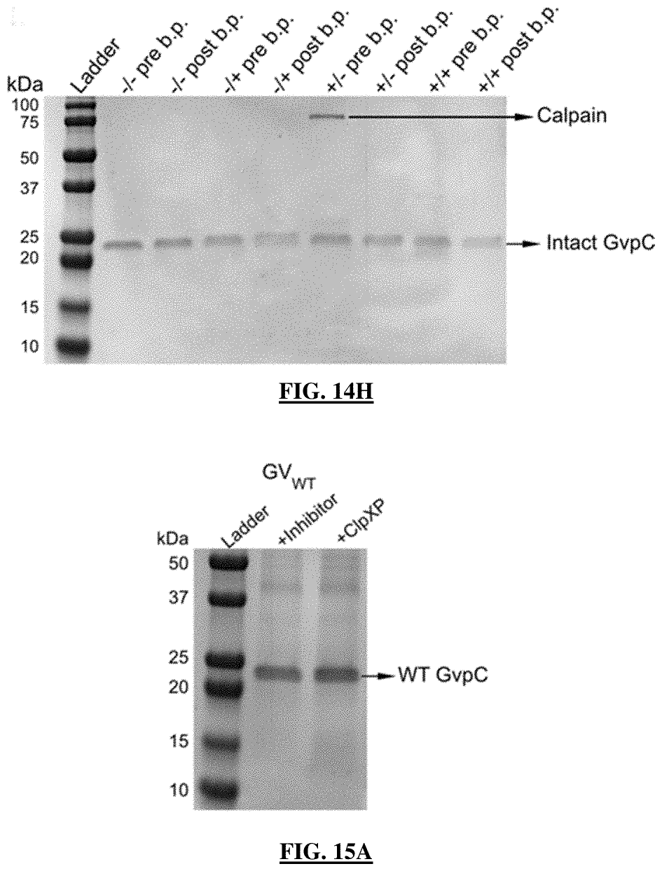

Ultrasound imaging of GVS_{calp} revealed a robust nonlinear acoustic response when both calpain and calcium were present (FIGS. 8E, 8G, 8I), but not in negative controls lacking either or both of these analytes. A slight clustering tendency of GVS_{calp} nanostructures, which was attenuated by incubation with activated calpain (FIGS. 13A-13D), resulted in a slightly higher B-mode signal for the negative controls. However, this did not significantly affect the maximal nonlinear sensor contrast of GVS_{calp} of approximately 7 dB (FIGS. 8E, 8G, 8I). This contrast increased steeply beyond an applied acoustic pressure of 320 kPa (FIGS. 8F, 8H, 8J). With this biosensor, ultrasound could be used to visualize the dynamic response of calpain to Ca²±, with a half-maximal response concentration of 140 μM (FIG. 8K). Additional control experiments performed on GVs with wild-type GvpC showed no proteolytic cleavage, change in GV collapse pressure or ultrasound response, after incubation with calcium-activated calpain (FIGS. 14A-14H).

These results show that acoustic biosensor designs based on GvpC cleavage can be generalized to a mammalian protease and used to sense the dynamics of a conditionally active enzyme.

### Example 3: Building an Acoustic Sensor of the Processive Protease ClpXP

This example describes the process of building an acoustic sensor of the processive protease ClpXp.

In addition to endopeptidases demonstrated in Examples 1-2, another important class of enzymes involved in cellular protein signaling and homeostasis is processive proteases, which unfold and degrade full proteins starting from their termini[47].

To determine whether GV-based biosensors could be developed for this class of enzymes, ClpXP was selected, a processive proteolytic complex from E. coli comprising the unfoldase ClpX and the peptidase ClpP[48]. ClpX recognizes and unfolds protein substrates containing specific terminal peptide sequences called degrons. The unfolded proteins are then fed into ClpP, which degrades them into small peptide fragments[48]. It was hypothesized that the addition of a degron to the C-terminus of GvpC would enable ClpXP to recognize and degrade this protein, while leaving the underlying GvpA shell intact, resulting in GVs with greater mechanical flexibility and nonlinear ultrasound contrast (FIG. 9A).

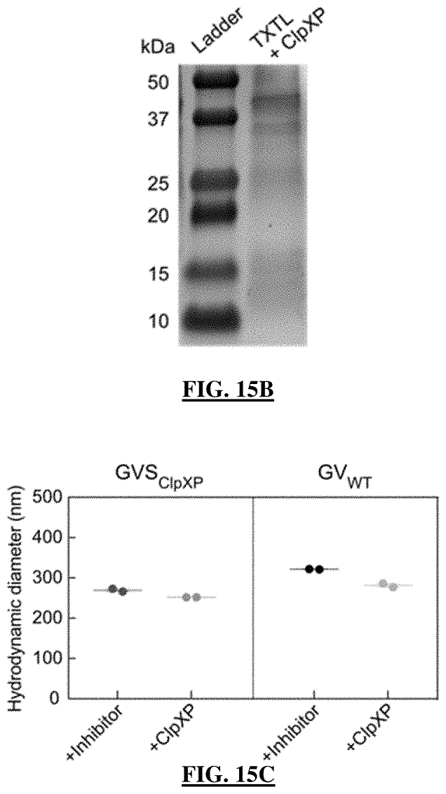

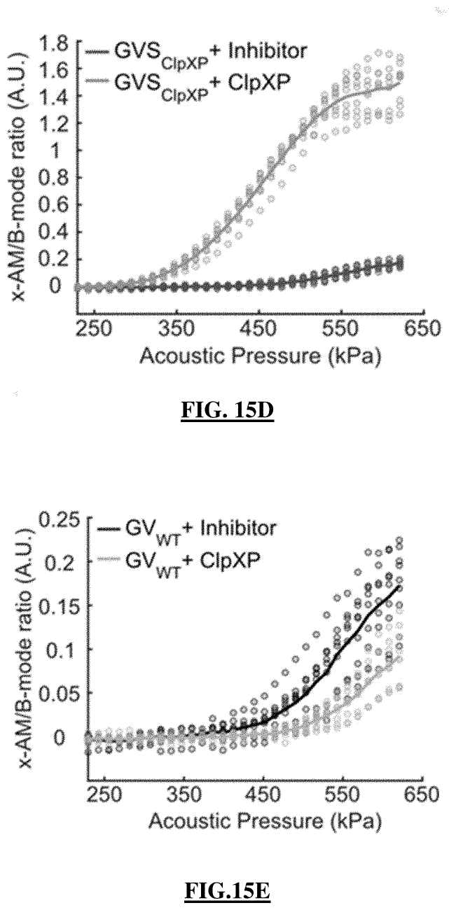

To test this hypothesis, the ssrA degron, AANDENYALAA, as appended via a short SG linker, to the C-terminus of Ana GvpC, resulting in a sensor named GVS_{ClpXP} (FIG. 9A). The performance of this biosensor was tested in vitro using a reconstituted cell-free transcription-translation system comprising E. coli extract, purified ClpX, and a ClpP plasmid. Gel electrophoresis performed after incubating GVS_{ClpXP} with this cell-free extract showed significant degradation of the engineered GvpC, compared to a negative control condition in which the extract was pre-treated with a protease inhibitor (FIG. 9B). TEM images showed intact GVs under both conditions, confirming that GvpC degradation left the underlying GV shell uncompromised (FIG. 9C). Pressurized absorbance spectroscopy indicated a substantial weakening of the GV shell upon ClpX exposure, with the hydrostatic collapse midpoint shifting by nearly 250 kPa (FIG. 9D). Ultrasound imaging revealed a 17 dB enhancement in the nonlinear contrast produced by GVS_{ClpXP} at an acoustic pressure of 477 kPa, in response to ClpXP activity (FIG. 9E-9F). Control GVs containing wild type GvpC showed no sensitivity to ClpXP (FIGS. 9G, 9H, 9I; FIGS. 15A-15E).

These results establish the ability of GV-based acoustic biosensors to visualize the activity of a processive protease as turn-on sensors.

### Example 4: Constructing Intracellular Acoustic Sensor Genes

This example demonstrates the ability of the acoustic biosensors to respond to enzymatic activity inside living cells.

After demonstrating the performance of acoustic biosensors in vitro, Applicant endeavored to show that they could respond to enzymatic activity inside living cells. As the cellular host, E. coli Nissle 1917 was selected. This probiotic strain of E. coli has the capacity to colonize the mammalian gastrointestinal tract, and is widely used as a chassis for the development of microbial therapeutics[49-51], making it a valuable platform for intracellular bio sensors.

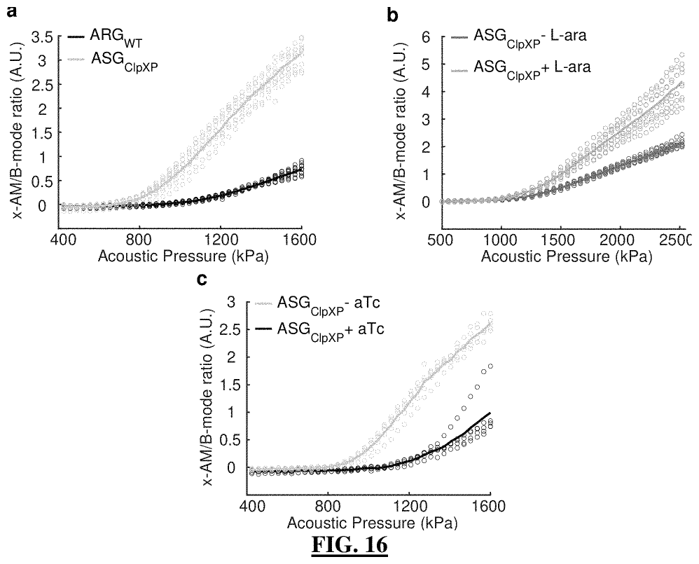

Recently, an engineered operon comprising GV-encoding genes from Anabaena flos-aquae and Bacillus megaterium was expressed in Nissle cells as acoustic reporter genes (ARGs), allowing gene expression to be imaged with linear B-mode ultrasound[10]. To develop an intracellular acoustic sensor gene targeting ClpXP (ASG_{ClpXP}), the wild type GvpC in the ARG gene cluster (ARG_{WT}) was swapped with the modified GvpC from GVS_{ClpXP} (dGvpC) (FIG. 10A), and transformed into wild-type (WT) Nissle cells, which natively express ClpXP. It was hypothesized that it would show a reduced intracellular collapse pressure and enhanced nonlinear contrast compared to ARG_{WT}. Indeed, pressurized absorbance spectroscopy on intact cells expressing ASG_{ClpXP} revealed a reduction in the hydrostatic collapse pressure midpoint of ˜160 kPa relative to cells expressing ARG_{WT} (FIG. 10B). In ultrasound imaging, live cells expressing ASG_{ClpXP} showed an enhancement in nonlinear contrast of approximately 13 dB (FIG. 10C), while linear B-mode signal was similar. The nonlinear response of ASG_{ClpXP} expressing cells was strongest beyond an acoustic pressure of 784 kPa (FIG. 10D; FIG. 16 panel a).

Next, to examine the ability of ASG_{ClpXP} to respond to intracellular enzymatic activity in a dynamic manner, a ClpXP-deficient strain of Nissle cells (ΔClpXP) was generated through genomic knock-out of the genes encoding ClpX and ClpP, and a plasmid containing these two genes under the control of an arabinose-inducible promoter was created (FIG. 4A). This allowed one to externally control the activity of the ClpXP enzyme. ΔClpXP Nissle cells were co-transformed with the inducible-ClpXP plasmid and ASG_{ClpXP}. ClpXP induction in these cells with L-arabinose resulted in an approximately 160 kPa reduction in the hydrostatic collapse pressure midpoint (FIG. 10E). Under ultrasound imaging, cells with induced ClpXP activity showed substantially stronger nonlinear contrast (+6.7 dB) compared to cells uninduced for this protease (FIG. 10F), while showing a similar B-mode signal. This enhancement in nonlinear signal was detectable with acoustic pressures above 950 kPa (FIG. 10G; FIG. 16 panel b).

These experiments demonstrated the ability of ASG_{ClpXP} to function as an intracellular acoustic sensor to monitor variable enzyme activity.

A major application of dynamic sensors in cells is to monitor the activity of natural or synthetic gene circuits[52-55]. To test if the acoustic sensors herein described could be used to track the output of a synthetic gene circuit in cells, Applicant co-transformed WT Nissle cells with ASG_{ClpXP}, and a separate wild-type GvpC gene controlled by anhydrotetracycline (aTc) (FIG. 10H). The hypothesis was that induction of this gene circuit only with IPTG would result in the production of GVs with ClpXP-degradable GvpC, resulting in nonlinear contrast, whereas the additional input of aTc would result in the co-production of non-degradable wild-type GvpC, which would take the place of any degraded engineered GvpC on the biosensor shell, leading to reduced nonlinear scattering (FIG. 10H).

Indeed, when cells were induced with just IPTG strong nonlinear contrast was observed. However, when aTc was added to the cultures after IPTG induction, this contrast was reduced by approximately 10 dB (FIGS. 10I, and 10J, FIG. 16 panel c).

These results, together with the findings in ΔClpXP cells with inducible ClpXP, show that acoustic biosensors can be used to visualize the output of synthetic gene circuits.

### Example 5: Ultrasound Imaging of Bacteria Expressing Acoustic Sensor Genes In Vivo

This example demonstrates the ability of the acoustic sensor constructs herein described to produce ultrasound contrast within a biologically relevant anatomical location in vivo.

Finally, after establishing the basic principles of acoustic biosensor engineering in vitro and demonstrating their performance in living cells, Applicant assessed the ability of the sensor constructs herein described to produce ultrasound contrast within a biologically relevant anatomical location in vivo.

In particular, approaches to imaging microbes in the mammalian GI tract[10, 56-58] are needed to support the study of their increasingly appreciated roles in health and disease[59-61] and the development of engineered probiotic agents[62, 63]. The GI tract is also an excellent target for ultrasound imaging due to its relatively deep location inside the animal, and the use of ultrasound in clinical diagnosis and animal models of GI pathology, with appropriate measures taken to minimize potential interference from air bubbles and solid matter[64, 65].

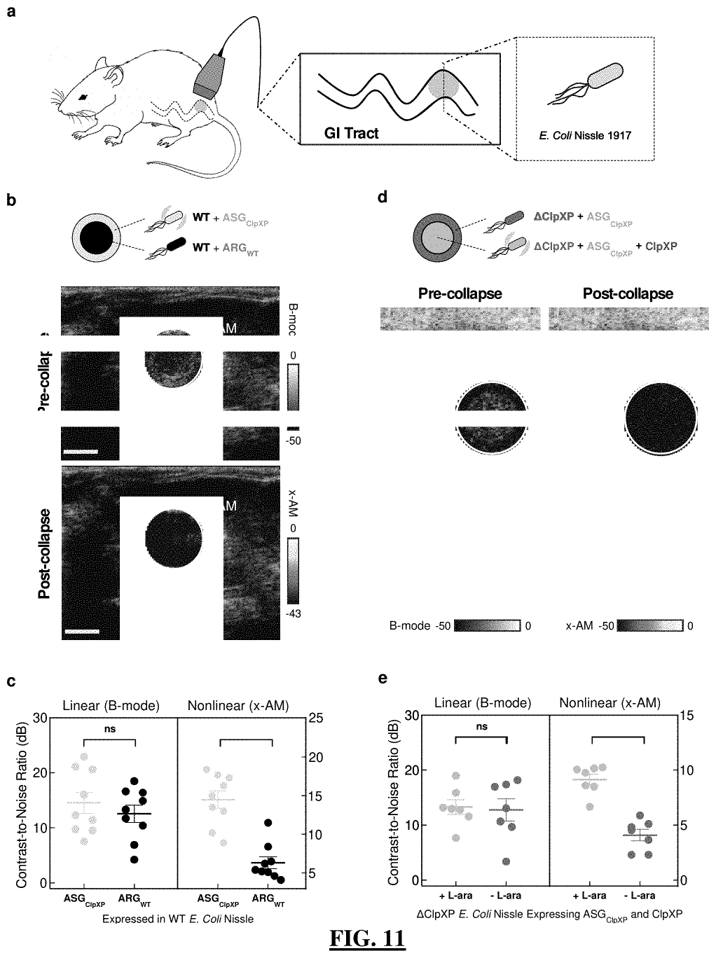

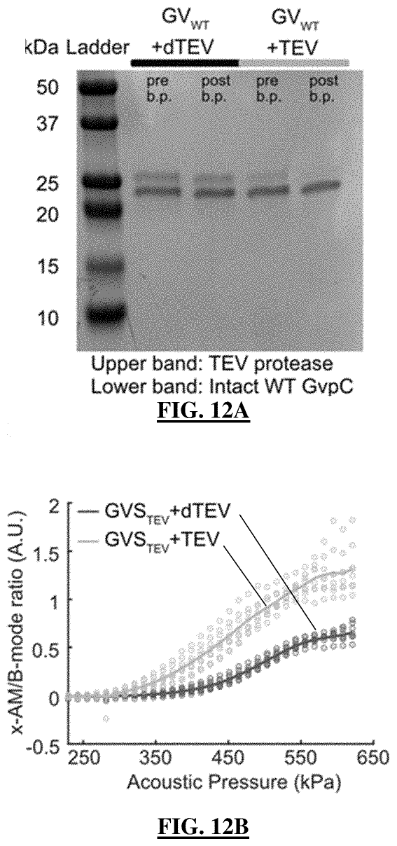

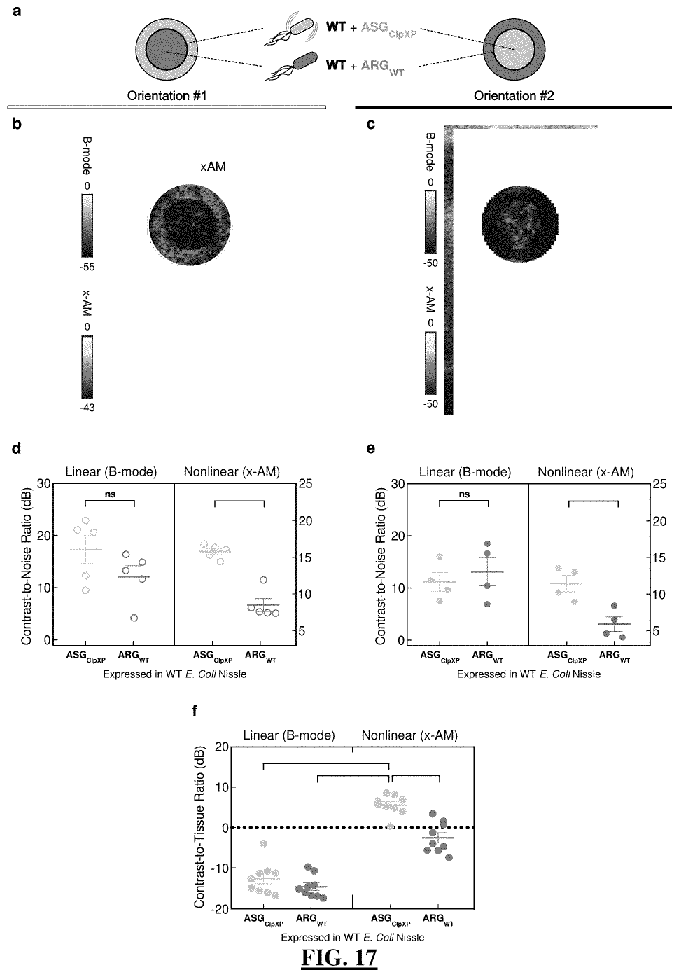

To demonstrate the ability of acoustic biosensors to produce nonlinear ultrasound contrast within the in vivo context of the mouse GI tract, WT Nissle cells expressing ASG_{ClpXP} and ARG_{WT} were first co-injected into the mouse colon (FIG. 11, panel a), distributing one cell population along the lumen wall and the other in the lumen center. In these proof-of-concept experiments, the cells are introduced into the colon in a rectally-injected agarose hydrogel to enable precise positioning and control over composition. Using nonlinear ultrasound imaging, one could clearly visualize the unique contrast generated by the protease-sensitive ASG as a bright ring of contrast lining the colon periphery (FIG. 11, panel b). When the spatial arrangement was reversed, the bright nonlinear contrast was concentrated in the middle of the lumen (FIG. 17). A comparison of ultrasound images acquired before and after acoustic collapse of the GVs, using a high-pressure pulse from the transducer, confirmed that the bright ring of nonlinear contrast was emanating from ASG_{ClpXP}-expressing cells (FIG. 11, panel b), and this result was consistent across independent experiments in 9 mice (FIG. 11, panel c).

To demonstrate in vivo imaging of enzyme activity, ΔClpXP Nissle cells expressing ASG_{ClpXP} were introduced into the mouse colon, with and without transcriptionally activating intracellular ClpXP. As above, the cells were contained in an agarose hydrogel. Cells induced to express this enzyme showed enhanced nonlinear contrast compared to cells not expressing ClpXP (FIG. 11, panel d). Acoustic collapse confirmed the acoustic biosensors as the primary source of nonlinear signal (FIG. 11, panel d). This performance was consistent across 7 mice and 2 spatial arrangements of the cells (FIG. 11, panel e).

These results demonstrate the ability of acoustic biosensors to visualize enzyme activity within the context of in vivo imaging.

Besides molecular sensing, one additional benefit of the nonlinear contrast generated by ASG_{ClpXP}-expressing cells is to make the cells easier to detect relative to background tissue compared to linear B-mode imaging. Indeed, the nonlinear contrast of WT Nissle cells expressing ASG_{ClpXP} had a significantly higher contrast-to-tissue ratio than either the nonlinear contrast of ARG_{WT}-expressing cells, or the B-mode contrast of either of these two species (FIG. 11, panel f).

### Example 6: Identification of Gyp Genes and Protein Sequences Through Alignment

Gyp genes and related protein can be identified through alignment of sequences in databases or identified through wet bench experiments with an approach and techniques identifiable by a skilled person.

Taking as gvpA/B as an example, the identification can be performed using consensus sequence: SSSLAEVLDRILDKGXVIDAWARVSLVGIEILTIEARVVIASVDTYLR (SEQ ID NO: 1) wherein X can be any amino acid. LDRILD (SEQ ID NO: 3), RILDKGXVIDAWARVS (SEQ ID NO: 4) wherein X can be any amino acid, and/or DTYLR (SEQ ID NO: 5), and/or of exemplary gvpA and gvpB protein sequences already identified, as it will be understood by a skilled person.

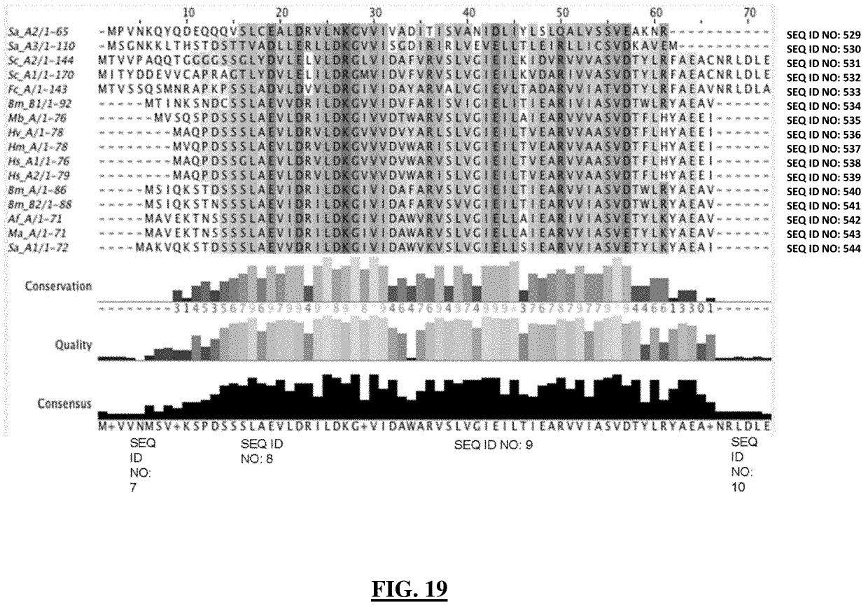

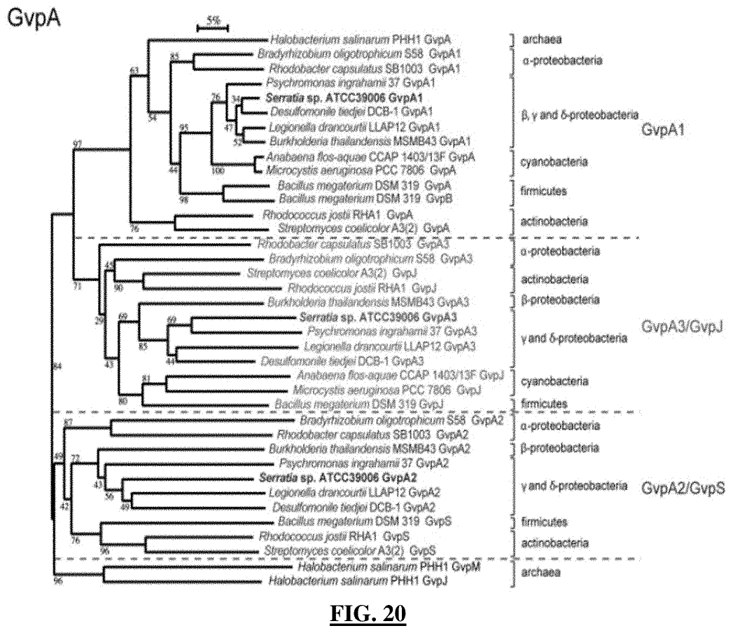

FIG. 19 shows an exemplary Clustal omega alignment of amino acid sequences of selected exemplary gvpA and gvpB proteins.

The gvpA and gvpB proteins shown are from the following species: Sa_A2, Serratia sp. ATCC 39006 gvpA2; Sa_A3, Serratia sp. ATCC 39006 gvpA3; Sc_A2, Streptomyces coelicolor gvpA2; Sc_A1, Streptomyces coelicolor gvpA1; Fc_A, Frankia sp. gvpA; Bm_B1, B. megaterium gvpB1; Mb_A, Methanosarcina barkeri gvpA; Hv_A, Halorubrum vacuolatum gvpA; Hm_A, Haloferax mediterranei gvpA; Hs_A1, Halobacterium sp. NRC-1 gvpA1; Hs_A2, Halobacterium sp. NRC-1 gvpA2; Bm_A, B. megaterium gvpA; Bm_B2, B. megaterium gvpB2; Af_A, A. flos-aquae gvpA; Ma_A,; Sa_A1, Serratia sp. ATCC 39006 gvpA1.

The bottom row of FIG. 19 indicated as “Consensus” shows an exemplary consensus sequence derived from alignment of the gvpA and gvpB amino acid sequences shown.

Homology-based searching (e.g., BLAST alignment) of sequences of proteins encoded in the genome of a prokaryotic organism compared to the exemplary consensus sequence shown in FIG. 19 can be used to identify gvpA and/or gvpB protein sequences in the prokaryotic organism.

### Example 7: Identification Gyp Genes and Protein Sequences Through Phylogenesis

Gyp genes and related protein can be identified based on phylogenetic relationships of sequences in databases or identified through wet bench experiments with an approach and techniques identifiable by a skilled person.

In particular, exemplary gvpA, gvpF and gvpN genes and proteins were identified phylogenetic relationships as shown below.

FIG. 20 shows exemplary phylogenetic relationships of the gvpA protein sequences from the indicated prokaryotic species [1]. Table 14 lists examples of GV protein sequences from a number of prokaryotic species.

Identification of a gvpA/B protein can be performed by comparing the sequence of an unknown protein in a prokaryotic cell with that of a known gvpA sequence from the closest phylogenetic relative of the prokaryotic species, such as those indicated in the exemplary phylogenetic tree diagram in FIG. 20. Alternatively, identification of gvpA/B can be done through protein alignment algorithms (e.g. BLAST) with the gvpA/B consensus sequence provided in this document, where the protein identity has 60% or higher to this sequence.

FIG. 21 shows exemplary phylogenetic relationships of the gvpF and gvpL protein sequences from the indicated prokaryotic species [1]. In some embodiments described herein, the identification of a gvpF protein can be performed by comparing the sequence of an unknown protein in a prokaryotic cell with that of a known gvpF sequence from the closest phylogenetic relative of the prokaryotic species, such as those indicated in the exemplary phylogenetic tree diagram in FIG. 21.

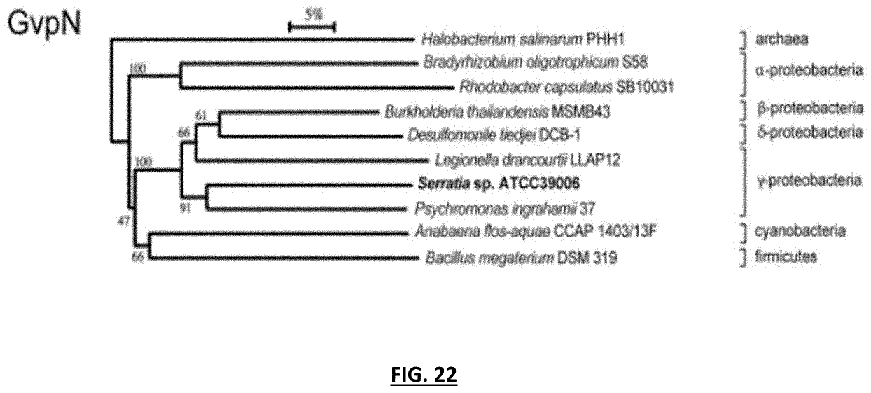

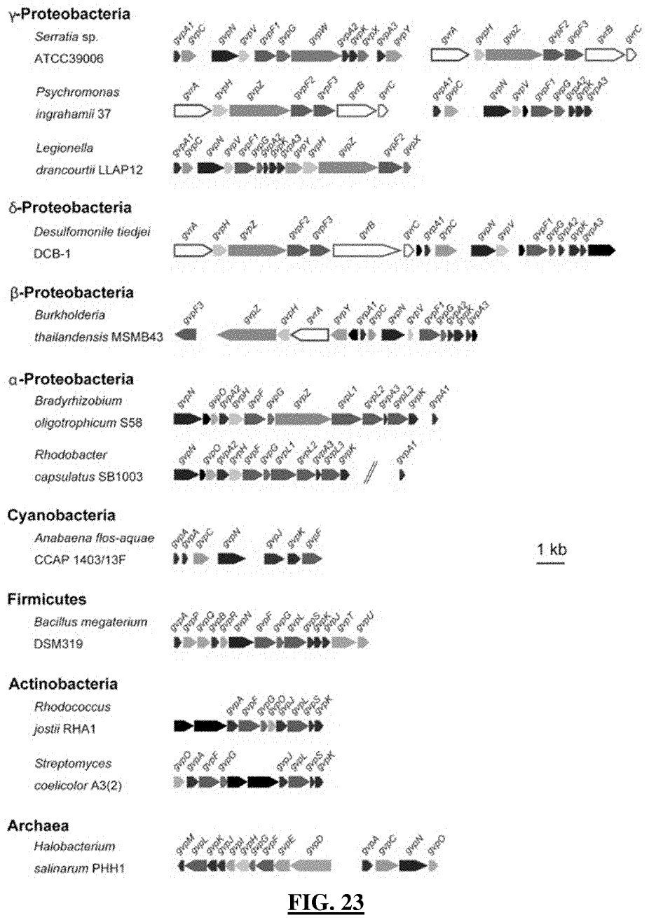

FIG. 22 shows exemplary phylogenetic relationships of the gvpN protein sequences from the indicated prokaryotic species [1]. In some embodiments described herein, the identification of a gvpN protein can be performed by comparing the sequence of an unknown protein in a prokaryotic cell with that of a known gvpN sequence from the closest phylogenetic relative of the prokaryotic species, such as those indicated in the exemplary phylogenetic tree diagram in FIG. 22.

The protein sequences provided in Table 14 can also be used with protein alignment algorithms to identify gyps. Where the using BLAST or other tools, if the top 100 based on protein identity or 100 lowest E-values are identified as “gas vesicle protein” or “gyp” or “gas vesicle structural protein”, the protein can be designated as a gas vesicle protein.

### Example 8: Identification of Gyp Genes and Proteins Through Analysis of Configuration Vesicle Gene Clusters in Prokaryotes

Identification of gyp genes and proteins can be performed also GV cluster configuration of gas vesicle gene clusters in prokaryotes which can be used to identify the specific genes forming a GV cluster in a microorganism, in combination with use of consensus sequences, alignment and/or phylogenetic analysis of GV clusters.

FIG. 23 shows diagrams illustrating the organization of exemplary gas vesicle gene clusters. Gas vesicle gene clusters from the indicated organisms are shown, with genes shown as block-shaped arrows, and genes of predicted similar function indicated in the same shade of grey. The direction of the transcription of genes within a gene cluster is indicated by the direction of the block-shaped arrows, and genes grouped together having block arrows pointed in the same direction are typically organized in the same operon. The scale bar indicates 1 kb [1].

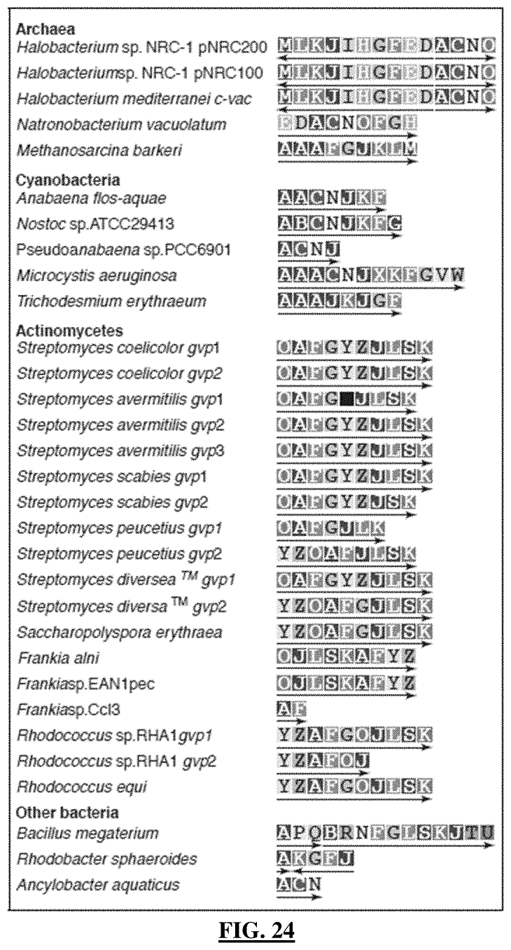

In addition, FIG. 24 shows diagrams illustrating organization of exemplary gyp gene clusters, wherein each letter indicates a gyp gene, and an arrow beneath a group of letters indicates an operon, with the direction of the arrow indicating the direction of transcription [2].

To identify gyp genes and gyp gene cluster, the following methodology can be used

1. Using the 60+% gvpA/B and/or 50%+gvpN consensus sequences and/or gyp sequences provided in Table 14, identify gyp genes on the genome of the prokaryote.

2. For a gyp gene identified, test the next 10 protein coding sequences on both side of the gene to determine if it is gyp gene. Using BLAST or other tools, if the top 100 based on protein identity or 100 lowest E-values are identified as “gas vesicle protein” or “gyp” or “gas vesicle structural protein”, the protein can be designated as a gas vesicle protein.

3. If the adjacent genes are labeled as gyp gene, continue testing the next 10 protein coding sequences on both sides of the protein, moving away from the labeled gyp genes. Use criterion 2 to continue identifying gyp genes. If the adjacent 10 genes are not marked as gyp genes continue to next part.

4. The genes at the extreme ends will mark the edge of the gene cluster and all the genes inside are part of the gene cluster than can be tested for heterologous expression gas vesicle in bacteria/mammalian cells. In some cases, there can be one or more gene clusters encoding gyp genes, therefore all the gene clusters are tested during heterologous expression.

In particular, the above methodology can be one way to identify gyp gene clusters in an unannotated or mis-annotated genome as will be understood by a skilled person.

### Example 9: Amino Acid Sequences of Exemplary GV Proteins Including GVS and GVA Proteins

Several gyp genes and related proteins have been identified and are available in accessible databases.

In particular, Tables 14-18 show amino acid sequences of exemplary GVS (gvpA/B or gvpC) and GVA proteins from several exemplary prokaryotic species. In particular, these exemplary amino acid sequences can be used as reference amino acid sequences in some embodiments for homology-based searches for related GVS and GVA proteins.

TABLE 14
Amino acid sequences of exemplary gvpA/B, gvpF, gvpF/L, gvpG, gvpJ, gvpK, gvpL, gvpN,
gvpV, gvpW, gvpR, gvpS, gvpT, and gvpU proteins

| | | SEQ ID |
|---|---|---|
| Species, protein; | Amino acid sequence | NO.: |
| gvpA/B | | |
| Ana-family- | MAVEKTNSSSSLAEVIDRILDKGIVIDAWVRVSLVGIELLAIEARXVIASVETYLKYAE | 130 |
| consensus_gvpA | AVGLTXSAAVPAX | |
| Aphanizomenon-flos- | MAVEKTNSSSSLAEVIDRILDKGIVIDAWVRVSLVGIELLAIEARIVIASVETYLKYAE | 131 |
| aquae_gvpA | AVGLTQSAAVPA* | |
| Aphanothece- | MAVEKTNSSSSLGEVVDRILDKGVVVDLWVRVSLVGIELLAVEARVVVASVETYLK | 132 |
| halophytica_gvpA | YAEAVGLTSSAAVPAE* | |
| Anabaena-flos- | MAVEKTNSSSSLAEVIDRILDKGIVIDAWVRVSLVGIELLAIEARIVIASVETYLKYAE | 133 |
| aquae_gvpA | AVGLTQSAAVPA* | |
| Ancylobacter- | MAVEKINASSSLAEVVDRILDKGVVVDAWVRVSLVGIELLAVEARVVVAGVDTYLK | 134 |
| aquaticus_gvpA | YAEAVGLTASAQAA* | |
| Aquabacter- | MAVEKINASSSLAEVVDRILDKGVVVDAWVRVSLVGIELLAVEARVVVAGVDTYLK | 135 |
| spiritensis_gvpA | YAEAVGLTAGAQAA* | |
| Arthrospira-sp-PCC- | MAVEKVNSSSSLAEVIDRILDKGIVIDAWVRVSLVGIELLSVEARVVIASVETYLKYA | 136 |
| 8005_gvpA | EAVGLTAQAAVPSV* | |
| Calothrix-sp-strain- | MAVEKTNSSSSLAEVIDRILDKGIVVDAWVRVSLVGIELLAIEARIVIASVETYLKYAE | 137 |
| PCC-7601_gvpA | AVGLTQSAAVPA* | |
| Dactylococcopsis- | MAVEKTNSSSSLGEVVDRILDKGVVVDLWVRVSLVGIELLAVEARVVIASVETYLKY | 138 |
| salina-PCC- | AEAVGLTSSAAVPAE* | |
| 8305_gvpA1 | | |
| Dolichospermum- | MAVEKTNSSSSLAEVIDRILDKGIVIDAWVRVSLVGIELLAIEARIVIASVETYLKYAE | 139 |
| circinale- | AVGLTQSAAVPA* | |
| AWQC131C_gvpA | | |
| Dolichospermum- | MAVEKTNSSSSLAEVIDRILDKGIVIDAWVRVSLVGIELLAIEARIVIASVETYLKYAE | 140 |
| lemmermannii_gvpA | AVGLTQSAAVPA | |
| Enhydrobacter- | MAVEKMNASSSLAEVVDRILDKGIVIDAWVRVSLVGIELLAVEARVVVAGVDTYLK | 141 |
| aerosaccus_gvpA1 | YAEAVGLTAGAEAA* | |
| Lyngbya-confervoides- | MAVEKVNSSSSLAEVVDRILDKGIVVDAWVRVSLVGIELLAIEARVVIASVETYLKY | 142 |
| BDU141951_gvpA | AEAVGLTAQAAVPAS* | |
| Nostoc-punctiforme- | MAVEKVNSSSSLAEVIDRILDKGIVIDAWVRVSLVGIELLSIEARIVIASVETYLRYAE | 143 |
| PCC-73102_gvpA | AVGLTSQAAVPSAA* | |
| Nostoc-sp-PCC- | MAVEKTNSSSSLAEVIDRILDKGIVVDAWVRVSLVGIELLAIEARIVIASVETYLKYAE | 144 |
| 7120_gvpA | AVGLTQSAAMPA* | |
| Microchaete- | MAVEKTNSSSSLAEVIDRILDKGIVVDAWVRVSLVGIELLAIEARIVIASVETYLKYAE | 145 |
| diplosiphon_gvpA | AVGLTQSAAVPA* | |
| Microcystis- | MAVEKTNSSSSLAEVIDRILDKGIVIDAWARVSLVGIELLAIEARVVIASVETYLKYAE | 146 |
| aeruginosa-NIES- | AVGLTQSAAVPA* | |
| 843_gvpA1 | | |
| Microcystis- | MAVEKTNSSSSLAEVIDRILDKGIVIDAWARVSLVGIELLAIEARVVIASVETYLKYAE | 147 |
| aeruginosa-NIES- | AVGLTQSAAVPA* | |
| 843_gvpA2 | | |
| Microcystis- | MAVEKTNSSSSLAEVIDRILDKGIVIDAWARVSLVGIELLAIEARVVIASVETYLKYAE | 148 |
| aeruginosa-NIES- | AVGLTQSAAVPA* | |
| 843_gvpA3 | | |
| Microcystis-flos- | MAVEKTNSSSSLAEVIDRILDKGIVIDAWARVSLVGIELLAIEARVVIASVETYLKYAE | 149 |
| aquae-TF09_gvpA | AVGLTQSAAVPA* | |
| Phormidium-tenue- | MAVEKVNSSSSLAEVVDRILDKGIVIDAWVRVSLVGIELLAIEARVVIASVDTYLKYA | 150 |
| NIES-30_gvpA | EAVGLTAQAAVPAA* | |
| Planktothrix- | MAVEKVNSSSSLAEVIDRILDKGIVIDAWVRVSLVGIELLSIEARIVIASVETYLKYAE | 151 |
| agardhii_gvpA | AVGLTAQAAVPSV | |
| Planktothrix- | MAVEKVNSSSSLAEVIDRILDKGIVIDAWVRVSLVGIELLSIEARIVIASVETYLKYAE | 152 |
| rubescens_gvpA | AVGLTAQAAVPSV* | |
| Pseudanabaena- | MAVEKVNSSSSLAEVIDRILDKGIVIDAWVRVSLVGIELLSIEARVVIASVETYLKYAE | 153 |
| galeata-PCC- | AVGLTASAAVPAA | |
| 6901_gvpA | | |
| Stella-vacuolata_ | MAVEKINASSSLAEVVDRILDKGVVVDAWVRVSLVGIELLAVEARVVVAGVDTYLK | 154 |
| gvpA | YAEAVGLTAGAQTA* | |
| Trichodesmium- | MAVEKVNSSSSLAEVIDRILDKGVVVDAWIRLSLVGIELLTIEARIVVASVETYLKYAE | 155 |
| erythraeum- | AVGLTTLAAAPGEAAA* | |
| IMS101_gvpA3 | | |
| Trichodesmium- | MAVEKVNSSSSLAEVIDRILDKGVVVDAWVRLSLVGIELLTIEARIVIASVETYLKYAE | 156 |
| erythraeum- | AVGLTTLAAEPAA* | |
| IMS101_gvpA4 | | |
| Tolypothrix-sp.-PCC- | MAVEKTNSSSSLAEVIDRILDKGIVVDAWVRVSLVGIELLAIEARIVIASVETYLKYAE | 157 |
| 7601_gvpA1 | AVGLTQSAAVPA* | |
| Tolypothrix-sp.-PCC- | MAVEKTNSSSSLAEVIDRILDKGIVVDAWVRVSLVGIELLAIEARIVIASVETYLKYAE | 158 |
| 7601_gvpA2 | AVGLTQSAAVPA* | |
| Halo-family- | MAQPDSSSLAEVLDRVLDKGVVVDVWARXSLVGIEILTVEARVVAASVDTFLHYA | 159 |
| consensus_gvpA | EEIAKIEQAELTAGAEA-XPAPEA | |
| Halobacterium- | MAQPDSSGLAEVLDRVLDKGVVVDVWARVSLVGIEILTVEARVVAASVDTFLHYA | 160 |
| salinarum_gvpA1 | EEIAKIEQAELTAGAEAAPEA | |
| Halobacterium- | MAQPDSSSLAEVLDRVLDKGVVVDVWARISLVGIEILTVEARVVAASVDTFLHYAE | 161 |
| salinarum_gvpA2 | EIAKIEQAELTAGAEAPEPAPEA | |
| Halobacterium- | MAQPDSSGLAEVLDRVLDKGVVVDVWARVSLVGIEILTVEARVVAASVDTFLHYA | 162 |
| salinarum-NRC- | EEIAKIEQAELTAGAEAAPEA* | |
| 1_gvpA1 | | |
| Halobacterium- | MAQPDSSSLAEVLDRVLDKGVVVDVWARISLVGIEILTVEARVVAASVDTFLHYAE | 163 |
| salinarum-NRC- | EIAKIEQAELTAGAEAPEPAPEA* | |
| 1_gvpA2 | | |
| Haloferax- | MVQPDSSSLAEVLDRVLDKGVVVDVWARISLVGIEILTVEARVVAASVDTFLHYAE | 164 |
| mediterranei-ATCC- | EIAKIEQAELTAGAEAAPTPEA* | |
| 33500_gvpA | | |
| Halogeometricum- | MAQPDSSSLAEVLDRVLDKGVVVDVWARVSLVGIEILTVEARVVAASVDTFLHYA | 165 |
| borinquense-DSM- | EEIAKIEQAELTATAEAAPTPEA* | |
| 11551_gvpA | | |
| Halopenitus-persicus- | MAQPDSSGLAEVLDRVLDKGVVVDVWARVSLVGIEILTVEARVVAASVDTFLHYA | 166 |
| strain-DC30_gvpA | EEIAKIEQAELTAGAEAAPEA | |
| Haloquadratum- | MAQPDSSSLAEVLDRVLDKGIVVDTFARISLVGIEILTVEARVVVASVDTFLHYAEEI | 167 |
| walsbyi-C23_gvpA | AKIEQAELTAGAEA* | |
| Halorubrum- | MAQPDSSSLAEVLDRVLDKGVVVDVYARLSLVGIEILTVEARVVAASVDTFLHYAE | 168 |
| vacuolatum-strain- | EIAKIEQAELTAGAEAAPTPEA* | |
| DSM-8800_gvpA | | |
| Halopiger- | MAQPQRRPDSSSLAEVLDRILDKGVVIDVWARISVVGIELLTIEARVVVASVDTFLH | 169 |
| xanaduensis_gvpA1 | YAEEIAKIEQATAEGDLEELEELEVEPRPESSPQSAAE* | |
| Natrialba-magadii- | MAQPQRRPDSSSLAEVLDRVLDKGVVIDIWARVSVVGIELLTVEARVVVASVDTFL | 170 |
| ATCC-43099_gvpA | HYAEEIAKIEQATAEGDLEDLEELEVEPRPESSPKSATE* | |
| Natrinema- | MAQPQRRPDSSSLAEVLDRVLDKGVVIDVWARISVVGIELLTIEARVVVASVDTFL | 171 |
| pellirubrum-DSM- | HYAEEIAKIEQATAEGDLDELEELEVEPRPESSPKSAE* | |
| 15624_gvpA1 | | |
| Natronobacterium- | MAQPQRRPDSSSLAEVLDRILDKGVVIDVWARVSVVGIELLTIEARVVVASVDTFL | 172 |
| gregoryi-SP2_gvpA1 | HYAEEIAKIEQATAEGDLEDLEELEVEPRPESSPQSATE* | |
| Methanosaeta- | MVTSTPDSSSLAEVLDRILDKGIVVDVWARVSLVGIEILTVEARVVVASVDTFLHYS | 173 |
| thermophila_gvpA1 | EEMAKIEQAAIAAAPSA* | |
| Methanosaeta- | MVTSTPDSSSLAEVLDRILDKGIVVDVWARVSLVGIEILTVEARVVVASVDTFLHYS | 174 |
| thermophila_gvpA2 | EEMAKIEQAAIAAAPGVPA* | |
| Methanosarcina- | MVSQSPDSSSLAEVLDRILDKGIVVDVWARVSLVGIEILAIEARVVVASVDTFLHYA | 175 |
| barkeri-3_gvpA1 | EEITKIEIAAKEEKPAIAA* | |
| Methanosarcina- | MVSQSPDSSSLAEVLDRILDKGIVVDTWARVSLVGIEILAIEARVVVASVDTFLHYA | 176 |
| vacuolata_gvpA1 | EEITKIEIAAREEKPVIAA* | |
| Methanosarcina- | MVSQSPDCSSLAEVLDRILDKGIVVDTWARVSLVGIEILAIEARVVVASVDTFLHYA | 177 |
| vacuolata_gvpA2 | EEITKIEIAAREEKPVIAA* | |
| Haladaptatus- | MVQAEPNSSSLADVLDRILDKGVVIDVWARISVVGIEVLTVEARVVVASVDTFLHY | 178 |
| paucihalophilus- | AKEMAKLERASSEDEIDFEQVEVASPEASTS* | |
| DX253_gvpA | | |
| Mega-family- | MSIQKSTXSSSLAEVIDRILDKGIVIDAFARVSXVGIEILTIEARVVIASVDTWLRYAEA | 179 |
| consensus_gvpA | VGLL-D-VEE-GLP-RX- | |
| Bacillus- | MSIQKSTDSSSLAEVIDRILDKGIVIDAFARVSLVGIEILTIEARVVIASVDTWLRYAEA | 180 |
| megaterium_gvpA | VGLLTDKVEEEGLPGRTEERGAGLSF* | |
| Bacillus- | MSIQKSTNSSSLAEVIDRILDKGIVIDAFARVSVVGIEILTIEARVVIASVDTWLRYAE | 181 |
| megaterium_gvpB | AVGLLRDDVEENGLPERSNSSEGQPRFSI* | |
| Serratia-family- | MAKVQKSTDSSSLAEVVDRILDKGIVIDAWXKVSLVGIELLSIEARVVIASVETYLKY | 182 |
| consensus | AEAIGLTAXAAAPA* | |
| Burkholderia-sp- | MAKVQKSTDSSSLAEVVDRILDKGIVIDVWAKVSLVGIELLSIEARVVIASVETYLKY | 183 |
| Bp5365_gvpA1 | AEAIGLTATAAAPTA* | |
| Desulfobacterium- | MAKVQKTTDSSSLAEVVDRILDKGIVVDAWAKISLVGIELISIEARVVIASVETYLKY | 184 |
| vacuolatum-DSM- | AEAIGLTAAAAAPA* | |
| 3385_gvpA | | |
| Desulfomonile-tiedjei- | MAKIAKSTDSSSLAEVVDRILDKGIVIDAWAKVSLVGIELLSVEARVVIASVETYLKY | 185 |
| DSM-6799_gvpA1 | AEAIGLTASAAAPA* | |
| Isosphaera-pallida- | MAKVTKSTDSSSLAEVVDRILDKGIVIDAFAKVSLVGIELLSVEARVVIASVETYLKYA | 186 |
| ATCC-43644_gvpA1 | EAIGLTASAATPA* | |
| Lamprocystis- | MAKVANSTDSSSLAEVVDRILDKGIVIDAWIKVSLVGIELLAIEARIVIASVETYLKYA | 187 |
| purpurea-DSM- | EAIGLTAPAAAPA* | |
| 4197_gvpA1 | | |
| Lamprocystis- | MAKVANSTDSSSLAEVVDRILDKGIVIDAWLKVSLVGIELLAVEARVVIASVETYLKY | 188 |
| purpurea-DSM- | AEAIGLTAPAAAPA* | |
| 4197_gvpA2 | | |
| Legionella-drancourtii- | MAKVQKSTDSSSLAEVIDRILDKGIVIDVWAKVSLVGIELLSIEARVVIASVETYLKYA | 189 |
| LLAP12_gvpA1 | EAIGLTATASHPA* | |
| Psychromonas- | MANVQKTTDSSGLAEVIDRILDKGIVIDAFVKVSLVGIELLSIEARVVIASVETYLKYA | 190 |
| Ingrahamii_gvpA1 | EAIGLTASAATPA* | |
| Psychromonas- | MANVQKSTDSSGLAEVVDRILEKGIVIDAFVKVSLVGIELLSIEARVVIASVETYLKYA | 191 |
| Ingrahamii_gvpA4 | EAIGLTASAATPA* | |
| Serratia-39006_gvpA1 | MAKVQKSTDSSSLAEVVDRILDKGIVIDAWVKVSLVGIELLSIEARVVIASVETYLKY | 192 |
| | AEAIGLTASAATPA* | |
| Thiocapsa-rosea- | MAKVANSTDSSSLAEVVDRILDKGIVIDAWVKVSLVGIELLAIEARVVIASVETYLKY | 193 |
| strain-DSM-235- | AEAIGLTAPAAAPA* | |
| Ga0242571-11_gvpA1 | | |
| Other gvpAs | | |
| Bradyrhizobium- | MAIEKATASSSLAEVIDRILDKGVVIDAFVRVSLVGIELLSIELRAVVASVETWLKYAE | 194 |
| oligotrophicum- | AIGLVAQPMPA* | |
| S58_gvpA1 | | |
| Desulfotomaculum- | MAVKHSVASSSLVEVIDRILEKGIVIDAWARVSLVGIELLAIEARVVVASVDTFLKYA | 195 |
| acetoxidans-DSM- | EAIGLTKFAAVPA* | |
| 771_gvpA1 | | |
| Octadecabacter- | MAVNKMNSSSSLAEVVDRILDKGVVIDAWVRVSLVGIELIAVEARVVIAGVDTYLK | 196 |
| antarcticus-307_gvpA1 | YAEAVGLTAEA* | |
| Octadecabacter- | MAVSKMNSSSSLAEVVDRILDKGVVIDAWVRVSLVGIELIAVEARVVIAGVDTYLK | 197 |
| arcticus-238_gvpA1 | YAEAVGLTAEA* | |
| Pelodictyon-luteolum- | MAVEKTIGSSSLVEVIDRILDKGVVVDAWVRMSLVGIELLAIEARVVVASVETYLKY | 198 |
| DSM-273_gvpA1 | AEAIGLTAKAA* | |
| Pelodictyon-luteolum- | MAVEKTIGSSSLVEVIDRILDKGVVVDAWVRVSLVGIELLAIEARVVVASVETYLKY | 199 |
| DSM-273_gvpA2 | AEAIGLTAKAA* | |
| Pelodictyon- | MSVEKTIGSSSLVEVIDRILDKGVVVDAWVRVSLVGIELLAIEARVVVASVETYLKYA | 200 |
| phaeoclathratiforme_ | EAIGLTAKAA* | |
| gvpA1 | | |
| Rhodobacter- | MAIEKSLASASIAEVIDRVLDKGIVVDAFVRISLVGIELLAIELRAVVASVETVVLKYAE | 201 |
| capsulatus-SB- | AIGLTVDPQTP* | |
| 1003_gvpA1 | | |
| Rhodobacter- | MAIEKSVASASIAEVIDRILDKGVVIDAFVRVSLVGIELIAIEVRAVVASIETWLKYAE | 202 |
| sphaeroides_gvpA1 | AVGLTVDPATT* | |
| gvpF | | |
| Anabaena-flos- | MSIPLYLYGIFPNTIPETLELEGLDKQPVHSQVVDEFCFLYSEARQEKYLASRRNLLT | 203 |
| aquae_gvpF | HEKVLEQTMHAGFRVLLPLRFGLVVKDWETIMSQLINPHKDQLNQLFQKLAGKR | |
| | EVSIKIFWDAKAELQTMMESHQDLKQQRDNMEGKKLSMEEVIQIGQLIEINLLAR | |
| | KQAVIEVFSQELNPFAQEIVVSDPMTEEMIYNAAFLIPWESESEFSERVEVIDQKFG | |
| | DRLRIRYNNFTAPYTFAQLDS* | |
| Ancylobacter | MSATLSAPGTANVAVEATAAADGKYLYGIIEAPAPATFDVPAIGGRGDVVHTIALG | 204 |
| aquaticus strain | RLAAVVSNSPRIDYDNSRRNMLAHTKVLEAVMARHTLLPVCFGTVGSDAEVIIEKI | |
| UV5_gvpF | LRERRDELAGLLGQMHGRMELGLKASWREEIIFEEVLAENPAIRKLRDALVGRSPD | |
| | QSHYERIQLGERIGQALQRKRQDDEERILERVRPFVHKTRLNKLIGDRMVINAAFL | |
| | VDAAVESRLDASIRAMDEEWGGRLAFKYVGPVPPYNFVTITIHW* | |
| Aphanizomenon flos- | MNTGLYLYGIFPDPIPETVDLQGLDKQSVHSQVVDGFSFLYSDACQEKYLASRRNL | 205 |
| aquae NIES-81_gvpF | LTHEKVLEQAMHEGFHVLLPLRFGLVVKDWETIQKQLIEPYKEQLNELFQKLAGQR | |
| | EVSIKILWDSKSELQAMMESNQDLKQQRDNMEGKKLKMEEIIQIGQLIESNLAAR | |
| | KQTVIQEFFNNLHPLAKEIIESEPMTEEMIYNAAFLIPWETESVFSERVEAIDRKFGD | |
| | RLRIRYNNFTAPYTFAQLAS* | |
| Aphanothece | MAEGFYLYGIFPPPGPQTIAVQGLDKQPIFSHTVEGFTFLYSEAQQSRYLASRRNLI | 206 |
| halophytica (strain | THTKVLEEAMEQGFRTLLPLQFGLVVPDWESVSQDLLQHQSETLQLLFQRLEGKR | |
| PCC 7418)_gvpF | EVSLKIYWETDAELNALLEENPDLKARRDNLEGKNLSMDEVIQIGQALEQAMERR | |
| | KQEVITRFEDALIPFAVETQENDVLTETMIYNTAFLIPWESEPEFGEAVETVDAEFA | |
| | PRLKIRYNNFTPPYNFVELRE* | |
| Aquabacter spiritensis | MMQTDTLAPAETVAEGKYLYCLIDAPAPDTFASPGIGGRGDVVHTITVGRLAAVV | 207 |
| strain DSM 9035_gvpF | SDSPRIEYENSRRNMMAHTKVLEEVMARHTMLPVCFGTVATGPDPISGKILEGRR | |
| | DELVGLLEQMRGRLELGLKATWREDVIFAEILQENPAIAKLRDSLVGRSPEKSHFER | |
| | IRLGEMIGQAMERKRRDDEERILERVRPFVHKTKLNKPIGDRMILNAAVLVEAARE | |
| | AGLDQAVRQMDAEWGARLSFKYVGPVPPYNFVTITIHW* | |
| Bacillus- | MSETNETGIYIFSAIQTDKDEEFGAVEVEGTKAETFLIRYKDAAMVAAEVPMKIYH | 208 |
| megaterium_gvpF | PNRQNLLMHQNAVAAIMDKNDTVIPISFGNVFKSKEDVKVLLENLYPQFEKLFPAI | |
| | KGKIEVGLKVIGKKEWLEKKVNENPELEKVSASVKGKSEAAGYYERIQLGGMAQK | |
| | MFTSLQKEVKTDVFSPLEEAAEAAKANEPTGETMLLNASFLINREDEAKFDEKVNE | |
| | AHENWKDKADFHYSGPWPAYNFVNIRLKVEEK* | |
| Bradyrhizobium | MSNQPIYVYGLIRAEDHQPLAVRAVGDSEQPVNIIGSGNVAALVSTIDLPEIMPTR | 209 |
| oligotrophicum | RHMLAHTKVLEAAMANGPVLPMRFGIIVPNPATLLRVIGFRHQELRARLDEIDGRI | |
| S58_gvpF | EVALKASWDEQFMWRQLASEHPDLAVSGRTMMGRGEQQSYYDRIELGRAIGA | |
| | ALEERRTAARLQLLQTVTPFAVQVKELTPVDDAMFAHLALLVEKGAEPSLYQTVEA | |
| | LERSNDSGLKFRYVAPIPPYNFVAVTLDWEQHEQAPRR* | |
| Burkholderia | MNSRNGARYLYAVQHARDVPASLPAGIGGAAVRALTDGDVAAIVSDTGLAKVRP | 210 |
| thailandensis sp. | ERRHLLAHHTVIQSLAAAGTVLPVAFGTIATSEVALRRMLRKHRNALAGELARLVD | |
| Bp5365 strain | HVEMSVRLNWDVTDLFRHLIDVRPDLKAARDAMLALGSAVTRDDKIELGSRFERV | |
| MSMB43_gvpF | LNEERARHAALVDEALDACCKEIRRDPPRHETEILHLTCLVRHAELGRFESGVAAAS | |
| | RELDDSLVLKYSGPCPPHHFVNLNMSL* | |
| Chlorobium luteolum | MERDGKYIYCIIGADCECDFGPIGIGGRGDLVSTIGFEGISMVVSDHPLNRFVVDPD | 211 |
| DSM 273_gvpF1 | GILAHQRVIEAVMKEHESVIPVRFGTVAATPDEIRNLLDRRYGELSELLLRLRNKVEF | |
| | NVTGRWHDMAAIYKEVERTHPEIKEQRARIESMRDGDGEALKQSLILDTGHQIEA | |
| | ALEVMKEEKFDAVASLFRKTAMASKMNRTTSPDMFMNAAFLIDRGREVEFDGI | |
| | MEILGQKDADRCDYRYSGPLAIFNFVDLRILPEKWEL* | |
| Chlorobium luteolum | MAHEAAEQDGLYIYGIINNSGELDFGPIGIGGREERVYAVIHNDIAAVVSRTVVKEF | 212 |
| DSM 273_gvpF2 | EPRRANMIAHQKVLEAVMVSHAVLPVRFSTVSPGHDDMKVEKILEEDYLRLKKLL | |
| | VKMEGKKEMGLKVMANEEKVYESIITGYDNIRYLRDKLINLPPEKTHYQRVKIGELV | |
| | AAALEKEVGTYKDAVLDALSPIAEEVKVNDSYGSMMVLNAAFLIRTAREEEFDRAV | |
| | NALDDRYHDMMTFKYVGTLPPYNFVNISINIKGR* | |
| Chlorobium luteolum | MNQSIYIYGIVNEPALAASFVETDPDIYAVASMGCSAIVENRPAIDLGELDRESLAR | 213 |
| DSM 273_gvpF3 | MLLQHQQTLERLMESGMQLIPLKLGTFVSSAADAACIIEDGYNLIERIFRETEDAHE | |
| | LEVVVKWSSFADLLQEVVSEGDVQELKREVEARQSSSTEDAIAVGRLIKEKIDRRNA | |
| | ALSASVLRQLGERASQSKRHETMDDEMVLNAAFLVNRGDVDAFVATVEALDSQY | |
| | LNALHFRIVGPLPCYSFYTLEVTALFEEFIAEKRAVLGLDARSCEADVKKAYHAKAKV | |
| | AHPDVHVPAGANNGADFTVLNEAYMTLHDYYSALRNSASSRHGHEGQDSSSVV | |
| | FSVKILN* | |
| Dactylococcopsis | MTEGFYLYGIFPPPGPKTIETQGLDKQPIFSHTVEGFTFLYSEAQQSRYLASRRNLIT | 214 |
| salina PCC 8305_gvpF | HTKVLEEAMENGSRTLLPLQFGLIVPDWETVVQDLLQHQAESLHFFLEKLEGKREV | |
| | SLKIYWETNAELNALLEENPALKARRDNLEGKQLSMDEVIQIGQALEQEMEGRKQ | |
| | DIISRFEEVLIPFAFEIKENDVLTETMIYNTAFLINWDAESDFGEQLEAIDAEFSPRLKI | |
| | RYNNFTPPYNFVELRE* | |
| Desulfobacterium | MSKKNLKRNGRYLYAIIEASEEKTFGSIGMDGSDVYLIVEDKTAAVVSDVPNKKIRP | 215 |
| vacuolatum_DSM | QRKNIAAHHAVLNKIMEEITPLPMAFGIIADGEQAIRKILADNRDVFREQFATVSG | |
| 3385_gvpF | KVEMGMRISYDVPNIFEYFISTDSEIRAARDQYFGGNREPSQEAKLELGRMFNRQL | |
| | NANREEYTNQVIEILDDYCDDIKENKCRNEQEVTSLACLINRSDQKRFEEGVFESAR | |
| | HFDNNFSFEYNGPWSPHNFVNILIEL* | |
| Desulfomonile tiedjei | MEKATIKTTGSNGRYLYAVVPGSQERVYGCLGINGGNVYTIAAKDVAAVVSDVPH | 216 |
| DSM 6799_gvpF | QKIRPERRHFAAHQAVLKRVMLDGDLLPMSFGIISQGPKAVRAILSRNNKSVQQQ | |
| | LKRISGKAEMGIKVTWDVPNIFEYFIDVNRELREARNKLVQPNYLPTQQEKIEIGR | |
| | MFEEILNLERERHTKQVERVMSKRCSEIKRSKCRTEIEVMNLSCLVDRTLLSDFEAG | |
| | VLEAASHFDDSFAFDFNGPWAPHNFVDLEIDV* | |
| Desulfotomaculum | MSTGRYVYCVINSIEPLTFMSGPVGNEPEGVFTVHYKELAAVVSQSSEEKYNVCRE | 217 |
| acetoxidans_DSM | NTIAHQKVLEEVLVSHPLLPVRFGTVAQNEEIVKKFLLQERYAELRSMLHNVTGKV | |
| 771_gvpF1 | QMGLKVLWTDMKTVYQEIVEENPQIKNLKKKLESKPAETIHYEMIDLGQMVNQA | |
| | LLRKKEKQKEMVLKPLQKIALETKESFLYGDQMFVNADFLISRSSLDDFNAKVNELG | |
| | EFFNEQALFKYIGPLPPYNFVTLYVNF* | |
| Desulfotomaculum | MVKNHNTDHLKELYIYGLIGGTPFKDELEKISVIQENTPIYGVWHKNIGFAVSAAPD | 218 |
| acetoxidans_DSM | YPLKDLSKESIIQLFVDHQQVLECLRQKFSLIPVKLGTVLESVTEAAAVLANNEEKFN | |
| 771_gvpF2 | DLLNYLKDKVELNLSVSWNDLNEVVAKIGEEDEVKKLKQSLLAQEQVSQEDLIKIGK | |
| | IISFQMQQKKQAAREYIISELRNLWEDYFINEVVDENSILNLTLLAITGKVDDVNKKI | |
| | EYLNQIYRDSLDFSLTKSLLPQGFSTVSIKKITMDQLLLAKDILKLPDTASLQDINAAR | |
| | RALLHCYHPDKNDHAAVNKVQEINAAYKLLEEYCQENSSDFNVDLITDYYIMKVIK | |
| | ADKSNVNSMNME* | |
| Dolichospermum | MNTDLAHKNFGLYLYGIFPDTIPETLEIKGLDGKSVHSQVVDGFTFLYSQACQEKYL | 219 |
| circinale_gvpF | ASRRNLLAHERVLEQTMHEGFHVLLPLRFGLVVKDWETIMSQLINPHKEQLHKLF | |
| | EKLAGQREVSIKILWDAKAELQAMMESNHDLRQQRDNMEGKKLSMEEVIQIGQ | |
| | LIESNLQARKQAVIEVFTRELNPLAQEIVVSEPMTEEMIYNAAFLIPWDSEPLFSERV | |
| | ESIDQKFGNRLRIRYNNFTAPYTFALLDS* | |
| Enhydrobacter | MNPPEAYIAGRTAAKSVEDRKARPQDLAEGKYVYAIIACDEPREFKNRGIGERGDK | 220 |
| aerosaccus strain | VHTINHRQMAAVVSDSPTIDYERSRRNMMAHTVVLEEVMKEFDLLPLRFGTVAS | |
| ATCC 27094_gvpF | SAESVERQLLVPRYGELSAMLEKMRGRSEFGLKAFWHEGVAFGEIVRENARVRKL | |
| | RDALQGRSLEESYYQRIQLGEEVEKALTAIRARDEELILSRLRPFMRDIRTNKIISDR | |
| | MVLNAAFLVERGDVPALDEAIRQLDQEFSERLMFKYVGPVPPYNFVNIAINWER* | |
| Isosphaera | MRNAPPTRPGSVTPASPGKPVIDGPARYLYAFTHDLPEGPLADLEGLPGARVVVV | 221 |
| pallida_ATCC- | ADGRVAAVVSPCPLGKVRPERQRVAGHHHVLKHLQDTLGKAILPASFGMVADSE | |
| 43644_gvpF | EDLRALLRHHSAAIAEGLVRVQGKVEMTVKLRWAPDNVAQAVLGRDPELRQLRD | |
| | QLYSNGQTPTRDQSLDLGRRFHHALERQRDHYAAYLRAALSPLLSELVEEDLRDER | |
| | DLVHWACLIENQRRAGFEAALDRLAEELEDDLVLELTGPWPPHHFVDLDLDDDH | |
| | DDDEEE* | |
| Legionella drancourtii | MDSTSKKPAASNLYLYAIASVNENQEPISFHGIEEQPIDLVPYKDIMLVVSNLSKKK | 222 |
| LLAP12_gvpF | VRPERKNVAVHHAVLNHLMKHNTSMLPIRFGMIADNRKEVQRLLTINYDMLHTK | |
| | LKMMAGRVEMGVSLSWDVPNIFEYLLNRHSQLRETRDKLLANPAHEPSRDEKIEI | |
| | GALFSQILDEEREVYTDTILSLLSPVCCDVVKSTYRNDTEIMNIFCLISAARRDEFEEKI | |
| | IEASTILDDNFVIKYTGPWPPHNFSKLNLSLE* | |
| Lyngbya confervoides | MPQLLYLYGIFPAPGPQDLEVQGLDQQPIHTHIIDEFVFLYSVAQQERYLASRKNLL | 223 |
| BDU141951_gvpF | GHERVLEAAMKVGYRTLLPLQFGLIIETWDRVIKELITPRGDALKRLFAKLEGRREV | |
| | SVKLLWGPDAELNQLMEEDAGLRAERDRLEGQQLSMDQIVDIGQAIETAMTERK | |
| | DDVINAFRQRLNALAIEVLENDPLTDAMIYNTAYLIPWEDEVKFSQAIEELDEQFED | |
| | RLRIRYNNFTAPYNFAQLDQLS* | |
| Microcystis aeruginosa | MTVGLYLYGIFPEPVPDGLVLQGIDNEPVHSEMIEGFSFLYSAAHKEKYLASRRYLIC | 224 |
| NIES-843_gvpF | HEKVLETVMEAGFTTLLPLRFGLVIKTWESVTEQUSPYKTQLKELFAKLSGQREVSI | |
| | KIFWDNQWELQAALESNPKLKQERDAMMGKNLNMEEIIHIGQLIEATVLQRKQD | |
| | IIQVFRDQLNHRAQEVIESDPMTDDMIYNAAYLIPWEQEPEFSQNVEAIDQQFGD | |
| | RLRIRYNNLTAPYTFAQLV* | |
| Nostoc punctiforme | MSFYIYGILTLPAPQNLNLEGLDRQPVQIKILDDFAVIYSEAQQERYLASRRNLLSHE | 225 |
| ATCC 29133_gvpF | KVLEEIMQAGDRYLLPVQFGLLVSSWETVSQQLIRPHQEELTQLLAKLSGCREVSV | |
| | KVFWDTEAEIQGLLAEHPNLKTERDKLVGQPLSMERVIQIGQVIEQGMSDRKQGII | |
| | DVFKGTLNSIAIEVVENTPQVDTMIYNSAYLIPWEAESQFSEHVESLDRQFENRLRI | |
| | RYNNFTAPYNFARLRLTTSN* | |
| Nostoc sp. PCC | MSSGLYLYGIFPDPIPETVTLQGLDSQLVYSQIIDGFTFLYSEAKQEKYLASRRNLISH | 226 |
| 7120_gvpF | EKVLEQAMHAGFRTLLPLRFGLVVKNWETVVTQLLQPYKAQLRELFQKLAGRREV | |
| | SVKIFWDSKAELQAMMDSHQDLKQKRDQMEGKALSMEEVIHIGQLIESNLLSRK | |
| | ESIIQVFFDELKPLADEVIESDPMTEDMIYNAAFLIPWENESIFSQQVESIDHKFDER | |
| | LRIRYNNFTAPYTFAQIS* | |
| Octadecabacter | MKREVVRMTDENTINSKYLYAIIKCREQREFIARGIGERGDAVHTIAYKGLAAVVSD | 227 |
| antarcticus 307_gvpF1 | SPVMEYDQSRRNMMAHTAVLEELMEEFTLLPVRFNTVAPEAGAIEERLLVPRHEE | |
| | FTQLLGQIDKRVELGIKAFWHDGMIFEEVLRENDSIRKMRDALEGKSVDGSYYERI | |
| | QLGEKIEQAMIKKRVEDEEIILSRIRQHVHKSRSNKTIGDRMVLNGAFLVDANKES | |
| | DFDKAVQLLDQDLGNRLMFKYVGPVPPYNFVNIVVNWGVV* | |
| Octadecabacter | MTVVAEENMTGSVGLYVCAIVAEWESNSALIKCANEAQGEIQLIGQGGITAVVM | 228 |
| antarcticus 307_gvpF2 | VPPEDQPVSRDRQELVRQLLVHQQLVERFTEIAPVLPVKFGTLAPDRESVELGLER | |
| | GREKFFTAFGGLSGKTQFEITVTWDVADVFAKIAKLPAVVKLKVDLVATSESDRPIN | |
| | LDRVGRLVKETLDHQRAQTGKVLLDALLPLGVDSIVNPILNDSIVLNLALLVDTDQA | |
| | DALDRCLDELDSTFHGALSFRCVGPMPPHSFATVEINYIEPTQVSHACCVLELDAA | |
| | HNFEEIRSAYHRLARQTQQDIAPDVVVDNKSSSVGIAVLNDAYKTLLSFVDAGGPV | |
| | VVSVQRQEDAYATDIPSSGG* | |
| Octadecabacter | MTDEKKVNSKYLYAIIQCREPRELKARGIGERGDVVHTVVHKGLAAVVSDSPVME | 229 |
| arcticus 238_gvpF1 | YDQSRRNMMAHTAVLEELMEEFTLLPVRFNTVAPEAVAIEERLLVPRHDEFTQLL | |
| | GQIDKRVELGLKAFWHDGMIFGEVLRENDSIRKMRDSLKGQSVDGSYYERIQLGE | |
| | KIEKALTEKRLEDEEMILSRIRPHVHKSRSNKTIGDRMVLNGAFLVDAEKESKFDEA | |
| | VQSLDQDLSDRLMFKYVGPVPPYNFVNIVVNWGES* | |
| Octadecabacter | MRAQKVIPAAEENISGNVGLYVCAIVAERVSCSALIQCANDAPGEIQLIGHGDFTA | 230 |
| arcticus 238_gvpF2 | VVMVPEKDQLVSPDRKELMQQLLVHQQLIEKFMEIAPVLPVKFATLAPNRESVEL | |
| | GLEVGSEKFSAAFNSLSGKVQFEVIVTWDVAEVFAEIAKEPAVAKLKVDLAAMPES | |
| | YGSVSLEQLGKLVKETLELRRAETGKVLLDALVQVGVDNVVNSILDDSIILNLALLVE | |
| | AKRADAFDRCLDELDSTYHGALTFRCVGPLPPHSFATVEITYLEPAKVTEACDILELD | |
| | VARSTEEVRSAYHRLARKSHPDIVPDVAVGETASVSMAVLTDAYKTLLSFVGAGGS | |
| | VVVSVQRQEASYAADIISSAG* | |
| Pelodictyon | MDIETTKEGRYIYGIIRNSEFIDFGQIGIGKRNDRVYGVIYKDICAVVSSTPIIQYEARR | 231 |
| phaeoclathratiforme_ | ANMIAHQKVLEEVMKRFNVLPVRFSTISPHDNDDAIIKILITDYSRFDELLIKMKGK | |
| gvpF1 | KELGLKVMADETRIYENIIQKYDNIRSLRDKLLNQPADKIHYQRVKIGEMVADALKK | |
| | EIESYKQQILDILSPIAEDIKITDNYGNLMILNAAFLIKEVKESEFDDSVNKLDEKYGNI | |
| | MTFKYVGTLPPYNFVNLSINTKGV* | |
| Pelodictyon | MEKDGKYVYCIIASTYECNFGAIGIGGRGDLVNTIGFQGLSMVVSDHPLNHFVLNP | 232 |
| phaeoclathratiforme_ | DNILAHQRVIEVVMSQFNSVIPVRFGTVAATPDEIRNLLDRRYGELSELLERFENKV | |
| gvpF2 | EYNLKASWRCMIDIYKEIDKEHVELKQLRREIEGLKDEEKRKLLIVEAGHIIENELQKK | |
| | KEVEAYEIVTYLRKTVVAHKHNKTTGEAMFMNTAFLLNKGREVEFDNIMNDLGE | |
| | QYKDRSDYYYTGPLPIFNFIDLRILPEKWEL* | |
| Pelodictyon | MDRQGIYIYGFIPNHYLTDIKTILIESGIYSIEYGSIAALVSDTMVDDIEYLNREDLAYL | 233 |
| phaeoclathratiforme_ | LVDHQKKIELIMSTGCSTIIPMQLGTIVNSGNDVIKIVKNGLRIINKTFDDIADIQEFD | |
| gvpF3 | LVVMWNNFPDLIKKISDTPQIRIMKEEIANKGSYDQADSINIGKIIKKKIDEKNSKVN | |
| | LDIMNSLSSLCICVKKHESMNDEMPLNSAFLIKKDKENSFIEMVNQLDIKYENLLRY | |
| | KIVGPLPCYSFYTLESKLLNKKEIEKAEKILGIDAYKSESDIKKAYRAKAAHAHPDKNN | |
| | TISAIDNDDFIEINKAYQILLEYSSVFKDSPDHKPDEPFYLVKIKK* | |
| Phormidium tenue | MADRYYLYGIFPAPGPAELPLMGLDEQVVQAQQLGDFTFLYSLACQKRYLSSRKN | 234 |
| NIES-30_gvpF | LLGHEKVLEAAMEQGHRTLLPLQFGLIVESWNQVQEDLVTPYAEDLTQLFGRLNG | |
| | CREVSIKVQWEPSTELEMMMAENADLRAQRDQLEGTQLGMEQVIFIGQQIESAL | |
| | EERKQGIVDQFRQALSPLAKDVLENAPQTDVMIYNAAFLIPWESEAEFSQAVDAI | |
| | DSTFGDRLRIRYNNFTAPYNFAQLN* | |
| Planktothrix agardhii | MGNGLYLYGILPTNRVRPLALHGLDKQPIQTHPVDEFSFLYSETQQERYLASRRNLL | 235 |
| str. 7805_gvpF | GHEDVLEKVMQHGYRSVLPLQFGLIVKDWDHVKAQLIIPYQDRLKELFHKLEGKR | |
| | EVGVKIFWEETEELDLLMTENQELREKRDSLEGKRLSMDEIIGIGQEIERAMQDRQ | |
| | QGIIDKFQQILNPLAQEIVENDNLTSAMIYNAAYLIPWDIEPQFGDKIEELDHHFNN | |
| | RLRIRYNNFTAPFNFAQLNP* | |
| Psychromonas | MAENKKKVRKSSSKVIAKPKVIYAITAGGLQDLGNLVGINKSDIYTIEKESISFVVSDL | 236 |
| ingrahamii 37_gvpF | SPSSPRPRPDRRNIMAHNEILKQLMSKTSVLPVRFGTVATGERAVNRFCSQYNAQ | |
| | LLEQLDRVQDRVEMGIKVTWNVPNIYDYFVDNHSELREERDRVYDGNKNPRRDD | |
| | RINLGHMYDALVTEARLSHQTDLEEIILPGCDEIHSIPPKDEKVVVNLACLVQRADL | |
| | EVFEERVVEAGKTLDNTYDIELNGPWAPHNFVELDLKTMTGRR* | |
| Serratia sp. ATCC | MMSIDKSRNHRAKVLYALCVSDDSTPNYKIRGLEAAPVYSIDQDGLRAVVSDTLST | 237 |
| 39006_gvpF | RLRPERRNITAHQAVLHKLTEEGTVLPMRFGVIARNAEAVKNLLVANQDTIREHFE | |
| | RLDGCVEMGLRVSWDVTNIYEYFVATYPVLSETRDEIWNGNSNANNHREEKIRLG | |
| | NLYESLRSGDRKESTEKVKEVLLDYCEEIIENPVKKEKDVMNLACLVARERMDEFAK | |
| | GVFEASKLFDNVYLFDYTGPWAPHNFVTLDLHAPTAKKKTLTRAGTLSD* | |
| Stella vacuolata_ATCC- | MQTEALAPAAVAAEGKYLYCIIDAPAPATFASPGIGGRGDVVHTLAVGRLAAVVS | 238 |
| 43931_gvpF | DTPRIEYENSRRNMMAHTKVLEEVMAHHTLLPVCFGTVGSGDDVIAEKILEGRRE | |
| | ELSRLLEEMRGRVELGLKATWREEVIFAEVLDEDPAVRKLRDSLVGRSPEKSHFERI | |
| | RLGELIGQALLRKRRDEEERILDRVRPFVRKTKLNKPIGDRMILNAAFLVETAREAAL | |
| | DQSVREMDADWGARLSFKYVGPVPPYNFVTITIHW* | |
| Thiocapsa rosea strain | MQQAKRQDVAAGRYIYAIIPDRGDHSLGRIGLDESEVYTIGDGRVAAVVSDLSGG | 239 |
| DSM 235 | RIRPQRRNMAAHQEVLKQVLREVSPLPAAFGLMADDEAAIIRILKDNQDAFLNQL | |
| Ga0242571_11_gvpF | ERVDGSLEMGLRMSWDVPNIFEYFVGAHPELQELRDDFFRDGSNLTQDQMITLG | |
| | RSFERLLEQDREEYTEQVESVMRSCCREIKRNKCRTEKEVLHLACLVDRDAAGRFE | |
| | QVVLQAARPFDNNYAFDFNGPWAPHNFVEMDIHV* | |
| Tolypothrix sp. PCC | MDAGLYLYGIFSDPIPPTVSLKGLDSQPVYSQVIEGFTFLYSDAKQEKYLASRRNLIS | 240 |
| 7601_gvpF | HEKVLEQAMQEGFRTLLPLRFGLVVKNWETVISQLIQPCERQLRDLFQKLAGKREV | |
| | SVKILWDTKAELQAMMQSNPDLKQKRDQMEGKNLSMEEVIEIGQLIESNLQQRK | |
| | EAVIKTFFDELKPLAEEVVESEPMMEEMIYNAAFLIPWDQEALFSQRVEAIDKKFG | |
| | DRLRIRYNNFTAPYTFAQIS* | |
| Trichodesmium | MEFGFYVYGLIQEKGKMDESKDESKNGLKGSNESKDELKGLDKEDVKIQDVDEFA | 241 |
| erythraeum | VLYSIAKKERYLASRRNLITHEKVLESAMEAGYRNLLPMQFGLVVSEWEKFSQDFT | |
| IMS101_gvpF | KPCEQQIHDLFTKLKNNREVGIKIYWEPDAELEKLLENDKDLKEERDSLKDKKLTM | |
| | DQVIDIGQKIEQGMNERKQN11EIFQETLNKMAIEVIENEVQTEKMIYNAAYLIPW | |
| | DQEEDFGEKVETIDSKLCERGNFTIRYNSFTAPYNFARIRQQD* | |
| gvpF/L | | |
| Ancylobacter | MTDLLVFAVVPADRFDPAILAEGDGLPPGLRAIAAGPLAAVVGAAPEGGLKGRER | 242 |
| aquaticus strain | SALLPWLLASQKVMERLLANAPVLPVALGTVVEDEGRVRHMLDAGAAILGEGFQ | |
| UV5_gvpFL1 | AVGDGIEMNLSVLWHLDTVVARLLPGVAPELRQAAAGGDAIERQALGVVLAGLV | |
| | SAERRRARARVIEALQAVTRDFAIGEPTEPGGVVNLALLVDRAAEEALGAALEALD | |
| | AEFDGALTFRLVGPLPPYSFASVQVHLSPAAAVCGARAALGVEPDASPETVKAAYR | |
| | RAARETHPDLVPMGGEDEEAPEATADETSRFVVLSDAYRVLEGEHAPVSLRRLDS | |
| | VLTE* | |
| Ancylobacter | MLYVYAITADYAAGANHLLPAKGIVPGVPVQRFGTGALGAVASPVPVTVFGKEAL | 243 |
| aquaticus strain | HALLDDADWTRARILAHQRVVSSLLPLATVLPLKFGTLVAGEASLAAALTSQHDAL | |
| UV5_gvpFL2 | DATVARLRGAREWGVKLFFEAPTRTIRAEEPVGAGAGLAFFRRKKEEQETRAAAE | |
| | AALDRCVAASHRRLASHARAAVANPLQPPELHGHPGTMGLNGAYLVAAENEAA | |
| | WRVCFSELEQAYAALGARYVRTGPWAAYNFTGGGLV* | |
| Aquabacter spiritensis | MSGLLVFAIVPADRIEPGLLAPAEGLPPGLETVVAAGFAAIVGTAPEGGLKGRDRG | 244 |
| strain DSM | SLLPWLLASQKVIERLMARGPVLPAALGSVLEDESRVRHMLVCGQAALAAAFETL | |
| 9035_gvpFL1 | NGCWQTDLSVRWDLSRTVAHLMTELPPGLRAAAETGDETARRSLGAALAGLVA | |
| | GERRRIQSRIGAVLGAVARDLIVSDPVEPEGVVGVALLVDAPASAQVDAALDRLD | |
| | GEFEGRLTFRLVGPLAPYSFATVQIHLGPAAGLAGAHAELGLEAGAPLEAVKAAYH | |
| | RLIVGLHPDLVPHGSPGDDADDAASGKGGRAARFAAVTAAYRTLQAEHAPVSLR | |
| | RQDGLSPG* | |
| Aquabacter spiritensis | MLYVYAITADHPGPHDAGSLPGEGIVPGAPVRLLPFGDLAAAVSPVSAVDFGPEA | 245 |
| strain DSM | LPARLQDVDWTGQRVLAHQRVVDSLVDVATVLPMKFCTLFSGAAALRAALADN | |
| 9035_gvpFL2 | RAALEATVVRLRGAREWGVKLFWEAPPAEPAPVERGPGAGAAFFQRKRDAQRL | |
| | RAEAEAALAHGVAESHRRLAARARAAVANPVQPAAVHRRRGEMALNGAYLVPR | |
| | ADEAAWRESLAELERTYAGAGIRYELTGPWGPYNFTGGGLAGS* | |
| Bradyrhizobium | MTMNLVGITTPDVAGAIAAAGGRLADVETRAVEAGGLVALLALSKAPFWHVLRR | 246 |
| oligotrophicum | SRTALRSMLTAQRILEAAAVYGPLLPARPGTLIRNDAEACMLLRSQCRHLAEGLRL | |
| S58_gvpFL1 | HGTSRQYQITISWDPVAALAARRDHQDLVEAAAASADGAADKAASMIQRFMSD | |
| | QQARFEAEAMRALAAVAEDVITLPVNQPDMLMNAVVLLAPGAEPELERVLEALD | |
| | RGLRGKNLIRLIGPLPPVSFAAVSIERPGRQRIAAARRLLGIGEATRTCDLRRAYLDK | |
| | AHAHHPDTGGHAADASIVGAAAEAFRLLARVAEARASAGQDDVILVDIRRQDQQ | |
| | RSLST* | |
| Bradyrhizobium | MSKANLGIGLVHGVVTAQSAALLPQIVDAFDATEIIVVNTEQQALLISDIPQYLRGH | 247 |
| oligotrophicum | VEADTLFSDPARISTLAMKHHRILQAAAVVTDVVPVRLGTLVRGPSGARDLLNREA | |
| S58_gvpFL2 | VRFAGHLVTIHNALEFSVRILPTEQPSRRVARPVPSSGRDYLRIRRDERCGQRPAVV | |
| | DITLQELASRAVAIRERQSASRSGGRTPALAEAAFLVDRHALAAFDDCAGRIERQIA | |
| | ENGLALDIFGPWPAYSFVDGARENLG* | |
| Bradyrhizobium | MSSPRLIGLLAADDVPADLADQIMSCGPVAAAIRFAPAAASSSESLDHHAAVVAW | 248 |
| oligotrophicum | CRRAAFLPSRAGIPISPELLQSIARSAWYHRSTIEHIEGRVEISVELERRDGVRDGGID | |
| S58_gvpFL3 | GGGRAYLRATAHDLRACEVGVATAANLLAMYSERADADLIARTAPLPAIRLRASVL | |
| | VRRAVAPRLARQFDSMLSAISDRLVCRVTGPWPPYSFSTIREPS* | |
| Burkholderia | MVWLTYAVLTPKRSITLPPGVAGARLEIVDGAHLRTIVSEHPRAPSATIPSALDFGQ | 249 |
| thailandensis sp. | TVAALFRHGAIVPMRFPTCLDSKQAVRDWLDDESDMYRDLLQRIDGCVEMGLRF | |
| Bp5365 strain | RLPEAPRAQPRPQAGGPGHAYLAARGAPNSVARSHGERIAAVLRNLYRDWRFDG | |
| MSMB43_gvpFL | LVEGFVSLSFLVRQTTLDDFVDRCRQAARETAFPLYMSGPWPPYSFATDERSSAPE | |
| | PHRALRLMRRPSTAVSISANVAAPEKKDSAR* | |
| Desulfobacterium | MTLHLLYCVFSSGEMEKTRKLVPPGIDGEPVHEICSNKISGVVSTLGKPPDTHVKSL | 250 |
| vacuolatum-DSM | LAYHGVIDSYHQNRTVIPMRFAAVFRTYAHMITALNNNEKSYLLQLKRLHDCTEM | |
| 3385_gvpFL | CVRFISNSPCCVKKKEPAISPKKISGTTFLQQRKAMYEQQNRLPPEIHEKTRDILQHF | |
| | RGLYMEFKQESQPLEKDCPSLSLQGAEKTDGNALLISLFFLISKKNISLFRSRFQNICG | |
| | SSSGRHMMNGPWPPFNFINTESNLTDPS* | |
| Desulfomonile tiedjei | MLGSLAAIQFLSISSYGADEMKFLMYCIFTENSIEPPHSLVGVNRSPVRIISCDGLAA | 251 |
| DSM 6799_gvpFL | AVSVITQKEIPRDPATGLDYHKVIQWFHERIGVIPLRLGTCLGHESDVVQLLHSHGA | |
| | RYKSLLKELDGCVEMGIRVIHDRPGPQELASKSPFISRFNGTESGTDYLMRRKVLFD | |
| | ADEFAISRNREIVERYHSPFTGLYVSFKAQTSKFSPLGTDRNSVLTSLYFLIPRQSADS | |
| | FRAIYGDLRSGLHERIMLSGPWPPYNFVLPEDCL* | |
| Enhydrobacter | MEGHRIYIYGIVRDAADGGPAPVPPVAGLDGGALRAIAGYGLAAIASAVDLSKAGI | 252 |
| aerosaccus strain | PFEEQLKDPDRATALVLEHHRVLQQAIDAQTVLPMRFGALFQDDRGVTDALEKN | |
| ATCC 27094_gvpFL | RCGLMDALGRIDGAREWGVKIFCDRAVAARQLSATSAVVQAAEKELSGLAEGRA | |
| | FFLRRRLERLRTEETDRAVAHEVDVSRQALCELARASAPLKLQPAAVHGRGEDMV | |
| | WNGAFLVPRSGEERFLSRLEVVVQSRSDLGLHYEVTGPWPPFSFVDGQLEGGGD | |
| | ACPDGA* | |
| Octadecabacter | MRSATSIVYAYGVLTNCSDIALDMPRSDLAGLVKNGPLRILPFGNIAAVVCDFVLP | 253 |
| antarcticus 307_gvpFL | NGSDLETLLEDSRSAERLILNHHQVLSYIVSQHTILPLRFGAAFTEDAGVIAALGGRC | |
| | SELQKALGRIDGALEWGVKTFCDRKLLKQRVRGTGSEISDLESEIAKQGEGKAFFLR | |
| | RRKERLILEEVEEILEQCVVGTQEQLEPSVIEEALVKLQPPTVHGHEHDMLSNISYLI | |
| | ARGTEDAFMQSLEDLRLAHAPYGLEYQMNGPWPAYSFSDQQLEGGVNDQ* | |
| Octadecabacter | MSSATSIVYVYGVLTNCSDLVLDFPPGDLAGIVESGPLRILPFGDIGALVCDFILPDG | 254 |
| arcticus 238_gvpFL | SDLKTILEDSRSAERMILNHHLVLADMVSRYTILPLRFGAVFAEDAGVIAALGGRYS | |
| | TLQKELDRIDGAIEWGVKSFCNRKMFSECVAETVSEISVLEKEIADQGEGKAFFLRR | |
| | RIQRLILDEVEKTLEQCLVGAQDQLKSRAIEETLVKLQPPTVHGHKHEMVSNRSYLI | |
| | ARGAEDAFMQSLDDLRVVYAPFGFDYQINGPWPAYSFSDQQLGGGVNDK* | |
| Rhodobacter | MGHYLYGLLAPPARGTLAQMQAAAAGVTSLGGPVALSAVEGMLLVHCPCDLAEI | 255 |
| capsulatus SB | SQTRRNMLAHTRMLEALMPLATCLPVRFGVIAQDLAEVARMIHERRAELVGHAQ | |
| 1003_gvpFL1 | RLLDPVEIGLRVRFPRDRALAQLMAETPDFVAERDRLMGQGAGAHFARADFGRR | |
| | LAEALDARRTRDQKRLLAALRPHVRDHVLRAPEEDVEVLRAEFLIPAAGVDAFSRIA | |
| | HDLAAALGFAGAAEPELQVIGPAPPYHFLSLSLAFDNTSEAA* | |
| Rhodobacter | MAHEIIAILPCEAAQLPSGLTGVVGRGATAVLAPAPGWAERLTGGPKQTAVRHHS | 256 |
| capsulatus SB | RLEALMAMGSVLPFAAGIACTPEEAALLLRLDAPLIARLAAEIGPRRHFQLALDWD | |
| 1003_gvpFL2 | ESRVLAAFRDSPELAPLFSGAAVTPEALRQAITALADRLSATALRLLDPVAEDPVEQ | |
| | PRAPGCLLNLVFLLRPEDEPRLDAALQAIDALWSEGLRLRLIGPSAPISHALVDIDRA | |
| | DVAALAAAADLLKVAPEAGPEAVTEAAKAALRSPDLAANAAEQIRAAARLLLRAG | |
| | DIAALGLSGAATLPHLVHLRPGGRKSGLTSSGEAA* | |
| Rhodobacter | MTGLALHGFVSPDGWSAAAAPPARCAVVLGGVAALVSEAGDALDTPETAQAAA | 257 |
| capsulatus SB | LAHHALISAWHRRGPVLPVRLGTVFSSQAALQTALAPKAAQLRAALDALADKEEM | |
| 1003_gvpFL3 | VLTIVPAARPPDLPPPAATGADWLRARKAVRDRGQARQTDRQQTLAGLQDALR | |
| | AQGVASLAAPAPREGGSRWHLLIARDDGAGLDRWLAAQADRFDAAGLDLTLDG | |
| | PWPPYRFAAEILEALDG* | |
| Rhodobacter | MSEPRISGLAPWRADLPDVIGCHGGWVLMGAAADETPEARLRRQVGWCRAAV | 258 |
| capsulatus SB | DVLPLSPRLAPTRAEAERLVATRGPDLERAHRHIRGRLQVIVQLEMCRTDLGLVRR | |
| 1003_gvpFL4 | EISGGRSWLQDRAERATREARANADFEAQVRRVVRALFPREGQVVTLAPSGTAG | |
| | QLRLRRAVLVPRAGLQAFAAALSADLDRDGRGGLWDVIAPLPPLAFAALEAGPGG | |
| | AVT* | |
| Rhodobacter | MIYLYGLLEEPASGHEVLAGMAGVTGPIALARLPGGILIYSSATEADILPRRRLLLAH | 259 |
| sphaeroides | TRVLEAAAWFGNLLPMRFGMMASTLAEVAAMLASRLTELCAAFDRVRGRVELGL | |
| 2.4.1_gvpFL1 | RLSFPREPALAATLATAPDLAAERARLLALRRPDPMAQAEFGRRLAERLDARRGET | |
| | QRLLFQSLRPLWVDHRLRVPDSDVQVIAVDVLVEDGAQDRLAAALVKAAADCSF | |
| | APTAEPSVRVIGPVPLFNFVDLVLSPRREEVA* | |
| Rhodobacter | MRLREVVAVLEGHPPSVLPEGTEAICEAGLTAILGMPPGLLSGRRALLEHAACRQA | 260 |
| sphaeroides | VLERLMAFGTVLPVLTGNCLTPAEAAAALAANSPRLRQELRRLAGRVQFQVLVQ | |
| 2.4.1_gvpFL2 | WHAALVPKRTDPDETAEDLRLRFTHRIADALARVAERHVNLPLREDMLANQALLL | |
| | LQTRTDDLDRSLEQIDALWTEGLRIRRIGPSPPVSFASLNFRRVSSAAIRRARHRFDL | |
| | EGPVDPIRLRALRRDLLLRASEAERAEILAAAAVLDLLTRCAASGGDLHLVRIWSEG | |
| | QAVPSDLEDAA* | |
| Rhodobacter | MSGLLLLGVVSGLGISPAITSPHLRLDGDGYAAILLSLDRLPPDPASPDWAVQAALA | 261 |
| sphaeroides | QNAILSAYAATEDVLPVALGAAFTGIAAVKRHLDAERATLDAGMERLAGRAEYVA | |
| 2.4.1_gvpFL3 | QLIAEQVADGAAPAPASGSAFLKARSARHEQRRHLARERTGFARATAEELASLSCS | |
| | ASARPLKPDGPLLDLSLLVARDRVPGLLEAAEASSRAGSRLALSVRLIGPCAPFSFLP | |
| | ETRGHD* | |
| Rhodobacter | MAGDARSRVRLHLAAMRDCETFLPFPPAATIAVDEAIAWCGRRTNALAEEIDRFS | 262 |
| sphaeroides | RQRQLTVSARLIAPLLPDAAASGAGWLRARRDASAHQARLRTVLMQIMSLLGEV | |
| 2.4.1_gvpFL4 | RCIPGRLQDEVQVNLLVPAAETHPVLHELRERLRVGDALWSACTVTGPWPPYAFI | |
| | SWETA* | |
| Rhodococcus hoagii | MSEQESAPDGGGPVVYVYGLVPADVEVKEDATGIGSPPRPLKIVHHEDVAALVSEI | 263 |
| 103S_gvpFL1 | DPDTPLGSSDDLRAHAAVLDSTATVAPVLPLRFGAVLTDTDAVVAELLEPYRDEFH | |
| | EALEQLEGKVEFVVKGKYVEDAILREILADDPEAARLRDVVREQPEDTTRDERLALG | |
| | ERISQALTAKREQDTGRIVEALQPAATAVAPREPTDDEEAGSVAVLISADGVDELD | |
| | KAVARLIDDWQGRVEVTVTGPLAAYDFVKTRAPGT* | |
| Rhodococcus hoagii | MTPDDGVWVYAVTGDGSFPGGISGIRGVAGEELRTVTDSGFTAVVGTVRLDTFG | 264 |
| 103S_gvpFL2 | EEALRRNLEDLDWLADTARRHDAVVAAICAGGATVPLRLATVYFDDDRVRTMLR | |
| | DNAEQLGEALQQIADRSEWGVRAYLERPRSEPRDAREKTGRPSGTAYLMQRRAQ | |
| | VAAREQAESAAGRRADEIFAELARWAVAGVRQPPSPPDLAGRRSQEILNTSFLVD | |
| | NGRHREFVTAVEELDARLSDVDLVLTGPWPPYSFTSVEASAR* | |
| Serratia sp. ATCC | MSLLLYGIVAEDTQLALEPDGSPHAGEEPMQLVKAATLAALVKPCEADVSREPAA | 265 |
| 39006_gvpFL | ALAFGQQIMHVHQQTTIIPIRYGCVLADEDAVTQHLLNHEAHYQTQLVELENCDE | |
| | MGIRLSLASAEDNAVTTPQASGLDYLRSRKLAYAVPEHAERQAALLNNAFTGLYRR | |
| | HCAEISMFNGQRTYLLSYLVPRTGLQAFRDQFNTLANNMTDIGVISGPWPPYNFA | |
| | S* | |
| Stella vacuolata-ATCC- | MSGLLVFAIVPADGIEPGILAPREELPANLRAVAADGFAAVVGAAPEGGLKGRDRS | 266 |
| 43931_gvpFL1 | VLLPRLLASQKVIERLMARGPVLPVTLGTVLEDEARVRHMLAAGAPMLEAAFGTL | |
| | GDCWQMDLSVRWDLNQVVARLMGEVPGDVRAAAGSGDEAARRALGEALAGL | |
| | AAGERRRVQSRLAAALRDVARDLIVSEPVEPESVVDIAILVERPALAEVEAALDRLD | |
| | AEFEGRLKFRLVGPLAPHSFATVQVHLAPEAALAGACAELGVERGAGLQDVKVAY | |
| | HRALVRFHPDLAPHGDDGGPEDEHDGGEGRASRLLTVTAAYRALQAEHAPISLRR | |
| | QDGIAVNQEQDASAAMGQQRGIVPGRELQALRM* | |
| Stella vacuolata-ATCC- | MLYVYAIAADHPDPDNAMFGGEGIVPDAPVRLLQLGDLAVAASLVSAADFAADA | 267 |
| 43931_gvpFL2 | LRAHLEDARWTALRVLAHQRVVDSLLPHATVLPMKFCTLFSGEAALKQALAHNRA | |
| | ALQATVERLRGAREWGVKLYWEAPRNPAPPSAGQGEAGAGAAFFQRKRDQQR | |
| | QRAEAEAAVARCVAASHRRLADAARAAVANPVQPPAVHRQPGEMALNGAYLV | |
| | ARAAEPAWREVLAELERTHADGGIRYELTGPWGPYNFTGSGLVGS* | |
| Thiocapsa rosea strain | MSDRPRPMLHCILRSPPGSIARAEAGLRWIERDGLAALVADREPSEIAGASSVGLQ | 268 |
| DSM 235 Ga0242571- | RYADIVAEIHACAAVIPVRFGCLLAGDEAVGKLLHRSRDRLHGLLDQVGDCLEFGIR | |
| 11_gvpFL | LLLPADAPAATDDDAAPRLHANAPSDPRADPDMGPGLSHLLAIRHRLDVEASLAA | |
| | RAREAREVIKGRVAGRFREVREELGQIDGRSLLSLYFLVPREQGEHFVECLRQDASS | |
| | LRGTGLLTGPWPPYNFVGAIDDDIRSLD* | |
| gvpG | | |
| Anabaena-flos- | MLTKLLLLPIMGPLNGVVWIAEQIQERTNTEFDAQENLHKQLLSLQLSFDIGEIGEE | 269 |
| aquae_gvpG | EFEIQEEEILLKIQALEEEARLELEAEQEEARLELEAEQEDFEYPPQFTAEVNKDQHL | |
| | VLLP* | |
| Bacillus- | VLHKLVTAPINLVVKIGEKVQEEADKQLYDLPTIQQKLIQLQMMFELGEIPEEAFQE | 270 |
| megaterium_gvpG | KEDELLMRYEIAKRREIEQWEELTQKRNEES* | |
| Ancylobacter | MGMLTDVVFAPAVGPLKGVLWLARIIAEQAERTLYDEGVIRAALLDLEQQLEAGEI | 271 |
| aquaticus strain | DEDAYETQETVLLERLKIARERMRSGL* | |
| UV5_gvpG | | |
| Aphanizomenon flos- | MLTKLLLLPIMGPLNGLVWIGEQIQERTNTEFDAQENLHKQLLNLQLSFDIGEISEE | 272 |
| aquae NIES-81_gvpG | DFEIQEEELLLKIQALEEEARLELELAEEEARLELELEQEEEEDFVVKPQLTTEIDRDKD | |
| | LVLLP* | |
| Aphanothece | MVFKLLLLPITGPIEGVTWLGEQILERANQELDEKENLNKRLLSLQLSLDLGEISEEEY | 273 |
| halophytica (strain | DEQEEEILLAMQAMEDEENNQAEEETD* | |
| PCC 7418)_gvpG | | |
| Aquabacter spiritensis | MSLVTDVLFAPAVGPLKGVLWLARLIAEQAERTLYDEDVLRAALLDLEQRFEAGEI | 274 |
| strain DSM 9035_gvpG | SEADYETEEDILLARLKIARERMRSGL* | |
| Bradyrhizobium | MLFQILTSPVSGPFRMVSWIGGAIRDAVDTKMNDPAEIKRALAALEQQLEAGSLS | 275 |
| oligotrophicum | EQDYERMEMELIERLQSSLRHGSGNGG* | |
| S58_gvpG | | |
| Burkholderia | MFILDNLLAAPIKGMFWIFEEIAQAAEEETIADIEMIKAALVELYRELESGQIDETEF | 276 |
| thailandensis sp. | ETRERALLDRLDSLETS* | |
| Bp5365 strain | | |
| MSM B43_gvpG | | |
| Chlorobium luteolum | MFILDDILLAPLSGMVFLGRKINEIVQNEMSDEGAVKEQLMKLQFRFEMDELSEEE | 277 |
| DSM 273_gvpG | YDRLEDELLSTLAEIRAQKENR* | |
| Dactylococcopsis | MVFKLLLLPITGPIEGITWLGEQILERADQELDSKENLNKRLLSLQLSLDLGEISEEEY | 278 |
| salina PCC 8305_gvpG | DEQEEEILLAMQAMEDEENEEEES* | |
| Desulfobacterium | MFLVDDILFFPAKSLVWVFRELHNAVQQEKTNESDALTTELSELYMMLETGKITEE | 279 |
| vacuolatum_DSM | EFDEREEQILDRLDEIQERDQ* | |
| 3385_gvpG | | |
| Desulfomonile tiedjei | MERYTMFLLDDILFLPMNGVLWICNEIHDAAEQELHNESDAITAQLQKLYTLLEAG | 280 |
| DSM 6799_gvp | GDIGESEFDVLEAELLDRLDAIQERGALLEA* | |
| Desulfotomaculum | MLGKLLLSPILGPVMGVKFIAEKIKQQADQELYDKSKIKQDLMELQIKLELEEITEEY | 281 |
| acetoxidans_DSM | YLQREEELLVRLDELASMETEEEEV* | |
| 771_gvpG | | |
| Dolichospermum | MLTQLLLLPIMGPLNGVVWIAEQIQERTNTEFDAQENLHKQLLSLQLSFDIGEISEE | 282 |
| circinale_gvpG | EFEIQEEEILLKIQALEEEARLELEAEQEEARLELEAEQEQARLELEAEQEELENQPQL | |
| | TPKIDTYRHLVKL* | |
| Enhydrobacter | MGMLARLLTLPVSAPVGGVLWIARKIEEEANAERWDRNKITGALSELELELDLGAI | 283 |
| aerosaccus strain | DVEEYDAREAVLLQKLKELQEVEND* | |
| ATCC 27094_gvpG | | |
| Isosphaera | MFLVDDILLAPAHSLMFLLREIHQAALEELRRDAQKVREELAECYRALETGALTDEE | 284 |
| pallida_ATCC- | FASLETDLLDRLDALEELARFNSDEDDDPEDEDWDVEDDDPAEAVW* | |
| 43644_gvpG | | |
| Legionella drancourtii | MLLLGSILMAPVHGLMAIFEKIKEAVDEEKQHDIERIKSELMALYTKLESGELSEADF | 285 |
| LLAP12_gvpG | EKQEKILLDKLDSLEDEDD* | |
| Microcystis aeruginosa | MFLDLLFLPVTGPIGGLIWIGEKIQERADIEYDEAENLHKLLLSLQLSYDMGNISEEE | 286 |
| NIES-843_gvpG | FEIQEEELLLKIQALEEEEAENESESSL* | |
| Nostoc punctiforme | MVLRFLLLPITGPLMGVTWLGEKILEQASTEIDDKENLSKQLLALQLAFDMGEIPEE | 287 |
| ATCC 29133_gvpG | EFEIQEEALLLAILEAEQEERDQTQEY* | |
| Nostoc sp. PCC | MLGKILLLPVMGPINGLMWIGEQIQERTNTEFDAQENLHKQLLSLQLKFDMGEIS | 288 |
| 7120_gvpG | EEEFDIQEEEILLKIQALEAEERLNAESEEDDDLDVQPIFILASEENPVYQDQSRFSEE | |
| | YEDKEDLVLSP* | |
| Octadecabacter | MGIILNTLMSPLIGPMKGVFWVAEQIKDQTDAEIYDDSKILVELSELELLLDLEKIEL | 289 |
| antarcticus 307_gvpG | KDFEAKEDVLLKRLQEIRKAKKNDSV* | |
| Octadecabacter | MSIILNTLMGPLIGPMKGLLWVAEQIKDQADAELYDDSKILVALSELELSFDLEQIEL | 290 |
| arcticus 238_gvpG | KEFEAQEDVLLQRLQAIRKAKQNDTD* | |
| Pelodictyon | MFILDDILFAPLNGLIFIAKKINDVVEKETSDEGVVKERLMALQLRFELDEIDEVEYD | 291 |
| phaeoclathratiforme_ | REEDELLQKLERIRLNKQNQ* | |
| gvpG | | |
| Phormidium tenue | MLFKLLFAPVLGPIEGISWVANKLLEQADVPTNDLESLQKQLLALQLAFDMGEVAE | 292 |
| NIES-30_gvpG | ADFEIQEEEILLAIQAIEDEEDEDE* | |
| Planktothrix agardhii | MILRLLLSPITAPFEGVIWIGEQLLERAEAELDDKENLGKRLLALQLAFDMGDIPEED | 293 |
| str. 7805_gvpG | FEVQEEELLLQIQALEDEANQENDEID* | |
| Psychromonas | MFILDDILLAPYSGIKWLFKEIQRQAQEELDGEADRITTDLTNLYRQFESNEITEQEF | 294 |
| ingrahamii 37_gvpG | EERETVLLDRLDELQEESNLLDEEYDEEYEDDDEEYEDDDEEYEDDDEEYEDDDEEY | |
| | EDDDKNDKDKNDDHDNDDDDENKDENDKYNDEER* | |
| Rhodobacter | MGLLRKLLLAPVELPITGALWIVEKIAETAESELTDPGTVRRLLRGLEQQLEAGEITE | 295 |
| capsulatus SB | EEYEFAEEILLDRLKRGQAAEARSGGP* | |
| 1003_gvpG | | |
| Rhodobacter | MGLLTSLLTLPFRGPFDGTLWIAARIGEAAEQSWNDPAALRAALVEAERQLLAGEL | 296 |
| sphaeroides | SEETYDAIELDLLERLKGTAR* | |
| 2.4.1_gvpG | | |
| Rhodococcus hoagii | MGLFSAIFGLPLAPVRGVVWIGEVVRRQVEEETTSPAAMRRDLEAIEEGRRSGEIS | 297 |
| 1035_gvpG | EDEAAQAEDEILHRVTRRRDAGASGEE* | |
| Serratia sp. ATCC | MLLIDDILFSPVKGVMWIFRQIHELAEDELAGEADRIRESLTDLYMLLETGQITEDE | 298 |
| 39006_gvpG | FEQQEAVLLDRLDALDEEDDMLGDEPGDDEDDEYEEDDDEEDDDEEDDDDEDD | |
| | DDEDDDDEEDDDDDEDDDDEDEPEGTTK* | |
| Stella vacuolata_ATCC- | MGLVTNVAFAPVVGPLKGVLWLARLIADQAERTLYDEDLVRAALLDLEQRLDAG | 299 |
| 43931_gvpG | QISEADYDAEEEILLARLKIARERMRSGL* | |
| Thiocapsa rosea strain | MLIVDDLLAAPFKGIIWVFEEIHKSATAEQRARRDEIMAALSALYRALEQGEITDDT | 300 |
| DSM 235 | FDTREQALLDELDALDAREDANELGSDEDEDDLDGAGEDAS* | |
| Ga0242571_11_gvpG | | |
| Tolypothrix sp. PCC | MEVMIMLGKILLFPVMGPISGLMWIGEQIQERTDTEFDAQENLHKQLLSLQLSFDI | 301 |
| 7601_gvpG | GEISEEDFEEQEEELLLKIQALEEEKARLEAESIEDEEDEVEPTYFIAEVEEDKVLAEAF | |
| | RGNKKYEDNENLVLSP* | |
| Trichodesmium | MLLRLLTLPISGPLEGVTWLGKKLQEQVDTEIDETENLSKKLLTLQLAFDMGEISEE | 302 |
| erythraeum | DFEDQEEELLLAIQALEEQKLKEEEEDA* | |
| IMS101_gvpG | | |
| gvpJ | | |
| Anabaena-flos- | MLPTRPQTNSSRTINTSTQGSTLADILERVLDKGIVIAGDISISIASTELVHIRIRLLISS | 303 |
| aquae_gvpi | VDKAKEMGINWWESDPYLSTKAQRLVEENQQLQHRLESLEAKLNSLTSSSVKEEIP | |
| | LAADVKDDLYQTSAKIPSPVDTPIEVLDFQAQSSGGTPPYVNTSMEILDFQAQTSA | |
| | ESSSPVGSTVEILDFQAQTSEESSSPVVSTVEILDFQAQTSEESSSPVGSTVEILDFQA | |
| | QTSEEIPSSVDPAIDV* | |
| Bacillus- | MAVEHNMQSSTIVDVLEKILDKGVVIAGDITVGIADVELLTIKIRLIVASVDKAKEIG | 304 |
| megaterium_gvpi | MDWWENDPYLSSKGANNKALEEENKMLHERLKTLEEKIETKR* | |
| Ancylobacter | MNEQRMEHSLQAVGLADILERVLDKGIVIAGDITISLVEVELLNIRLRLVVASVDRA | 305 |
| aquaticus strain | MSMGINWWQSDPHLNSHARELAEENKLLRERLDRLEAAVVPSALPADAALEPSL | |
| UV5_gvpJ1 | AGEDARHGG* | |
| Ancylobacter | MPSRHSGEIAVADLLDRALHKGLVVWGEATISVAGVDLVYLGLKLLLTSTDTVNR | 306 |
| aquaticus strain | MREAANAPPDERHLHAD* | |
| UV5_gvpJ2 | | |
| Aphanizomenon flos- | VTSTPILPTRPQTNSSRAINTSTQGSTLADILERVLDKGIVIAGDISISIASTELIHIRIRL | 307 |
| aquae NIES-81_gvpJ | LIASVDKAKEMGINWWETDPYLSTKAQRLVEENQQLQNRLENLESQINLLTSAKV | |
| | QEQISLVETTEDNTHQTTEDNTHQTHEESIPLPIDSQLDV* | |
| Aphanothece | MVNPNTNKPKSYQSKGITNSTQSSSLADILERVLDKGIVIAGDITVSVGSTELLSIRIR | 308 |
| halophytica (strain | LLVSSVDKARELGINWWEGDPYLSSQANLLKEENQALQNRLENMEAELRRLKGET | |
| PCC 7418)_gvpJ | NPEPSFLSESEDNS* | |
| Aquabacter spiritensis | MSEQRMEHSLQAVGLADILERVLDKGIVIAGDISISLVEVDLLNIRLRLVVASVDRA | 309 |
| strain DSM | MSMGINWWQSDPHLNSHARQLEEENRLLRERLDRLEAALAPPEGGMLRAEVEV | |
| 9035_gvpJ1 | AHGG* | |
| Aquabacter spiritensis | MPDPEPIIPRTSGDVALADLLDRALHKGLVLWGEATISVAGVDLVYLGLKVLLASTD | 310 |
| strain DSM | TANRMRDAAAASAAGSHLPGG* | |
| 9035_gvpJ2 | | |
| Arthrospira platensis | MTLQSRSSSPQRGVPMSTSGSSLADILERVLDKGIVIAGDISVSVGSTELLSIRIRLLI | 311 |
| NIES-39_gvpJ | ASVDKAKEIGINWWESDPYLSSQAQQLSQSNQQLLEEVKRLQEEVRSLKALTSQSS | |
| | QPVTPPNSENDD* | |
| Bradyrhizobium | MTFTVHQPTGGDRLADILERVLDKGIVVAGDVTISLVGIELLNIKIRLIVATVDRALE | 312 |
| oligotrophicum | LGINWWEADPRLTTRASELSVENEELKKRLALLEADAGRNQRPRKRRVRSIAATSG | |
| S58_gvpJ1 | ASHER* | |
| Bradyrhizobium | MTYRADLDYLEPAASSEGSLLELLDHLLDRGVLLWGELRISVADVELIEVGLKLMLA | 313 |
| oligotrophicum | SARTADRWRQTTTQRASIAPGDCP* | |
| S58_gvpJ2 | | |
| Burkholderia | MRSADGEPVSAELAQRLSLCESLDRILNKGAVISAQVVVSVADVDLLYLHLRLLLTS | 314 |
| thailandensis sp. | VETALVGRAMPREEASR* | |
| Bp5365 strain | | |
| MSMB43_gvpJ1 | | |
| Burkholderia | MADLLERVLDKGVVITGDIRINLVDVELLTIRIRLLVCSVDKAKELGIDWWNADTFF | 315 |
| thailandensis sp. | LGPDRGQSALPGRASAVDVAAGSAVHADAAHR* | |
| Bp5365 strain | | |
| MSMB43_gvpJ2 | | |
| Chlorobium luteolum | MPELKHAVNATGLADILERVLDKGIVIAGDIKIQIADIDLLTIKIRLMVASVDKAIEM | 316 |
| DSM 273_gvpJ1 | GINWWQEDPYLSTGAKTSEQTRLLGEINQRIEKLESINR* | |
| Chlorobium luteolum | MQEDLYTANRQVTLLDILDRVLNKGVVISGDIIISVAGIDLVYVGLRVLLSSVETMER | 317 |
| DSM 273_gvpJ2 | LDAARAEGLQQ* | |
| Chlorobium luteolum | MAVEKTIGSSSLVEVIDRILDKGVVVDAWVRVSLVGIELLAIEARVVVASVETYLKY | 318 |
| DSM 273_gvpJ3 | AEAIGLTAKAA* | |
| Chlorobium luteolum | MAVEKTIGSSSLVEVIDRILDKGVVVDAWVRVSLVGIELLAIEARVVVASVETYLKY | 319 |
| DSM 273_gvpJ4 | AEAIGLTAKAA* | |
| Dactylococcopsis | MVNSNTNQPKSYQSKGITNSTQSSSLADILERVLDKGIVIAGDISVSVGSTELLTIRIR | 320 |
| salina PCC 8305_gvpJ | LLISSVDRAREIGINWWESDPYLSSQAHLMKEENQALQSRLENMEAELRRLKGET | |
| | NLDQSSLGESDQRSLQ* | |
| Desulfobacterium | MAYIDIDNDASKQISICEALDRVLNKGAVITGELTISVADIDLIYLSLQAVLTSVETAR | 321 |
| vacuolatum_DSM | HMFDSQINDAVKEVK* | |
| 3385_gvpJ1 | | |
| Desulfobacterium | MPIQRTAQHSIESTNIADLLERVLDKGIVIAGDIKISLVDIELLSIQLRLVICSVDKAKE | 322 |
| vacuolatum_DSM | MGMDWWVNNPVFMPNKGTQNDEIADTLTKINSRLEHLEKATISGS* | |
| 3385_gvpJ2 | | |
| Desulfomonile tiedjei | MMDEEEHVSLCEALDRVLNKGAVIAGEVTISVANVDLIYLGLQVVLASVDTIRGKR | 323 |
| DSM 6799_gvpJ1 | NELLRHDVGLHLTADNA* | |
| Desulfomonile tiedjei | MSIQASTRHSIQSTNLADLLERVLDKGVVIAGDIKIKLVDVELLTIQIRLVVCSVDKAK | 324 |
| DSM 6799_gvpJ2 | EMGMDWWTNNPAFQPALAQISE* | |
| Desulfotomaculum | MGPQMGPIKSTGNLSLLDVIDRILDKGLVINADISVSIVGVELLGIKIKAAVASFETA | 325 |
| acetoxidans_DSM | AKYGLQFPTGTEINEKVSEAAKQLKEICPECGKKSGRDELLHEGCPWCGWISARAL | |
| 771_gvpi 1 | RLETEHSQR* | |
| Desulfotomaculum | MLPIREERATLTDLLDRVLDKGLLLNADILISVAGVPLIGITLKAAIAGMETMKKYGL | 326 |
| acetoxidans_DSM | LIDWDQESRLAERRLRSSRH* | |
| 771_gvpJ2 | | |
| Enhydrobacter | MAVTNGRMEHSIQGSSLADILDRILDKGIVIAGDVTISLVGVELLNIRLRLLVASVDK | 327 |
| aerosaccus strain | AIEMGINWWEADPYLTSQTKASSEQTELLQQRLERIEGLLAGQATKEQPL* | |
| ATCC 27094_gvpJ1 | | |
| Enhydrobacter | MPVQTAHDGELALADLLDRALNKGVVLWGDATISLAGVELVYVGLRVLVASCST | 328 |
| aerosaccus strain | MEKYRSSPRKGSMPIARGES* | |
| ATCC 27094_gvpJ2 | | |
| Isosphaera | MIVCSSSTPERIGPPMNLPPPHHAPWCYDSPDLETLPLDPAERIALCEVLDRVLNK | 329 |
| pallida_ATCC- | GVVIHGEITISVAGVDLVYLGLNLLLTSVETAQSWKFRGMIE* | |
| 43644_gvpJ1 | | |
| Isosphaera | MAITRSSRPDVTHSTSGATLADVLERVLDKGLVIAGDIKIKLVDVELLTIQIRLVVASV | 330 |
| pallida_ATCC- | DKAREMGLDWWTRSPELSSLAATTCPALTPPKQEATPPATRIQAPTESAQTTPDQ | |
| 43644_gvpJ2 | SHPSDPSASNIDEVAELRRHIELMQLRDEARQRAHREELAALRAQLTRLTELLDSPR | |
| | * | |
| Legionella drancourtii | MIIEDKPVSLCETLDRVLNKGVVVAGTVTISVADVDLLYLDLHCLLSSMKGMNLIGS | 331 |
| LLAP12_gvpJ1 | ERER* | |
| Legionella drancourtii | MELQKSPTHSIGSTTIADLLERILDKGIVIAGDIKVNLVQVELLTIQIRLLICSVDKAKEI | 332 |
| LLAP12_gvpJ2 | GMDWWTHQNDVQSKNGSMPIQEYVTQMEERLKNLENTLASSKNAI* | |
| Lyngbya confervoides | MTGQSLSRSSSANRQMATATQGSTLVDVLERVLDKGIVIAGDISVSVGSTELLTIRI | 333 |
| BDU141951_gvpJ | RLLVASVDKAREMGINWWENDPYLSARSQELLTANEQLQSRIESLEQELKSLRSQE | |
| | D* | |
| Microcystis aeruginosa | MTSSTFAGSLRNQSNNSLKTATQGSSLADILERVLDKGIVIAGDISVSIASTELINIRIR | 334 |
| NI ES-843_gvpJ | LLIASVDKAREMGINWWEGDPYLHSQSQALLAENRELSLRLQTLETELETLKSLTQL | |
| | SAMESHDTSPNDEAHSSDA* | |
| Nostoc punctiforme | MSTNTNRGAITTSTQGSTLADILERVLDKGIVIAGDISISVGSTELLNIRIRLLISSVDK | 335 |
| ATCC 29133_gvpJ | AKEIGINWWESDPYLNSQTRTLLATNQQLQERLASLETELQSLKALNPINHQNAG | |
| | D* | |
| Nostoc sp. PCC | MTTTPIHPTRPQTNSNRVIPTSTQGSTLADILERVLDKGIVIAGDISISIASTELIHIRIR | 336 |
| 7120_gvpJ | LLISSVDKAREMGINWWENDPYLSSKSQRLVEENQQLQQRLESLETQLRLLTSAAK | |
| | EETTLTANNPEDLQPMYEVNSQEGDNSQLEA* | |
| Octadecabacter | MNDGKMEHSLNATNLADILERVLDKGIVIAGDVTISLVGVELLNIKLRLLIASVDKA | 337 |
| antarcticus 307_gvpJ1 | MEMGINWWAHDPFLTAGAQAPAVADPAMLERMDRLEAALATALASNQTTPM | |
| | KGHK* | |
| Octadecabacter | MTNKAQGGQDLALADLLDRALSTGVVIWGEATISLAGVDLVYVGLKVLVASVDA | 338 |
| antarcticus 307_gvpJ2 | AERMKAASLVDRPTDRGQQI* | |
| Octadecabacter | MNNGKMEHSLDATNLADILERVLDKGIVIAGDVTISLVGVELLNIKLRLLIASVDKA | 339 |
| arcticus 238_gvpJ1 | MEMGINWWAHDPYLTAGAQAPVGVDPAMLERMDRLEAALAKALASNQTTPA | |
| | EGQSS* | |
| Octadecabacter | MTNETQGGQDLALADLLDRALSTGVVIWGEATISLAGVDLVYVGLKVLVASVDAA | 340 |
| arcticus 238_gvpJ2 | QRMKDASLVDRPTDGGQ* | |
| Pelodictyon | MPELKHAVNATGLADILERVLDKGIVIAGDIKIQIADIDLLTIKIRLLIASVDKAMEM | 341 |
| phaeoclathratiforme_ | GINWWQEDTYLSTKAKDKEQQLLRDDLQQRIEKLEALTKIT* | |
| gvpJ1 | | |
| Pelodictyon | MQDEFYSKNKEITILDVLDRVLTKGVVITGDIVISVADIDLVYVGLRLLLSSVETMEK | 342 |
| phaeoclathratiforme_ | NKQNSIKM* | |
| gvpJ2 | | |
| Phormidium tenue | MATATQGSSLVDVIERVLDKGIVIAGDISVSVGSTELLSIRIRLIISSVDKAREIGINW | 343 |
| NIES-30_gvpJ | WESDPYLSSRTNELLEANQQLQSRLETLEAELKALRSAEPVS* | |
| Planktothrix agardhii | MNSQQLPSNIQRGVPTSTQGSSLADILERVLDKGIVIAGDISVSVGSTELLNIRIRLLI | 344 |
| str. 7805_gvpJ | ASVDKAREIGINWWESDPYLSSQTKVLTESNQQLLEQVKFLQEEVKALKALLPQEN | |
| | QPNPISDPHK* | |
| Planktothrix | MNSQQRPSNIQRGVPTSTQGSSLADILERVLDKGIVIAGDISVSVGSTELLNIRIRLLI | 345 |
| rubescens_gvpJ | ASVDKAREIGINWWESDPYLSSQTKVLTESNQELLEQVKLLQEEVKALKALLPQEN | |
| | QPKEME* | |
| Psychromonas | MANVQKSTDSSGLAEVVDRILEKGIVIDAFVKVSLVGIELLSIEARVVIASVETYLKYA | 346 |
| ingrahamii 37_gvpJ1 | EAIGLTASAATPA* | |
| Psychromonas | MPMANVSINPELTAQECEKISLCDALDRIINKGVVIHGEITISVANVDLISLGVRLILS | 347 |
| ingrahamii 37_gvpJ2 | NVETREQSNTPKEEV* | |
| Psychromonas | MATGKPQSMTHSVKSTTVADLLERILDKGIVVTGDIKIKLVDVELLTVELRLVICSVD | 348 |
| ingrahamii 37_gvpJ3 | KAVEMGMDWWNNNPAFAPQAPAQEGELSSIEKRLEKIEKALVK* | |
| Rhodobacter | MGYRSASQPEGLADVLERILDKGIVIAGDVSVSLVGIELLTIRLRLLIATVDKAREMG | 349 |
| capsulatus SB | IDWWSHDPYLNGRLRPGEPAPETETETAALRDRLAQLEAQLSALGAQVGAAPAL | |
| 1003_gvpJ1 | AEPALRGLAAAGSSALCAAPEASSADVVQPVFRRYKEAP* | |
| Rhodobacter | MDDRFSLRLFGPEEVFDAPSGGLADLLDGLLGHGIVLHGDLWLTVADVELVYVGL | 350 |
| capsulatus SB | SAVLASPEALRSHE* | |
| 1003_gvpJ2 | | |
| Rhodobacter | MSFQMQSPLQQDSLADVLERILDKGIVIAGDISISLVGIELLTIRLRLLVATVDKARE | 351 |
| sphaeroides | MGINWWESDPRLCITQAPASDGSAALLDRLERIETQIGQLAAAREG* | |
| 2.4.1_gvpJ1 | | |
| Rhodobacter | MTDSAPTLQFATAEEALQSSETRLVDVVDALLSQGIAIRGELWLTIADVDLVFLGLD | 352 |
| sphaeroides | LLLANPDRLQCRVPDAA* | |
| 2.4.1_gvpJ2 | | |
| Rhodococcus hoagii | MTRSGSGANYPQQYSQGLGGAGHEPANLGDILERVLDKGIVIAGDIRVNLLDIELL | 353 |
| 103S_gvpJ | TIKLRLVIASLETAREVGIDWWEHDPWLSGNNRDLELENERLRARIEALESGERRV | |
| | ADVTDPHRAVQPAESPAAEVRDDDA* | |
| Serratia sp. ATCC | MPVNKQYQDEQQQVSLCEALDRVLNKGVVIVADITISVANIDLIYLSLQALVSSVE | 354 |
| 39006_gvpJ1 | AKNRLPGRE* | |
| Serratia sp. ATCC | MSGNKKLTHSTDSTTVADLLERLLDKGVVISGDIRIRLVEVELLTLEIRLLICSVDKAV | 355 |
| 39006_gvpJ2 | EMGLDWWSGNPAFDSRARVSSSAPAPELEERLQRLEARLEAAPSVIEETHL | |
| Stella vacuolata_ATCC- | MSGQRMEHSVQAVGLADILERVLDKGIVIAGDISISLVEVELLTIRLRLVVASVDRA | 356 |
| 43931_gvpJ1 | MSMGINWWQSDPNLNSHARQLEEDNRLLRERLDRLEAALALPEMAGERLADAG | |
| | QGGGAEQGVTHGR* | |
| Stella vacuolata_ATCC- | MSDPEPIIPRTSGDIALADLLDRALHKGLVLWGEATISVAGVDLVYLGLKVLVASTE | 357 |
| 43931_gvpJ2 | TADRMRAAAASQSADPKVRAG* | |
| Thiocapsa rosea strain | MMLAIGEHPDCPEEIQRVSLCEALDRILNKGAVVSGELTIAVANVDLLYLSLQLVITS | 358 |
| DSM 235 | VETAKREMLYVRH* | |
| Ga0242571_11_gvpJ1 | | |
| Thiocapsa rosea strain | MSVQRSTLTHSTNSTSVADLLERVLDKGIVIAGDIRIKLVDIELLTIQLRLVICSVDKA | 359 |
| DSM 235 | REMGIDWWSDNAMFKGLSSQASAASLPGTAAASGIEDRLARLESLLVKQSAAAE | |
| Ga0242571_11_gvpJ2 | TVL* | |
| Tolypothrix sp. PCC | MADILERVLDKGIVIAGDISVSIASTELLHIRIRLLISSVDKAKELGINWWENDPYLSS | 360 |
| 7601_gvpJ | KSQRLVEENQQLQQRLESLEAQLRSLTAAKINNPELFPVNAEDNGQSDEENVPLP | |
| | MNYQPND* | |
| Trichodesmium | MFIRVDFLLDKGVIVDAWVRLSLVVIELLTIEAKIVIASVEAYLKYSEAFCFNY* | 361 |
| erythraeum | | |
| IMS101_gvpJ1 | | |
| Trichodesmium | MAVEKVNSSSSLAEVIDRILDKGVVVDAWIRLSLVGIELLTIEARIVVASVETYLKYAE | 362 |
| erythraeum | AVGLTTLAAAPGEAAA* | |
| IMS101_gvpJ2 | | |
| Trichodesmium | MAVEKVNSSSSLAEVIDRILDKGVVVDAWVRLSLVGIELLTIEARIVIASVETYLKYAE | 363 |
| erythraeum | AVGLTTLAAEPAA* | |
| IMS101_gvpJ3 | | |
| Trichodesmium | MKTSANIATSASGNGLADVLERVLDKGVVIAGDISVSIASTELLNIKIRLLISSVERAK | 364 |
| erythraeum | EIGINWWESDPYFSSQNNSLVQANEKLLERVASLESEIKALRSN* | |
| IMS101_gvpJ4 | | |
| Trichodesmium | MKTSANIAKSAGGDSLADVLERVLDKGIVIAGDISVSIASTELLNIKIRLLISSVERAKE | 365 |
| erythraeum | IGINWWESDPSLSSQNNSLVQVNQKLLERVASLESEIEALKYSQ* | |
| IMS101_gvpJ5 | | |
| gvpK | | |
| Anabaena-flos- | MVCTPAENFNNSLTIASKPKNEAGLAPLLLTVLELVRQLMEAQVIRRMEEDLLSEP | 366 |
| aquae_gvpK | DLERAADSLQKLEEQILHLCEMFEVDPADLNINLGEIGTLLPSSGSYYPGQPSSRPS | |
| | VLELLDRLLNTGIVVDGEIDLGIAQIDLIHAKLRLVLTSKPI* | |
| Bacillus- | MQPVSQANGRIHLDPDQAEQGLAQLVMTVIELLRQIVERHAMRRVEGGTLTDE | 367 |
| megaterium_gvpK | QIENLGIALMNLEEKMDELKEVFGLDAEDLNIDLGPLGSLL* | |
| Ancylobacter | MTAPCTAETLENALRGRIDIDPEKVEQGLVKLVLMLVETVRQVVERQAIRRVEGG | 368 |
| aquaticus strain | TLTEEETERLGLALMRLEEKMAELRLHFGLEDGDLDLKLQLPLGEL* | |
| UV5_gvpK | | |
| Aphanizomenon flos- | MVYSPVENSNDFLNVIPVENSNEFLNTSPKKKSNSETGLAPLLLTVLELIRQLMEAQ | 369 |
| aquae NIES-81_gvpK | IIRRMEEDLLSESDLERTAESLQKLEEQILNLCQIFDIDPADLNINLGDFGSLLPASGS | |
| | YYPGETGNRPSILELLDRLLNTGIVVDGEIDIGVAQLDLIHAKLRLVLTSKPI* | |
| Aphanothece | MSADESNLSQVNLNPATSNSDAGLAPLLLTVTELIRQLMEAQVIRRMDGGLLNEE | 370 |
| halophytica (strain | ELDRAGDSLQRLEAEIIRLCEIFEIDPKDLNVDLGELGTLMPKNGGYYPGESSDDPSI | |
| PCC 7418)_gvpK | LELLDRILHKGVVIDGNLDLGIAQLSLIQARLHLVLTSQPINGK* | |
| Aquabacter spiritensis | MTGFAGGPAVTETLESVLQGRVDIDPERVEQGLVKLVLMVVETLRQVIERQAIRR | 371 |
| strain DSM 9035_gvpK | VEAGALTDEEIERLGLTLLRLEEKMAELRVQFNLSEADLSLKLRLPLGEL* | |
| Bradyrhizobium | MSASSHSEAPGLRLQLGDLDTALAAVFTDAAPNGSINLDPDKIEHDLARLVLTLIEF | 372 |
| oligotrophicum | LRRLLELQAIRRMEANELSEDEEERVGLALMRAAAQVSRLARELGVDPRELNLQLG | |
| S58_gvpK | PLGRLL* | |
| Burkholderia | MNAPHAAAVSDAAALAAALEQALAQQQAPPPRATQRFDVATASAGNGLAKLVL | 373 |
| thailandensis sp. | ALMKLLHELLERQALRRIEAGSLNDDEIERLGLALMRQAEEIERLAAQFGFTDADL | |
| Bp5365 strain | NLDLGPLGRLF* | |
| MSM B43_gvpK | | |
| Chlorobium luteolum | MHEDKVQFQASSVEEALRQLEGMKQGKESRIEANPDNVESGLARLVLTLIELLRKL | 374 |
| DSM 273_gvpK | MEKQAMRRIDGGSLDEAQIDELGETLMKLEMKMDELKKTFNLTDSDLNLNLGPL | |
| | GDLM* | |
| Dactylococcopsis | MSEEESNLSRVDLNPASSNSDAGLAPLLLTVTELIRQLMEAQVIRRMDAELLTEAEL | 375 |
| salina PCC 8305_gvpK | DRAGESLQRLEEEILRLCEIFDVDPADLNVHLGELGTLLPKEGGYYPGETSDQPSILE | |
| | LLDRVLHTGVVIDGNLDLGIAQLNLIQAKLHLVLTSQPINN* | |
| Desulfobacterium | MIKDPEAKDFKIESDSIDAFARVMHADTSSCSSSSVTAGQRQQRLKIDEENIKNGL | 376 |
| vacuolatum_DSM | AQLVMTLIKLLHELLERQAIRRIESGSLDDDQIERLGLTLMQQCEEIDRLRKLFDLEE | |
| 3385_gvpK | EDLNLDLGPLGKLL* | |
| Desulfomonile tiedjei | MNPMNIAKVESDSLGDFAEIMQTDWISSLHSDKEEKRLNLNQDSVKNGLGQLVL | 377 |
| DSM 6799_gvpK | TLVKLLHDLLERQAIRRMEAGTLTDTEIDRLGTTLMMQAQEIERLRSEFGLEEEDLN | |
| | LDLGPLGKLL* | |
| Desulfotomaculum | MYIDISEGSLKQGVLGLLLALVEIIKDALKIQALKRIEGDSLTEDEIERLGNALHELEEA | 378 |
| acetoxidans_DSM | LVEIEMEHNLQNVVQNIREGLDNVVNEVVDTFNPERWIAENEFN* | |
| 771_gvpK | | |
| Dolichospermum | MLSTPADNFDESLTTVSKSKNEAGLAPLLLTVLELLRQLMEAQVIRRMEDNLLSESE | 379 |
| circinale_gvpK | LERAADSIQKLEEQILHLCETFEVDPAELNINLGDFGTLLPQSGSYYPGETGSRPSVL | |
| | ELLDRLLNTGVVLDGEIDLGLAQLDLIHAKLRLVLTSKPI* | |
| Enhydrobacter | MTKLLEAKTVDPDKAGDDLVKLVLALVETLRQLVERQAIRRVDSGVLNDDEVERL | 380 |
| aerosaccus strain | GLALLRLEEKMSELKAHFGFGDEELTLKLGSLGELARDV* | |
| ATCC 27094_gvpK | | |
| Isosphaera | MSDSLFEVRSPSAAPPSPVNPGVADEWTAVLKDWDTLTAQLRQATAPPNAENS | 381 |
| pallida_ATCC- | ARSHATTGRIDLDPEQVGDGLAKLVLTLLELIRQLLERQAIRRLDAGSLDHEQTERL | |
| 43644_gvpK | GLTLMRLAQRMEELKTHFGLQGEDLNLDLGPLGKLL* | |
| Legionella drancourtii | MNDKREEDNALPQRINLQPDDVKNGLGKLVLILIQLIHELLERQAIGRIEAGDLSDE | 382 |
| LLAP12_gvpK | QIDRLGITLMKQAEEIDKLREVFGLTQEDLNLDLGPLGKLL* | |
| Microcystis aeruginosa | MTLACTPYDSDNQALLTRPESNSQAGLAPLLLTVVELVRQLLEAQIIRRMEKGVLSE | 383 |
| NIES-843_gvpK | SDLDRAAESIQKLQEQILYLCEIFEVEPEELNVHLGEFGTLLPEAGSYYPGEEGIKPSV | |
| | LELVDRLLNTGVVVEGNVDLGLAQLDLIHLKLRLVLTSQPV* | |
| | MQAISKSKGSDSGLAPLLLTVVELIRQLMEAQVIRRMDAGTLNDSELDRAAESLQK | 384 |
| Nostoc punctiforme | LEQQVVQLCEIFDIDPADLNINLGEMGNLLPQSGGYYPGETSSQPSILELLDRLLNT | |
| ATCC 29133_gvpK | GVVVEGDLDLGLAQLSLVHAKLRLVLTSKPL* | |
| | MVCTPVEKSPNLLPTTSKANSKAGLAPLLLTVVELIRQLMEAQVIRRMEQDCLSES | 385 |
| Nostoc sp. PCC | ELEQASESLQKLEEQVLNLCHIFEIEPADLNINLGDVGTLLPSPGSYYPGEIGNKPSVL | |
| 7120_gvpK | ELLDRLLNTGIVVDGEIDLGLAQLNLIHAKLRLVLTSRPL* | |
| | MKTTSDSQFDSMKKILTDSSKEDSASCDPTDLLPNKSLPPSLSTSPETAADDLVKLV | 386 |
| Octadecabacter | LAVIDTVRQVMEKQAIRRVESGALAEAEIERLGLTLMRLEARMVELKSHFGLSNED | |
| antarcticus 307_gvpK | LNLHFGTVQDLKDILNDEE* | |
| | MKTQNDTQFDSMKKILTDSGGGDPNPNGSPDQTQHASLPSNLSTDPETAADDL | 387 |
| Octadecabacter | VKLVLAVIDTVRQVMERQAIRRVDSGALADEEIERLGLTLMRLEERMADLKSHFGL | |
| arcticus 238_gvpK | SNEDLNLNFGTVQDLKDILNDEE* | |
| Pelodictyon | MDSDKILYYAGSADEIIEELEKLKPGIQGRINATPDNVESGLAKLVLTLIELIRKLIEKQ | 388 |
| phaeoclathratiforme_ | AMRRIDGNSLSESQIEELGETLMKLEKKMEELKGIFNLTDKDLNLNLGPLGDLM* | |
| gvpK | | |
| Phormidium tenue | MTSENAEPDLSTTLALQPPAKTDAGLAPLLLTVIELVRQLMEAQVIRRMESGDLDD | 389 |
| NIES-30_gvpK | NDLERAADSLRKLEEQVVSMCEIFDVDPADLNIDLGEIGTLLPKEGNYYPGQKNQN | |
| | PTILELLDRLLDTGVVVEGDVDLGMAQLNLIHAKLRLVLTSKPI* | |
| Planktothrix agardhii | MSSSEPSIETIITPKSSRKDAGLAPLVLTLVELIRQLMEAQVIRRMEGNTLSEEELDR | 390 |
| str. 7805_gvpK | AAQSLQQLEIQVLKLCEIFEIDPTDLNIELSEFGTLLPKSGSYYPGENTQNPSILELLDR | |
| | LMNTGIVVEGSVDLGLAQLNLIHAKLRLVLTSKPL* | |
| Psychromonas | MPFEHFKSNNQADVNSDTKPAASVGGLNLESDDLKNGLGRLVLTLVKLLHELLER | 391 |
| ingrahamii 37_gvpK | QALRRMDAGSLQDDEIERLGLAFMKQAEEIDRLRKEFGLEVEDLNLDLGPLGRLL* | |
| Rhodobacter | MSAAMHLELGDVDAVLSQAARSLAAGGRLTLDPERVEQDLARLVLGIVELLRKLM | 392 |
| capsulatus SB | ELQAIRRMEAGSLTPEQEETLGLTLMRAEAALHEVAAKFGLQPADLILDLGPLGRS | |
| 1003_gvpK | V* | |
| Rhodobacter | MTYPFPPLLLRDDRLPPTEAPVTAPRIALDPDRLEHDLARILLGLMEMLRQIMELQ | 393 |
| sphaeroides | AIRRMEAGSLSESQQEQLGTTLMRAEAAIHEMAARFGLTPADLSLDLGPLGRTI* | |
| 2.4.1_gvpK | | |
| Rhodococcus hoagii | MRRRIDSDPESVERGLVALVLTLVELLRQLMERQALRRVDAGDLSDDQIERIGTTL | 394 |
| 1035_gvpK | MLLEEKMEELREHFGLEPEDLNIDLGPLGPLLAED* | |
| Serratia sp. ATCC | MTTNQLSHHSPVFGPTSPAIQRPITEANRHKIDIDGERVRDGLAQLVLTLVKLLHEL | 395 |
| 39006_gvpK | LERQAIRRMDSGSLSDEEVERLGLALMRQAEELTHLCDVFGFKDDDLNLDLGPLG | |
| | RLL* | |
| Stella vacuolata_ATCC- | MTGFLNGPADVETLETALRGRVDIDPERVEQGLVKLVLMVVETLRQVIERQAIRR | 396 |
| 43931_gvpK | VESGSLTDDEVERLGLTLMRLEEKMDQLRRQFDLGEEDLSMRLRLPLQEL* | |
| Thiocapsa rosea strain | MSDTRTGTAPSSAASAAPDTSTLQRANLLADLLETKVAAAGRRIDIDPERVQRGLG | 397 |
| DSM 235 | QLVLTVVKLLHVLLERQAIRRVDGGDLDEDEIEQLGLALMRQSEEIERLRRLLGLEE | |
| Ga0242571_11_gvpK | QDLNLDLGPLGKLF* | |
| Tolypothrix sp. PCC | MAMVCTPSENSNDLLATNSKANNQAGLVPLLLTVVELIRQLMEAQVIRRMEEECL | 398 |
| 7601_gvpK | SESDLERAAESLQKLEEQVLNLCQIFEIDPADLNIHLGELGSLLPAAGSYYPGETGNT | |
| | PSVLELLDRLLNTGVVVDGELDLGVAQLNLIHAKLRLVLTSKPLNTK* | |
| Trichodesmium | MSLENSPEESLIVPIDKSKSNPEAGLAPLLLTVIELLRELMQAQVIRRMDAGILSDEQ | 399 |
| erythraeum | LERAAEGLRQLEEQVIKLCKVFDIPTEDLNLDLGEIGTLLPKSGEYYPGEKSENPSVLE | |
| IMS101_gvpK | LLDRILNTGVVLDGTVDLGLAELDLIHARLRLVLTA* | |
| gvpL | MLYLYAILESPPPQKPLPPGIGGAAPLFVESHALVCAASEAADAAIAREPSQIWRH | |
| Ancylobacter | QEVVAALMEGRPVLPLRFGTVVEDSAACLRLLARHHAELSAQLDRVRHCVEFALR | 400 |
| aquaticus strain | VAGLSELADPGLDPNATPAGLGPGASHLRTLVRRERGWPVSSAAFPHDTLTAHAA | |
| UV5_gvp1_ | SRLLWARSPSQPDLRASFLVQRRSASAFLDDVNALQRLRPDLGITVTGPWPPYSFS | |
| | DPDLSGGRE* | |
| Aphanothece | MLYTYCFLFSPEKTLSLPQGFKGDLQMIEKGAIAAVVEPNLPKAELEEDDQKLVQA | 401 |
| halophytica (strain | VVHHDWVICELFRGLTVLPLRFGTYFRGEADLRSHLAAYEESYQQKLTALTGKVEV | |
| PCC 7418)_gvpL | TLKLTPIPFSEEGSSSTAKGKAYLQAKKQRYQQQSNYQTQQQEALEKLQEEIKKTYP | |
| | QLIHDEPKENTERFYLLIDSHSFSVFGEKMEQWKQFLSSWSIEISDPLPPYHFL* | |
| Aquabacter spiritensis | MLYLYAVLEAPPPARSLPPGIGGGAPHFIEAFELVCAASETPNRSVAPEPAEVWRH | 402 |
| strain DSM 9035_gvpL | QQVVEALIDRAPALPLRFGTLVEDASACRRLLTRHRDALGAQLGRVRHCVEFALRV | |
| | SGLPEEVAPDPGIGGGPGTSYLRTLARREAGWPPSTAVFPHDGLAAHAAERLLWA | |
| | RSTSQPDLRASFLVRKPNVAAFLADVSALQRVRPDLGITCTGPWPPYSFSDPDLSG | |
| | VSP* | |
| Bacillus- | MGELLYLYGLIPTKEAAAIEPFPSYKGFDGEHSLYPIAFDQVTAVVSKLDADTYSEKV | 403 |
| megaterium_gvpL | IQEKMEQDMSWLQEKAFHHHETVAALYEEFTIIPLKFCTIYKGEESLQAAIEINKEKI | |
| | ENSLTLLQGNEEWNVKIYCDDTELKKGISETNESVKAKKQEISHLSPGRQFFEKKKI | |
| | DQLIEKELELHKNKVCEEIHDKLKELSLYDSVKKNWSKDVTGAAEQMAWNSVFLL | |
| | PSLQITKFVNEIEELQQRLENKGWKFEVTGPWPPYHFSSFA* | |
| Burkholderia | MNDALYLFCFARAEPLAPAWAKRAPGEPRLQLLHEGNLAAVLCDVSRSEFAGAD | 404 |
| thailandensis sp. | AERRLADPAWIAGRVAVHAAAIEWTMRYSPVIPAQFGTLFSGAGRVIALMESCH | |
| Bp5365 strain | AHIGRVLDHVEGKTEWAVKGWLDRQAAADSQAALLRADEPESAARTAGARYLR | |
| MSMB43_gvpL | ERQLQARAGQNLRDWLEQSVPPISARLQRHAVEMCSRPCRASDSEHEIVANWAF | |
| | LVRNRDVPAFRRQAEAIDAEFATWGLHFDFSGPWPPYSFCAPLTEETTWSG* | |
| Chlorobium luteolum | MPCRLTVTWKSLRTAGLLPTAKGIQGRTERMAQNILYVYCIVRQLPGADIVARYP | 405 |
| DSM 273_gvpL | DLVFIEAGSAYVAAKYVSPLEYSDASMKLKLADEEWLDRNAREHLSVNVMIMAQ | |
| | QTIIPFNFGTIFKSRESLSGFLGDYGRKLDESFDALEGREEWAVKAYCNESFLLKNLH | |
| | LESPAIAAIEQEIQAASPGKAYLLKKKKEAMSASALEGVHQGHAKAVWGELAALSK | |
| | EHVLNRLIPEDVSGVDGRMIVNGVFLIANTDVGAFIRTTEDLGERYRDAGVFLDVT | |
| | GPWPPYDFVDIPY* | |
| Dactylococcopsis | MLYTYCLIASSPSALSLPSGFRGELQLIKQGAIAAIVEAELPLEELEENDQKLIQAVIH | 406 |
| salina PCC 8305_gvpL | HDAVICEIFQQIPLLPLRFGTYFPTEKDLLEHLDFKAEKYQKKLQEIQDKVELTLKLTP | |
| | LPFSTENASPMEKQGKNYLKAKKQRYQEQTNYQSQQQAELNQLQTQINQDYPQ | |
| | FIHGEPKENIERFYLLIKERDRSVFSEQLEQWKKDFPTWTIEVSDPLPPYHFIE* | |
| Desulfobacterium | MEKKKAVYLYCVTRANKFNAPGITGIDANTPVCFEHLENFVAVYNIIPLNTFVGTSA | 407 |
| vacuolatum-DSM | EENMKNIDWIGPRAMRHENVIERMMQESSVYPARFATLFSSMENLRETLHLKSG | |
| 3385_gvpL | LISRFLNQTQHKCEYSLKGFINRKQLLEFLIKTKFKQEKKQLDGLSPGKKYFAQHQF | |
| | NKKVETGINQWIKRRCGIFLDHLTKRNPEVSPRELFTEKTEKNNLEMMFNLAFLIH | |
| | NDSKSAFLQEISQAEKEFSQTGISLVVSGPWAPYSFCKTTRGEGL* | |
| Desulfomonile tiedjei | MSNVLYLFCLARTGLVDHIEGTGITGTEDLILKNFSGVTAVTCEVPEDDFSGESAEIK | 408 |
| DSM 6799_gvpL | LQDLAWVGPRAVRHDRIIEEIMQYSPVFPAPFGSLFSSEKRLGTLIESNIDAIREFLD | |
| | HTADKQEWSVKGLVCKSKAVDEIFTGKLKILSETLSSSPAGMRYFKERQMRSEAEK | |
| | ELSGKVKAACTVVGEKLLACSNNFRQRKNISFGKAEGDKQLVVNWAFLVDHSRIS | |
| | YFLDQVEHANSNYQAGGLAFECSGPWPPYSFCPSLHMEPTR* | |
| Desulfotomaculum | MNLIDDCKAKYIYCIGENPGNWPSEVMGVEGSLVYHVVYRDIAAVVHDCAEQPY | 409 |
| acetoxidans-DSM | NSDDNNKVIDWVLGHQLVVDKACSCYSSVLPFTFNSIVKGKEDLSSHEILVNWLED | |
| 771_gvpL | NYDNFKLKLGKIKGKKEYSVQLFLDKQVSLSLLQSESDILELQVELLGSAKGKAYFVQ | |
| | EKINKKIGELMANRADSYCRQFYHEISSVVSECKLCKLKQAGRNEIMIINLVCLAGD | |
| | NEVEVLGDVLEKIKSNDIAIKIKFSGPWPAYSFV* | |
| Enhydrobacter | MLYVYGIADNAFEVLRGAGLLNSDVFAVPAGCLAAAASKLAQGGIETTPQGVWR | 410 |
| aerosaccus strain | HEQVLRQLMQDHAVLPLRFGTICRDRETLTDRLMEASDDLVRGLGRVRGKVEIAL | |
| ATCC 27094_gvpL | RIVDEREHEAHPVPSETPTVDAIGGGRGTAYLRARRRHHAAEMGREARAERVGK | |
| | MLSAYIDVGAEDLVCSVAPEGDHAVSVSCLLGRDQLATLQAALERFQSDHPAIGLS | |
| | WTGPWTPYSFVAPSLFGVGLP* | |
| Legionella drancourtii | MNKALYLFCLTPASDLPMMEGELLPNFSPLFIHPFQTFNAILSWVPAKEYQEQSTD | 411 |
| LLAP12_gvpL | SNLINTEEFMQRVFFHELVVEKIMRDEAVFPIGFGTLFSSIASLEEQILTHQTLISSCL | |
| | ANLNQKDEYAVRVYLNQDKALESLLSVMLQERESSWASSSPGVQYLKKQQLHNEI | |
| | QRNLNQHLGGMLDEVLSMFQRHATDFKSRENTAQSSDIHGTSILHWAFLIPRVVS | |
| | SIFKEQVDLMNAKYNPFGLHFVLTGPWPAYSFCTLQSVEAP* | |
| Lyngbya confervoides | MRWHRSEAVISYCDLSMIYLYALCPNSTETNNLPEGIGTAQVEVLTVGTLGAVIER | 412 |
| BDU141951_gvpL | DVDIAQIQKDDAQLMAAVLAHDRILSHLFTYSPLLPLRFGTQFSNSEAVTTFLKTQ | |
| | GETYRQKLSHLQDRAEYLVKLIPQPLDLPAIASDLKGREYFLAKKQRLQDHTAALN | |
| | QQADELQTFLTDLATQDIPLVRSAPQDHEERLHVLLSRDTDTTEQVIMTWQEQLP | |
| | NWQVVCSEPLPPYHFAA* | |
| Octadecabacter | MKRLYVYGIVGATSFDDPLPNGHDEASVFALVSGDIAVAVSFVERSAVEASAANV | 413 |
| antarcticus 307_gvpL | WLHDNVLSALMTRYAVLPMRFGTIAVGATQLLEGIVKRQKQLMKDLMRLNENV | |
| | EIALHISGKNWEKVNQKVTKKNTDQAITQGTAYLLGRQQSLYGSDKTQLLVQNVR | |
| | RAIRSGLDPLMKDVIWPIDKPQALPFKASCLINRNDVASFVQIVNDIAAQNLDARV | |
| | TCTGPWAPYSFVGKSGVEGET* | |
| Octadecabacter | MTKLYVYGIVGATHFDVKLPNGHDEAPVFAIVSGDLAVAVSSLERSAVEASAANV | 414 |
| arcticus 238_gvpL | WLHENVLSALMEGHAVLPMRFGTIATGAAQLLGDIVKRRGQLMKDLTRLDGKVE | |
| | IALRISGKNREKVEQRIAGQIVDTNVTQGVAYLQEKQQNLYGSFYTQSSVQCARRA | |
| | IRSQLDPFIVEAIWPTDEPQMLPFRASCLIKKGDIARFVQTVDDVVVKVSDIRVTCT | |
| | GPWAPYSFVGQSGSEAET* | |
| Pelodictyon | MVAIQERLIYIFCVTSEPPLLQQYQLQKGICVVDVDGLFVTTMDVTDNDFAENQL | 415 |
| phaeoclathratiforme_ | QSNLSDVVWLDTKVREHLDVITSIMQHVKSLIPFNFGTLYKSESSLMQFIIKYAEEFK | |
| gvpL1 | KNLVYLEEKEEWAVKLYCNKNKIVENITHLSKKVSDINALIQNSSIGKAYILGKKKNEI | |
| | IENEIINIYNTYSKKIFTKFSILSEEFRFNPIPNNETLEKEDDMILNVVLLLNKANVESFI | |
| | ETSDQUIQHQNIGLNIEITGPWPCYSFINISH* | |
| Pelodictyon | MPLIIYAIFDSINYIDSFSSYVDAISLKSKIKLEIISTSTLSAIVSRTTDEKKQACQNDVM | 416 |
| phaeoclathratiforme_ | IYATIIGDIAAKYSILPMRYGSIVSSPFDVTELLKNHNETFVTIIKKITDKEEYSLRILYSH | |
| gvpL2 | QDKEKNNIEDLFDLPQNVPDILHGNTDSKKYLLNKYIKHLSEEKRLQYIDKIQSIVAC | |
| | NLQKITDLIVYNKQTTTGFIVDAVFMIERSKKSELLDLVIQMQTLFSEHNVVLSGPW | |
| | PPYNFSNINIG* | |
| Psychromonas | MKNSNHSGLDPNQALYLYCFVHADSIQSVTSQAIEKDSPVFIYQWQDIAAVLSHV | 417 |
| ingrahamii 37_gvpL1 | PTSYFTGYDDEEPEQTIARILPRTQLHEQVIEEVMRQSPVFPAQFGTLFSSQESLEQ | |
| | EISQQYLAITHTLKEVSGSVEWAVKGVLDRGVAEKALYSQQLTEQQNSLSSSPGM | |
| | RHLQEQRLRRETQSKLNSWLHQLYTDIATPLSELSGDFFQRKIPSSIEEGKEVILNW | |
| | AFLVPESAGDDFHAQIDKLNQRLNSFGLVIQCSGPWPPYSFCNQSS* | |
| Psychromonas | MKNSNHSGLDPNQALYLYCFVHADSIQSVTSQAIEKDSPVFIYQWQDIAAVLSHV | 418 |
| ingrahamii 37_gvpL2 | PTSYFTGYDDEEPEQTIARILPRTQLHEQVIEEVMRQSPVFPAQFGTLFSSQESLEQ | |
| | EISQQYLAITHTLKEVSGSVEWAVKGVLDRGVAEKALYSQQLTEQQNSLSSSPGM | |
| | RHLQEQRLRRETQSKLNSWLHQLYTDIATPLSELSGDFFQRKIPSSIEEGKEVILNW | |
| | AFLVPESAGDDFHAQIDKLNQRLNSFGLVIQCSGPWPPYSFCNQSS* | |
| Serratia sp. ATCC | MTMNTEAQTEQAIYLYGLTLPDLAAPPILGVDNQHPINTHQCAGLNAVISPVALS | 419 |
| 39006_gvpL | DFTGEKGEDNVQNVTWLTPRICRHAQIIDSLMAQGPVYPLPFGTLFSSQNALEQE | |
| | MKSRATDVFVSLRRITGCQEWALEATLDRKQAVDVLFTEGLDSGRFCLPEAIGRRH | |
| | LEEQKLRRRLTTELSDWLAHALTAMQNELHPLVRDFRSRRLLDDKILHWAYLLPVE | |
| | DVAAFQQQVADIVERYEAYGFSFRVTGPWAAYSFCQPDES* | |
| Stella vacuolata-ATCC- | MLYLYAVLEALPAARTLPAGIGGGELLFVEAFELVCAASETPERAIAPEPTQVWRH | 420 |
| 43931_gvpL | QQVVEALIDCAAALPLRFGTLVEDAVACRRLLTRHREALCAQLDRVRHCVEFALRV | |
| | SGLREEVGSDHVIGGGPGVSYMRALARREASWPPSTGTFPHDGLAAHAADRLLW | |
| | SRSASQPDLRASFLVLKPNVAAFLADVSALQRMRPDLGITCTGPWPPYSFSDPDLS | |
| | GMSP* | |
| Thiocapsa rosea strain | MDAFYCFCFAPACLASDLRFDDCGWEDPIEIRRLAGLDVILSRVPLGRFAGAEAEQ | 421 |
| DSM 235 Ga0242571- | RLADLEWLVPRAQAHDRVITRTMERSTVFPLTFATLFSSLPALALEVAARRRALLDF | |
| 11_gvpL | FERMAGREEWAVKVSMDRERVIATRMQSLYPEGGDVPAGGRGYLLKQRRRGEA | |
| | EQAIGPWLKGQIGCLDEALRPSCETLLIRPLRDEMVASRACLVARDLGPSLSEAIER | |
| | SREAFADQGLDLHCSGPWPLYSFCGTP* | |
| Trichodesmium | MSYYVYGFLYLPESCLALPKGMEKEVELVPYQNIAAVVEANVSIEAIQETEEKLLEAI | 422 |
| erythraeum | LAHDRVVREIFQQVSMLPLRFGNAFALRENIINDLQNNQQQYLNILTKLQQQAEY | |
| IMS101_gvpL | TITFTPVSYPSTLEVSKVRGKAYLLAKKQQFEQQQAFQTKQRQQWENIRQLIFKNY | |
| gvpN | PKAVFRDSTESKIKQVHLLANRDARVITTEELSTWQTECSYWQITLSEQLPPYHFV* | |
| Anabaena-flos- | MTTTKVNHKRAVLRLRPGQFVVTPAIERVAIRALRYLKSGFPVHLRGPAGTGKTTL | 423 |
| aquae_gvpN | AMHLANCLDRPVMLLFGDDQFKSSDLIGSESGYTHKKVLDNYIHSVVKLEDEFKQ | |
| | NWVDSRLTLACREGFTLVYDEFNRSRPEVNNVLLSALEEKILSLPPSSNQPEYLSVN | |
| | PQFRVIFTSNPEEYAGVHSTQDALMDRLVTISMPEPDEITQTEILIQKTNIDRESAN | |
| | FIVRLVKSFRLATGAEKTSGLRSCLMIAKVCADNNIPVTTESLDFPDIAIDILFNRSHL | |
| | SMSESTNIFLELLDKFSAEELEILNNRVTGDNDFLIDNSQFVSQQLAGQPN* | |
| Ancylobacter | MTSEAASKDPISLLSGFGAGAASSGPKAGGRSTPSALTPRPRTGFVEAEQVRDLTR | 424 |
| aquaticus strain | RGLGFLNAGYPLHFRGPAGTGKTTLALHVAAQLGRPVIIITGDNELGTADLVGSQR | |
| UV5_gvpN | GYHYRKVVDQFIHNVTKLEETANQHWTDHRLTTACREGFTLVYDEFTRSRPETHN | |
| | VLLGVFEERMLFLPAQAREECYIKVHPEFRAIFTSNPQEYAGVHASQDALADRLATI | |
| | DVDYPDRAMELAVASARTGMPEASAARIIDLVRAFRASGDYQQTPTMRAGLMIA | |
| | RVAAQEGFEVSVDDPRFVQLCSDALESRIFSGQRAEEVAREQRRAALHALIDTHCP | |
| | SAAKPRARRAGGAVRASIEGAQS* | |
| Aphanizomenon flos- | MTKTNHKRAVLRVRPGQFVVTPAIEQVAIRALLYLKSGFPIHLRGPAGTGKTTLAL | 425 |
| aquae NIES-81_gvpN | HLAHCLDRPVMLLFGDDEFKSSDLIGSESGYTHKKLLDNYIHSVVKVEDEFKQNWV | |
| | DSRLTLACREGFTLVYDEFNRSRPEVNNVLLSALEEKILSLPPSSNQPEYLSVSPQFR | |
| | AIFTSNPEEYCGVHSTQDALMDRLVTINMPEPDEITQTEILIQKTNIQKESAHLIVRL | |
| | VKSFRIATGAEKTSGLRSCLMIAKVCADNNLVAEPENSFFQEIAMEILSNRTHLSVN | |
| | ESTDIFLDVISQFSNKEIEILNDAELGSLPTMDTLANTDLGNDVPLEKEASDYVIQQK | |
| | NNEFKGFQKPSTKVLN* | |
| Aphanothece | MTTVLHARPKGFVSTPTIDRISRRAWRYLQSGFSIHLRGPAGTGKTTLAMHLADLL | 426 |
| halophytica (strain | NRPIMLLYGDDEFKSTDLIGSNTGYTRKKVVDNYIHSVVKEEDELRQQWVDSRLT | |
| PCC 7418)_gvpN | MACREGFTLVYDEFNRSPPEVNNVLLSALEEKLLVLPPDSHRSEYVRVSPNFRAIFT | |
| | SNPEEYWGVHGTQDALLDRVVTINVPEPDLETQREIIVQKVGINADDGDMIVNFV | |
| | RNFRDRAEMENSSGLRSCLMIAQVCHQHEIPVQTSNEDFQDICYDILTSRCPLSTQ | |
| | ESISLLEQLFREYELELVVEDEDEDVPSVIVEGETEDLSSDEKPHLRLSHPFGNTEND | |
| | * | |
| Aquabacter spiritensis | MSTEPAPLVSPSQDVETTPQRPARPEPAEALAVGYRLSARPASPATLTPRPRADFV | 427 |
| strain DSM 9035_gvpN | ETDQVKDLTRRGLGFLRAGYPLHFRGPAGTGKTTLALHVAAQLGRPVIVITGDNEL | |
| | GTADLVGSQRGYHYRKVVDQFIHNVTKLEETANQRWTDHRLTTACREGYTLVYD | |
| | EFTRSRPETHNVLLGVFEEKILFLPAQAREECYIRVHPDFRAIFTSNPQEYAGVHAS | |
| | QDALADRLATIDVDYPDRGMELAVASARTGLGETEAARIIDLVRAFRASGDYQQT | |
| | PTMRASLMIARVAAQEGLRVSIDDPGFVQLCMDALESRMFSGARLEAATRETSR | |
| | AALLALLAVHCPSEAPIVRVTAARRAKKADAS* | |
| Arthrospira platensis | MTTVLRAVPKGFVNTPAIERITVRALRYLQSGFSVHLRGPAGTGKTTLALHLADLLN | 428 |
| NIES-39_gvpN | RPIMLIFGDDELKSSDMIGNQTGYTRKKVVDNFIHSVVKLEDSLKQNWIDSRLTLA | |
| | CREGFTLVYDEFNRSRPEVNNVLLSALEEKLLVLPPNNSRSEYIRVNPHFRAIFTSNP | |
| | EEYCGVYSTQDALLDRLITMNMPEPDEATQQEILIQKVAVTPEEAQTIVTLVQQFR | |
| | EATHAIAPSKIQTVARQQTNADKASGLRPSLMLARICQEHNIPIVPIDPDFQEVCR | |
| | DILLSRAIGDITELESRLHQIFDHLSGLENDQIIALPPREELTTSSVPNNLSDTEQKIYT | |
| | YIKDSDGARVSEIEIALGLNRVQTTDALRSLLRKSYLTQQDNRLFVVYEGD* | |
| Bacillus- | MTVLTDKRKKGSGAFIQDDETKEVLSRALSYLKSGYSIHFTGPAGGGKTSLARALAK | 429 |
| megaterium_gvpN | KRKRPVMLMHGNHELNNKDLIGDFTGYTSKKVIDQYVRSVYKKDEQVSENWQD | |
| | GRLLEAVKNGYTLIYDEFTRSKPATNNIFLSILEEGVLPLYGVKMTDPFVRVHPDFRV | |
| | IFTSNPAEYAGVYDTQDALLDRLITMFIDYKDIDRETAILTEKTDVEEDEARTIVTLVA | |
| | NVRNRSGDENSSGLSLRASLMIATLATQQDIPIDGSDEDFQTLCIDILHHPLTKCLD | |
| | EENAKSKAEKIILEECKNIDTEEK* | |
| Bradyrhizobium | MLRSDRAAIAGGQRGSRAQGDAVARNDAAAGSRAAIAQISPRPDADNAALSPA | 430 |
| oligotrophicum | PRTDLFENPQLASMAARALTYLNAGIPVHLRGPAGTGKTTMAMQLAARLGRPVV | |
| S58_gvpN | LLTGDDGLTAAHLVGREIGTKSRQVVDRYVHSVRRVETETSSMWCDAVLAQAVV | |
| | EGLTFVYDEFTRSPPQANNPLLSVVEERILIFPAGSRKERLVHAHPEFRAILTSNPEEY | |
| | AGVSRPQDALLDRLITFDLDDYDRETEIGIVSNRTGLAYAEAGVIVDLVRGVRRWP | |
| | KAHHPPSMRSAIMIARIVARELITPSVDDPRFVRLCLDVLAAKAKPTDRDDRDRFA | |
| | ATLLRLMNNHCPAGAIDGG* | |
| Burkholderia | MEASAEFVQTPAVRNLTERALTYLGAGYGVHLAGPSGTGKTTLAFHIAAQLGRQV | 431 |
| thailandensis sp. | VLMHGDDELGSADLVGRGAGYRRSRVVDNFIHSVVKTEEEMTTTWIDNRLTTAC | |
| Bp5365 strain | QHGLTLIYDEFNRSRPEANNALLPVLSEGILNLPNRMTGAGYLTVHPGFRAIFTSNP | |
| MSMB43_gvpN | EEYVGVHKTQNALMGRLITIQVGHYDRETEVEIVRARSGIARADAERIVDLTRRLR | |
| | DADDNGHHPSIRAAIALARALSYCGGEATPDNAGYVWACRDILGVDLEQDARTR | |
| | SQAGRRTKARR* | |
| Chlorobium luteolum | MRAAVNDNEMNTVLAPRPMANFVETEYIRDITERGLTYLKAGFPVHFRGPSGTG | 432 |
| DSM 273_gvpN | KTTVAMHLAGKIGRPVVVIHGDSEYKTSDLIGSEQGYKFRRLNDNFIHSVHKYEED | |
| | MSKQWVNNRLSIAIKKGFTLVYDEFTRSRPEANNILLPILQEKMLSTSASNEEDYY | |
| | MKVHPEFRAIFTSNPEEYAGVNRTQDALRDRMVTMDLDYFDYETELRVTHAKSEL | |
| | TLEDSEKIVQVVRGLRESGKTEFDPTVRGSIMIARTLHIMQVRPEKTNDAVRKVFQ | |
| | DILTSETSRVGSKTNQEKVRAIVNDLIEAYL* | |
| Dactylococcopsis | MTTVLHARPKGFVSTPTIDRISGRAWRYLQSGFSIHLRGPAGTGKTTLAMHLADLL | 433 |
| salina PCC 8305_gvpN | NRPIMLLYGDDEFKSTDLIGSNTGYTRKKVVDNYIHSVVKEEDELRQQWVDSRLT | |
| | MACREGFTLVYDEFNRSPPEVNNVLLSALEEKLLVLPPDSNRSEYVRVSPNFRAIFT | |
| | SNPEEYWGVHGTQDALLDRVVTINVPEPDLETQQEIITQKVGINANDGEKIVNFV | |
| | RQFRDRAAVKNSSGLRSCLMIAQVCHQHEIPVQTSDEGFRDICYDILSSR | |
| Desulfobacterium | MSASMSSMKETRQRMSAPEQDNVVPEAGSDFVETPYVKDITDRALAYLHVGYP | 434 |
| vacuolatum_DSM | VHFSGPAGTGKTTLAFHVAAKLKRTVMLIHGDDEFGSSDLIGKDSGYRKAKVVDN | |
| 3385_gvpN | YIHSVVKTEESMNTVWADNRLTIACQQGCTLVYDEFTRSRPEANNAFLSVLEEKIL | |
| | NIPSLRDIDQGYLQVHPEFRAIFTSNPEEYAGVHKTQDAMMDRLITITLDHFDRDT | |
| | EVQVTMSKSDLPQKDAEKIVDIVRKLRKTGVNNHRPTIRACIAIGKILKHMGGGAS | |
| | KDNFVFKQICRDVLNVDTTKVTRDGEPLLPRKIDELINSL* | |
| Desulfomonile tiedjei | MNGAELRIASIETEVITANNENIVPEAGDRFVNTPHVEELTARAMAYLEVGYSVHF | 435 |
| DSM 6799_gvpN | SGVAGTGKTTLAFHAAAKLGRPVILVHGDHEFGSSDLIGRDAGYKKSRLVDNFIHS | |
| | VVKTEEEMRSLWVDNRLTTACRDGYTLIYDEFTRSRPEANNVLLSILEEKILNLPSLR | |
| | RTGEGYLEVHPSFRAIFTSNPEEYAGVHKTQDALMDRIITINVDHYDRETEIEITRAK | |
| | SGVCKQDATVIVDIIRELRLLGVNNHRPTIRAAIAIARVLAHTGEHADQHNSVFQW | |
| | LCKDVLSTDTVKVSRGGSPLMAKKVEEVIRKVCGRTGGKRSGKPVGSKEETSE* | |
| Desulfotomaculum | MQLNGLDKNSIINPVVLSDFVVTDYISNVVDRALAYIKAGFAIHLRGRSGTGKTSIA | 436 |
| acetoxidans_DSM | MYISSKLNRPTLVIHGDEEFRTSDLIGGRYGYRIRKTIDNFVQSVVKVEEDLVERWV | |
| 771_gvpN | DSRLTTACKNGYTLVYDEFTRSRPEANNILLSVLQERLLDISVARGAEEGYVKVHPD | |
| | FTAIFTSNPEDYAGVYGSQDALRDRMVTLDLDNYDKETEISIIKSKSKLSREDSERVV | |
| | NILRDLRELGDCEYGPTIRGGIMIAKTLQVLGAPVDKNNEMFRQICEEVLASETSRA | |
| | GNLQALRKVRKVINELFNKYA* | |
| Dolichospermum | MSITKVNHKRAVLRLRPGQFVVTPAIERVVIRALRYLRSGFPIHLRGPAGTGKTTLG | 437 |
| circinale_gvpN | MHLANCLDRPVMLLFGDDQFKSSDLIGSESGYTHKKLLDNYIHSVVKVEDEFKQN | |
| | WVDSRLTLACREGFTLVYDEFNRSRPEVNNVLLSALEEKILSLPPSSNQPEYLSVNP | |
| | QFRVIFTSNPEEYCGVHSTQDALMDRLVTINMPEPDEITQTEILIQKTNIGRESANLI | |
| | VRLVKSFRLATGAEKTSGLRSCLMIAKICADHDIPASTEDLDFREIAIDILFNRAQLSIS | |
| | ESTDIFMGLLEQFSAEEIKVLNDTHFPTDELLINNSQFITQELVTQPNTELATDIPQE | |
| | LRKTEQN* | |
| Enhydrobacter | MSMDQAEEIGVVTTIEPRPRADFVRTQSVEATARRALGYLNAGFSVHFRGPAGT | 438 |
| aerosaccus strain | GKTTLALHLAALLGRPMVMITGDEEMLTSTLVGTQHGYHFRRVVDRFIHTVTKTE | |
| ATCC 27094_gvpN | ETADKRWADHRLTTACREGYTLIYDEFTRSRPEANNVLLSVLEEGLLVLPAQNQNE | |
| | PYIKVHPNFRVIFTSNPQEYAGVHDAQDALGDRIVTIDMGHADRELELAIAAARSG | |
| | LPPTQVAPIVDMVREFRETGEYDQTPTLRTSIMICRMMSQERLAPTIEDQQFVQIC | |
| | MDILGGKSLPGGKGDNKRAQQQKMLLSLIEHHCPARSFTSVGEV* | |
| Isosphaera | MDYESTALQLKPRPDFVATPWVRELADRALGYLTAGYPVHFSGPAGTGKTTLAM | 439 |
| pallida_ATCC- | HLAALVNRPVVLLHGDDEFGSSDLVGDHLGFRSTKVVDNFIHSVVKTEQSVSKTW | |
| 43644_gvpN | VDHRLTTACRHGFTLIYDEFNRSRPEANNILLTILEERLLELPPIAGGRDGSGPLRVH | |
| | PEFRAIFTSNPEEYAGVHKTQDALLDRMITISMGGHDEATETEITAAKSGLSRDEA | |
| | ARIVELARAVRALKPLRHPPTIRSCLMIAKVAALRKVPIDPNDALFLAICRDVLRIDAL | |
| | PVDDPEATFAELIRRVFAPTPAVAPPRVPTTGFAANRVVPIPRRPLAASASPPPGA | |
| | NGHAHLR* | |
| Legionella drancourtii | MMTQENNGSLTDSKNNDKLIRFVNNRSDNILLEASEEFTETPHIRGISERALAYLDI | 440 |
| LLAP12_gvpN | GYPIHLLGPAGTGKTTVALHIAAQLGRPVILIHGDDEFTGADLVGRGTGYHHSKLV | |
| | DNFIHSVLKTEEEMTTMWTDNRLTTACEQGYTLIYDEFNRSRAEANNALLSVLSEG | |
| | ILNLPGRRERDGIGYVDVHSNFRAIFTSNSEEYVGIHKTQNALADRLIAIKMDYPDQ | |
| | QSEIQIIEKKSTLPRKDIEIIVNLARELRLKSEKRPSIRGCIAIARVLAYHNRHAHADDPI | |
| | FQAVCQDIFGISKEFLKQLLHPMDSGLQKRSEKNQESIKKYKTKNQKL* | |
| Lyngbya confervoides | MSTVLQARPRNFVSTPAVERIARRALRYLQSGYSVHLRGPAGTGKTTLALHLADLL | 441 |
| BDU141951_gvpN | SRPIMLVFGDDEFKTSDLIGNQSGYTRKKVVDNYIHSVVKVEDELRHNWVDSRLTL | |
| | ACREGFTLVYDEFNRSRPEVNNVLLSALEEKLLVLPPSGHRPEYLRVNPHFRAIFTS | |
| | NPEEYAGVHGTQDALLDRLITIHMPEPDELTQQQILIQKVGIEPADALMIVRLVKA | |
| | FKSQMGNHSATSLRPSLMIANICHEHGVAMMTEDADFRDVCSDVLLSRVTNELS | |
| | PATHTLWDLFNELTASADVLGPESNSTDVSPQPEADKPVETKGSKGKSTTKSKAKE | |
| | SAKASEEADEAGDDSASAPELDEIESSILTFLTARESASLSEIESELSLTRFKAVDALRS | |
| | LVEAGYLQKQNGAGKPAIYGLVPEES* | |
| Microcystis aeruginosa | MTVTETQTRRAVLSLRPGQFVVTPSIDQIATRALRYLNSGFSIHLCGPAGTGKTTLA | 442 |
| NIES-843_gvpN | MHLANCLARPVMLIFGDDDFTSSDLIGSQSGYTHKKLMDNYIHSVLKVEDELKHN | |
| | WVDSRLTMACREGFTLVYDEFNRSRPEVNNVLLSALEEKILTLPPTSHQPDYLQVN | |
| | SQFRAIFTSNPEEYCGVHATQDALMDRLVTINMPEPDQLTQTEILAQKTGIGRED | |
| | ALFIVNLVKTFRVKTATEKTSGLRSCLMIAKVCASHDIAANSADSDFRDICADVLLSR | |
| | TNLSVDKSRAILWEILEDNPLESLSFLEEEEPSDAQVSTSEPSTGNQSLKAIQSLLRG | |
| | NLPQRKD* | |
| punctiforme | MTTVLNASPQRFVNTPAVQRIAQRALRYLQSGFSIHLRGAAGVGKTTLAMHLADL | 443 |
| ATCC 29133_gvpN | LNQPIILLFGDDEFKTSDLIGNQLGYTRKKVVDNFIHSVIKVEDEVRQHWVDARLTL | |
| | ACKEGFTLVYDEFNRSHPEVNNVLLSVLEERLLVLPTNQHRAEYIRVHPQFRAILTS | |
| | NPQEYCGVHATQDALMDRVITIDMPTPDELSQQEIVVHKTGIDSEKAEVIVRIVRT | |
| | FWSRSGSGQGGGLRSCLMIAKICHEHEISVNPGDPSFQDICADILLSRTNQPLIEAT | |
| | RLLEEVLSEFYHRINTQSQPSEIIPNNQNQIVLEQRVPYEHEVYNYLCNSPGRRFSEL | |
| | AVELGIDRSQIVAALKSLREQGVLVQMQGNAESPSISQTVAFDSGHLINK* | |
| Nostoc sp. PCC | MTLTANNKKRAVLRVRPGQFVVTPAIEQVAIRALRYLTSGFAIHLRGPAGTGKTTL | 444 |
| 7120_gvpN | AMHLANCLDRPIMLIFGDDEFKSSDLIGSESGYTHKKLLDNYIHSVLKVEDEFKQN | |
| | WVDSRLTLACREGFTLVYDEFNRSRPEVNNVLLSALEEKILTLPPSSNQPEYLHVNP | |
| | QFRAIFTSNPEEYCGVHSTQDALMDRLVTINMPEPDELTQTEILAQKTALNRADAL | |
| | LIVRLVKAFRSRTGGEKTSGLRSCLMIAKVCAEHNILVSPQSSDFREICADVLFNRTN | |
| | WSASEAATIFLELLNHLDLQQIEEFKNSIIPEDTDAIAEGGFPTIIDSHFGTLDSEVLE | |
| | QPGVEDSIPFEQEIYLYLQQYKSAALALVQQEFELSRTVATNALNSLEQKGLVSKNN | |
| | HVYTIEEPNQS* | |
| Octadecabacter | MNSNLRATNSGGPDISKTMMPEAREDFVQTESVKSISRRALAYINAGYSVHFRGP | 445 |
| antarcticus 307_gvpN | AGTGKTTMAMHTAALLGRPVVLITGDEEMITSNLVGAESGYNYRKVTDNYIHTVS | |
| | KIEESSDRSWNDHRLTTACREGYTLIYDEFTRSRAEANNVLLSVLEEGILVLPAQNR | |
| | GEPFIKVHPNFRVIFTSNPQEYAGVHEAQDALSDRIVTIDIGEADRELEVSIASSRSG | |
| | LEVAKTEPIVDMVRAFRDTGEYDQTPTLRACIVICRMVANEKLNTTIDDPFFVQICL | |
| | DVLGSKSTFGGKEHDKRTQQRKLLLDNLKHYCPSKVSTKPSAKDDESKSTLIQVSSR | |
| | GSL* | |
| Octadecabacter | MMPEARKDFVQTDSVKSVSRRALAYINAGYSVHFRGPAGTGKTTMAMHTAALL | 446 |
| arcticus 238_gvpN | GRPVVMITGDEEMVTSNLVGAESGYNYRKVTDNYIHTVSKVEESSDRSWNDHRL | |
| | TTACREGYTLIYDEFTRSRAEANNVLLSVLEEGILVLPAQNRGEPFIKVHPDFRVIFTS | |
| | NPQEYAGVHDAQDALSDRIVTIDIGAADRELEVSIASSRSGLEVAKTAPIVDMVRA | |
| | FRDTGEYDQTPTLRACIMICRMVANEKLNPTIDDSYFVQICLDVLGSKSMFGAKEQ | |
| | GKRTQQEKLLLDNLSHHCPSPPPSKPSAKEAEAKPRSIQATSRGPA* | |
| Pelodictyon | MRRQGCDSEMNTVLEPKPMPNFVETDYIRDITSRGLTYMKAGFPVHFRGPSGTG | 447 |
| phaeoclathratiforme_ | KTTVALHLASKIGRPVVIIHGDSEYKTSDLIGSEQGYKYRRLDDNFIHSVHKYEEDM | |
| gvpN | TKQWVNNRLTIAIKKGFTLVYDEFTRSRPEANNILLPILQEKMMSTSSSNEEEYYM | |
| | KVHPEFRAIFTSNPEEYAGVNRTQDALRDRMVTMDLDYFDYETELMITHAKSGM | |
| | SLDDAEKIVKIVRGLRESGKTEFDPTIRGSIMIAKTLNVLNARPDKTNELFKKVCQDI | |
| | LTSETSRVGSKTNQERVRGIVNELIDLHS* | |
| Phormidium tenue | MNTVLQARPRNFVSTPTLERTSIRALRYLQSGYSIHLKGPAGTGKTTLALHLADLLA | 448 |
| NIES-30_gvpN | RPIMLLFGDDEFKTSDLIGNQSGYTRKKVVDNYIHSVVKVEDELRHNWTDSRLTLA | |
| | CREGFTMVYDEFNRSRPEVNNVLLSALEEKLLVLPPSNNRAEYIRVSPHFRAILTSN | |
| | PEEYCGVHGTQDALQDRLITINMPEPDELAQQQILVQKVGIDSSAALQIVQLVKAF | |
| | QSAVAPDMVSSLRPSLMIATICHDHDILPLAENADFRDVCSDILLARSKEPAPDATR | |
| | HLWNLFNRFVVSQAALVNDLSLKPEAHPTARFHGEEEDDAPLQPLEALVESDIDD | |
| | VAVEDQPVIGPQDLQGETLPEAVIPEPQGETVVETPAEAEALPEEIARVQVSPDDIE | |
| | TRIFDYLDATGTASLVNIEAALDLNRFQAVNAVKSMLDQGLIEKQETDGQLQGYQ | |
| | LSSN* | |
| Planktothrix agardhii | MTTVLQARPKGFVNTPTIEQLTIRALRYLQSGFSLHLRGPAGTGKTTLAMHLADLL | 449 |
| str. 7805_gvpN | NRPIVLIFGDDELKSSDLIGNQLGYTRKKVVDNFIHSVVKLEDELRQNWIDSRLTLA | |
| | CKEGFTLVYDEFNRSRPEVNNVLLSALEEKLLVLPPNNSRSEYIRVNPHFRAIFTSNP | |
| | EEYCGVYGTQDALLDRLITIDMPEPDDETQQEILIQKIGISPEDAKNIIEIVKIYLEITT | |
| | QKKEIKPVQNGKAARPHIDKASGLRPGLIIAKICHEHDISIQENNQDFIKVCADILLS | |
| | RTNLSLTEAQNKLEKVIKTVLTDGDTSNNSFLPPSETQLTENNSLEIEEQVYQYLQKT | |
| | TSARVSEIEVALGLNRVQTTNVLRSLLKQGHLKQQDNRFFAVNKQGELIQP* | |
| Planktothrix | MTTVLQARPKGFVNTPTIEQLTIRALRYLQSGFSLHLRGPAGTGKTTLAMHLADLL | 450 |
| rubescens_gvpN | NRPIVLIFGDDELKSSDLIGNQLGYTRKKVIDNFIHSVVKLEDELRQNWIDSRLTLAC | |
| | KEGFTLVYDEFNRSRPEVNNVLLSALEEKLLVLPPNNSRSEYIRVNPHFRAIFTSNPE | |
| | EYCGVYGTQDALLDRLITIDMPEPDDETQQEILIQKIGISPEDAKNIIEIVKIYLEITTQ | |
| | KKEIKPVQNGKAARPHIDKASGLRPGLIIAKICHEHDISIQENNQDFIKVCADILLSRT | |
| | NLSLTEAQNKLEKVIKTVLTDGDTSTNSFLPLSETQLTENNSLEIEEQVYQYLQKTTS | |
| | ARVSEIEVALGLNRVQTTNVLRSLLKQGHLKQQDNRFFAVNKQGELIQP* | |
| Psychromonas | MSIENLNNVSEIKIEQSDDDHIYPEASEDFVETPYIKEVTERAMLYLDAGYPVHFAG | 451 |
| ingrahamii 37_gvpN1 | PAGTGKTTLAFHIAALRQRPVTLIHGNHEFGTSDLIGKESGYRRHRVVDNYVHSVV | |
| | KEEEELQSLWSDNRLTTCCRNGDTLVYDEFNRSTPEANNVLLSILEEGILNLPSSRSD | |
| | GYLEVHPQFRAIFTSNPQEYAGTHATQDALVDRMITIMLHYPDRHTEVRVAIAKS | |
| | GINSDEAGSIVDIVNEFRELCGSKIVSSGPKTMPTVRASIAIARVLVQKGEHAFRDN | |
| | TFFHRICRDVLCMYTQQVSFSNRSVLDKQLEDLIMKFCPATYKSSGSKIRA* | |
| Psychromonas | MSINNLNISTIKIEQPENDNIYPEASAEFVQTPYIQEVTERALLYLDAGYPVHFAGPA | 452 |
| ingrahamii 37_gvpN2 | GTGKTTLAFHIAALRKRPVTLIHGNHEFGSSDLIGKESGYRRHRLVDNYVHSVMKE | |
| | EEELKSLWVDNRLTTCCRNGDTLVYDEFNRSTPEANNVLLSILEEGILNLPSLRSMG | |
| | DGYLEVHPSFRAIFTSNPQEYAGTHATQDALVDRMITIMLNYPDRDTEVRVAVAK | |
| | SGISNEEAGFIVDIVNEFRELSNHKSLSSGQKSMPTVRASIAISRVLIQKGEHAFRDN | |
| | VFFHRVCHDVLCMYIQKISPSNRSFLDKQLEVLIGKFCPAAKSALVPKVVK* | |
| Rhodobacter | MTIPRDLPWGDARTPLFEDEELRSLLDRAEIYLREGIAIHFRGPAGVGKTTLALHLA | 453 |
| capsulatus SB | QRFARPVTFFVGNDWLGRADIFGRDLGETVSTVQDHYISSVRRAERKSRIDWQEA | |
| 1003_gvpN | PLARAMRDGHVLVYDEFSRSRPEANAALLSVIEEGVLPLSDPAAGRSHIVAHPDFR | |
| | VILTSNPRDYVGVQAVPDALLDRMITFSLDGMSFETEVGIVATAARTDPADARAIC | |
| | ALIHLLRAEKPGTMEISMRSGIMIARLARAAGVAPDPADPVFVQICADVLGTRMR | |
| | GSDIDDVMALLLRPDPAPAACAGGAR* | |
| Rhodobacter | MTVLSPSLPHAAGIDAALVENPWLGLRRSGRYFQNAETEALFARALGYARAGVCV | 454 |
| sphaeroides | HLAGPAGLGKTTLALRIAQALGRPVAFMTGNEWLGSRDFIGGEIGQTVTSVVDRY | |
| 2.4.1_gvpN | IQSVRRTEQSARIDWKESILGQAMRCGQTFIYDEFTRASPEANAALLSVLEEGVLVS | |
| | TDGASRHQYIEAHPDFRVLLTSNPHEYQGVKAAPDALIDRMVTLRLEEPSAPTLAG | |
| | IVALRSGLDPATARRIVDLILSVQRSGEMQAPPSMRTAILVARLAAPLRLAGRLSDA | |
| | ALAEIAADVLRGRGLEADAAAFEAKLAAPTPGETAR* | |
| Serratia sp. ATCC | MIKQNTVSQYTVDDDLVVPEASEHFVATSYVNDIIERALVYLRAGYPVHFAGPSGI | 455 |
| 39006_gvpN | GKTTLAFHLAALWGRPVTMLQGNEEFVSSDLTGKDIGYRKSSLVDNYIHSVLKTEE | |
| | QMNRMWVDNRLTTACRNGDMLIYDEFNRSKAETNNVLLSVLSEGILNLPGLRGV | |
| | GEGYLDVHPEFRAIFTSNPEEYAGTHKTQDALMDRMITINIGLVDRDTELQILHAR | |
| | SELELKEAAYIVDIIRELRGNEHETKHGLRAGIAIAHILHQQGIKPRYGDKLFHAICYD | |
| | VLSMDAAKIQHAGRSIYREMVDGVIRKICPPIGSDTVKASTQKIKAVE* | |
| Stella vacuolata_ATCC- | MSTEPAPVMPPSTDIEFGSQRPARPKPAEALAVGYRLSARPAAPSTLTLRPRADFV | 456 |
| 43931_gvpN | ETDQVKDLTRRGLGFLRAGYPLHFRGPAGTGKTTLALHVAAQLGRPVIVITGDNEL | |
| | GTADLVGSQRGYHYRKVVDQFIHNVTKLEETANQRWTDHRLTTACREGYTLVYD | |
| | EFTRSRPETHNVLLGVFEEKILFLPAEAREECYIRVHPDFRAIFTSNPQEYAGVHASQ | |
| | DALADRLATIDVDYPNRAMELAVASARTGLAEAEAARIIDLVRAFRASGDYQQTPT | |
| | MRASLMIARVAAQEGLRISVDDPGFVQLCMDALESRIFSGARQEADARARHRVA | |
| | LLGLLATHCPSEAPVARVATVARAKRKSAS* | |
| Thiocapsa rosea strain | MSAKPLQDASEVSALNNDNVQPEASDTFVCTPSVEALAERASAYLQAGYPVHLA | 457 |
| DSM 235 | GPAGTGKTTLAFHAAAKRGRPVKLIHGNDELGLADMVGQDNGYRRNTLVDNYIH | |
| Ga0242571_11_gvpN | SVVKTQEEVRTFWIDNRVTTACLNGETLIYDEFNRSRPEVNNIFLSILGEGILNLPNR | |
| | RHQGAGYLEVHPEFRVIFTSNPEEYAGTHKTQDALMDRMITMKIGHYDRETEIRV | |
| | TRAKSGLPPSEVAIVVDIVRELRGQSVNHHRPTLRACIAIARIMADRRISARSNNSF | |
| | FRDICRDILDMDSAKVRRDGNALGESPVDDVVASISARARRPKIVEPKGLHKEI* | |
| Tolypothrix sp. PCC | MTNTENHKKRAVLRVRPGQFVVTPAIEKVAIRALRYLTSGFAIHLRGPAGTGKTTL | 458 |
| 7601_gvpN | AMHLANCLDRPIMLIFGDDEFKSSDLIGSESGYTHKKLLDNYIHNVLKVEDELKQN | |
| | WVDSRLTLACREGLTLVYDEFNRSRPEVNNVLLSALEEKILTLPPSSNQPEYLHVHP | |
| | KFRAIFTSNPEEYCGVHSTQDALMDRLVTINMPEPDEQTQIEILTHKTGIHHEYAQ | |
| | LIARLVKAFRSATGAEKTSGLRSCLMVAKVCAEHDILVTPENTDFREICADVLFNRT | |
| | NLSASDATTLFLELLNHVQVKPVEPVDDSDPYDVAEAEIVGAAEPQTDAIAEPVTL | |
| | DESLLSDQPN* | |
| Trichodesmium | MTTVLNVSPDRFVSTPGVERVTQRASRYLESGYSVHLRGPAGVGKTTLALHLAHL | 459 |
| erythraeum | RQQPIFLMIGDDEFKTSDLIGNKSGYTRKKLVDNYIHTVLKVEDELRDNWIDSRLTL | |
| IMS101_gvpN1 | ACKEGFTLIYDEFNRSRPEVNNVLLSVLEEKMLVLPPSQNQSEYIQVHPQFRVILTS | |
| | NSEEWTGVHATQDALLDRVVTIGMEQPDISTEQNIVIQKTGINPLKAEVIIKLVRSV | |
| | RQRVDKEDLGSLRSALMISKVCHDHDIPLDGKDSSFSDLCADILISRPNLPRQEALQ | |
| | QLDEVLEEFFPADQPSSSDVGLEKEGSL* | |
| Trichodesmium | MTTVLNVSPDRFVSTPSVERVTQRASRYLESGYSVHLRGPAGVGKTTLALHLAHLR | 460 |
| erythraeum | QQPIFLMIGDDEFKTSDLIGNKSGYTRKKLVDNYIHTVLKVEDELKHNWIDSRLTLA | |
| IMS101_gvpN2 | CKEGFTLIYDEFNRSRPEVNNVLLSVLEEKMLVLPPSQNQSEYIQVHPQFRVILTSN | |
| | SEEWTGVHATQDALLDRVVTIGMGQPDISTEQNIIIQKTGINPLKAEVIIKLVRSVR | |
| | ERLETEDLGSLRSALMISKVCHDHDIPLGGKDSNFSDLCADILISRANLPRQEALKQL | |
| | DEVLEELFPADQLSISDIGLKKEGSL* | |
| gvpV | | |
| Anabaena-flos- | MIKNIQVFFMKTISNRSISRAKISTMPRPKSDASSQLDLYKMVTEKQRIQRDMYSIK | 461 |
| aquae_gvpV | ERMGLLQQRLDILNQQIEATEKTIHKLRQPHSNTAQNIVRSNIFVESNNYQTFEVE | |
| | Y* | |
| Aphanizomenon flos- | MKSFRHRSIIRAKISTMPRHISEASSQLELYKMVAEKQRISRELSSIKERMATLQKRL | 462 |
| aquae NIES-81_gvpV | DSLNNEIDNTEKTIHKLRQPHSSTAQNIVRSKNVVESNNYQTFEIEY* | |
| Arthrospira platensis | MRYKYHRQIQPKLSAIPRQKSQANLYRNSYLLAVEKKRLTEELEVLQSRSHIIEQRLA | 463 |
| NIES-39_gvpV | LIEDQLGELEKDVTQLSVPPSPKPQNNLPVNNPEPPPQSNPTNSSHINTFMVDY* | |
| Burkholderia | MPIPKKGLHDIRFRHAPGATPLPVHSMYMRISCIEMEKSRRTIERRAAQRRIAAVD | 464 |
| thailandensis sp. | SRVADLEREKARLYAAIDNEAPQAGDIRGSFRIRY* | |
| Bp5365 strain | | |
| MSMB43_gvpV | | |
| Desulfobacterium | MLKNRNRSIKGVQNIKTHAGKVDHVSHPHMAYMRISCLEMEKARKNKEKSGAQ | 465 |
| vacuolatum_DSM | KRIDMINQRLMEIEKEKAHIQRILGDTSIALESSNVDHDSEIKGGFKIKY* | |
| 3385_gvpV | | |
| Desulfomonile tiedjei | MNIRMKGNSRGLRDIRTHSGKVDRVGLPYMAYMSISCLEMEKARREKERLSALTR | 466 |
| DSM 6799_gvp | VIKNIEQRIREIEAEKDLLLKGVGERTRTDLQKASTPRDQSAQCKGGFKIRY* | |
| Legionella drancourtii | MMPALVKGLRNIKTMSNRLDKVQSPHEAFISAAALHREKQRHLQELAILRNRLDEI | 467 |
| LLAP12_gvpV | NLRLEQINEQQNQVAEAFDISPPRAVKSALRTGIQSKTGSTSHGFKIKY* | |
| Microcystis aeruginosa | MTTTRPPRPIRSKISTMPRKQSEADHQLELYKLITEKQRIQEKLEMMERQIQQLKN | 468 |
| NIES-843_gvpV | RLTFVTEQIETTEQSIQNLRTANPPSVAKKPDSPKTVAHSSNNSSNFQTFYLEY* | |
| Nostoc punctiforme | MHRTPNRRQIQAKLSTMPPQRSQATVYLNAYKMMLEKERLEEELEKLEARRHQI | 469 |
| ATCC 29133_gvpV | QQRLAILNSQTIPEENMTHQQANTDLENNTPKFNTLTLEY* | |
| Nostoc sp. PCC | MLSIIQVFPMTKVRNRGIIRPKITTMPRNKSEASSQLELYKLVTEQQRIKQELAFIEQ | 470 |
| 7120_gvpV | RTVLLKQRLSTLKTQIEGTERSINHLRHSELKYSRIALPKIFSETNNYQAFDIEY* | |
| Planktothrix agardhii | MRPFRSQPPILPKISTMPRQKTEATLYRSLYQLAVEKKRLQEELESLGQRFETVTQR | 471 |
| str. 7805_gvpV | LQQIETQIQGLETDVKQIAPPKPPETKPNQPSTPTPTKAEPGSVSTFTLDY* | |
| Psychromonas | MTAAKRKTLRGLADIRTISSCGTSGQEAYQMYLKRGVLEMEKLRRQKEKNSALER | 472 |
| ingrahamii 37_gvpV1 | VTNINRRLMAIDTDIDFLCQSLKVIEKRTNQENSIVEKSVSRGFKLRY* | |
| Psychromonas | MIFSKKKNALRGLADIRTLSGCGTSGQEAYQMYLKRGVLEMEKLRRQKEKNSALE | 473 |
| ingrahamii 37_gvpV2 | RVRNINYRLMAIDADIDFLCQSLKVIEERTNKENSISNESVTYKKGFKLRY* | |
| Serratia sp. ATCC | MAISTRPLRTLSDIKTHSGRVSGEHQTYRDYFQIGALELERWRRTREREAASSRIASI | 474 |
| 39006_gvpV | DERIADIDKEKAALLADATAASAVAENNDKSEAAEKKKKSSGLRIKY* | |
| Thiocapsa rosea strain | MSKFTQPSRSVRDIKTLAGMADDVRAPHKMYMRLFALETERHRRLQERASAMLR | 475 |
| DSM 235 | VDNIDARCAEIAEEMEQLLQILGVEAVAPGGPPANARPGSGRVPTQPHRGRGKG | |
| Ga0242571_11_gvpV | TGAGRQTTSGETSVGEAVKIRY* | |
| gvpW | | |
| Anabaena-flos- | MELENLYTYAFLEIPSSPLILPQGAANQVVLINGTELAAIVEPGIFLESFQNNDEKIIQ | 476 |
| aquae_gvpW | MALSHDRVICELFQQITVLPLRFGTYFTSTNNLLNHLKSHEKEYQNKLEKINGKNEF | |
| | TLKLIPRMIEEIVPSEGGGKDYFLAKKQRYQNQNNFSIAQAAEKQNLIDLITKVNQL | |
| | PVVVQEQEEQIQIYLLVSCQDKTLLLEQFLTWQKACPRWDLLLGDCLPPYHFI* | |
| Aphanizomenon flos- | MELENLYTYAFLKTPSFSLHLPQGSTTSVIQIDGNGLSAIVEPGISLDSFQDDDEKIV | 477 |
| aquae NIES-81_gvpW | QMAIEHDRVICDIFRQITVLPLRFGTYFANTDNLLTHLESYGQEYLDKLEKINCKTEFI | |
| | LKLIPRMITEESPVLESGRHYFLAKKQHYQRQKNFILAQASEKEILINFISKINQIPVII | |
| | QEQEEEVRIYLLVNYQDKTLLLEQFLTWQQTCPRWDLFLGEGIPPYHFI* | |
| Arthrospira platensis | MYVYAFIKSQSISWKSVQGIYEPVVLLEAGALAAVVEPNLQAENLSADNEEELMR | 478 |
| NIES-39_gvpW | AVLTHDRIVCQIFEETTVLPVRFGTCFDSEARLCEHLTTEGDRYFRQLEKLTGRAEYL | |
| | LEAIPQPFNQEKPSSDTTAPPTKGRDYFLQKKRLHQQRLNFEQQQEQQWQDFIN | |
| | AIASKYPIVQGKATEDAERIYLLIPRSQEVALVEWVAQQQQNIDLWEFSLGNAVPA | |
| | YHFL* | |
| Dolichospermum | MKLENFYTYAFLEIPRFPLVLPQGAASQVILINGSGMSAIVEPGISLESFQNNDEKII | 479 |
| circinale_gvpW | QMALSHDRVICELFQQVTVLPLRFGTCFTSTNNLLNYLELHRQEYQEKLEKINGKIE | |
| | FTLKLIPQTMEEPAPLERGGRDYFLAKKQRYQDQNNFRIAQAAEKQNLIDSISKVN | |
| | QLPFVIQEKEEEVNIYLLVKSEDKTLLLEQFLNWQKACPRWDLLLGEPLPPYHFI | |
| Microcystis aeruginosa | MKLYNLYTYAFLKTPIESLKLPVGMANPLLLITGGELSAVVEPEVGLDTLQNDDERLI | 480 |
| NIES-843_gvpW | QSVLCHDRVICQLFQQTTILPLRFGTSFLEAENLLTHLCSHGQEYQEKIEELEGKGEY | |
| | LLKCIPRKPEEPVLFSESKGRQYFLAKKQLYEAQQDFYTLQGSEWQNLVNLITQSYP | |
| | STRIITAPGTESRIYLLVNLQEEPLLIEQVLHWQKACPRWELQLGQVSPPYHFT* | |
| Nostoc punctiforme | MSIYAYALLVPTASPLVLPLGMERNTELVYSSGLAALVEPEISLEAIQATDERLLQAV | 481 |
| ATCC 29133_gvpW | LNHDHVIRELFQQTPLLPLRFGRGFTSVEKLLNHLENHQEQYLETLTQLADKVEYSV | |
| | KVTACSLLDDSDTIDARGKAYLLAKKQRYQTQQAFQAQQCEQWELLNELILKTYT | |
| | NVICETRQSDVRQIHFLAQRNDSTLSTQLFSLWQVQCSHWQLALSEPLPPYHFLK | |
| | NTLI* | |
| Nostoc sp. PCC | MRSPNFYTYAFLNTPDIPLRLPSGNLGQLLLIHGHKLSAVVEPGISLESSQNNDEEVI | 482 |
| 7120_gvpW | KMVLAHDRVICELSQQTTVLPLRFGTYFNSEETLLNHIESHAQEYQKKLDHIQGKTE | |
| | YTLKLIPRKFEELAKVSGGNGRDYFLAKKLHYEHQKNFIGDQNREKNHLINLIMDVY | |
| | RSSAIIQDYVEEVRLHLLVDRHDKTLLFKQVLTLQEKCPHWNLILGEPLPPYHFV* | |
| gvpR | | |
| Bacillus- | MEIKKIMQAVNDFFGEHVAPPHKITSVEATEDEGWRVIVEVIEEREYMKKYAKDE | 483 |
| megaterium_gvpR | MLGTYECFVNKEKEVISFKRLDVRYRSAIGIEA* | |
| gvpS | | |
| Bacillus- | MSLKQSMENKDIALIDILDVILDKGVAIKGDLIISIAGVDLVYLDLRVLISSVETLVQA | 484 |
| megaterium_gvpS | KEGNHKPITSEQFDKQKEELMDATGQPSKWTNPLGS* | |
| Rhodococcus hoagii | MSATPDRRIALVDLLDRVLGGGVVVAGEITLSIADVDMVHISLRTLVSSVSALTRPP | 485 |
| 103S_gvpS | DEKPENDG* | |
| gvpT | | |
| Bacillus- | MATETKLDNTQAENKENKNAENGSKEKNGSKASKTTSSGPIKRAVAGGIIGATIGY | 486 |
| megaterium_gvpT | VSTPENRKSLLDRIDTDELKSKASDLGTKVKEKSKSSVASLKTSAGSLFKKDKDKSKD | |
| | DEENVNSSSSETEDDNVQEYDELKEENQTLQDRLSQLEEKMNMLVELSLNKNQD | |
| | EEAEDTDSDEEENDENDENDENEQDDENEEETSKPRKKDKKEAEEEESESDEDSE | |
| | EEEEDSRSNKKNKKVKTEEEDEDESEEEKKEAKPKKSTAKKSKNTKAKKNTDEEDD | |
| | EATSLSSEDDTTA* | |
| gvpU | | |
| Bacillus- | MSTGPSFSTKDNTLEYFVKASNKHGFSLDISLNVNGAVISGTMISAKEYFDYLSETF | 487 |
| megaterium_gvpU | EEGSEVAQALSEQFSLASEASESNGEAEAHFIHLKNTKIYCGDSKSTPSKGKIFWRG | |
| | KIAEVDGFFLGKISDAKSTSKKSS* | |

TABLE 15
Protein sequences of gypC from exemplary species:

| | UniProt | | SEQ |
|---|---|---|---|
| Species | ID No. | Amino acid Sequence | ID NO: |
| Anabaena flos- | P09413 | MISLMAKIRQEHQSIAEKVA | 488 |
| aquae | | ELSLETREFLSVTTAKRQEQ | |
| | | AEKQAQELQAFYKDLQETSQ | |
| | | QFLSETAQARIAQAEKQAQE | |
| | | LLAFHKELQETSQQFLSATA | |
| | | QARIAQAEKQAQELLAFYQE | |
| | | VRETSQQFLSATAQARIAQA | |
| | | EKQAQELLAFHKELQETSQQ | |
| | | FLSATADARTAQAKEQKESL | |
| | | LKFRQDLFVSIFG | |
| Halobacterium | P24574 | MSVTDKRDEMSTARDKFAES | 489 |
| salinarum | | QQEFESYADEFAADITAKQD | |
| | | DVSDLVDAITDFQAEMTNTT | |
| | | DAFHTYGDEFAAEVDHLRAD | |
| | | IDAQRDVIREMQDAFEAYAD | |
| | | IFATDIADKQDIGNLLAAIE | |
| | | ALRTEMNSTHGAFEAYADDF | |
| | | AADVAALRDISDLVAAIDDF | |
| | | QEEFIAVQDAFDNYAGDFDA | |
| | | EIDQLHAAIADQHDSFDATA | |
| | | DAFAEYRDEFYRIEVEALLE | |
| | | AINDFQQDIGDFRAEFETTE | |
| | | DAFVAFARDFYGHEITAEEG | |
| | | AAEAEAEPVEADADVEAEAE | |
| | | VSPDEAGGESAGTEEEETEP | |
| | | AEVETAAPEVEGSPADTADE | |
| | | AEDTEAEEETEEEAPEDMVQ | |
| | | CRVCGEYYQAITEPHLQTHD | |
| | | MTIQEYRDEYGEDVPLRPDD | |
| | | KT | |
| Halobacterium | Q02228 | MSVKDKREKMTATREEFAEV | 490 |
| mediterranei | | QQAFAAYADEFAADVDDKRD | |
| | | VSELVDGIDTLRTEMNSTND | |
| | | AFRAYSEEFAADVEHFHTSV | |
| | | ADRRDAFDAYADIFATDVAE | |
| | | MQDVSDLLAAIDDLRAEMDE | |
| | | THEAFDAYADAFVTDVATLR | |
| | | DVSDLLTAISELQSEFVSVQ | |
| | | GEFNGYASEFGADIDQFHAV | |
| | | VAEKRDGHKDVADAFLQYRE | |
| | | EFHGVEVQSLLDNIAAFQRE | |
| | | MGDYRKAFETTEEAFASFAR | |
| | | DFYGQGAAPMATPLNNAAET | |
| | | AVTGTETEVDIPPIEDSVEP | |
| | | DGEDEDSKADDVEAEAEVET | |
| | | VEMEFGAEMDTEADEDVQSE | |
| | | SVREDDQFLDDETPEDMVQC | |
| | | LVCGEYYQAITEPHLQTHDM | |
| | | TIKKYREEYGEDVPLRPDDK | |
| | | A | |
| Microchaete | P08041 | MTPLMIRIRQEHRGIAEEVT | 491 |
| diplosiphon | | QLFKDTQEFLSVTTAQRQAQ | |
| | | AKEQAENLHQFHKDLEKDTE | |
| | | EFLTDTAKERMAKAKQQAED | |
| | | LFQFHKEMAENTQEFLSETA | |
| | | KERMAQAQEQARQLREFHQN | |
| | | LEQTTNEFLADTAKERMAQA | |
| | | QEQKQQLHQFRQDLFASIFG | |
| | | TF | |
| Nostoc sp. | Q8YUS9 | MTALMVRIRQEHRSIAEEVT | 492 |
| | | QLFRETHEFLSATTAHRQEQ | |
| | | AKQQAQQLHQFHQNLEQTTH | |
| | | EFLTETTTQRVAQAEAQANF | |
| | | LHKFHQNLEQTTQEFLAETA | |
| | | KNRTEQAKAQSQYLQQFRKD | |
| | | LFASIFGTF | |

TABLE 16
Amino acid sequences of exemplary GVS and
GVA proteins from B. megaterium.

| GVA | | SEQ ID |
|---|---|---|
| Protein | Amino acid sequence | NO.: |
| gvpB | MSIQKSTNSSSLAEVIDRILDKGIVIDAF | 493 |
| | ARVSVVGIEILTIEARVVIASVDTWLRYA | |
| | EAVGLLRDDVEENGLPERSNSSEGQPRFS | |
| | I | |
| gvpR | MEIKKIMQAVNDFFGEHVAPPHKITSVEA | 494 |
| | TEDEGWRVIVEVIEEREYMKKYAKDEMLG | |
| | TYECFVNKEKEVISFKRLDVRYRSAIGIE | |
| | A | |
| gvpN | MTVLTDKRKKGSGAFIQDDETKEVLSRAL | 495 |
| | SYLKSGYSIHFTGPAGGGKTSLARALAKK | |
| | RKRPVMLMHGNHELNNKDLIGDFTGYTSK | |
| | KVIDQYVRSVYKKDEQVSENWQDGRLLEA | |
| | VKNGYTLIYDEFTRSKPATNNIFLSILEE | |
| | GVLPLYGVKMTDPFVRVHPDFRVIFTSNP | |
| | AEYAGVYDTQDALLDRLITMFIDYKDIDR | |
| | ETAILTEKTDVEEDEARTIVTLVANVRNR | |
| | SGDENSSGLSLRASLMIATLATQQDIPID | |
| | GSDEDFQTLCIDILHHPLTKCLDEENAKS | |
| | KAEKIILEECKNIDTEEK | |
| gvpF | MSETNETGIYIFSAIQTDKDEEFGAVEVE | 496 |
| | GTKAETFLIRYKDAAMVAAEVPMKIYHPN | |
| | RQNLLMHQNAVAAIMDKNDTVIPISFGNV | |
| | FKSKEDVKVLLENLYPQFEKLFPAIKGKI | |
| | EVGLKVIGKKEWLEKKVNENPELEKVSAS | |
| | VKGKSEAAGYYERIQLGGMAQKMFTSLQK | |
| | EVKTDVFSPLEEAAEAAKANEPTGETMLL | |
| | NASFLINREDEAKFDEKVNEAHENWKDKA | |
| | DFHYSGPWPAYNFVNIRLKVEEK | |
| gvpG | MLHKLVTAPINLWKIGEKVQEEADKQLYD | 497 |
| | LPTIQQKLIQLQMMFELGEIPEEAFQEKE | |
| | DELLMRYEIAKRREIEQWEELTQKRNEES | |
| gvpL | MGELLYLYGLIPTKEAAAIEPFPSYKGFD | 498 |
| | GEHSLYPIAFDQVTAVVSKLDADTYSEKV | |
| | IQEKMEQDMSWLQEKAFHHHETVAALYEE | |
| | FTIIPLKFCTIYKGEESLQAAIEINKEKI | |
| | ENSLTLLQGNEEWNVKIYCDDTELKKGIS | |
| | ETNESVKAKKQEISHLSPGRQFFEKKKID | |
| | QLIEKELELHKNKVCEEIHDKLKELSLYD | |
| | SVKKNWSKDVTGAAEQMAWNSVFLLPSLQ | |
| | ITKFVNEIEELQQRLENKGWKFEVTGPWP | |
| | PYHFSSFA | |
| gvpS | MSLKQSMENKDIALIDILDVILDKGVAIK | 499 |
| | GDLIISIAGVDLVYLDLRVLISSVETLVQ | |
| | AKEGNHKPITSEQFDKQKEELMDATGQPS | |
| | KWTNPLGS | |
| gvpK | MQPVSQANGRIHLDPDQAEQGLAQLVMTV | 500 |
| | IELLRQIVERHAMRRVEGGTLTDEQIENL | |
| | GIALMNLEEKMDELKEVFGLDAEDLNIDL | |
| | GPLGSLL | |
| gvpJ | MAVEHNMQSSTIVDVLEKILDKGVVIAGD | 501 |
| | ITVGIADVELLTIKIRLIVASVDKAKEIG | |
| | MDWWENDPYLSSKGANNKALEEENKMLHE | |
| | RLKTLEEKIETKR | |
| gvpT | MATETKLDNTQAENKENKNAENGSKEKNG | 502 |
| | SKASKTTSSGPIKRAVAGGIIGATIGYVS | |
| | TPENRKSLLDRIDTDELKSKASDLGTKVK | |
| | EKSKSSVASLKTSAGSLFKKDKDKSKDDE | |
| | ENVNSSSSETEDDNVQEYDELKEENQTLQ | |
| | DRLSQLEEKMNMLVELSLNKNQDEEAEDT | |
| | DSDEEENDENDENDENEQDDENEEETSKP | |
| | RKKDKKEAEEEESESDEDSEEEEEDSRSN | |
| | KKNKKVKTEEEDEDESEEEKKEAKPKKST | |
| | AKKSKNTKAKKNTDEEDDEATSLSSEDDT | |
| | TA | |
| gvpU | MSTGPSFSTKDNTLEYFVKASNKHGFSLD | 503 |
| | ISLNVNGAVISGTMISAKEYFDYLSETFE | |
| | EGSEVAQALSEQFSLASEASESNGEAEAH | |
| | FIHLKNTKIYCGDSKSTPSKGKIFWRGKI | |
| | AEVDGFFLGKISDAKSTSKKSS | |

TABLE 17
Amino acid sequences of exemplary GVS and GVA proteins from Serratia sp..

| GVA | | SEQ ID |
|---|---|---|
| Protein | Amino acid sequence | NO.: |
| gvpA1 | MAKVQKSTDSSSLAEVVDRILDKGIVIDAWVKVSLVGIELLSIEARVVIASVETYLKYAEAIGLT | 504 |
| | ASAATPA | |
| gvpA2 | MPVNKQYQDEQQQVSLCEALDRVLNKGVVIVADITISVANIDLIYLSLQALVSSVEAKNRLPGRE | 505 |
| gvpA3 | MPVNKQYQDEQQQVSLCEALDRVLNKGVVIVADITISVANIDLIYLSLQALVSSVEAKNRLPGRE | 506 |
| gvpC | MGCLTDGMAQLRKNIDDSHESRIAQQNARVSSVSAQIAGFSTTRARNAAQDARARATFVADNVR | 507 |
| | GVNRMLSDFCHTREVMSRQQSEERATFVTDMSKKTLALLDGFNAERKSMAERCAKERADFIANV | |
| | ANDVAAFLSASEKDRMAAHAVFFGMTLAKKKTSLAV | |
| gvpN | MIKQNTVSQYTVDDDLVVPEASEHFVATSYVNDIIERALVYLRAGYPVHFAGPSGIGKTTLAFHL | 508 |
| | AALWGRPVTMLQGNEEFVSSDLTGKDIGYRKSSLVDNYIHSVLKTEEQMNRMWVDNRLTTACRNG | |
| | DMLIYDEFNRSKAETNNVLLSVLSEGILNLPGLRGVGEGYLDVHPEFRAIFTSNPEEYAGTHKTQ | |
| | DALMDRMITINIGLVDRDTELQILHARSELELKEAAYIVDIIRELRGNEHETKHGLRAGIAIAHI | |
| | LHQQGIKPRYGDKLFHAICYDVLSMDAAKIQHAGRSIYREMVDGVIRKICPPIGSDTVKASTQKI | |
| | KAVE | |
| gvpV | MAISTRPLRTLSDIKTHSGRVSGEHQTYRDYFQIGALELERWRRTREREAASSRIASIDERIADI | 509 |
| | DKEKAALLADATAASAVAENNDKSEAAEKKKKSSGLRIKY | |
| gvpF1 | MMSIDKSRNHRAKVLYALCVSDDSTPNYKIRGLEAAPVYSIDQDGLRAVVSDTLSTRLRPERRNI | 510 |
| | TAHQAVLHKLTEEGTVLPMRFGVIARNAEAVKNLLVANQDTIREHFERLDGCVEMGLRVSWDVTN | |
| | IYEYFVATYPVLSETRDEIWNGNSNANNHREEKIRLGNLYESLRSGDRKESTEKVKEVLLDYCEE | |
| | IIENPVKKEKDVMNLACLVARERMDEFAKGVFEASKLFDNVYLFDYTGPWAPHNFVTLDLHAPTA | |
| | KKKTLTRAGTLSD | |
| gvpF2 | MTMNTEAQTEQAIYLYGLTLPDLAAPPILGVDNQHPINTHQCAGLNAVISPVALSDFTGEKGEDN | 511 |
| | VQNVTWLTPRICRHAQIIDSLMAQGPVYPLPFGTLFSSQNALEQEMKSRATDVFVSLRRITGCQE | |
| | WALEATLDRKQAVDVLFTEGLDSGRFCLPEAIGRRHLEEQKLRRRLTTELSDWLAHALTAMQNEL | |
| | HPLVRDFRSRRLLDDKILHWAYLLPVEDVAAFQQQVADIVERYEAYGFSFRVTGPWAAYSFCQPD | |
| | ES | |
| gvpF3 | MSLLLYGIVAEDTQLALEPDGSPHAGEEPMQLVKAATLAALVKPCEADVSREPAAALAFGQQIMH | 512 |
| | VHQQTTIIPIRYGCVLADEDAVTQHLLNHEAHYQTQLVELENCDEMGIRLSLASAEDNAVTTPQA | |
| | SGLDYLRSRKLAYAVPEHAERQAALLNNAFTGLYRRHCAEISMFNGQRTYLLSYLVPRTGLQAFR | |
| | DQFNTLANNMTDIGVISGPWPPYNFAS | |
| gvpG | MLLIDDILFSPVKGVMWIFRQIHELAEDELAGEADRIRESLTDLYMLLETGQITEDEFEQQEAVL | 513 |
| | LDRLDALDEEDDMLGDEPGDDEDDEYEEDDDEEDDDEEDDDDEDDDDEDDDDEEDDDDDEDDDDE | |
| | DEPEGTTK | |
| gvpW | MKPAIYPKFLLESPLKLVFFGGKGGVGKSTCATSTALRLAQEQPQHHFLLVSTDPAHSLQNILSD | 514 |
| | LVLPKNLDVRELNAAASLHEFKSQHEGVLKEIAYRGTVLDQNDVQGLMDTALPGMDELAAYLEIA | |
| | EWIQKDTYYRIIIDTAPTGHTLRLLEMPDLIYRWLTALDTLLAKQRYIRKRFAGDNRLDHLDHFL | |
| | LDMNDSLKAMHELVTDSTRCCFVLVMLAEAMSVEESIDLAGALNQQRVFLSDLVVNRLFPENDCP | |
| | TCCVERNRQMLALQNGYQRLPGHVFWTLPLLAIEPRGALLHEFWSGVRLLDENEVMATTCHHQLP | |
| | LRVESSISLPASTFRLLIFAGKGGVGKTTLACATALRLNSEYPELRILLFSADPAHSLSDCLGVT | |
| | LQQQPISVLVNIDAQEINAQADFDKIRQGYRAELEAFLLDTLPNLDITFDREVLEHLLDLAPPGL | |
| | DEIMALTAIMDHLDSGRYDMVIVDGAPSGHLLRLLELPELIRDWLKQFFSLLLKYRKVMRFPHLS | |
| | ERLVQLSRELKNLRALLQDTKQTGLYAVTVPTHLALEKTYEMTCALQRLGLTANALFINQITPPS | |
| | DCTLCQAITSRESLELKCADEMFPSQPHAQIFRQTEPTGLSKLKTLGSALFL | |
| gvpK | MTTNQLSHHSPVFGPTSPAIQRPITEANRHKIDIDGERVRDGLAQLVLTLVKLLHELLERQAIRR | 515 |
| | MDSGSLSDEEVERLGLALMRQAEELTHLCDVFGFKDDDLNLDLGPLGRLL | |
| gvpX | MVNTTNDINAATRGLLLRMGNAWFEQDELRQAVDIYLKIIEQYPDSKESKTAQTALLTISQRYER | 516 |
| | DGLFRLSLDILERVGEITPTSI | |
| gvpY | MRALIHFPIIHSPKDLGTLSEAASHLRTETQTRAYLAAVEGFWTMITTTIEGLDLDYTHLKLYQD | 517 |
| | GLPVCGKENEIVTDVANAGSQNYKLLLTLQHKGAILMGTESPELLLQERDLMTQLLQSTEQTEAS | |
| | LETAKTLLNRRDDYIAQRIDETLQDGEMAILFLGLMHNIEAKLPADIVFIQPLGKPPGGESI | |
| gvpH | MTGNVEGILRGLGDLVEKLVETGEQIKRSGAFDIDTNDGKNAKAVYGFSIKMGLDGNQENRVEPF | 518 |
| | GNIRRDEQTGEATVQEVSEPLVDVIEESDHVLVLAEMPGVADEDVQVELNGDILTLHSERGSKKY | |
| | HKEIVLPCSFDDKAMERSCRNGILEVKLGK | |
| gvpZ | MSEELKLKVAEALPKDAGRGYARLDPADMARLNLAVGDIVQLTSKKGTGIAKLMPTYPDMRNKGI | 519 |
| | VQLDGLTRRNTSLSLDEKVQIEPASCKHATQIVLIPTTITPNQRDLDYIGSLLDGLPVQKGDLLR | |
| | AHLFGSRSADFKVESTIPDGAVLIDPTTTLVIGKSNAVGNSSHSTQRLSYEDVGGLKNQVRRIRE | |
| | MIELPLRYPEVFERLGIDAPKGVLLSGPPGCGKTLIARIIAQETDAQFFTISGPEIVHKFYGESE | |
| | AHLRKIFEEAGRKGPSIIFLDEIDSIAPHRDKVVGDVEKRIVAQLLALMDGLKNRGKVIVIAATN | |
| | LPNAIDPALRRPGRFDREISIPIPDREGRREIIEIHSTGMPLNADVDLNVLADITHGFVGADLEA | |
| | LCREAAMSALRRLLPEIDFSSAELPYDRLAELTVMMDDFRAALCEVSPSAIRELFVDIPDVRWED | |
| | VGGLDDVRRRLIESVEWPIKYPELYEQAGVKPPKGLLLAGPPGVGKTLIAKAVANESGVNVISVK | |
| | GPALMSRYVGDSEKGVRELFLKARQAAPCIIFLDEVDSVIPARNEGAIDSHVAERVLSQFLSEMD | |
| | GLEELKGVFVMGATNRADLIDPAMLRPGRFDEIIELGLPDEDARRQILAVHLRNKPLGDNIHADD | |
| | LAERCDGASGAELAAVCNRAALAALRRAIQQSEEAVLSPSTVGETPVALTVRIEQHDFAEVIAEM | |
| | FGDDA | |

TABLE 18
Amino Acid Sequences of GV proteins
from Anabaena flos-aquae

| gyp | | SEQ ID |
|---|---|---|
| gene | Sequence | NO: |
| gvpA | MAVEKTNSSSSLAEVIDRILDKGIVIDAWVRVSLV | 520 |
| | GIELLAIEARIVIASVETYLKYAEAVGLTQSAAVP | |
| | A | |
| gvpC | MISLMAKIRQEHQSIAEKVAELSLETREFLSVTTA | 521 |
| | QKRQEQAEKQAQELQAFYKDLQETSQQFLSETAAR | |
| | IAQAEKQAQELLAFHKELQETSQQFLSATAQARIA | |
| | QAEKQAQELLAFYQEVRETSQQFLSATAQARIAQA | |
| | EKQAQELLAFHKELQETSQQFLSATADARTAQAKE | |
| | QKESLLKFRQDLFVSIFG | |
| gvpN | MTTTKVNHKRAVLRLRPGQFVVTPAIERVAIRALR | 522 |
| | YLKSGFPVHLRGPAGTGKTTLAMHLANCLDRPVML | |
| | LFGDDQFKSSDLIGSESGYTHKKVLDNYIHSVVKL | |
| | EDEFKQNWVDSRLTLACREGFTLVYDEFNRSRPEV | |
| | NNVLLSALEEKILSLPPSSNQPEYLSVNPQFRVIF | |
| | TSNPEEYAGVHSTQDALMDRLVTISMPEPDEITQT | |
| | EILIQKTNIDRESANFIVRLVKSFRLATGAEKTSG | |
| | LRSCLMIAKVCADNNIPVTTESLDFPDIAIDILFN | |
| | RSHLSMSESTNIFLELLDKFSAEELEILNNRVTGD | |
| | NDFLIDNSQFVSQQLAGQPN | |
| gvpJ | MLPTRPQTNSSRTINTSTQGSTLADILERVLDKGI | 523 |
| | VIAGDISISIASTELVHIRIRLLISSVDKAKEMGI | |
| | NWWESDPYLSTKAQRLVEENQQLQHRLESLEAKLN | |
| | SLTSSSVKEEIPLAADVKDDLYQTSAKIPSPVDTP | |
| | IEVLDFQAQSSGGTPPYVNTSMEILDFQAQTSAES | |
| | SSPVGSTVEILDFQAQTSEESSSPVVSTVEILDFQ | |
| | AQTSEESSSPVGSTVEILDFQAQTSEEIPSSVDPA | |
| | IDV | |
| gvpK | MVCTPAENFNNSLTIASKPKNEAGLAPLLLTVLEL | 524 |
| | VRQLMEAQVIRRMEEDLLSEPDLERAADSLQKLEE | |
| | QILHLCEMFEVDPADLNINLGEIGTLLPSSGSYYP | |
| | GQPSSRPSVLELLDRLLNTGIVVDGEIDLGIAQID | |
| | LIHAKLRLVLTSKPI | |
| gvpF | MSIPLYLYGIFPNTIPETLELEGLDKQPVHSQVVD | 525 |
| | EFCFLYSEARQEKYLASRRNLLTHEKVLEQTMHAG | |
| | FRVLLPLRFGLVVKDWETIMSQLINPHKDQLNQLF | |
| | QKLAGKREVSIKIFWDAKAELQTMMESHQDLKQQR | |
| | DNMEGKKLSMEEVIQIGQLIEINLLARKQAVIEVF | |
| | SQELNPFAQEIVVSDPMTEEMIYNAAFLIPWESES | |
| | EFSERVEVIDQKFGDRLRIRYNNFTAPYTFAQLDS | |
| gvpG | MLTKLLLLPIMGPLNGVVWIAEQIQERTNTEFDAQ | 526 |
| | ENLHKQLLSLQLSFDIGEIGEEEFEIQEEEILLKI | |
| | QALEEEARLELEAEQEEARLELEAEQEDFEYPPQF | |
| | TAEVNKDQHLVLLP | |
| gvpV | MIKNIQVFFMKTISNRSISRAKISTMPRPKSDASS | 527 |
| | QLDLYKMVTEKQRIQRDMYSIKERMGLLQQRLDIL | |
| | NQQIEATEKTIHKLRQPHSNTAQNIVRSNIFVESN | |
| | NYQTFEVEY | |
| gvpW | MELENLYTYAFLEIPSSPLILPQGAANQVVLINGT | 528 |
| | ELAAIVEPGIFLESFQNNDEKIIQMALSHDRVICE | |
| | LFQQITVLPLRFGTYFTSTNNLLNHLKSHEKEYQN | |
| | KLEKINGKNEFTLKLIPRMIEEIVPSEGGGKDYFL | |
| | AKKQRYQNQNNFSIAQAAEKQNLIDLITKVNQLPV | |
| | VVQEQEEQIQIYLLVSCQDKTLLLEQFLTWQKACP | |
| | KWDLLLGDCLPPYHFI | |

Summary provided herein are engineered protease sensitive gas vesicles and related engineered protease genetically GvpCconstructs, vectors, gas vesicles gene clusters, genetic circuits, cells, compositions, methods and systems, which in several embodiments can be used together with contrast-enhanced imaging technique, to detect and report protease activity and related biological events in an imaging target site.

The examples set forth above are provided to give those of ordinary skill in the art a complete disclosure and description of how to make and use the embodiments of the hybrid GVGCs, and related GVR genetic circuits, vectors, genetically engineered prokaryotic cells, compositions, methods and systems of the disclosure, and are not intended to limit the scope of what the inventors regard as their disclosure. Those skilled in the art will recognize how to adapt the features of the exemplified hybrid GVGCs, and related genetic circuits, vectors, genetically engineered prokaryotic cells, compositions, methods and systems herein disclosed to additional hybrid GVGCs, and related genetic circuits, vectors, genetically engineered prokaryotic cells, compositions, methods and systems according to various embodiments and scope of the claims.

All patents and publications mentioned in the specification are indicative of the levels of skill of those skilled in the art to which the disclosure pertains.

The entire disclosure of each document cited (including patents, patent applications, journal articles, abstracts, laboratory manuals, books, or other disclosures) in the Background, Summary, Detailed Description, and Examples is hereby incorporated herein by reference. All references cited in this disclosure are incorporated by reference to the same extent as if each reference had been incorporated by reference in its entirety individually. However, if any inconsistency arises between a cited reference and the present disclosure, the present disclosure takes precedence. Further, the computer readable form of the sequence listing of the ASCII text file P2513-US-Sequence-Listing_ST25 is incorporated herein by reference in its entirety.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the disclosure has been specifically disclosed by embodiments, exemplary embodiments and optional features, modification and variation of the concepts herein disclosed can be resorted to by those skilled in the art, and that such modifications and variations are considered to be within the scope of this disclosure as defined by the appended claims.

It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

When a Markush group or other grouping is used herein, all individual members of the group and all combinations and possible sub-combinations of the group are intended to be individually included in the disclosure. Every combination of components or materials described or exemplified herein can be used to practice the disclosure, unless otherwise stated. One of ordinary skill in the art will appreciate that methods, system elements, and materials other than those specifically exemplified may be employed in the practice of the disclosure without resort to undue experimentation. All art-known functional equivalents, of any such methods, device elements, and materials are intended to be included in this disclosure. Whenever a range is given in the specification, for example, a temperature range, a frequency range, a time range, or a composition range, all intermediate ranges and all subranges, as well as, all individual values included in the ranges given are intended to be included in the disclosure. Any one or more individual members of a range or group disclosed herein may be excluded from a claim of this disclosure. The disclosure illustratively described herein suitably may be practiced in the absence of any element or elements, limitation or limitations which is not specifically disclosed herein.

A number of embodiments of the disclosure have been described. The specific embodiments provided herein are examples of useful embodiments of the disclosure and it will be apparent to one skilled in the art that the disclosure can be carried out using a large number of variations of the genetic circuits, genetic molecular components, and methods steps set forth in the present description. As will be obvious to one of skill in the art, methods and systems useful for the present methods and systems may include a large number of optional composition and processing elements and steps.

In particular, it will be understood that various modifications may be made without departing from the spirit and scope of the present disclosure. Accordingly, other embodiments are within the scope of the following claims.

### REFERENCES

1. Tashiro, Y., et al., Molecular genetic and physical analysis of gas vesicles in buoyant enterobacteria. Environmental microbiology, 2016. 18(4): p. 1264-1276.
2. Van Keulen, G., et al., Gas vesicles in actinomycetes: old buoys in novel habitats?Trends in microbiology, 2005. 13(8): p. 350-354.
3. Walsby, A. E., Gas vesicles. Microbiol. Rev., 1994. 58(1): p. 94-144.
4. Walsby, A. E., Gas-vacuolate bacteria (apart from cyanobacteria), in The Prokaryotes. 1981, Springer. p. 441-447.
5. Walsby, A. E., Cyanobacteria: planktonic gas-vacuolate forms. The Prokaryotes, a Handbook on Habitats, Isolation, and Identification of Bacteria, 2013. 1: p. 224-235.
6. Woese, C. R., Bacterial evolution. Microbiological reviews, 1987. 51(2): p. 221.
7. Walsby, A. E., Gas vesicles. Microbiol Rev, 1994. 58(1): p. 94-144.
8. Pfeifer, F., Distribution, formation and regulation of gas vesicles. Nat. Rev. Microbiol., 2012. 10(10): p. 705-15.
9. Yi, G., S.-H. Sze, and M. R. Thon, Identifying clusters of functionally related genes in genomes. Bioinformatics, 2007. 23(9): p. 1053-1060.
10. Bourdeau, R. W., et al., Acoustic reporter genes for noninvasive imaging of microorganisms in mammalian hosts. Nature, 2018. 553(7686): p. 86-90.
11. Lakshmanan, A., et al., Preparation of biogenic gas vesicle nanostructures for use as contrast agents for ultrasound and MRI. Nat Protoc, 2017. 12(10): p. 2050-2080.
12. Hayes, P. and R. Powell, The gvpA/C cluster of Anabaena flos-aquae has multiple copies of a gene encoding GvpA. Archives of microbiology, 1995. 164(1): p. 50-57.
13. Kinsman, R. and P. Hayes, Genes encoding proteins homologous to halobacterial Gvps N, J, K, F &L are located downstream of gvpC in the cyanobacterium Anabaena flos-aquae. DNA Sequence, 1997. 7(2): p. 97-106.
14. Myers, E. W. and W. Miller, Optimal alignments in linear space. Computer applications in the biosciences: CABIOS, 1988. 4(1): p. 11-17.
15. Smith, T. F. and M. S. Waterman, Comparison of biosequences. Advances in applied mathematics, 1981. 2(4): p. 482-489.
16. Needleman, S. B. and C. D. Wunsch, A general method applicable to the search for similarities in the amino acid sequence of two proteins. Journal of molecular biology, 1970. 48(3): p. 443-453.
17. Pearson, W. R. and D. J. Lipman, Improved tools for biological sequence comparison. Proceedings of the National Academy of Sciences, 1988. 85(8): p. 2444-2448.
18. Karlin, S. and S. F. Altschul, Methods for assessing the statistical significance of molecular sequence features by using general scoring schemes. Proceedings of the National Academy of Sciences, 1990. 87(6): p. 2264-2268.
19. Karlin, S. and S. F. Altschul, Applications and statistics for multiple high-scoring segments in molecular sequences. Proceedings of the National Academy of Sciences, 1993. 90(12): p. 5873-5877.
20. Lu, G. J., et al., Acoustically modulated magnetic resonance imaging of gas-filled protein nanostructures. Nat Mater, 2018. 17(5): p. 456-463.
21. Maresca, D., et al., Nonlinear Ultrasound Imaging of Nanoscale Acoustic Biomolecules. Applied Physics Letters, 2017. 110(7).
22. Maresca, D., et al., Nonlinear ultrasound imaging of nanoscale acoustic biomolecules. Appl Phys Lett, 2017. 110(7): p. 073704.
23. David Maresca, D. P. S., Guillaume Renaud, Audrey Lee-Gosselin, and Mikhail G. Shapiro, Nonlinear X-Wave Ultrasound Imaging of Acoustic Biomolecules. Phys. Rev. X, 2018. 8(4): p. 041002.
24. Collins, D. E. C. J. J., Tunable protein degradation in bacteria. Nature Biotechnology volume 2014. 32: p. 1276-1281.
25. Hélene Chassin, M. M., Marcel Tigges, Leo Scheller, Moritz Lang & Martin Fussenegger, A modular degron library for synthetic circuits in mammalian cells. Nature Communications 2019. 10: p. 2013.
26. Baker, R. T. S. a. T. A., AAA+Proteases: ATP-Fueled Machines of Protein Destruction. Annual Review of Biochemistry, 2011. 80: p. 587-612.
27. al., H. C.-M. e., Modulation of SQSTM1/p62 activity by N-terminal arginylation of the endoplasmic reticulum chaperone HSPA5/GRP78/BiP. Autophagy, 2016. 12(2): p. 426-428.
28. al., T. A. A. e., Systemic in vivo distribution of activatable cell penetrating peptides is superior to cell penetrating peptides. Integr Biol (Camb). 2009. 1(5-6): p. 371-381.
29. al., L. Y. e., Quantitatively Visualizing Tumor-Related Protease Activity in Vivo Using a Ratiometric Photoacoustic Probe. J. Am. Chem. Soc., 2019. 141(7): p. 3265-3273.
30. Zordan, R. E., et al., Avoiding the ends: internal epitope tagging of proteins using transposon Tn7. Genetics, 2015. 200(1): p. 47-58.
31. Lakshmanan, A., et al., Molecular Engineering of Acoustic Protein Nanostructures. ACS Nano, 2016. 10(8): p. 7314-7322.
32. Lakshmanan, A., et al., Preparation of biogenic gas vesicle nanostructures for use as contrast agents for ultrasound and MRI. Nature Protocols, 2017. 12(10): p. 2050.
33. Pfeifer, F., Distribution, formation and regulation of gas vesicles. Nat Rev Microbiol, 2012. 10(10): p. 705-15.
34. Li, N. and M. C. Cannon, Gas vesicle genes identified in Bacillus megaterium and functional expression in Escherichia coli. J Bacteriol, 1998. 180(9): p. 2450-8.
35. Tashiro, Y., et al., Molecular genetic and physical analysis of gas vesicles in buoyant enterobacteria. Environ Microbiol, 2016. 18(4): p. 1264-76.
36. Ramsay, J. P., et al., A quorum-sensing molecule acts as a morphogen controlling gas vesicle organelle biogenesis and adaptive flotation in an enterobacterium. Proc Natl Acad Sci USA, 2011. 108(36): p. 14932-7.
37. Datsenko, K. A. and B. L. Wanner, One-step inactivation of chromosomal genes in Escherichia coli K-12 using PCR products. Proceedings of the National Academy of Sciences, 2000. 97(12): p. 6640-6645.
38. St-Pierre, F., et al., One-step cloning and chromosomal integration of DNA. ACS synthetic biology, 2013. 2(9): p. 537-541.
39. Phan, J., et al., Structural basis for the substrate specificity of tobacco etch virus protease. Journal of Biological Chemistry, 2002. 277(52): p. 50564-50572.
40. Parks, T. D., et al., Release of proteins and peptides from fusion proteins using a recombinant plant virus proteinase. Analytical Biochemistry, 1994. 216(2): p. 413-417.
41. Maresca, D., et al., Nonlinear X-Wave Ultrasound Imaging of Acoustic Biomolecules. Physical Review X, 2018. 8(4): p. 041001-0410012.
42. Shapiro, M. G., et al., Biogenic gas nanostructures as ultrasonic molecular reporters. Nature Nanotechnology, 2014. 9(4): p. 311-316.
43. Goll, D. E., et al., The calpain system. Physiological Reviews, 2003. 83(3): p. 731-801.
44. Ono, Y. and H. Sorimachi, Calpains—an elaborate proteolytic system. Biochimica et Biophysica Acta (BBA)-Proteins and Proteomics, 2012. 1824(1): p. 224-236.
45. Ono, Y., T. C. Saido, and H. Sorimachi, Calpain research for drug discovery: challenges and potential. Nature Reviews Drug Discovery, 2016. 15(12): p. 854-876.
46. Suzuki, S., et al., Development of an artificial calcium-dependent transcription factor to detect sustained intracellular calcium elevation. ACS Synthetic Biology, 2014. 3(10): p. 717-722.
47. Sauer, R. T., et al., Sculpting the proteome with AAA(+) proteases and disassembly machines. Cell, 2004. 119(1): p. 9-18.
48. Baker, T. A. and R. T. Sauer, ClpXP, an ATP-powered unfolding and protein-degradation machine. Biochimica et Biophysica Acta (BBA)-Molecular Cell Research, 2012. 1823(1): p. 15-28.
49. Sonnenborn, U. and J. Schulze, The non-pathogenic Escherichia coli strain Nissle 1917-features of a versatile probiotic. Microbial Ecology in Health and Disease, 2009. 21(3-4): p. 122-158.
50. Danino, T., et al., Programmable probiotics for detection of cancer in urine. Science Translational Medicine, 2015. 7(289): p. 289ra84.
51. Blum-Oehler, G., et al., Development of strain-specific PCR reactions for the detection of the probiotic Escherichia coli strain Nissle 1917 in fecal samples. Research in Microbiology, 2003. 154(1): p. 59-66.
52. Elowitz, M. B. and S. Leibler, A synthetic oscillatory network of transcriptional regulators. Nature, 2000. 403(6767): p. 335-338.
53. Gardner, T. S., C. R. Cantor, and J. J. Collins, Construction of a genetic toggle switch in Escherichia coli. Nature, 2000. 403(6767): p. 339-342.
54. Khalil, A. S. and J. J. Collins, Synthetic biology: applications come of age. Nature Reviews Genetics, 2010. 11(5): p. 367-379.
55. Tigges, M., et al., A tunable synthetic mammalian oscillator. Nature, 2009. 457(7227): p. 309-312.
56. Mark Welch, J. L., et al., Spatial organization of a model 15-member human gut microbiota established in gnotobiotic mice. Proceedings of the National Academy of Sciences, 2017. 114(43): p. E9105-E9114.
57. Geva-Zatorsky, N., et al., In vivo imaging and tracking of host-microbiota interactions via metabolic labeling of gut anaerobic bacteria. Nature Medicine, 2015. 21(9): p. 1091-100.
58. Foucault, M. L., et al., In vivo bioluminescence imaging for the study of intestinal colonization by Escherichia coli in mice. Applied and Environmental Microbiology, 2010. 76(1): p. 264-74.
59. Donaldson, G. P., S. M. Lee, and S. K. Mazmanian, Gut biogeography of the bacterial microbiota. Nature Reviews Microbiology, 2016. 14(1): p. 20-32.
60. Round, J. L. and S. K. Mazmanian, The gut microbiota shapes intestinal immune responses during health and disease. Nature Reviews Immunology, 2009. 9(5): p. 313-323.
61. Derrien, M. and J. E. T. V. Vlieg, Fate, activity, and impact of ingested bacteria within the human gut microbiota. Trends in Microbiology, 2015. 23(6): p. 354-366.
62. Steidler, L., et al., Treatment of murine colitis by Lactococcus lactis secreting interleukin-10. Science, 2000. 289(5483): p. 1352-1355.
63. Daniel, C., et al., Recombinant lactic acid bacteria as mucosal biotherapeutic agents. Trends in Biotechnology, 2011. 29(10): p. 499-508.
64. Muradali, D. and D. R. Goldberg, US of gastrointestinal tract disease. Radiographics, 2015. 35(1): p. 50-68.
65. Machtaler, S., et al., Assessment of inflammation in an acute on chronic model of inflammatory bowel disease with ultrasound molecular imaging. Theranostics, 2015. 5(11): p. 1175.

1. A method to provide a protease sensitive gas vesicle, the method comprising
i) providing one or more engineered gas vesicles in which a gas is enclosed by a protein shell, the engineered gas vesicles comprising a gas vesicle, a GvpA/B protein and an engineered GvpC protein,
the engineered GvpC protein comprising:
multiple repeat regions within a central portion of the GvpC flanked by an N-terminal region having an N-terminus and a C-terminal region having a C-terminus, and
at least one protease recognition site inserted within the central portion and/or attached to at least one of the N-terminus and the C-terminus of the GvpC,
each of the one or more engineered gas vesicles exhibiting an initial collapse pressure and an initial ultrasound response up to collapse, the initial ultrasound response having a baseline nonlinearity,
ii) contacting the one or more engineered gas vesicles with a protease capable of binding the at least one protease recognition site to allow cleavage of the protease recognition site,
iii) following the contacting, detecting a protease induced collapse pressure and/or a protease induced ultrasound response of the one or more engineered gas vesicles; and
iv) following the detecting, selecting engineered gas vesicles having a detected protease induced collapse pressure lower than the initial collapse pressure and/or a protease induced ultrasound response having a nonlinearity enhanced with respect to the baseline nonlinearity, the selecting performed to provide the protease sensitive gas vesicles.

2. The method of claim 1, wherein the at least one protease recognition site further comprises a linker polypeptide attached at at least one of an N-terminus and the C-terminus.

3. The method of claim 1, wherein the at least one protease recognition site further comprises at least one protease recognition site inserted within the second repeat region in an N-terminus to C-terminus direction, between repeat regions, after the last repeat region before the C-terminal region and/or before the first repeat after the N-terminal region.

4. The method of claim 1, wherein the at least one protease recognition site further comprises a protease recognition site attached at the N-terminus and/or C-terminus of the engineered GvpC protein.

5. The method of claim 1, wherein the at least one protease recognition site is selected from an endoprotease recognition site, an exoprotease recognition site and a processive protease recognition site.

6. The method of claim 1, wherein the protease recognition site comprises at least one of Human Rhinovirus (HRV) 3C Protease recognition site, Enterokinase recognition site, Factor Xa recognition site, Tobacco etch virus protease (TEV protease) recognition site, Thrombin recognition site, Calpain recognition site, MMP2/recognition site, Urokinase recognition site, ClpXP recognition site, mflon recognition site, and ubiquitin recognition site.

7. The method of claim 1, wherein the engineered GvpC is selected from an engineered GvpC of Anabaena flos-aquae, an engineered GvpC of Halobacterium salinarum, an engineered GvpC of Haloferax mediterranei, an engineered GvpC of Microchaete diplosiphon, and an engineered GvpC of Nostoc sp.

8. The method of claim 1, wherein the one or more one or more engineered gas vesicles are provided by engineering a naturally occurring or a hybrid gas vesicle to add the engineered GvpC or replace an existing GvpC with the engineered GvpC.

9. The method of claim 8, wherein the naturally occurring gas vesicle is selected from a naturally occurring gas vesicle of Anabaena flos-aquae, Halobacterium salinarum, Halobacterium mediterranei, Microchaete diplosiphon Nostoc sp and Bacillus Megaterium.

10. The method of claim 8 wherein the hybrid gas vesicle is selected from
a hybrid gas vesicle encoded by a gas vesicle gene cluster comprising -gvpA, and gvpC from Anabaena flos-aquae, and gvpN, gvpF, gvpG, gvpL, gvpS, gvpK, gvpJ, and gvpU from B. megaterium,
a hybrid gas vesicle gene cluster comprising—gvpA, gvpC and gvpN from Anabaena flos-aquae, gvpF, gvpG, gvpL, gvpS, gvpK, gvpJ, and gvpU from B. megaterium.

11. The method of claim 1, wherein the providing is performed by:
engineering a GvpC protein to attach the at least one protease recognition site; and
assembling the engineered GvpC protein with other gas vesicle proteins to provide the engineered gas vesicle.

12. The method of claim 11, wherein the GvpC protein is selected from SEQ ID NO 488 to SEQ ID NO 492.

13. The method of claim 11, wherein the protease recognition site is selected from LEVLFQ/GP (SEQ ID NO 53), DDDDK/(SEQ ID NO 54), IEGR/(SEQ ID NO 55), ENLYFQ/G(SEQ ID NO 56), LVPR/GS (SEQ ID NO 57), QQEVY/GMMPRD (SEQ ID NO: 58), PLG/LAG (SEQ ID NO: 59), PQG/IAAQ (SEQ ID NO: 60), GPLGVRGY(SEQ ID NO: 61), SGR/SAG (SEQ ID NO 62) and LGGSGR/SANAILEGSG (SEQ ID NO 63).

14. The method of claim 11, wherein the protease recognition site further comprises a linker polypeptide at at least one of the N-terminus and C-terminus.

15. The method of claim 14 wherein the linker polypeptide is selected from GSGSGSG(SEQ ID NO: 64), GGGGS (SEQ ID NO: 65), GSGSG (SEQ ID NO: 66), GGGG (SEQ ID NO: 67), GGG(SEQ ID NO: 68), GG(SEQ ID NO 69), GS (SEQ ID NO: 70), GSGS(SEQ ID NO: 71), GGGS(SEQ ID NO: 72), GGS(SEQ ID NO: 73), GTS (SEQ ID NO: 74)₁ GGSGGS (SEQ ID NO: 75), GGG (SEQ ID NO: 76), GGGGGG (SEQ ID NO: 77), GGGGGGGGG (SEQ ID NO: 78), GGGGGGGGGGGG (SEQ ID NO: 79), GGGGGGGGGGGGGGG (SEQ ID NO: 80), GGS(SEQ ID NO: 81), GGSGGS(SEQ ID NO: 82), GGSGGSGGS (SEQ ID NO: 83), GGSGGSGGSGGS (SEQ ID NO: 84), GGSGGSGGSGGSGGS (SEQ ID NO: 85), GSG (SEQ ID NO: 86), GSGGSG (SEQ ID NO: 87), GSGGSGGSG(SEQ ID NO: 88), GSGGSGGSGGSG (SEQ ID NO: 89), GSGGSGGSGGSGGSG (SEQ ID NO: 90), GGGGS(SEQ ID NO: 91), GGGGSGGGGS (SEQ ID NO: 92), GGGGSGGGGSGGGGS (SEQ ID NO: 93), GGGGSGGGGSGGGGSGGGGS (SEQ ID NO: 94), GGGGSGGGGSGGGGSGGGGSGGGGS (SEQ ID NO: 95).

16. The method of claim 1, wherein the detecting is performed by using nonlinear ultrasound imaging revealing a presence of an increase in ultrasound nonlinear imaging response signal for the one or more engineered gas vesicles after exposure to the protease.

17. The method of claim 16, wherein the increase in the ultrasound imaging response signal is a maximal increase in the contrast to noise ratio among the one or more engineered Gas Vesicles between before and after exposure to the protease.

18. The method of claim 16, wherein the increase in ultrasound nonlinear imaging response signal corresponds to an increase in contrast to noise of at least 40%.

19. The method of claim 16, wherein said revealing further comprises determining a maximal increase in nonlinear signal among the one or more engineered gas vesicles between before and after exposure to the protease.

20. The method of claim 16, wherein the nonlinear ultrasound imaging comprises cross-amplitude modulation ultrasound imaging.

21. The method of claim 1, wherein the detecting is performed by measuring the acoustic collapse pressure of the one or more engineered gas vesicles and determining a greatest decrease in collapse pressure among the one or more engineered gas vesicles between before and after exposure to the protease.

22. The method of claim 21, wherein the decrease in collapse pressure corresponds to a decrease in 50% collapse pressure of at least 50%.

23. An engineered protease sensitive gas vesicle provided by the method of claim 1, the engineered protease sensitive gas vesicle comprising
a gas enclosed by a protein shell in which a gas vesicle GvpA/B protein and an engineered protease sensitive GvpC protein are arranged in a configuration in which the engineered protease sensitive GvpC protein binds the gas vesicle GvpA/B protein to form the protein shell, wherein at least one protease recognition site is presented on the protein shell of the engineered protease sensitive gas vesicle,
wherein the engineered protease sensitive gas vesicle has an initial collapse pressure, an initial ultrasound baseline line nonlinearity, a protease induced collapse pressure lower than the initial collapse pressure and a protease induced ultrasound response having an increased nonlinearity compared to the baseline nonlinearity.

24. The engineered protease sensitive gas vesicle of claim 23, wherein the engineered gas vesicle is selected from an engineered Anabaena flos-aquae Gas Vesicle, an engineered Halobacterium salinarum Gas Vesicle, and engineered Halobacterium mediterranei Gas Vesicle, an engineered Microchaete diplosiphon Gas Vesicle, an engineered Nostoc sp Gas Vesicle, an engineered Serratia Gas Vesicle, and an engineered Bacillus Megaterium Gas Vesicle.

25. An engineered protease sensitive gas vesicle protein GvpC comprising multiple repeat regions within a central portion of the GvpC flanked by an N-terminal region having an N-terminus and a C-terminal region having a C-terminus, the engineered gas vesicle protein GvpC further comprising at least one protease recognition site inserted within the central portion and/or attached to at least one of the N-terminus and the C-terminus,
wherein the central portion, the N-terminal region and the C-terminal region are configured to bind a gas vesicle GvpA/B protein of a gas vesicle to form a gas vesicle protein shell of the engineered gas vesicle of claim 23 and to present the at least one protease recognition site on the gas vesicle protein shell upon assembly and
wherein the multiple repeat region, N-terminal region, C-terminal region and protease recognition site are in a configuration associated upon assembly of the engineered protease sensitive GvpC in a gas vesicle having an initial collapse pressure, an initial ultrasound response, a protease induced collapse pressure lower than the initial collapse pressure and a protease induced ultrasound response having a higher nonlinearity than the initial ultrasound response.

26. The engineered protease sensitive gas vesicle protein GvpC of claim 25, wherein the engineered protease sensitive GvpC is selected from Anabaena flos-aquae (TEV sensitive) GvpC having sequence MISLMAKIRQEHQSIAEKVAELSLETREFLSVTTAKRQEQAEKQAQELQAFYKDLQETS QQFLSETAGSGSGSGENLYFQGSGSGSGFHKELQETSQQFLSATAQARIAQAEKQAQ ELLAFYQEVRETSQQFLSATAQARIAQAEKQAQELLAFHKELQETSQQFLSATADART AQAKEQKESLLKFRQDLFVSIFG (SEQ ID NO: 101) Anabaena flos-aquae (calpain sensitive) having sequence MGISLMAKIRQEHQSIAEKVAELSLETREFLSVTTAKRQEQAEKQAQELQAFYKDLQET SQGSGSGQQEVYGMMPRDGSGSGQAQELLAFHKELQETSQQFLSATAQARIAQAEK QAQELLAFYQEVRETSQQFLSATAQARIAQAEKQAQELLAFHKELQETSQQFLSATAD ARTAQAKEQKESLLKFRQDLFVSIFG (SEQ ID NO: 102) Anabaena flos-aquae (ClpXP sensitive) having sequence MGSGISLMAKIRQEHQSIAEKVAELSLETREFLSVTTAKRQEQAEKQAQELQAFYKDLQ ETSQQFLSETAQARIAQAEKQAQELLAFHKELQETSQQFLSATAQARIAQAEKQAQEL LAFYQEVRETSQQFLSATAQARIAQAEKQAQELLAFHKELQETSQQFLSATADARTAQ AKEQKESLLKFRQDLFVSIFGSGAANDENYALAA (SEQ ID NO: 2).

27. A protease sensitive gas vesicle gene cluster (GVGC) encoding for the protease sensitive gas vesicles of claim 23, the protease sensitive gas vesicle gene cluster (GVGC) comprising gas vesicle assembly (GVA) genes and gas vesicle structural (GVS) genes configured to form a gas vesicle type in a host cell, the GVS genes of the protease sensitive GVGC comprising a gas vesicle GvpA/B protein, a genetically engineered protease sensitive gvpC gene encoding for a protease sensitive GvpC protein, configured to bind the gas vesicle GvpA/B protein and to present the at least one protease recognition site on the gas vesicle type upon assembly.

28. A method to detect a protease and/or image a protease-associated biochemical event in a host cell comprised in an imaging target site, the method comprising:
expressing a protease sensitive gas vesicle of claim 23 in the host cell; and
imaging the target site comprising the host cell by applying an ultrasound to obtain a nonlinear ultrasound image of the target site to image the protease-associated biochemical event.

29. A system to detect a protease and/or image a protease-associated biochemical event in a host cell, the system comprising
a protease sensitive gvpC gene expression cassette encoding for the protease sensitive GvpC of claim 25,
a genetically engineered protease sensitive gas vesicle expression system (GVES) comprising the protease sensitive GvpC expression cassette, and/or a host cell,
in a combination with a device configured to apply ultrasound for simultaneous, combined or sequential use in an imaging method to detect a protease and/or image a protease associated biochemical event in a host cell.

30. A method to detect a protease and/or image a protease associated event in a target site, the method comprising:
introducing into the target site, the protease sensitive gas vesicle of claims 23 and/or an engineered protease sensitive host cell configured for expression of the protease sensitive gas vesicle of claim 23, the introducing performed under conditions resulting in presence of protease sensitive gas vesicles in a target site of the host organism; and
imaging the target site comprising the protease sensitive gas vesicle and/or the engineered protease sensitive host cell by applying ultrasound to obtain a nonlinear ultrasound image of the target site.

31. The method of claim 30, wherein the target site is a tissue or an organ within a host organism.

32. A system to detect a protease and/or image a protease associated event in a target site, the system comprising
the engineered protease sensitive gas vesicle of claim 23, and/or
an engineered protease sensitive cell configured to express the engineered protease sensitive gas vesicle,
in a combination with a device configured to apply ultrasound for simultaneous, combined or sequential use in a method to detect a protease and/or image a protease associated event in a target site.

33. A method to detect a protease and/or image a protease-associated biochemical event in a host cell comprised in an imaging target site, the method comprising:
expressing the protease sensitive gas vesicle of claim 23 in the host cell; and
imaging the target site comprising the host cell by applying ultrasound to obtain a nonlinear ultrasound image of the target site to image the protease-associated biochemical event.

34. A system to detect a protease and/or image a protease-associated biochemical event in a host cell comprised in an imaging target site, the system comprising
a protease sensitive gvpC gene expression cassette comprising a gene encoding for the protease sensitive GvpC of claim 25,
a genetically engineered protease sensitive gas vesicle expression system (GVES) comprising the protease sensitive GvpC expression cassette and/or
a host cell,
in a combination with a device configured to apply ultrasound for simultaneous, combined or sequential use in a method to detect a protease and/or image a protease-associated biochemical event in a host cell comprised in an imaging target site.

35. A method to detect a protease and/or image a protease associated event in a target site, the method comprising:
introducing into the target site the protease sensitive gas vesicle of claim 23 and/or an engineered protease-sensitive host cell configured for expression of the protease sensitive gas vesicle, the introducing performed under conditions resulting in presence of protease-sensitive gas vesicles in a target site of the host organism; and
imaging the target site comprising the protease-sensitive gas vesicle by applying ultrasound to obtain a nonlinear ultrasound image of the target site.

36. A system to detect a protease and/or image a protease associated event in a target site, the system comprising
the engineered protease sensitive Gas Vesicle of claim 25, and/or
a cell configured to comprise or express the engineered protease sensitive gas vesicle,
in combination with a device configured to apply ultrasound for simultaneous combined or sequential use in a method to detect a protease and/or image a protease associated event in a target site.

37. A method for producing and screening protease sensitive gas vesicles (GVs), the method comprising:
designing a plurality of protease sensitive GvpC;
cloning the plurality of protease sensitive GvpC;
producing GVs with the plurality of protease sensitive GvpC, creating GV and GvpC combinations;
measuring the mechanical stiffness of the GVs over a range of pressures for each of the plurality of GvpCs;
and determining which GV and GvpC combination provides a largest shift in collapse pressure based on the measuring.

38. The method of claim 34, further comprising identifying which GV and GvpC combination has a maximum nonlinear contrast to noise ratio under nonlinear ultrasound imaging.

39. A method for producing and screening protease sensitive GVs comprising:
designing a plurality of protease sensitive GvpC;
cloning the plurality of protease sensitive GvpC;
producing GVs with the plurality of protease sensitive GvpC, creating GV and GvpC combinations;
measuring the nonlinear ultrasound response over a range of pressures for each of the plurality of GvpCs;
and determining which GV and GvpC combination provides the maximum nonlinear ultrasound imaging contrast to noise ratio before and after exposure to the protease.
